SEQUENCE LISTING (38 sequences)

SEQ ID NO: 1 - protein, 2431 aa, Homo sapiens
MTTLLWVFVTLRVITAAVTVETSDHDNSLSVSIPQPSPLRVLLGTSLTIPCYFIDPMHPVTTAPSTAPLAPRIKWSRVSKEKEVVLLVATEGRVRVNSAYQDKVSLPNYPAIPSDATLEVQSLRSNDSGVYRCEVMHGIEDSEATLEVVVKGIVFHYRAISTRYTLDFDRAQRACLQNSAIIATPEQLQAAYEDGFHQCDAGWLADQTVRYPIHTPREGCYGDKDEFPGVRTYGIRDTNETYDVYCFAEEMEGEVFYATSPEKFTFQEAANECRRLGARLATTGQLYLAWQAGMDMCSAGWLADRSVRYPISKARPNCGGNLLGVRTVYVHANQTGYPDPSSRYDAICYTGEDFVDIPENFFGVGGEEDITVQTVTWPDMELPLPRNITEGEARGSVILTVKPIFEVSPSPLEPEEPFTFAPEIGATAFAEVENETGEATRPWGFPTPGLGPATAFTSEDLVVQVTAVPGQPHLPGGVVFHYRPGPTRYSLTFEEAQQACLRTGAVIASPEQLQAAYEAGYEQCDAGWLRDQTVRYPIVSPRTPCVGDKDSSPGVRTYGVRPSTETYDVYCFVDRLEGEVFFATRLEQFTFQEALEFCESHNATLATTGQLYAAWSRGLDKCYAGWLADGSLRYPIVTPRPACGGDKPGVRTVYLYPNQTGLPDPLSRHHAFCFRGISAVPSPGEEEGGTPTSPSGVEEWIVTQVVPGVAAVPVEEETTAVPSGETTAILEFTTEPENQTEWEPAYTPVGTSPLPGILPTWPPTGAATEESTEGPSATEVPSASEEPSPSEVPFPSEEPSPSEEPFPSVRPFPSVELFPSEEPFPSKEPSPSEEPSASEEPYTPSPPVPSWTELPSSGEESGAPDVSGDFTGSGDVSGHLDFSGQLSGDRASGLPSGDLDSSGLTSTVGSGLPVESGLPSGDEERIEWPSTPTVGELPSGAEILEGSASGVGDLSGLPSGEVLETSASGVGDLSGLPSGEVLETTAPGVEDISGLPSGEVLETTAPGVEDISGLPSGEVLETTAPGVEDISGLPSGEVLETTAPGVEDISGLPSGEVLETTAPGVEDISGLPSGEVLETTAPGVEDISGLPSGEVLETAAPGVEDISGLPSGEVLETAAPGVEDISGLPSGEVLETAAPGVEDISGLPSGEVLETAAPGVEDISGLPSGEVLETAAPGVEDISGLPSGEVLETAAPGVEDISGLPSGEVLETAAPGVEDISGLPSGEVLETAAPGVEDISGLPSGEVLETAAPGVEDISGLPSGEVLETAAPGVEDISGLPSGEVLETAAPGVEDISGLPSGEVLETAAPGVEDISGLPSGEVLETAAPGVEDISGLPSGEVLETAAPGVEDISGLPSGEVLETAAPGVEDISGLPSGEVLETAAPGVEDISGLPSGEVLETTAPGVEEISGLPSGEVLETTAPGVDEISGLPSGEVLETTAPGVEEISGLPSGEVLETSTSAVGDLSGLPSGGEVLEISVSGVEDISGLPSGEVVETSASGIEDVSELPSGEGLETSASGVEDLSRLPSGEEVLEISASGFGDLSGLPSGGEGLETSASEVGTDLSGLPSGREGLETSASGAEDLSGLPSGKEDLVGSASGDLDLGKLPSGTLGSGQAPETSGLPSGFSGEYSGVDLGSGPPSGLPDFSGLPSGFPTVSLVDSTLVEVVTASTASELEGRGTIGISGAGEISGLPSSELDISGRASGLPSGTELSGQASGSPDVSGEIPGLFGVSGQPSGFPDTSGETSGVTELSGLSSGQPGISGEASGVLYGTSQPFGITDLSGETSGVPDLSGQPSGLPGFSGATSGVPDLVSGTTSGSGESSGITFVDTSLVEVAPTTFKEEEGLGSVELSGLPSGEADLSGKSGMVDVSGQFSGTVDSSGFTSQTPEFSGLPSGIAEVSGESSRAEIGSSLPSGAYYGSGTPSSFPTVSLVDRTLVESVTQAPTAQEAGEGPSGILELSGAHSGAPDMSGEHSGFLDLSGLQSGLIEPSGEPPGTPYFSGDFASTTNVSGESSVAMGTSGEASGLPEVTLITSEFVEGVTEPTISQELGQRPPVTHTPQLFESSGKVSTAGDISGATPVLPGSGVEVSSVPESSSETSAYPEAGFGASAAPEASREDSGSPDLSETTSAFHEANLERSSGLGVSGSTLTFQEGEASAAPEVSGESTTTSDVGTEAPGLPSATPTASGDRTEISGDLSGHTSQLGVVISTSIPESEWTQQTQRPAETHLEIESSSLLYSGEETHTVETATSPTDASIPASPEWKRESESTAADQEVCEEGWNKYQGHCYRHFPDRETWVDAERRCREQQSHLSSIVTPEEQEFVNNNAQDYQWIGLNDRTIEGDFRWSDGHPMQFENWRPNQPDNFFAAGEDCVVMIWHEKGEWNDVPCNYHLPFTCKKGTATTYKRRLQKRSSRHPRRSRPSTAH

SEQ ID NO: 2 - protein, 2045 aa, Homo sapiens
MAGRSHPGPLRPLLPLLVVAACVLPGAGGTCPERALERREEEANVVLTGTVEEILNVDPVQHTYSCKVRVWRYLKGKDLVARESLLDGGNKVVISGFGDPLICDNQVSTGDTRIFFVNPAPPYLWPAHKNELMLNSSLMRITLRNLEEVEFCVEDKPGTHFTPVPPTPPDACRGMLCGFGAVCEPNAEGPGRASCVCKKSPCPSVVAPVCGSDASTYSNECELQRAQCSQQRRIRLLSRGPCGSRDPCSNVTCSFGSTCARSADGLTASCLCPATCRGAPEGTVCGSDGADYPGECQLLRRACARQENVFKKFDGPCDPCQGALPDPSRSCRVNPRTRRPEMLLRPESCPARQAPVCGDDGVTYENDCVMGRSGAARGLLLQKVRSGQCQGRDQCPEPCRFNAVCLSRRGRPRCSCDRVTCDGAYRPVCAQDGRTYDSDCWRQQAECRQQRAIPSKHQGPCDQAPSPCLGVQCAFGATCAVKNGQAACECLQACSSLYDPVCGSDGVTYGSACELEATACTLGREIQVARKGPCDRCGQCRFGALCEAETGRCVCPSECVALAQPVCGSDGHTYPSECMLHVHACTHQISLHVASAGPCETCGDAVCAFGAVCSAGQCVCPRCEHPPPGPVCGSDGVTYGSACELREAACLQQTQIEEARAGPCEQAECGSGGSGSGEDGDCEQELCRQRGGIWDEDSEDGPCVCDFSCQSVPGSPVCGSDGVTYSTECELKKARCESQRGLYVAAQGACRGPTFAPLPPVAPLHCAQTPYGCCQDNITAARGVGLAGCPSACQCNPHGSYGGTCDPATGQCSCRPGVGGLRCDRCEPGFWNFRGIVTDGRSGCTPCSCDPQGAVRDDCEQMTGLCSCKPGVAGPKCGQCPDGRALGPAGCEADASAPATCAEMRCEFGARCVEESGSAHCVCPMLTCPEANATKVCGSDGVTYGNECQLKTIACRQGLQISIQSLGPCQEAVAPSTHPTSASVTVTTPGLLLSQALPAPPGALPLAPSSTAHSQTTPPPSSRPRTTASVPRTTVWPVLTVPPTAPSPAPSLVASAFGESGSTDGSSDEELSGDQEASGGGSGGLEPLEGSSVATPGPPVERASCYNSALGCCSDGKTPSLDAEGSNCPATKVFQGVLELEGVEGQELFYTPEMADPKSELFGETARSIESTLDDLFRNSDVKKDFRSVRLRDLGPGKSVRAIVDVHFDPTTAFRAPDVARALLRQIQVSRRRSLGVRRPLQEHVRFMDFDWFPAFITGATSGAIAAGATARATTASRLPSSAVTPRAPHPSHTSQPVAKTTAAPTTRRPPTTAPSRVPGRRPPAPQQPPKPCDSQPCFHGGTCQDWALGGGFTCSCPAGRGGAVCEKVLGAPVPAFEGRSFLAFPTLRAYHTLRLALEFRALEPQGLLLYNGNARGKDFLALALLDGRVQLRFDTGSGPAVLTSAVPVEPGQWHRLELSRHWRRGTLSVDGETPVLGESPSGTDGLNLDTDLFVGGVPEDQAAVALERTFVGAGLRGCIRLLDVNNQRLELGIGPGAATRGSGVGECGDHPCLPNPCHGGAPCQNLEAGRFHCQCPPGRVGPTCADEKSPCQPNPCHGAAPCRVLPEGGAQCECPLGREGTFCQTASGQDGSGPFLADFNGFSHLELRGLHTFARDLGEKMALEVVFLARGPSGLLLYNGQKTDGKGDFVSLALRDRRLEFRYDLGKGAAVIRSREPVTLGAWTRVSLERNGRKGALRVGDGPRVLGESPVPHTVLNLKEPLYVGGAPDFSKLARAAAVSSGFDGAIQLVSLGGRQLLTPEHVLRQVDVTSFAGHPCTRASGHPCLNGASCVPREAAYVCLCPGGFSGPHCEKGLVEKSAGDVDTLAFDGRTFVEYLNAVTESEKALQSNHFELSLRTEATQGLVLWSGKATERADYVALAIVDGHLQLSYNLGSQPVVLRSTVPVNTNRWLRVVAHREQREGSLQVGNEAPVTGSSPLGATQLDTDGALWLGGLPELPVGPALPKAYGTGFVGCLRDVVVGRHPLHLLEDAVTKPELRPCPTP

SEQ ID NO: 3 - protein, 394 aa, Homo sapiens [sequence omitted]

SEQ ID NO: 4 - protein, 911 aa, Homo sapiens
MAQLFLPLLAALVLAQAPAALADVLEGDSSEDRAFRVRIAGDAPLQGVLGGALTIPCHVHYLRPPPSRRAVLGSPRVKWTFLSRGREAEVLVARGVRVKVNEAYRFRVALPAYPASLTDVSLALSELRPNDSGIYRCEVQHGIDDSSDAVEVKVKGVVFLYREGSARYAFSFSGAQEACARIGAHIATPEQLYAAYLGGYEQCDAGWLSDQTVRYPIQTPREACYGDMDGFPGVRNYGVVDPDDLYDVYCYAEDLNGELFLGDPPEKLTLEEARAYCQERGAEIATTGQLYAAWDGGLDHCSPGWLADGSVRYPIVTPSQRCGGGLPGVKTLFLFPNQTGFPNKHSRFNVYCFRDSAQPSAIPEASNPASNPASDGLEAIVTVTETLEELQLPQEATESESRGAIYSIPIMEDGGGGSSTPEDPAEAPRTLLEFETQSMVPPTGFSEEEGKALEEEEKYEDEEEKEEEEEEEEVEDEALWAWPSELSSPGPEASLPTEPAAQEKSLSQAPARAVLQPGASPLPDGESEASRPPRVHGPPTETLPTPRERNLASPSPSTLVEAREVGEATGGPELSGVPRGESEETGSSEGAPSLLPATRAPEGTRELEAPSEDNSGRTAPAGTSVQAQPVLPTDSASRGGVAVVPASGDCVPSPCHNGGTCLEEEEGVRCLCLPGYGGDLCDVGLRFCNPGWDAFQGACYKHFSTRRSWEEAETQCRMYGAHLASISTPEEQDFINNRYREYQWIGLNDRTIEGDFLWSDGVPLLYENWNPGQPDSYFLSGENCVVMVWHDQGQWSDVPCNYHLSYTCKMGLVSCGPPPELPLAQVFGRPRLRYEVDTVLRYRCREGLAQRNLPLIRCQENGRWEAPQISCVPRRPARALHPEEDPEGRQGRLLGRWKALLIPPSSPMPGP

SEQ ID NO: 5 - protein, 1464 aa, Homo sapiens
MFSFVDLRLLLLLAATALLTHGQEEGQVEGQDEDIPPITCVQNGLRYHDRDVWKPEPCRICVCDNGKVLCDDVICDETKNCPGAEVPEGECCPVCPDGSESPTDQETTGVEGPKGDTGPRGPRGPAGPPGRDGIPGQPGLPGPPGPPGPPGPPGLGGNFAPQLSYGYDEKSTGGISVPGPMGPSGPRGLPGPPGAPGPQGFQGPPGEPGEPGASGPMGPRGPPGPPGKNGDDGEAGKPGRPGERGPPGPQGARGLPGTAGLPGMKGHRGFSGLDGAKGDAGPAGPKGEPGSPGENGAPGQMGPRGLPGERGRPGAPGPAGARGNDGATGAAGPPGPTGPAGPPGFPGAVGAKGEAGPQGPRGSEGPQGVRGEPGPPGPAGAAGPAGNPGADGQPGAKGANGAPGIAGAPGFPGARGPSGPQGPGGPPGPKGNSGEPGAPGSKGDTGAKGEPGPVGVQGPPGPAGEEGKRGARGEPGPTGLPGPPGERGGPGSRGFPGADGVAGPKGPAGERGSPGPAGPKGSPGEAGRPGEAGLPGAKGLTGSPGSPGPDGKTGPPGPAGQDGRPGPPGPPGARGQAGVMGFPGPKGAAGEPGKAGERGVPGPPGAVGPAGKDGEAGAQGPPGPAGPAGERGEQGPAGSPGFQGLPGPAGPPGEAGKPGEQGVPGDLGAPGPSGARGERGFPGERGVQGPPGPAGPRGANGAPGNDGAKGDAGAPGAPGSQGAPGLQGMPGERGAAGLPGPKGDRGDAGPKGADGSPGKDGVRGLTGPIGPPGPAGAPGDKGESGPSGPAGPTGARGAPGDRGEPGPPGPAGFAGPPGADGQPGAKGEPGDAGAKGDAGPPGPAGPAGPPGPIGNVGAPGAKGARGSAGPPGATGFPGAAGRVGPPGPSGNAGPPGPPGPAGKEGGKGPRGETGPAGRPGEVGPPGPPGPAGEKGSPGADGPAGAPGTPGPQGIAGQRGVVGLPGQRGERGFPGLPGPSGEPGKQGPSGASGERGPPGPMGPPGLAGPPGESGREGAPGAEGSPGRDGSPGAKGDRGETGPAGPPGAPGAPGAPGPVGPAGKSGDRGETGPAGPAGPVGPVGARGPAGPQGPRGDKGETGEQGDRGIKGHRGFSGLQGPPGPPGSPGEQGPSGASGPAGPRGPPGSAGAPGKDGLNGLPGPIGPPGPRGRTGDAGPVGPPGPPGPPGPPGPPSAGFDFSFLPQPPQEKAHDGGRYYRADDANVVRDRDLEVDTTLKSLSQQIENIRSPEGSRKNPARTCRDLKMCHSDWKSGEYWIDPNQGCNLDAIKVFCNMETGETCVYPTQPSVAQKNWYISKNPKDKRHVWFGESMTDGFQFEYGGQGSDPADVAIQLTFLRLMSTEASQNITYHCKNSVAYMDQQTGNLKKALLLQGSNEIEIRAEGNSRFTYSVTVDGCTSHTGAWGKTVIEYKTTKTSRLPIIDVAPLDVGAPDQEFGFDVGPVCFL

SEQ ID NO: 6 - protein, 1160 aa, Homo sapiens
MIRLGAPQSLVLLTLLVAAVLRCQGQDVRQPGPKGQKGEPGDIKDIVGPKGPPGPQGPAGEQGPRGDRGDKGEKGAPGPRGRDGEPGTPGNPGPPGPPGPPGPPGLGGNFAAQMAGGFDEKAGGAQLGVMQGPMGPMGPRGPPGPAGAPGPQGFQGNPGEPGEPGVSGPMGPRGPPGPPGKPGDDGEAGKPGKAGERGPPGPQGARGFPGTPGLPGVKGHRGYPGLDGAKGEAGAPGVKGESGSPGENGSPGPMGPRGLPGERGRTGPAGAAGARGNDGQPGPAGPPGPVGPAGGPGFPGAPGAKGEAGPTGARGPEGAQGPRGEPGTPGSPGPAGASGNPGTDGIPGAKGSAGAPGIAGAPGFPGPRGPPGPQGATGPLGPKGQTGEPGIAGFKGEQGPKGEPGPAGPQGAPGPAGEEGKRGARGEPGGVGPIGPPGERGAPGNRGFPGQDGLAGPKGAPGERGPSGLAGPKGANGDPGRPGEPGLPGARGLTGRPGDAGPQGKVGPSGAPGEDGRPGPPGPQGARGQPGVMGFPGPKGANGEPGKAGEKGLPGAPGLRGLPGKDGETGAAGPPGPAGPAGERGEQGAPGPSGFQGLPGPPGPPGEGGKPGDQGVPGEAGAPGLVGPRGERGFPGERGSPGAQGLQGPRGLPGTPGTDGPKGASGPAGPPGAQGPPGLQGMPGERGAAGIAGPKGDRGDVGEKGPEGAPGKDGGRGLTGPIGPPGPAGANGEKGEVGPPGPAGSAGARGAPGERGETGPPGPAGFAGPPGADGQPGAKGEQGEAGQKGDAGAPGPQGPSGAPGPQGPTGVTGPKGARGAQGPPGATGFPGAAGRVGPPGSNGNPGPPGPPGPSGKDGPKGARGDSGPPGRAGEPGLQGPAGPPGEKGEPGDDGPSGAEGPPGPQGLAGQRGIVGLPGQRGERGFPGLPGPSGEPGKQGAPGASGDRGPPGPVGPPGLTGPAGEPGRQGSPGADGPPGRDGAAGVKGDRGETGAVGAPGTPGPPGSPGPAGPTGKQGDRGEAGAQGPMGPSGPAGARGIQGPQGPRGDKGEAGEPGERGLKGHRGFTGLQGLPGPPGPSGDQGASGPAGPSGPRGPPGPVGPSGKDGANGIPGPIGPPGPRGRSGETGPAGPPGNPGPPGPPGPPGPGIDMSAFAGLGPR

SEQ ID NO: 7 - protein, 1466 aa, Homo sapiens
MMSFVQKGSWLLLALLHPTIILAQQEAVEGGCSHLGQSYADRDVWKPEPCQICVCDSGSVLCDDIICDDQELDCPNPEIPFGECCAVCPQPPTAPTRPPNGQGPQGPKGDPGPPGIPGRNGDPGIPGQPGSPGSPGPPGICESCPTGPQNYSPQYDSYDVKSGVAVGGLAGYPGPAGPPGPPGPPGTSGHPGSPGSPGYQGPPGEPGQAGPSGPPGPPGAIGPSGPAGKDGESGRPGRPGERGLPGPPGIKGPAGIPGFPGMKGHRGFDGRNGEKGETGAPGLKGENGLPGENGAPGPMGPRGAPGERGRPGLPGAAGARGNDGARGSDGQPGPPGPPGTAGFPGSPGAKGEVGPAGSPGSNGAPGQRGEPGPQGHAGAQGPPGPPGINGSPGGKGEMGPAGIPGAPGLMGARGPPGPAGANGAPGLRGGAGEPGKNGAKGEPGPRGERGEAGIPGVPGAKGEDGKDGSPGEPGANGLPGAAGERGAPGFRGPAGPNGIPGEKGPAGERGAPGPAGPRGAAGEPGRDGVPGGPGMRGMPGSPGGPGSDGKPGPPGSQGESGRPGPPGPSGPRGQPGVMGFPGPKGNDGAPGKNGERGGPGGPGPQGPPGKNGETGPQGPPGPTGPGGDKGDTGPPGPQGLQGLPGTGGPPGENGKPGEPGPKGDAGAPGAPGGKGDAGAPGERGPPGLAGAPGLRGGAGPPGPEGGKGAAGPPGPPGAAGTPGLQGMPGERGGLGSPGPKGDKGEPGGPGADGVPGKDGPRGPTGPIGPPGPAGQPGDKGEGGAPGLPGIAGPRGSPGERGETGPPGPAGFPGAPGQNGEPGGKGERGAPGEKGEGGPPGVAGPPGGSGPAGPPGPQGVKGERGSPGGPGAAGFPGARGLPGPPGSNGNPGPPGPSGSPGKDGPPGPAGNTGAPGSPGVSGPKGDAGQPGEKGSPGAQGPPGAPGPLGIAGITGARGLAGPPGMPGPRGSPGPQGVKGESGKPGANGLSGERGPPGPQGLPGLAGTAGEPGRDGNPGSDGLPGRDGSPGGKGDRGENGSPGAPGAPGHPGPPGPVGPAGKSGDRGESGPAGPAGAPGPAGSRGAPGPQGPRGDKGETGERGAAGIKGHRGFPGNPGAPGSPGPAGQQGAIGSPGPAGPRGPVGPSGPPGKDGTSGHPGPIGPPGPRGNRGERGSEGSPGHPGQPGPPGPPGAPGPCCGGVGAAAIAGIGGEKAGGFAPYYGDEPMDFKINTDEIMTSLKSVNGQIESLISPDGSRKNPARNCRDLKFCHPELKSGEYWVDPNQGCKLDAIKVFCNMETGETCISANPLNVPRKHWWTDSSAEKKHVWFGESMDGGFQFSYGNPELPEDVLDVQLAFLRLLSSRASQNITYHCKNSIAYMDQASGNVKKALKLMGSNEGEFKAEGNSKFTYTVLEDGCTKHTGEWSKTVFEYRTRKAVRLPIVDIAPYDIGGPDQEFGVDVGPVCFL

SEQ ID NO: 8 - protein, 1669 aa, Homo sapiens
MGPRLSVWLLLLPAALLLHEEHSRAAAKGGCAGSGCGKCDCHGVKGQKGERGLPGLQGVIGFPGMQGPEGPQGPPGQKGDTGEPGLPGTKGTRGPPGASGYPGNPGLPGIPGQDGPPGPPGIPGCNGTKGERGPLGPPGLPGFAGNPGPPGLPGMKGDPGEILGHVPGMLLKGERGFPGIPGTPGPPGLPGLQGPVGPPGFTGPPGPPGPPGPPGEKGQMGLSFQGPKGDKGDQGVSGPPGVPGQAQVQEKGDFATKGEKGQKGEPGFQGMPGVGEKGEPGKPGPRGKPGKDGDKGEKGSPGFPGEPGYPGLIGRQGPQGEKGEAGPPGPPGIVIGTGPLGEKGERGYPGTPGPRGEPGPKGFPGLPGQPGPPGLPVPGQAGAPGFPGERGEKGDRGFPGTSLPGPSGRDGLPGPPGSPGPPGQPGYTNGIVECQPGPPGDQGPPGIPGQPGFIGEIGEKGQKGESCLICDIDGYRGPPGPQGPPGEIGFPGQPGAKGDRGLPGRDGVAGVPGPQGTPGLIGQPGAKGEPGEFYFDLRLKGDKGDPGFPGQPGMTGRAGSPGRDGHPGLPGPKGSPGSVGLKGERGPPGGVGFPGSRGDTGPPGPPGYGPAGPIGDKGQAGFPGGPGSPGLPGPKGEPGKIVPLPGPPGAEGLPGSPGFPGPQGDRGFPGTPGRPGLPGEKGAVGQPGIGFPGPPGPKGVDGLPGDMGPPGTPGRPGFNGLPGNPGVQGQKGEPGVGLPGLKGLPGLPGIPGTPGEKGSIGVPGVPGEHGAIGPPGLQGIRGEPGPPGLPGSVGSPGVPGIGPPGARGPPGGQGPPGLSGPPGIKGEKGFPGFPGLDMPGPKGDKGAQGLPGITGQSGLPGLPGQQGAPGIPGFPGSKGEMGVMGTPGQPGSPGPVGAPGLPGEKGDHGFPGSSGPRGDPGLKGDKGDVGLPGKPGSMDKVDMGSMKGQKGDQGEKGQIGPIGEKGSRGDPGTPGVPGKDGQAGQPGQPGPKGDPGISGTPGAPGLPGPKGSVGGMGLPGTPGEKGVPGIPGPQGSPGLPGDKGAKGEKGQAGPPGIGIPGLRGEKGDQGIAGFPGSPGEKGEKGSIGIPGMPGSPGLKGSPGSVGYPGSPGLPGEKGDKGLPGLDGIPGVKGEAGLPGTPGPTGPAGQKGEPGSDGIPGSAGEKGEPGLPGRGFPGFPGAKGDKGSKGEVGFPGLAGSPGIPGSKGEQGFMGPPGPQGQPGLPGSPGHATEGPKGDRGPQGQPGLPGLPGPMGPPGLPGIDGVKGDKGNPGWPGAPGVPGPKGDPGFQGMPGIGGSPGITGSKGDMGPPGVPGFQGPKGLPGLQGIKGDQGDQGVPGAKGLPGPPGPPGPYDIIKGEPGLPGPEGPPGLKGLQGLPGPKGQQGVTGLVGIPGPPGIPGFDGAPGQKGEMGPAGPTGPRGFPGPPGPDGLPGSMGPPGTPSVDHGFLVTRHSQTIDDPQCPSGTKILYHGYSLLYVQGNERAHGQDLGTAGSCLRKFSTMPFLFCNINNVCNFASRNDYSYWLSTPEPMPMSMAPITGENIRPFISRCAVCEAPAMVMAVHSQTIQIPPCPSGWSSLWIGYSFVMHTSAGAEGSGQALASPGSCLEEFRSAPFIECHGRGTCNYYANAYSFWLATIERSEMFKKPTPSTLKAGELRTHVSRCQVCMRRT

SEQ ID NO: 9 - protein, 1838 aa, Homo sapiens
MDVHTRWKARSALRPGAPLLPPLLLLLLWAPPPSRAAQPADLLKVLDFHNLPDGITKTTGFCATRRSSKGPDVAYRVTKDAQLSAPTKQLYPASAFPEDFSILTTVKAKKGSQAFLVSIYNEQGIQQIGLELGRSPVFLYEDHTGKPGPEDYPLFRGINLSDGKWHRIALSVHKKNVTLILDCKKKTTKFLDRSDHPMIDINGIIVFGTRILDEEVFEGDIQQLLFVSDHRAAYDYCEHYSPDCDTAVPDTPQSQDPNPDEYYTEGDGEGETYYYEYPYYEDPEDLGKEPTPSKKPVEAAKETTEVPEELTPTPTEAAPMPETSEGAGKEEDVGIGDYDYVPSEDYYTPSPYDDLTYGEGEENPDQPTDPGAGAEIPTSTADTSNSSNPAPPPGEGADDLEGEFTEETIRNLDENYYDPYYDPTSSPSEIGPGMPANQDTIYEGIGGPRGEKGQKGEPAIIEPGMLIEGPPGPEGPAGLPGPPGTMGPTGQVGDPGERGPPGRPGLPGADGLPGPPGTMLMLPFRFGGGGDAGSKGPMVSAQESQAQAILQQARLALRGPAGPMGLTGRPGPVGPPGSGGLKGEPGDVGPQGPRGVQGPPGPAGKPGRRGRAGSDGARGMPGQTGPKGDRGFDGLAGLPGEKGHRGDPGPSGPPGPPGDDGERGDDGEVGPRGLPGEPGPRGLLGPKGPPGPPGPPGVTGMDGQPGPKGNVGPQGEPGPPGQQGNPGAQGLPGPQGAIGPPGEKGPLGKPGLPGMPGADGPPGHPGKEGPPGEKGGQGPPGPQGPIGYPGPRGVKGADGIRGLKGTKGEKGEDGFPGFKGDMGIKGDRGEIGPPGPRGEDGPEGPKGRGGPNGDPGPLGPPGEKGKLGVPGLPGYPGRQGPKGSIGFPGFPGANGEKGGRGTPGKPGPRGQRGPTGPRGERGPRGITGKPGPKGNSGGDGPAGPPGERGPNGPQGPTGFPGPKGPPGPPGKDGLPGHPGQRGETGFQGKTGPPGPPGVVGPQGPTGETGPMGERGHPGPPGPPGEQGLPGLAGKEGTKGDPGPAGLPGKDGPPGLRGFPGDRGLPGPVGALGLKGNEGPPGPPGPAGSPGERGPAGAAGPIGIPGRPGPQGPPGPAGEKGAPGEKGPQGPAGRDGLQGPVGLPGPAGPVGPPGEDGDKGEIGEPGQKGSKGDKGEQGPPGPTGPQGPIGQPGPSGADGEPGPRGQQGLFGQKGDEGPRGFPGPPGPVGLQGLPGPPGEKGETGDVGQMGPPGPPGPRGPSGAPGADGPQGPPGGIGNPGAVGEKGEPGEAGEPGLPGEGGPPGPKGERGEKGESGPSGAAGPPGPKGPPGDDGPKGSPGPVGFPGDPGPPGEPGPAGQDGPPGDKGDDGEPGQTGSPGPTGEPGPSGPPGKRGPPGPAGPEGRQGEKGAKGEAGLEGPPGKTGPIGPQGAPGKPGPDGLRGIPGPVGEQGLPGSPGPDGPPGPMGPPGLPGLKGDSGPKGEKGHPGLIGLIGPPGEQGEKGDRGLPGPQGSSGPKGEQGITGPSGPIGPPGPPGLPGPPGPKGAKGSSGPTGPKGEAGHPGPPGPPGPPGEVIQPLPIQASRTRRNIDASQLLDDGNGENYVDYADGMEEIFGSLNSLKLEIEQMKRPLGTQQNPARTCKDLQLCHPDFPDGEYWVDPNQGCSRDSFKVYCNFTAGGSTCVFPDKKSEGARITSWPKENPGSWFSEFKRGKLLSYVDAEGNPVGVVQMTFLRLLSASAHQNVTYHCYQSVAWQDAATGSYDKALRFLGSNDEEMSYDNNPYIRALVDGCATKKGYQKTVLEIDTPKVEQVPIVDIMFNDFGEASQKFGFEVGPACFMG

SEQ ID NO: 10 - protein, 1028 aa, Homo sapiens
MRAARALLPLLLQACWTAAQDEPETPRAVAFQDCPVDLFFVLDTSESVALRLKPYGALVDKVKSFTKRFIDNLRDRYYRCDRNLVWNAGALHYSDEVEIIQGLTRMPGGRDALKSSVDAVKYFGKGTYTDCAIKKGLEQLLVGGSHLKENKYLIVVTDGHPLEGYKEPCGGLEDAVNEAKHLGVKVFSVAITPDHLEPRLSIIATDHTYRRNFTAADWGQSRDAEEAISQTIDTIVDMIKNNVEQVCCSFECQPARGPPGLRGDPGFEGERGKPGLPGEKGEAGDPGRPGDLGPVGYQGMKGEKGSRGEKGSRGPKGYKGEKGKRGIDGVDGVKGEMGYPGLPGCKGSPGFDGIQGPPGPKGDPGAFGLKGEKGEPGADGEAGRPGSSGPSGDEGQPGEPGPPGEKGEAGDEGNPGPDGAPGERGGPGERGPRGTPGTRGPRGDPGEAGPQGDQGREGPVGVPGDPGEAGPIGPKGYRGDEGPPGSEGARGAPGPAGPPGDPGLMGERGEDGPAGNGTEGFPGFPGYPGNRGAPGINGTKGYPGLKGDEGEAGDPGDDNNDIAPRGVKGAKGYRGPEGPQGPPGHQGPPGPDECEILDIIMKMCSCCECKCGPIDLLFVLDSSESIGLQNFEIAKDFVVKVIDRLSRDELVKFEPGQSYAGVVQYSHSQMQEHVSLRSPSIRNVQELKEAIKSLQWMAGGTFTGEALQYTRDQLLPPSPNNRIALVITDGRSDTQRDTTPLNVLCSPGIQVVSVGIKDVFDFIPGSDQLNVISCQGLAPSQGRPGLSLVKENYAELLEDAFLKNVTAQICIDKKCPDYTCPITFSSPADITILLDGSASVGSHNFDTTKRFAKRLAERFLTAGRTDPAHDVRVAVVQYSGTGQQRPERASLQFLQNYTALASAVDAMDFINDATDVNDALGYVTRFYREASSGAAKKRLLLFSDGNSQGATPAAIEKAVQEAQRAGIEIFVVVVGRQVNEPHIRVLVTGKTAEYDVAYGESHLFRVPSYQALLRGVFHQTVSRKVALG

SEQ ID NO: 11 - protein, 347 aa, Homo sapiens
MKATIILLLLAQVSWAGPFQQRGLFDFMLEDEASGIGPEVPDDRDFEPSLGPVCPFRCQCHLRVVQCSDLGLDKVPKDLPPDTTLLDLQNNKITEIKDGDFKNLKNLHALILVNNKISKVSPGAFTPLVKLERLYLSKNQLKELPEKMPKTLQELRAHENEITKVRKVTFNGLNQMIVIELGTNPLKSSGIENGAFQGMKKLSYIRIADTNITSIPQGLPPSLTELHLDGNKISRVDAASLKGLNNLAKLGLSFNSISAVDNGSLANTPHLRELHLDNNKLTRVVYLHNNNISVVGSSDFCPPGHNTKKASYSGVSLFSNPVQYWEIQPSTFRCVYVRSAIQLGNYK

SEQ ID NO: 12 - protein, 724 aa, Homo sapiens
MAGLTAAAPRPGVLLLLLSILHPSRPGGVPGAIPGGVPGGVFYPGAGLGALGGGALGPGGKPLKPVPGGLAGAGLGAGLGAFPAVTFPGALVPGGVADAAAAYKAAKAGAGLGGVPGVGGLGVSAGAVVPQPGAGVKPGKVPGVGLPGVYPGGVLPGARFPGVGVLPGVPTGAGVKPKAPGVGGAFAGIPGVGPFGGPQPGVPLGYPIKAPKLPGGYGLPYTTGKLPYGYGPGGVAGAAGKAGYPTGTGVGPQAAAAAAAKAAAKFGAGAAGVLPGVGGAGVPGVPGAIPGIGGIAGVGTPAAAAAAAAAAKAAKYGAAAGLVPGGPGFGPGVVGVPGAGVPGVGVPGAGIPVVPGAGIPGAAVPGVVSPEAAAKAAAKAAKYGARPGVGVGGIPTYGVGAGGFPGFGVGVGGIPGVAGVPGVGGVPGVGGVPGVGISPEAQAAAAAKAAKYGVGTPAAAAAKAAAKAAQFGLVPGVGVAPGVGVAPGVGVAPGVGLAPGVGVAPGVGVAPGVGVAPGIGPGGVAAAAKSAAKVAAKAQLRAAAGLGAGIPGLGVGVGVPGLGVGAGVPGLGVGAGVPGFGAVPGALAAAKAAKYGAAVPGVLGGLGALGGVGIPGGVVGAGPAAAAAAAKAAAKAAQFGLVGAAGLGGLGVGGLGVPGVGGLGGIPPAAAAKAAKYGAAGLGGVLGGAGQFPLGGVAARPGFGLSPIFPGGACLGKACGRKRK

SEQ ID NO: 13 - protein, 807 aa, Homo sapiens
MRLSPAPLKLSRTPALLALALPLAAALAFSDETLDKVPKSEGYCSRILRAQGTRREGYTEFSLRVEGDPDFYKPGTSYRVTLSAAPPSYFRGFTLIALRENREGDKEEDHAGTFQIIDEEETQFMSNCPVAVTESTPRRRTRIQVFWIAPPAGTGCVILKASIVQKRIIYFQDEGSLTKKLCEQDSTFDGVTDKPILDCCACGTAKYRLTFYGNWSEKTHPKDYPRRANHWSAIIGGSHSKNYVLWEYGGYASEGVKQVAELGSPVKMEEEIRQQSDEVLTVIKAKAQWPAWQPLNVRAAPSAEFSVDRTRHLMSFLTMMGPSPDWNVGLSAEDLCTKECGWVQKVVQDLIPWDAGTDSGVTYESPNKPTIPQEKIRPLTSLDHPQSPFYDPEGGSITQVARVVIERIARKGEQCNIVPDNVDDIVADLAPEEKDEDDTPETCIYSNWSPWSACSSSTCDKGKRMRQRMLKAQLDLSVPCPDTQDFQPCMGPGCSDEDGSTCTMSEWITWSPCSISCGMGMRSRERYVKQFPEDGSVCTLPTEETEKCTVNEECSPSSCLMTEWGEWDECSATCGMGMKKRHRMIKMNPADGSMCKAETSQAEKCMMPECHTIPCLLSPWSEWSDCSVTCGKGMRTRQRMLKSLAELGDCNEDLEQVEKCMLPECPIDCELTEWSQWSECNKSCGKGHVIRTRMIQMEPQFGGAPCPETVQRKKCRIRKCLRNPSIQKLRWREARESRRSEQLKEESEGEQFPGCRMRPWTAWSECTKLCGGGIQERYMTVKKRFKSSQFTSCKDKKEIRACNVHPC

SEQ ID NO: 14 - protein, 866 aa, Homo sapiens
MFSMRIVCLVLSVVGTAWTADSGEGDFLAEGGGVRGPRVVERHQSACKDSDWPFCSDEDWNYKCPSGCRMKGLIDEVNQDFTNRINKLKNSLFEYQKNNKDSHSLTTNIMEILRGDFSSANNRDNTYNRVSEDLRSRIEVLKRKVIEKVQHIQLLQKNVRAQLVDMKRLEVDIDIKIRSCRGSCSRALAREVDLKDYEDQQKQLEQVIAKDLLPSRDRQHLPLIKMKPVPDLVPGNFKSQLQKVPPEWKALTDMPQMRMELERPGGNEITRGGSTSYGTGSETESPRNPSSAGSWNSGSSGPGSTGNRNPGSSGTGGTATWKPGSSGPGSTGSWNSGSSGTGSTGNQNPGSPRPGSTGTWNPGSSERGSAGHWTSESSVSGSTGQWHSESGSFRPDSPGSGNARPNNPDWGTFEEVSGNVSPGTRREYHTEKLVTSKGDKELRTGKEKVTSGSTTTTRRSCSKTVTKTVIGPDGHKEVTKEVVTSEDGSDCPEAMDLGTLSGIGTLDGFRHRHPDEAAFFDTASTGKTFPGFFSPMLGEFVSETESRGSESGIFTNTKESSSHHPGIAEFPSRGKSSSYSKQFTSSTSYNRGDSTFESKSYKMADEAGSEADHEGTHSTKRGHAKSRPVRDCDDVLQTHPSGTQSGIFNIKLPGSSKIFSVYCDQETSLGGWLLIQQRMDGSLNFNRTWQDYKRGFGSLNDEGEGEFWLGNDYLHLLTQRGSVLRVELEDWAGNEAYAEYHFRVGSEAEGYALQVSSYEGTAGDALIEGSVEEGAEYTSHNNMQFSTFDRDADQWEENCAEVYGGGWWYNNCQAANLNGIYYPGGSYDPRNNSPYEIENGVVWVSFRGADYSLRAVRMKIRPLVTQ

SEQ ID NO: 15 - protein, 2355 aa, Homo sapiens
MLRGPGPGLLLLAVQCLGTAVPSTGASKSKRQAQQMVQPQSPVAVSQSKPGCYDNGKHYQINQQWERTYLGNALVCTCYGGSRGFNCESKPEAEETCFDKYTGNTYRVGDTYERPKDSMIWDCTCIGAGRGRISCTIANRCHEGGQSYKIGDTWRRPHETGGYMLECVCLGNGKGEWTCKPIAEKCFDHAAGTSYVVGETWEKPYQGWMMVDCTCLGEGSGRITCTSRNRCNDQDTRTSYRIGDTWSKKDNRGNLLQCICTGNGRGEWKCERHTSVQTTSSGSGPFTDVRAAVYQPQPHPQPPPYGHCVTDSGVVYSVGMQWLKTQGNKQMLCTCLGNGVSCQETAVTQTYGGNSNGEPCVLPFTYNGRTFYSCTTEGRQDGHLWCSTTSNYEQDQKYSFCTDHTVLVQTRGGNSNGALCHFPFLYNNHNYTDCTSEGRRDNMKWCGTTQNYDADQKFGFCPMAAHEEICTTNEGVMYRIGDQWDKQHDMGHMMRCTCVGNGRGEWTCIAYSQLRDQCIVDDITYNVNDTFHKRHEEGHMLNCTCFGQGRGRWKCDPVDQCQDSETGTFYQIGDSWEKYVHGVRYQCYCYGRGIGEWHCQPLQTYPSSSGPVEVFITETPSQPNSHPIQWNAPQPSHISKYILRWRPKNSVGRWKEATIPGHLNSYTIKGLKPGVVYEGQLISIQQYGHQEVTRFDFTTTSTSTPVTSNTVTGETTPFSPLVATSESVTEITASSFVVSWVSASDTVSGFRVEYELSEEGDEPQYLDLPSTATSVNIPDLLPGRKYIVNVYQISEDGEQSLILSTSQTTAPDAPPDPTVDQVDDTSIVVRWSRPQAPITGYRIVYSPSVEGSSTELNLPETANSVTLSDLQPGVQYNITIYAVEENQESTPVVIQQETTGTPRSDTVPSPRDLQFVEVTDVKVTIMWTPPESAVTGYRVDVIPVNLPGEHGQRLPISRNTFAEVTGLSPGVTYYFKVFAVSHGRESKPLTAQQTTKLDAPTNLQFVNETDSTVLVRWTPPRAQITGYRLTVGLTRRGQPRQYNVGPSVSKYPLRNLQPASEYTVSLVAIKGNQESPKATGVFTTLQPGSSIPPYNTEVTETTIVITWTPAPRIGFKLGVRPSQGGEAPREVTSDSGSIVVSGLTPGVEYVYTIQVLRDGQERDAPIVNKVVTPLSPPTNLHLEANPDTGVLTVSWERSTTPDITGYRITTTPTNGQQGNSLEEVVHADQSSCTFDNLSPGLEYNVSVYTVKDDKESVPISDTIIPAVPPPTDLRFTNIGPDTMRVTWAPPPSIDLTNFLVRYSPVKNEEDVAELSISPSDNAVVLTNLLPGTEYVVSVSSVYEQHESTPLRGRQKTGLDSPTGIDFSDITANSFTVHWIAPRATITGYRIRHHPEHFSGRPREDRVPHSRNSITLTNLTPGTEYVVSIVALNGREESPLLIGQQSTVSDVPRDLEVVAATPTSLLISWDAPAVTVRYYRITYGETGGNSPVQEFTVPGSKSTATISGLKPGVDYTITVYAVTGRGDSPASSKPISINYRTEIDKPSQMQVTDVQDNSISVKWLPSSSPVTGYRVTTTPKNGPGPTKTKTAGPDQTEMTIEGLQPTVEYVVSVYAQNPSGESQPLVQTAVTNIDRPKGLAFTDVDVDSIKIAWESPQGQVSRYRVTYSSPEDGIHELFPAPDGEEDTAELQGLRPGSEYTVSVVALHDDMESQPLIGTQSTAIPAPTDLKFTQVTPTSLSAQWTPPNVQLTGYRVRVTPKEKTGPMKEINLAPDSSSVVVSGLMVATKYEVSVYALKDTLTSRPAQGVVTTLENVSPPRRARVTDATETTITISWRTKTETITGFQVDAVPANGQTPIQRTIKPDVRSYTITGLQPGTDYKIYLYTLNDNARSSPVVIDASTAIDAPSNLRFLATTPNSLLVSWQPPRARITGYIIKYEKPGSPPREVVPRPRPGVTEATITGLEPGTEYTIYVIALKNNQKSEPLIGRKKTDELPQLVTLPHPNLHGPEILDVPSTVQKTPFVTHPGYDTGNGIQLPGTSGQQPSVGQQMIFEEHGFRRTTPPTTATPIRHRPRPYPPNVGQEALSQTTISWAPFQDTSEYIISCHPVGTDEEPLQFRVPGTSTSATLTGLTRGATYNIIVEALKDQQRHKVREEVVTVGNSVNEGLNQPTDDSCFDPYTVSHYAVGDEWERMSESGFKLLCQCLGFGSGHFRCDSSRWCHDNGVNYKIGEKWDRQGENGQMMSCTCLGNGKGEFKCDPHEATCYDDGKTYHVGEQWQKEYLGAICSCTCFGGQRGWRCDNCRRPGGEPSPEGTTGQSYNQYSQRYHQRTNTNVNCPIECFMPLDVQADREDSRE

SEQ ID NO: 16 - protein, 135 aa, Homo sapiens
MACGLVASNLNLKPGECLRVRGEVAPDAKSFVLNLGKDSNNLCLHFNPRFNAHGDANTIVCNSKDGGAWGTEQREAVFPFQPGSVAEVCITFDQANLTVKLPDGYEFKFPNRLNLEAINYMAADGDFKIKCVAFD

SEQ ID NO: 17 - protein, 250 aa, Homo sapiens [sequence omitted]

SEQ ID NO: 18 - protein, 130 aa, Homo sapiens
PLPGGVVPRMLITILGTVKPNANRIALDFQRGNDVAFHFNPRFNENNRRVIVCNTKLDNNWGREERQSVFPFESGKPFKIQVLVEPDHFKVAVNDAHLLQYNHRVKKLNEISKLGISGDIDLTSASYTMI

SEQ ID NO: 19 - protein, 323 aa, Homo sapiens
MAYVPAPGYQPTYNPTLPYYQPIPGGLNVGMSVYIQGVASEHMKRFFVNFVVGQDPGSDVAFHFNPRFDGWDKVVFNTLQGGKWGSEERKRSMPFKKGAAFELVFIVLAEHYKVVVNGNPFYEYGHRLPLQMVTHLQVDGDLQLQSINFIGGQPLRPQGPPMMPPYPGPGHCHQQLNSLPTMEGPPTFNPPVPYFGRLQGGLTARRTIIIKGYVPPTGKSFAINFKVGSSGDIALHINPRMGNGTVVRNSLLNGSWGSEEKKITHNPFGPGQFFDLSIRCGLDRFKVYANGQHLFDFAHRLSAFQRVDTLEIQGDVTLSYVQI

SEQ ID NO: 20 - protein, 317 aa, Homo sapiens
MMLSLNNLQNIIYNPVIPYVGTIPDQLDPGTLIVICGHVPSDADRFQVDLQNGSSVKPRADVAFHFNPRFKRAGCIVCNTLINEKWGREEITYDTPFKREKSFEIVIMVLKDKFQVAVNGKHTLLYGHRIGPEKIDTLGIYGKVNIHSIGFSFSSDLQSTQASSLELTEISRENVPKSGTPQLSLPFAARLNTPMGPGRTVVVKGEVNANAKSFNVDLLAGKSKDIALHLNPRLNIKAFVRNSFLQESWGEEERNITSFPFSPGMYFEMIIYCDVREFKVAVNGVHSLEYKHRFKELSSIDTLEINGDIHLLEVRSW

SEQ ID NO: 21 - protein, 644 aa, Homo sapiens
MSRQFSSRSGYRSGGGFSSGSAGIINYQRRTTSSSTRRSGGGGGRFSSCGGGGGSFGAGGGFGSRSLVNLGGSKSISISVARGGGRGSGFGGGYGGGGFGGGGFGGGGFGGGGIGGGGFGGFGSGGGGFGGGGFGGGGYGGGYGPVCPPGGIQEVTINQSLLQPLNVEIDPEIQKVKSREREQIKSLNNQFASFIDKVRFLEQQNQVLQTKWELLQQVDTSTRTHNLEPYFESFINNLRRRVDQLKSDQSRLDSELKNMQDMVEDYRNKYEDEINKRTNAENEFVTIKKDVDGAYMTKVDLQAKLDNLQQEIDFLTALYQAELSQMQTQISETNVILSMDNNRSLDLDSIIAEVKAQYEDIAQKSKAEAESLYQSKYEELQITAGRHGDSVRNSKIEISELNRVIQRLRSEIDNVKKQISNLQQSISDAEQRGENALKDAKNKLNDLEDALQQAKEDLARLLRDYQELMNTKLALDLEIATYRTLLEGEESRMSGECAPNVSVSVSTSHTTISGGGSRGGGGGGYGSGGSSYGSGGGSYGSGGGGGGGRGSYGSGGSSYGSGGGSYGSGGGGGGHGSYGSGSSSGGYRGGSGGGGGGSSGGRGSGGGSSGGSIGGRGSSSGGVKSSGGSSSVKFVSTTYSGVTR

SEQ ID NO: 22 - protein, 1193 aa, Homo sapiens [sequence omitted]

SEQ ID NO: 23 - protein, 3122 aa, Homo sapiens
MPGAAGVLLLLLLSGGLGGVQAQRPQQQRQSQAHQQRGLFPAVLNLASNALITTNATCGEKGPEMYCKLVEHVPGQPVRNPQCRICNQNSSNPNQRHPITNAIDGKNTWWQSPSIKNGIEYHYVTITLDLQQVFQIAYVIVKAANSPRPGNWILERSLDDVEYKPWQYHAVTDTECLTLYNIYPRTGPPSYAKDDEVICTSFYSKIHPLENGEIHISLINGRPSADDPSPELLEFTSARYIRLRFQRIRTLNADLMMFAHKDPREIDPIVTRRYYYSVKDISVGGMCICYGHARACPLDPATNKSRCECEHNTCGDSCDQCCPGFHQKPWRAGTFLTKTECEACNCHGKAEECYYDENVARRNLSLNIRGKYIGGGVCINCTQNTAGINCETCTDGFFRPKGVSPNYPRPCQPCHCDPIGSLNEVCVKDEKHARRGLAPGSCHCKTGFGGVSCDRCARGYTGYPDCKACNCSGLGSKNEDPCFGPCICKENVEGGDCSRCKSGFFNLQEDNWKGCDECFCSGVSNRCQSSYWTYGKIQDMSGWYLTDLPGRIRVAPQQDDLDSPQQISISNAEARQALPHSYYWSAPAPYLGNKLPAVGGQLTFTISYDLEEEEEDTERVLQLMIILEGNDLSISTAQDEVYLHPSEEHTNVLLLKEESFTIHGTHFPVRRKEFMTVLANLKRVLLQITYSFGMDAIFRLSSVNLESAVSYPTDGSIAAAVEVCQCPPGYTGSSCESCWPRHRRVNGTIFGGICEPCQCFGHAESCDDVTGECLNCKDHTGGPYCDKCLPGFYGEPTKGTSEDCQPCACPLNIPSNNFSPTCHLDRSLGLICDGCPVGYTGPRCERCAEGYFGQPSVPGGSCQPCQCNDNLDFSIPGSCDSLSGSCLICKPGTTGRYCELCADGYFGDAVDAKNCQPCRCNAGGSFSEVCHSQTGQCECRANVQGQRCDKCKAGTFGLQSARGCVPCNCNSFGSKSFDCEESGQCWCQPGVTGKKCDRCAHGYFNFQEGGCTACECSHLGNNCDPKTGRCICPPNTIGEKCSKCAPNTWGHSITTGCKACNCSTVGSLDFQCNVNTGQCNCHPKFSGAKCTECSRGHWNYPRCNLCDCFLPGTDATTCDSETKKCSCSDQTGQCTCKVNVEGIHCDRCRPGKFGLDAKNPLGCSSCYCFGTTTQCSEAKGLIRTWVTLKAEQTILPLVDEALQHTTTKGIVFQHPEIVAHMDLMREDLHLEPFYWKLPEQFEGKKLMAYGGKLKYAIYFEAREETGFSTYNPQVIIRGGTPTHARIIVRHMAAPLIGQLTRHEIEMTEKEWKYYGDDPRVHRTVTREDFLDILYDIHYILIKATYGNFMRQSRISEISMEVAEQGRGTTMTPPADLIEKCDCPLGYSGLSCEACLPGFYRLRSQPGGRTPGPTLGTCVPCQCNGHSSLCDPETSICQNCQHHTAGDFCERCALGYYGIVKGLPNDCQQCACPLISSSNNFSPSCVAEGLDDYRCTACPRGYEGQYCERCAPGYTGSPGNPGGSCQECECDPYGSLPVPCDPVTGFCTCRPGATGRKCDGCKHWHAREGWECVFCGDECTGLLLGDLARLEQMVMSINLTGPLPAPYKMLYGLENMTQELKHLLSPQRAPERLIQLAEGNLNTLVTEMNELLTRATKVTADGEQTGQDAERTNTRAKSLGEFIKELARDAEAVNEKAIKLNETLGTRDEAFERNLEGLQKEIDQMIKELRRKNLETQKEIAEDELVAAEALLKKVKKLFGESRGENEEMEKDLREKLADYKNKVDDAWDLLREATDKIREANRLFAVNQKNMTALEKKKEAVESGKRQIENTLKEGNDILDEANRLADEINSIIDYVEDIQTKLPPMSEELNDKIDDLSQEIKDRKLAEKVSQAESHAAQLNDSSAVLDGILDEAKNISFNATAAFKAYSNIKDYIDEAEKVAKEAKDLAHEATKLATGPRGLLKEDAKGCLQKSFRILNEAKKLANDVKENEDHLNGLKTRIENADARNGDLLRTLNDTLGKLSAIPNDTAAKLQAVKDKARQANDTAKDVLAQITELHQNLDGLKKNYNKLADSVAKTNAVVKDPSKNKIIADADATVKNLEQEADRLIDKLKPIKELEDNLKKNISEIKELINQARKQANSIKVSVSSGGDCIRTYKPEIKKGSYNNIVVNVKTAVADNLLFYLGSAKFIDFLAIEMRKGKVSFLWDVGSGVGRVEYPDLTIDDSYWYRIVASRTGRNGTISVRALDGPKASIVPSTHHSTSPPGYTILDVDANAMLFVGGLTGKLKKADAVRVITFTGCMGETYFDNKPIGLWNFREKEGDCKGCTVSPQVEDSEGTIQFDGEGYALVSRPIRWYPNISTVMFKFRTFSSSALLMYLATRDLRDFMSVELTDGHIKVSYDLGSGMASVVSNQNHNDGKWKSFTLSRIQKQANISIVDIDTNQEENIATSSSGNNFGLDLKADDKIYFGGLPTLRNLSMKARPEVNLKKYSGCLKDIEISRTPYNILSSPDYVGVTKGCSLENVYTVSFPKPGFVELSPVPIDVGTEINLSFSTKNESGIILLGSGGTPAPPRRKRRQTGQAYYVILLNRGRLEVHLSTGARTMRKIVIRPEPNLFHDGREHSVHVERTRGIFTVQVDENRRYMQNLTVEQPIEVKKLFVGGAPPEFQPSPLRNIPPFEGCIWNLVINSVPMDFARPVSFKNADIGRCAHQKLREDEDGAAPAEIVIQPEPVPTPAFPTPTPVLTHGPCAAESEPALLIGSKQFGLSRNSHIAIAFDDTKVKNRLTIELEVRTEAESGLLFYMARINHADFATVQLRNGLPYFSYDLGSGDTHTMIPTKINDGQWHKIKIMRSKQEGILYVDGASNRTISPKKADILDVVGMLYVGGLPINYTTRRIGPVTYSIDGCVRNLHMAEAPADLEQPTSSFHVGTCFANAQRGTYFDGTGFAKAVGGFKVGLDLLVEFEFRTTTTTGVLLGISSQKMDGMGIEMIDEKLMFHVDNGAGRFTAVYDAGVPGHLCDGQWHKVTANKIKHRIELTVDGNQVEAQSPNPASTSADTNDPVFVGGFPDDLKQFGLTTSIPFRGCIRSLKLTKGTGKPLEVNFAKALELRGVQPVSCPAN

SEQ ID NO: 24 - protein, 1255 aa, Homo sapiens
MTPGTQSPFFLLLLLTVLTVVTGSGHASSTPGGEKETSATQRSSVPSSTEKNAVSMTSSVLSSHSPGSGSSTTQGQDVTLAPATEPASGSAATWGQDVTSVPVTRPALGSTTPPAHDVTSAPDNKPAPGSTAPPAHGVTSAPDTRPAPGSTAPPAHGVTSAPDTRPAPGSTAPPAHGVTSAPDTRPAPGSTAPPAHGVTSAPDTRPAPGSTAPPAHGVTSAPDTRPAPGSTAPPAHGVTSAPDTRPAPGSTAPPAHGVTSAPDTRPAPGSTAPPAHGVTSAPDTRPAPGSTAPPAHGVTSAPDTRPAPGSTAPPAHGVTSAPDTRPAPGSTAPPAHGVTSAPDTRPAPGSTAPPAHGVTSAPDTRPAPGSTAPPAHGVTSAPDTRPAPGSTAPPAHGVTSAPDTRPAPGSTAPPAHGVTSAPDTRPAPGSTAPPAHGVTSAPDTRPAPGSTAPPAHGVTSAPDTRPAPGSTAPPAHGVTSAPDTRPAPGSTAPPAHGVTSAPDTRPAPGSTAPPAHGVTSAPDTRPAPGSTAPPAHGVTSAPDTRPAPGSTAPPAHGVTSAPDTRPAPGSTAPPAHGVTSAPDTRPAPGSTAPPAHGVTSAPDTRPAPGSTAPPAHGVTSAPDTRPAPGSTAPPAHGVTSAPDTRPAPGSTAPPAHGVTSAPDTRPAPGSTAPPAHGVTSAPDTRPAPGSTAPPAHGVTSAPDTRPAPGSTAPPAHGVTSAPDTRPAPGSTAPPAHGVTSAPDTRPAPGSTAPPAHGVTSAPDTRPAPGSTAPPAHGVTSAPDTRPAPGSTAPPAHGVTSAPDTRPAPGSTAPPAHGVTSAPDTRPAPGSTAPPAHGVTSAPDTRPAPGSTAPPAHGVTSAPDTRPAPGSTAPPAHGVTSAPDTRPAPGSTAPPAHGVTSAPDTRPAPGSTAPPAHGVTSAPDTRPAPGSTAPPAHGVTSAPDNRPALGSTAPPVHNVTSASGSASGSASTLVHNGTSARATTTPASKSTPFSIPSHHSDTPTTLASHSTKTDASSTHHSSVPPLTSSNHSTSPQLSTGVSFFFLSFHISNLQFNSSLEDPSTDYYQELQRDISEMFLQIYKQGGFLGLSNIKFRPGSVVVQLTLAFREGTINVHDVETQFNQYKTEAASRYNLTISDVSVSDVPFPFSAQSGAGVPGWGIALLVLVCVLVALAIVYLIALAVCQCRRKNYGQLDIFPARDTYHPMSEYPTYHTHGRYVPPSSTDRSPYEKVSAGNGGSSLSYTNPAVAAASANL

SEQ ID NO: 25 - protein, 1247 aa, Homo sapiens
MLASSSRIRAAWTRALLLPLLLAGPVGCLSRQELFPFGPGQGDLELEDGDDFVSPALELSGALRFYDRSDIDAVYVTTNGIIATSEPPAKESHPGLFPPTFGAVAPFLADLDTTDGLGKVYYREDLSPSITQRAAECVHRGFPEISFQPSSAVVVTWESVAPYQGPSRDPDQKGKRNTFQAVLASSDSSSYAIFLYPEDGLQFHTTFSKKENNQVPAVVAFSQGSVGFLWKSNGAYNIFANDRESVENLAKSSNSGQQGVWVFEIGSPATTNGVVPADVILGTEDGAEYDDEDEDYDLATTRLGLEDVGTTPFSYKALRRGGADTYSVPSVLSPRRAATERPLGPPTERTRSFQLAVETFHQQHPQVIDVDEVEETGVVFSYNTDSRQTCANNRHQCSVHAECRDYATGFCCSCVAGYTGNGRQCVAEGSPQRVNGKVKGRIFVGSSQVPIVFENTDLHSYVVMNHGRSYTAISTIPETVGYSLLPLAPVGGIIGWMFAVEQDGFKNGFSITGGEFTRQAEVTFVGHPGNLVIKQRFSGIDEHGHLTIDTELEGRVPQIPFGSSVHIEPYTELYHYSTSVITSSSTREYTVTEPERDGASPSRIYTYQWRQTITFQECVHDDSRPALPSTQQLSVDSVFVLYNQEEKILRYALSNSIGPVREGSPDALQNPCYIGTHGCDTNAACRPGPRTQFTCECSIGFRGDGRTCYDIDECSEQPSVCGSHTICNNHPGTFRCECVEGYQFSDEGTCVAVVDQRPINYCETGLHNCDIPQRAQCIYTGGSSYTCSCLPGFSGDGQACQDVDECQPSRCHPDAFCYNTPGSFTCQCKPGYQGDGFRCVPGEVEKTRCQHEREHILGAAGATDPQRPIPPGLFVPECDAHGHYAPTQCHGSTGYCWCVDRDGREVEGTRTRPGMTPPCLSTVAPPIHQGPAVPTAVIPLPPGTHLLFAQTGKIERLPLEGNTMRKTEAKAFLHVPAKVIIGLAFDCVDKMVYWTDITEPSIGRASLHGGEPTTIIRQDLGSPEGIAVDHLGRNIFWTDSNLDRIEVAKLDGTQRRVLFETDLVNPRGIVTDSVRGNLYWTDWNRDNPKIETSYMDGTNRRILVQDDLGLPNGLTFDAFSSQLCWVDAGTNRAECLNPSQPSRRKALEGLQYPFAVTSYGKNLYFTDWKMNSVVALDLAISKETDAFQPHKQTRLYGITTALSQCPQGHNYCSVNNGGCTHLCLATPGSRTCRCPDNTLGVDCIEQK

SEQ ID NO: 26 - protein, 1375 aa, Homo sapiens
MEGDRVAGRPVLSSLPVLLLLQLLMLRAAALHPDELFPHGESWGDQLLQEGDDESSAVVKLANPLHFYEARFSNLYVGTNGIISTQDFPRETQYVDYDFPTDFPAIAPFLADIDTSHGRGRVLYREDTSPAVLGLAARYVRAGFPRSARFTPTHAFLATWEQVGAYEEVKRGALPSGELNTFQAVLASDGSDSYALFLYPANGLQFLGTRPKESYNVQLQLPARVGFCRGEADDLKSEGPYFSLTSTEQSVKNLYQLSNLGIPGVWAFHIGSTSPLDNVRPAAVGDLSAAHSSVPLGRSFSHATALESDYNEDNLDYYDVNEEEAEYLPGEPEEALNGHSSIDVSFQSKVDTKPLEESSTLDPHTKEGTSLGEVGGPDLKGQVEPWDERETRSPAPPEVDRDSLAPSWETPPPYPENGSIQPYPDGGPVPSEMDVPPAHPEEEIVLRSYPASDHTTPLSRGTYEVGLEDNIGSNTEVFTYNAANKETCEHNHRQCSRHAFCTDYATGFCCHCQSKFYGNGKHCLPEGAPHRVNGKVSGHLHVGHTPVHFTDVDLHAYIVGNDGRAYTAISHIPQPAAQALLPLTPIGGLFGWLFALEKPGSENGFSLAGAAFTHDMEVTFYPGEETVRITQTAEGLDPENYLSIKTNIQGQVPYVPANFTAHISPYKELYHYSDSTVTSTSSRDYSLTFGAINQTWSYRIHQNITYQVCRHAPRHPSFPTTQQLNVDRVFALYNDEERVLRFAVTNQIGPVKEDSDPTPVNPCYDGSHMCDTTARCHPGTGVDYTCECASGYQGDGRNCVDENECATGFHRCGPNSVCINLPGSYRCECRSGYEFADDRHTCILITPPANPCEDGSHTCAPAGQARCVHHGGSTFSCACLPGYAGDGHQCTDVDECSENRCHPAATCYNTPGSFSCRCQPGYYGDGFQCIPDSTSSLTPCEQQQRHAQAQYAYPGARFHIPQCDEQGNFLPLQCHGSTGFCWCVDPDGHEVPGTQTPPGSTPPHCGPSPEPTQRPPTICERWRENLLEHYGGTPRDDQYVPQCDDLGHFIPLQCHGKSDFCWCVDKDGREVQGTRSQPGTTPACIPTVAPPMVRPTPRPDVTPPSVGTFLLYTQGQQIGYLPLNGTRLQKDAAKTLLSLHGSIIVGIDYDCRERMVYWTDVAGRTISRAGLELGAEPETIVNSGLISPEGLAIDHIRRTMYWTDSVLDKIESALLDGSERKVLFYTDLVNPRAIAVDPIRGNLYWTDWNREAPKIETSSLDGENRRILINTDIGLPNGLTFDPFSKLLCWADAGTKKLECTLPDGTGRRVIQNNLKYPFSIVSYADHFYHTDWRRDGVVSVNKHSGQFTDEYLPEQRSHLYGITAVYPYCPTGRK

SEQ ID NO: 27 - protein, 300 aa, Homo sapiens
MRIAVICFCLLGITCAIPVKQADSGSSEEKQLYNKYPDAVATWLNPDPSQKQNLLAPQTLPSKSNESHDHMDDMDDEDDDDHVDSQDSIDSNDSDDVDDTDDSHQSDESHHSDESDELVTDFPTDLPATEVFTPVVPTVDTYDGRGDSVVYGLRSKSKKFRRPDIQYPDATDEDITSHMESEELNGAYKAIPVAQDLNAPSDWDSRGKDSYETSQLDDQSAETHSHKQSRLYKRKANDESNEHSDVIDSQELSKVSREFHSHEFHSHEDMLVVDPKSKEEDKHLKFRISHELDSASSEVN

SEQ ID NO: 28 - protein, 2355 aa, Homo sapiens; identical to SEQ ID NO: 15

SEQ ID NO: 29 - protein, 302 aa, Homo sapiens [sequence omitted]

SEQ ID NO: 30 - protein, 2201 aa, Homo sapiens
MGAMTQLLAGVFLAFLALATEGGVLKKVIRHKRQSGVNATLPEENQPVVFNHVYNIKLPVGSQCSVDLESASGEKDLAPPSEPSESFQEHTVDGENQIVFTHRINIPRRACGCAAAPDVKELLSRLEELENLVSSLREQCTAGAGCCLQPATGRLDTRPFCSGRGNFSTEGCGCVCEPGWKGPNCSEPECPGNCHLRGRCIDGQCICDDGFTGEDCSQLACPSDCNDQGKCVNGVCICFEGYAGADCSREICPVPCSEEHGTCVDGLCVCHDGFAGDDCNKPLCLNNCYNRGRCVENECVCDEGFTGEDCSELICPNDCFDRGRCINGTCYCEEGFTGEDCGKPTCPHACHTQGRCEEGQCVCDEGFAGVDCSEKRCPADCHNRGRCVDGRCECDDGFTGADCGELKCPNGCSGHGRCVNGQCVCDEGYTGEDCSQLRCPNDCHSRGRCVEGKCVCEQGFKGYDCSDMSCPNDCHQHGRCVNGMCVCDDGYTGEDCRDRQCPRDCSNRGLCVDGQCVCEDGFTGPDCAELSCPNDCHGQGRCVNGQCVCHEGFMGKDCKEQRCPSDCHGQGRCVDGQCICHEGFTGLDCGQHSCPSDCNNLGQCVSGRCICNEGYSGEDCSEVSPPKDLVVTEVTEETVNLAWDNEMRVTEYLVVYTPTHEGGLEMQFRVPGDQTSTIIQELEPGVEYFIRVFAILENKKSIPVSARVATYLPAPEGLKFKSIKETSVEVEWDPLDIAFETWEIIFRNMNKEDEGEITKSLRRPETSYRQTGLAPGQEYEISLHIVKNNTRGPGLKRVTTTRLDAPSQIEVKDVTDTTALITWFKPLAEIDGIELTYGIKDVPGDRTTIDLTEDENQYSIGNLKPDTEYEVSLISRRGDMSSNPAKETFTTGLDAPRNLRRVSQTDNSITLEWRNGKAAIDSYRIKYAPISGGDHAEVDVPKSQQATTKTTLTGLRPGTEYGIGVSAVKEDKESNPATINAATELDTPKDLQVSETAETSLTLLWKTPLAKFDRYRLNYSLPTGQWVGVQLPRNTTSYVLRGLEPGQEYNVLLTAEKGRHKSKPARVKASTEQAPELENLTVTEVGWDGLRLNWTAADQAYEHFIIQVQEANKVEAARNLTVPGSLRAVDIPGLKAATPYTVSIYGVIQGYRTPVLSAEASTGETPNLGEVVVAEVGWDALKLNWTAPEGAYEYFFIQVQEADTVEAAQNLTVPGGLRSTDLPGLKAATHYTITIRGVTQDFSTTPLSVEVLTEEVPDMGNLTVTEVSWDALRLNWTTPDGTYDQFTIQVQEADQVEEAHNLTVPGSLRSMEIPGLRAGTPYTVTLHGEVRGHSTRPLAVEVVTEDLPQLGDLAVSEVGWDGLRLNWTAADNAYEHFVIQVQEVNKVEAAQNLTLPGSLRAVDIPGLEAATPYRVSIYGVIRGYRTPVLSAEASTAKEPEIGNLNVSDITPESFNLSWMATDGIFETFTIEIIDSNRLLETVEYNISGAERTAHISGLPPSTDFIVYLSGLAPSIRTKTISATATTEALPLLENLTISDINPYGFTVSWMASENAFDSFLVTVVDSGKLLDPQEFTLSGTQRKLELRGLITGIGYEVMVSGFTQGHQTKPLRAEIVTEAEPEVDNLLVSDATPDGFRLSWTADEGVFDNFVLKIRDTKKQSEPLEITLLAPERTRDITGLREATEYEIELYGISKGRRSQTVSAIATTAMGSPKEVIFSDITENSATVSWRAPTAQVESFRITYVPITGGTPSMVTVDGTKTQTRLVKLIPGVEYLVSIIAMKGFEESEPVSGSFTTALDGPSGLVTANITDSEALARWQPAIATVDSYVISYTGEKVPEITRTVSGNTVEYALTDLEPATEYTLRIFAEKGPQKSSTITAKFTTDLDSPRDLTATEVQSETALLTWRPPRASVTGYLLVYESVDGTVKEVIVGPDTTSYSLADLSPSTHYTAKIQALNGPLRSNMIQTIFTTIGLLYPFPKDCSQAMLNGDTTSGLYTIYLNGDKAEALEVFCDMTSDGGGWIVFLRRKNGRENFYQNWKAYAAGFGDRREEFWLGLDNLNKITAQGQYELRVDLRDHGETAFAVYDKFSVGDAKTRYKLKVEGYSGTAGDSMAYHNGRSFSTFDKDTDSAITNCALSYKGAFWYRNCHRVNLMGRYGDNNHSQGVNWFHWKGHEHSIQFAEMKLRPSNFRNLEGRRKRA

SEQ ID NO: 31 - protein, 1358 aa, Homo sapiens [sequence omitted]

SEQ ID NO: 32 - protein, 439 aa, Homo sapiens
MPAIAVLAAAAAAWCFLQVESRHLDALAGGAGPNHGNFLDNDQWLSTVSQYDRDKYWNRFRDDDYFRNWNPNKPFDQALDPSKDPCLKVKCSPHKVCVTQDYQTALCVSRKHLLPRQKKGNVAQKHWVGPSNLVKCKPCPVAQSAMVCGSDGHSYTSKCKLEFHACSTGKSLATLCDGPCPCLPEPEPPKHKAERSACTDKELRNLASRLKDWFGALHEDANRVIKPTSSNTAQGRFDTSILPICKDSLGWMFNKLDMNYDLLLDPSEINAIYLDKYEPCIKPLFNSCDSFKDGKLSNNEWCYCFQKPGGLPCQNEMNRIQKLSKGKSLLGAFIPRCNEEGYYKATQCHGSTGQCWCVDKYGNELAGSRKQGAVSCEEEQETSGDFGSGGSVVLLDDLEYERELGPKDKEGKLRVHTRAVTEDDEDEDDDKEDEVGYIW

SEQ ID NO: 33 - protein, 424 aa, Homo sapiens
MRAPGCGRLVLPLLLLAAAALAEGDAKGLKEGETPGNFMEDEQWLSSISQYSGKIKHWNRFRDEVEDDYIKSWEDNQQGDEALDTTKDPCQKVKCSRHKVCIAQGYQRAMCISRKKLEHRIKQPTVKLHGNKDSICKPCHMAQLASVCGSDGHTYSSVCKLEQQACLSSKQLAVRCEGPCPCPTEQAATSTADGKPETCTGQDLADLGDRLRDWFQLLHENSKQNGSASSVAGPASGLDKSLGASCKDSIGWMFSKLDTSADLFLDQTELAAINLDKYEVCIRPFFNSCDTYKDGRVSTAEWCFCFWREKPPCLAELERIQIQEAAKKKPGIFIPSCDEDGYYRKMQCDQSSGDCWCVDQLGLELTGTRTHGSPDCDDIVGFSGDFGSGVGWEDEEEKETEEAGEEAEEEEGEAGEADDGGYIW

SEQ ID NO: 34 - protein, 961 aa, Homo sapiens
MLAPRGAAVLLLHLVLQRWLAAGAQATPQVFDLLPSSSQRLNPGALLPVLTDPALNDLYVISTFKLQTKSSATIFGLYSSTDNSKYFEFTVMGRLSKAILRYLKNDGKVHLVVFNNLQLADGRRHRILLRLSNLQRGAGSLELYLDCIQVDSVHNLPRAFAGPSQKPETIELRTFQRKPQDFLEELKLVVRGSLFQVASLQDCFLQQSEPLAATGTGDFNRQFLGQMTQLNQLLGEVKDLLRQQVKETSFLRNTIAECQACGPLKFQSPTPSTVVAPAPPAPPTRPPRRCDSNPCFRGVQCTDSRDGFQCGPCPEGYTGNGITCIDVDECKYHPCYPGVHCINLSPGFRCDACPVGFTGPMVQGVGISFAKSNKQVCTDIDECRNGACVPNSICVNTLGSYRCGPCKPGYTGDQIRGCKVERNCRNPELNPCSVNAQCIEERQGDVTCVCGVGWAGDGYICGKDVDIDSYPDEELPCSARNCKKDNCKYVPNSGQEDADRDGIGDACDEDADGDGILNEQDNCVLIHNVDQRNSDKDIFGDACDNCLSVLNNDQKDTDGDGRGDACDDDMDGDGIKNILDNCPKFPNRDQRDKDGDGVGDACDSCPDVSNPNQSDVDNDLVGDSCDTNQDSDGDGHQDSTDNCPTVINSAQLDTDKDGIGDECDDDDDNDGIPDLVPPGPDNCRLVPNPAQEDSNSDGVGDICESDFDQDQVIDRIDVCPENAEVTLTDFRAYQTVGLDPEGDAQIDPNWVVLNQGMEIVQTMNSDPGLAVGYTAFNGVDFEGTFHVNTQTDDDYAGFIFGYQDSSSFYVVMWKQTEQTYWQATPFRAVAEPGIQLKAVKSKTGPGEHLRNSLWHTGDTSDQVRLLWKDSRNVGWKDKVSYRWFLQHRPQVGYIRVRFYEGSELVADSGVTIDTTMRGGRLGVFCFSQENIIWSNLKYRCNDTIPEDFQEFQTQNFDRFDN

SEQ ID NO: 35 - protein, 478 aa, Homo sapiens
MAPLRPLLILALLAWVALADQESCKGRCTEGFNVDKKCQCDELCSYYQSCCTDYTAECKPQVTRGDVFTMPEDEYTVYDDGEEKNNATVHEQVGGPSLTSDLQAQSKGNPEQTPVLKPEEEAPAPEVGASKPEGIDSRPETLHPGRPQPPAEEELCSGKPFDAFTDLKNGSLFAFRGQYCYELDEKAVRPGYPKLIRDVWGIEGPIDAAFTRINCQGKTYLFKGSQYWRFEDGVLDPDYPRNISDGFDGIPDNVDAALALPAHSYSGRERVYFFKGKQYWEYQFQHQPSQEECEGSSLSAVFEHFAMMQRDSWEDIFELLFWGRTSAGTRQPQFISRDWHGVPGQVDAAMAGRIYISGMAPRPSLAKKQRFRHRNRKGYRSQRGHSRGRNQNSRRPSRATWLSLFSSEESNLGANNYDDYRMDWLVPATCEPIQSVFFFSGDKYYRVNLRTRRVDTVDPPYPRSIAQYWLGCPAPGHL

SEQ ID NO: 36 - DNA, 97 bp, Artificial Sequence
  integrin beta-1 shorthairpin DNA base sequence
tgctgttgacagtgagcgcggctctcaaactataaagaaatagtgaagccacagatgtatttctttatagtttgagagccttgcctactgcctcgga

SEQ ID NO: 37 - DNA, 97 bp, Artificial Sequence
  alpha-3 integrin short hairpin DNA base sequence
tgctgttgacagtgagcgccggatggacatttcagagaaatagtgaagccacagatgtatttctctgaaatgtccatccgttgcctactgcctcgga

SEQ ID NO: 38 - DNA, 103 bp, Artificial Sequence
  luciferase control DNA base sequence
aaggtatattgctgttgacagtgagcgagctcccgtgaattggaatcctagtgaagccacagatgtaggattccaattcagcgggagcctgcctactgcctcg

(19) United States
(12) Patent Application Publication
(10) Pub. No.: US 2013/0274124 A1
(43) Pub. Date: Oct. 17, 2013

(54) ADHESION SIGNATURES

(71) Applicants: Sangeeta N. Bhatia, Lexington, MA (US); David Fernandes Braga Malta, Linda-a-Velha (PT); Nathan Edward Reticker-Flynn, Cambridge, MA (US); Gregory H. Underhill, Champaign, IL (US); Robert Edward Schwartz, Newton, MA (US)

(72) Inventors: Sangeeta N. Bhatia, Lexington, MA (US); David Fernandes Braga Malta, Linda-a-Velha (PT); Nathan Edward Reticker-Flynn, Cambridge, MA (US); Gregory H. Underhill, Champaign, IL (US); Robert Edward Schwartz, Newton, MA (US)

(73) Assignee: Massachusetts Institute of Technology, Cambridge, MA (US)

(21) Appl. No.: 13/794,772

(22) Filed: Mar. 11, 2013

Related U.S. Application Data
(60) Provisional application No. 61/609,115, filed on Mar. 9, 2012.

(51) Int. Cl. G01N 33/50; C12N 5/0735; C12N 5/09; G01N 33/68; C12N 5/071

(52) U.S. Cl. CPC G01N 33/5032; G01N 33/6887; C12N 5/067; C12N 5/0676; C12N 5/0693; C12N 5/0606; C12N 5/0657
USPC 506/9; 506/18; 435/377; 435/395; 435/370

(57) ABSTRACT

The present invention provides arrays comprising polypeptides associated with extracellular matrix that can be used to isolate, differentiate, or culture certain cell types including stem cells, cancer cells, and/or primary hepatocytes. The array comprises at least a pair of polypeptides that comprise a polypeptide associated with extracellular matrix or functional fragments thereof. The invention also provides for methods of diagnosing and/or prognosing a certain disease or disorder through contacting a cell sample from a patient with an array comprising at least a pair of polypeptides that comprise a polypeptide sequence associated with extracellular matrix or functional fragments thereof.

### CROSS-REFERENCE TO RELATED APPLICATIONS

This application is an international application designating the United States of America and filed under 35 U.S.C. §120, which claims priority to U.S. Provisional Ser. No. 61/609,115, filed on Mar. 9, 2012, which is herein incorporated by reference in its entirety.

### FIELD OF THE INVENTION

The invention relates generally to devices that culture, differentiate, or isolate cells based upon the surface expression of cellular ligands that associate and respond to polypeptides immobilized to a surface of the device. In some embodiments, the polypeptides mimic the extracellular matrix microenvironment. The invention also generally relates to methods of diagnosing patients with particular disorders based upon the presence or absence of cultured or isolated cells or the presence of absence of the display of certain cellular phenotypes.

### BACKGROUND OF THE INVENTION

There are a wide variety of contexts in biology and medicine when it is important or useful to be able to distinguish cells of different types from one another, to separate cells of different types from one another, and/or to identify, characterize, or define particular cells as members of one cell type or another. Current methods of isolation, differentiation, and characterization can be improved. Identification and characterization of certain cellular properties or expression profiles can be used for diagnosis or prognosis for certain disease states.

### SUMMARY OF INVENTION

The present invention encompasses the recognition that cells can be identified and/or characterized by “adhesion signatures” that embody a cell's affinity for extracellular matrix components. In some embodiments, an adhesion signature embodies, or displays, an affinity for one or more such extracellular matrix components and is sufficient to distinguish or characterize relevant cells as compared with at least one other reference cell.

For example, in some embodiments, an adhesion signature is sufficient to distinguish or characterize cells of a particular cell type (e.g., host or tissue type, state of development, etc) from cells of one or more other types. In some embodiments, adhesion signatures allow isolating and/or culturing cells of a particular type and/or under defined conditions. In some embodiments, a cell sample that contains metastatic cells may bind an adhesion set comprising fibronectin in combination with galectin-3, galectin-8 or laminin more consistently than a cell sample that does not contain metastatic cells.

In accordance with the present invention, adhesion signatures are defined for particular cells or cell types relative to appropriate reference cells or cell types. In some embodiments, the particular cells differ from reference cells in that they are progeny of a different source (e.g., different cell lineage, organism, tissue type, etc.) as compared with the reference cells, the cells are at a different developmental stage than the reference cells, the cells suffer from or are susceptible to a particular disease, disorder, or condition. In some embodiments, cells are identical to reference cells with the exception of a characteristic or characteristics that results in a difference identifiable by differing adhesion signatures.

Further in accordance with the present invention, adhesion signatures are used to identify and/or characterize cells. For example, in some embodiments adhesion signatures are used to distinguish cells suffering from or susceptible to a particular disease, disorder, or condition from those that are not. In further embodiments, adhesion signatures are used to identify and/or characterize cells of a particular developmental stage, cell lineage, or tissue type.

The invention provides an array of polypeptides, the array comprising: a solid support and a plurality of adhesion sets, wherein each adhesion set comprises two or more different polypeptides comprising a polypeptide sequence associated with the extracellular matrix or a functional fragment thereof, and wherein the adhesion sets are attached to the solid support at an addressable location of the array. In some embodiments, the solid support is a slide optionally coated with a polymer. In some embodiments, the solid support is coated with a polymer. In some embodiments, the polymer is polyacrylamide. In some embodiments, the solid support is a material chosen from: polystyrene (TCPS), glass, quarts, quartz glass, poly(ethylene terephthalate) (PET), polyethylene, polyvinyl difluoride (PVDF), polydimethylsiloxane (PDMS), polytetrafluoroethylene (PTFE), polymethylmethacrylate (PMMA), polycarbonate, polyolefin, ethylene vinyl acetate, polypropylene, polysulfone, polytetrafluoroethylene, silicones, poly(meth)acrylic acid, polyamides, polyvinyl chloride, polyvinylphenol, and copolymers and mixtures thereof. In some embodiments, the at least one adhesion set comprises two different polypeptides attached to a solid support.

The invention further relates to an array of polypeptides, the array comprising: a solid support and a plurality of adhesion sets, wherein each adhesion set comprises two or more different polypeptides comprising a polypeptide sequence associated with the extracellular matrix or a functional fragment thereof, and wherein the adhesion sets are attached to the solid support at an addressable location of the array; and wherein the two or more of the different polypeptides sequences are chosen from: collagen I, collagen II, collagen III, collagen IV, collagen V, collagen VI, fibronectin, laminin, merosin, tenascin-R, chondroitin sulfate, agreccan, elastin, keratin, mucin, superfibronectin, F-spondin, nidogen-2, heparin sulfate, biglycan, decorin, galectin 1, galectin 3, galectin 3c, galectin 4, galectin 8, thrombospondin-4, osteopontin, osteonectin, testican 1, testican 2, fibrin, tenascin-C, nidogen-1, vitronectin, rat agrin, hyaluronan, brevican, or functional fragments thereof. In some embodiments, the at least one adhesion set comprises at least two different polypeptide sequences chosen from: osteopontin, thrombospondin-4, fibronectin, laminin, galectin 3, galectin 8, or functional fragments thereof. In some embodiments, the at least one adhesion set comprises at least two different polypeptide sequences chosen from: fibronectin, laminins and functional fragments thereof. In some embodiments, the at least one adhesion set comprises at least two different polypeptide sequences chosen from: fibronectin, galectin 3, and functional fragments thereof. In some embodiments, the at least one adhesion set comprises at least two different polypeptide sequences chosen from: fibronectin, galectin 8, and functional fragments thereof.

In some embodiments, the at least one adhesion set comprises at least two different polypeptide sequences chosen from: thrombospondin-4, galectin 8, and functional fragments thereof.

In some embodiments, wherein an array or system disclosed herein comprises at least one adhesion set comprising two polypeptide sequences associated with the extracellular matrix chosen from: Collagen 1 and Agreccan, Collagen IV and Nidogen-1, or a functional fragment thereof. In some embodiments, the at least one adhesion set comprises at least one polypeptide sequence that is osteopontin or a functional fragment thereof. In some embodiments, each adhesion set consists of a pair of different polypeptides associated with the extracellular matrix. In some embodiments, the array comprises at least about 700, about 750, or about 800 different adhesion sets. In some embodiments, the array comprises at least about 700, about 750, or about 800 different adhesion sets positioned at different discrete locations on the array.

The invention further relates to an array of polypeptides, the array comprising: a solid support and a plurality of adhesion sets, wherein each adhesion set comprises two or more different polypeptides comprising a polypeptide sequence associated with the extracellular matrix or a functional fragment thereof, and wherein the adhesion sets are attached to the solid support at an addressable location of the array; and wherein the array is free of animal-derived ECM material, embryonic fibroblasts, material deposited from Engelbreth-Holm-Swarm (EHS) mouse sarcoma cells, or any combination thereof. In some embodiments, the array is free of serum derived or sourced from any animal species.

The invention relates to an array of polypeptides, the array comprising: a solid support and a plurality of adhesion sets, wherein each adhesion set comprises two or more different polypeptides comprising a polypeptide sequence associated with the extracellular matrix or a functional fragment thereof, and wherein the array further comprises one or a plurality of mammalian cells. In some embodiments, the one or a plurality of mammalian cells contains at least one lung cell.

The invention further provides an array or kit comprising at least one cell or at least one cell sample. In some embodiments, the cell sample contains at least one cancer cell or one stem cell.

In some embodiments, the cancer cell is derived from the cancer of the adrenal gland, bladder, bone, bone marrow, brain, spine, breast, cervix, gall bladder, ganglia, gastrointestinal tract, stomach, colon, heart, kidney, liver, lung, muscle, ovary, pancreas, parathyroid, penis, prostate, salivary glands, skin, spleen, testis, thymus, thyroid, or uterus. In some embodiments, the array or kit comprises a stem cell that is an embryonic stem cell, an adipose-derived stem cell, a mesenchymal stem cell, an umbilical stem cell or a pluripotent stem cell.

The invention relates to an array of polypeptides, the array comprising: a solid support and a plurality of adhesion sets, wherein each adhesion set comprises two or more different polypeptides comprising a polypeptide sequence associated with the extracellular matrix or a functional fragment thereof, wherein the two or more different polypeptides are attached to the solid support via passive electrostatic non-covalent binding.

The invention provides a system comprising: an array of polypeptides, the array comprising: a solid support and a plurality of adhesion sets, wherein each adhesion set comprises two or more different polypeptides comprising a polypeptide sequence associated with the extracellular matrix or a functional fragment thereof; and a cell culture vessel. In some embodiments, the system further comprises at least one or a plurality of cells. In some embodiments, the system further comprises at least one or a plurality of cells derived from cancer cells chosen from: cancer of the adrenal gland, bladder, bone, bone marrow, brain, spine, breast, cervix, gall bladder, ganglia, gastrointestinal tract, stomach, colon, heart, kidney, liver, lung, muscle, ovary, pancreas, parathyroid, penis, prostate, salivary glands, skin, spleen, testis, thymus, thyroid, or uterus. In some embodiments, the system further comprises cell media free of at least one of: serum, animal-derived ECM material, embryonic fibroblasts, or material deposited from Engelbreth-Holm-Swarm (EHS) mouse sarcoma cell. In some embodiments, the system further comprises cell media free of: serum, animal-derived ECM material, embryonic fibroblasts, and material deposited from Engelbreth-Holm-Swarm (EHS) mouse sarcoma cell. In some embodiments, the system further comprises at least one or a plurality of cells is a stem cell chosen from: an embryonic stem cell, an adipose-derived stem cell, a mesenchymal stem cell, an umbilical stem cell or a pluripotent stem cell.

The invention also provides a kit comprising: an array of polypeptides, the array comprising: a solid support and a plurality of adhesion sets, wherein each adhesion set comprises two or more different polypeptides comprising a polypeptide sequence associated with the extracellular matrix or a functional fragment thereof; and optionally comprising a cell culture vessel. In some embodiments, the kit further comprises at least one of the following: cell media, a volume of fluorescent stain or dye, a cell sample, and a set of instructions, optionally accessible remotely through an electronic medium.

The invention further provides a method of identifying an adhesion signature of a cell sample comprising: contacting a cell sample to an array or system disclosed herein; and determining a quantity of cells bound to one or a plurality of adhesion sets. In some embodiments, the cell sample contains at least one cell from a biopsy. The invention also provides a method of inducing differentiation of a cell comprising contacting a cell sample to an array or a system disclosed herein. In some embodiments, the method includes inducing differentiation of a stem cell chosen from: an embryonic stem cell, an adipose-derived stem cell, a mesenchymal stem cell, an umbilical stem cell or a pluripotent stem cell. In some embodiments, the step of contacting a cell or cell sample comprises exposing the cell or cell sample to the array or the system for a sufficient period of time for differentiation of a cell to a hepatic or pancreatic lineage.

The invention also provides for a method of culturing a cell comprising contacting a cell or a cell sample to an array or a system disclosed herein in the presence of cell media. In some embodiments, the cell media is serum free. In some embodiments, the cell media is free of at least one or a combination of: serum, animal-derived ECM material, embryonic fibroblasts, or material deposited from Engelbreth-Holm-Swarm (EHS) mouse sarcoma cell. In some embodiments, the cell or cell sample is derived from a primary lineage of a cancer cells or stem cells. In some embodiments, the invention relates to a method of culturing a cell or cell sample wherein the cell or the cell sample comprises one or a plurality of stem cells chosen from: an embryonic stem cell, an adipose-derived stem cell, a mesenchymal stem cell, an umbilical stem cell or a pluripotent stem cell is a pluripotent stem cell or embryonic stem cell. In some embodiments, the invention relates to a method of culturing a cell comprising contacting a cell or a cell sample to an array or a system disclosed herein in the presence of cell media comprises a contacting cell wherein the cell is passaged at least about 30 times, at least 40 times, or at least 50 times.

The invention further provides a method of culturing one or a plurality of primary hepatocytes, the method comprising contacting one or a plurality of primary hepatocytes with an array or system disclosed herein.

The invention further relates to a method of diagnosing a hyperproliferative disease comprising: (a) contacting a cell sample to an array or system disclosed herein; (b) quantifying one or more adhesion values; (c) determining one or more adhesion signatures of the cell sample based upon the adhesion values; and (d) comparing the adhesion signature of the cell sample to an adhesion signature of a control cell sample. In some embodiments, the hyperproliferative disease is metastatic lung cancer. In some embodiments, the hyperproliferative disease is metastatic breast cancer.

The invention relates to a method of prognosing a clinical outcome of a subject comprising: (a) contacting a cell or cell sample to an array or system disclosed herein; (b) quantifying one or more adhesion values; (c) determining one or more adhesion signatures of the cell sample based upon the adhesion values; and (d) correlating the adhesion signature to an adhesion signature of a cell sample associated with a clinical outcome.

The invention further provides a method of determining patient responsiveness to a therapy comprising: (a) contacting a cell or cell sample to an array or system disclosed herein; (b) quantifying one or more adhesion values; (c) determining one or more adhesion signatures of the cell sample based at least partially upon the adhesion values; and (d) comparing the one or more adhesion signatures to one or more adhesion signature of a control cell sample.

The invention further provides a method of determining patient responsiveness to a therapy comprising: (a) contacting a cell or cell sample to an array or system disclosed herein; (b) quantifying one or more adhesion values by detecting fluorescence of cells through a computer-program product disclosed herein; (c) determining one or more adhesion signatures of the cell sample based at least partially upon the adhesion values; and (d) comparing the one or more adhesion signatures to one or more adhesion signature of a control cell sample.

The invention provides a method of isolating a cell comprising: contacting a cell sample to an array or system disclosed herein. In some embodiments, the method of isolating a cell comprises contacting a cell sample to an array or system disclosed herein for a sufficient time period and under sufficient conditions for a cell to adhere to the array or the system more tightly than other components of the cell sample. In some embodiments, the method of isolating a cell further comprises rinsing the array or system with a buffer that that washes other components of the cell sample from the cell.

The invention also provides a method of adhering hepatocytes derived from a primary lineage of human liver cells comprising contacting the hepatocytes to an array or system disclosed herein. The invention also provides a method of maintaining a culture of hepatocytes derived from a primary lineage of human liver cells comprising contacting the hepatocytes to an array or system disclosed herein.

The invention provides a method of sorting a mixture of cell types comprising: contacting a mixture of cell types to an array or system disclosed herein. In some embodiment, the method of sorting a mixture of cell types further comprises the step of determining one or more adhesion signatures of the cell sample based upon a calculated adhesion value. In some embodiments, the method further comprises the step of comparing the one or more adhesion signatures to one or more adhesion signature of a control cell type, and sorting the cell types based upon their similarities or differences to a phenotype of a the control cell type.

In some embodiments, particular cells are isolated from a composition also comprising other cells based on an adhesion signature common to the isolated cells and different from the other cells. For example, in some embodiments, a composition comprising more than one type of cells is contacted with one or more ECM components, wherein the affinity of the particular cells to be isolated for the ECM components constitutes part of an adhesion signature for the cells. In further embodiments, cells are cultured in media containing the ECM component composition.

In some embodiments, the present disclosure provides methods comprising contacting a sample comprising cells with a collection of extracellular matrix (ECM) components and detecting presence or level of interactions between cells in the sample and ECM components in the collection. In some embodiments, provided methods comprise determining that a particular set of detected interactions defines an adhesion signature that is characteristic of particular cells in the sample in that it distinguishes them from other cells in the sample or from reference cells. In some embodiments, detecting comprises detecting presence or level of a set of interactions that is characteristic of particular cells in the sample in that it distinguishes them from other cells in the sample or from reference cells.

In some embodiments, the present disclosure provides methods comprising contacting a sample comprising cells with a collection of extracellular matrix (ECM) components under conditions and for a time sufficient for a set of interactions to occur between particular cells in the sample and ECM components in the collection sufficient to isolate the cells from other components of the sample. In some embodiments, the other components of the sample from which the particular cells are isolated include other cells.

In certain embodiments, the other cells are cells that make a different set of interactions with the ECM components than do the isolated cells. In certain embodiments, the step of contacting comprises contacting with ECM components attached to a solid phase, under conditions and for a time sufficient for the set of interactions to occur on the solid phase. In some embodiments, provided methods comprise a step of separating solid phase from sample, so that particular cells making interactions with the solid phase are separated from the sample.

In some embodiments, the present disclosure provides methods for determining the effects on cells of interacting with extracellular matrix components comprising exposing a first population of cells to a first set of conditions that include contacting with a collection of extracellular matrix components, exposing a second population of cells, which second population of cells is comparable to the first population of cells, to a second set of conditions, which second set of conditions is comparable to the first set of conditions except that some or all of the extracellular matrix components are absent from the contacting; and determining one or more cell population features that differs between the first and second populations of cells after the exposing has occurred.

In some embodiments, the present disclosure provides methods of culturing a cell type of interest comprising contacting a sample comprising cells of a cell type of interest with a collection of extracellular matrix (ECM) components appropriate to promote growth and/or replication of cells of the cell type of interest as compared with cells of one or more different cell types. In some embodiments, the collection of ECM components is suspended in media. In certain embodiments, the collection of ECM components is attached to a solid phase. In some embodiments, the method further comprises isolating cells of the cell type of interest from the solid phase.

In some embodiments, the present disclosure provides kits for cell isolation and growth comprising a substrate coated with a collection of ECM components characterized in that, when a sample containing cells of a plurality of different cell types, which plurality of different cell types includes at least one cell type of interest, is contacted with the substrate, cells of the cell type of interest form a set of interactions with ECM components in the collection sufficient to isolate the cells of the cell type of interest from other cells in the sample. In some embodiments, isolation comprises growth of the cells of the cell type of interest. In some embodiments, growth comprises proliferation. In some embodiments, growth is sufficient to overpopulate the sample with the cell type of interest as compared with other cell types. In certain embodiments, the kit further comprises medium. In some embodiments, the kit further comprises cells of the cell type of interest.

In some embodiments, the present disclosure provides systems for culturing cells comprising a substrate coated with a collection of ECM components characterized in that, when a sample containing cells of a plurality of different cell types, which plurality of different cell types includes at least one cell type of interest, is contacted with the substrate, cells of the cell type of interest form a set of interactions with ECM components in the collection sufficient to isolate the cells of the cell type of interest from other cells in the sample. In some embodiments, isolation comprises growth of the cells of the cell type of interest. In certain embodiments, growth comprises proliferation. In some embodiments, growth is sufficient to overpopulate the sample with the cell type of interest as compared with other cell types.

In some embodiments, the present disclosure provides kits for cancer diagnosis comprising a substrate coated with a collection of ECM components characterized in that, when a sample containing cells of a plurality of different cell types, which plurality of different cell types includes at least one cell type of interest is contacted with the substrate, cells of the cell type of interest form a set of interactions with ECM components in the collection sufficient to isolate the cells of the cell type of interest from other cells in the sample. In some embodiments, the cell type of interest is cancer cells of a particular stage of metastasis. In some embodiments, isolation comprises growth of the cancer cells of a particular stage. In some embodiments, growth comprises proliferation. In some embodiments, the growth is sufficient to overpopulate the sample with the cancer cells of a particular stage as compared with other cell types. In some embodiments, the kit further comprises medium. In some embodiments, the kit further comprises a means for assessing abundance of the cancer cells of a particular stage.

The invention provides a method comprising steps of: contacting a sample comprising cells with a collection of extracellular matrix (ECM) components; detecting presence or level of interactions between cells in the sample and ECM components in the collection. In some embodiments, the method further comprises determining that a particular set of detected interactions defines an adhesion signature that is characteristic of particular cells in the sample in that it distinguishes them from other cells in the sample or from reference cells. In some embodiments, the step of detecting comprises detecting presence or level of a set of interactions that is characteristic of particular cells in the sample in that it distinguishes them from other cells in the sample or from reference cells. In some embodiments, the collection of ECM components is attached to a solid phase. In some embodiments, the invention provides a method comprising steps of: contacting a sample comprising cells with a collection of extracellular matrix (ECM) components; and detecting presence or level of interactions between cells in the sample and ECM components in the collection, wherein the ECM components in the collection are separately attached in discrete locations to the solid phase. In some embodiments, the step of detecting comprises quantifying binding levels at one or more of the discrete locations. In some embodiments, the step of detecting comprises quantifying binding levels at all of the discrete locations.

The invention further provides a method comprising steps of: contacting a sample comprising cells with a collection of extracellular matrix (ECM) components; detecting presence or level of interactions between cells in the sample and ECM components in the collection; and determining that a particular set of detected interactions defines an adhesion signature that is characteristic of particular cells in the sample in that it distinguishes them from other cells in the sample or from reference cells. In some embodiments, the step of detecting comprises determining presence or level of a predetermined set of interactions between cells in the sample and ECM components in the collection. In some embodiments, the method further comprises comparing the determined presence or level with reference presence or level of the predetermined set, so that identity with, similarity to, or difference from the reference presence or level is determined. In some embodiments, the reference presence or level is or comprises an adhesion signature that is characteristic of a particular cell type in that it distinguishes cells of the particular cell type from cells of at least one other cell type. In some embodiments, the reference presence or level is or comprises an adhesion signature of cells in a particular stage of development in that it distinguishes them from otherwise comparable cells in a different stage of development.

The invention also provides for a method comprising steps of: contacting a sample comprising cells with a collection of extracellular matrix (ECM) components under conditions and for a time sufficient for a set of interactions to occur between particular cells in the sample and ECM components in the collection sufficient to isolate the cells from other components of the sample. In some embodiments, the other components of the sample from which the particular cells are isolated include other cells. In some embodiments, the other cells are cells that make a different set of interactions with the ECM components than do the isolated cells.

The invention also provides for a method comprising steps of: contacting a sample comprising cells with a collection of extracellular matrix (ECM) components under conditions and for a time sufficient for a set of interactions to occur between particular cells in the sample and ECM components in the collection sufficient to isolate the cells from other components of the sample, wherein the step of contacting comprises contacting with ECM components attached to a solid phase, under conditions and for a time sufficient for the set of interactions to occur on the solid phase. In some embodiments, the method further comprises a step of separating the solid phase from the sample, so that the particular cells are separated from the sample.

The invention provides for a method comprising steps of: contacting a sample comprising cells with a collection of extracellular matrix (ECM) components, wherein the collection of extracellular matrix components comprises one or more of aggrecan, agrin, biglycan, brevican, chondroitin sulfate, collagen I, collagen II, collagen III, collagen IV, collagen V, collagen VI, decorin, elastin, f-spondin, fibrin, fibronectin, galectin 1, galectin 3, galectin 3c, galectin 4, galectin 8, heparan sulfate, hyaluronic acid, keratin, laminin, merosin, mucin, nidogen-1, nidogen-2, osteopontin, SPARC/osteonectin, superfibronectin, tenascin-C, tenascin-R, testican 1/SPOCKI, testican 2/SPOCK2, thrombospondin-4, vitronectin, and functional fragments thereof. The invention provides for a method comprising steps of: contacting a sample comprising cells with a collection of extracellular matrix (ECM) components, wherein the collection of extracellular matrix components comprises at least two ECM components selected from: agrin and collagen IV, agrin and fibrin, biglycan and collagen II, biglycan and fibrin, collagen I and thrombospondin-4, collagen II and decorin, collagen II and tenascin-C, collagen II and testican 2, collagen III and collagen VI, collagen III and thrombospondin-4, collagen IV and galectin 4, collagen IV and SPARC, collagen IV and vitronectin, collagen V and galectin 1, collagen VI and galectin 3, fibrin and galectin 3c, fibrin and galectin 4, fibrin and keratin, fibrin and osteopontin, fibrin and SPARC, f-spondin and fibronectin, fibronectin and galectin 3, fibronectin and galectin 8, fibronectin and laminin, fibronectin and testican 1, and or functional fragments thereof. In some embodiments, the invention provides for a method comprising steps of: contacting a sample comprising cells with a collection of extracellular matrix (ECM) components, wherein the collection of extracellular matrix components comprises at least two ECM components selected from: agrin and collagen II, agrin and laminin, biglycan and collagen II, brevican and fibronectin, collagen I and testican 2, collagen II and collagen IV, collagen II and laminin, collagen II and nidogen-1, collagen II and testican 2, collagen III and galectin 8, collagen III and superfibronectin, collagen V and fibronectin, collagen V and galectin 1, collagen VI and fibronectin, collagen VI and nidogen-1, collagen VI and tenascin-C, decorin and fibronectin, decorin and galectin 8, decorin and laminin, elastin and galectin 4, fibrin and galectin 3, fibronectin and galectin 1, fibronectin and galectin 3, fibronectin and galectin 4, fibronectin and mucin, fibronectin and SPARC, fibronectin and testican 2, galectin 1 and galectin 3, galectin 1 and keratin, galectin 3 and heparan sulfate, galectin 3 and superfibronectin, galectin 4 and nidogen-1, galectin 8 and tenascin-C, keratin and laminin, laminin and merosin, laminin and thrombospondin-4, SPARC and superfibronectin, superfibronectin and testican 1, and/or functional fragments thereof.

In some embodiments, the invention provides for a method comprising steps of: contacting a sample comprising cells with a collection of extracellular matrix (ECM) components, wherein the collection of extracellular matrix components comprises at least two ECM components selected from: at least two ECM components selected from biglycan and collagen IV, biglycan and galectin 4, brevican and collagen I, brevican and collagen IV, brevican and galectin 3c, collagen I and galectin 1, collagen I and galectin 3, collagen I and galectin 3c, collagen I and galectin 8, collagen I and nidogen-2, collagen I and SPARC, collagen I and tenascin-C, collagen I and testican 1, collagen I and vitronectin, collagen II and galectin 3, collagen II and galectin 8, collagen II and nidogen-1, collagen II and nidogen-2, collagen IV and decorin, collagen IV and galectin 8, collagen IV and nidogen-1, collagen IV and nidogen-2, collagen IV and testican 1, collagen IV and testican 2, collagen VI and f-spondin, collagen VI and galectin 3, collagen VI and galectin 8, collagen VI and tenascin-C, collagen VI and testican 2, collagen VI and thrombospondin-4, f-spondin and vitronectin, fibrin and galectin 4, fibronectin and galectin 4, fibronectin and nidogen-1, fibronectin and tenascin-C, fibronectin and testican 1, fibronectin and testican 2, galectin 3 and vitronectin, galectin 3c and merosin, galectin 3c and superfibronectin, galectin 4 and superfibronectin, galectin 8 and superfibronectin, galectin 8 and vitronectin, laminin and vitronectin, SPARC and testican 1, and/or superfibronectin and vitronectin.

The invention further provides for a method comprising steps of: contacting a sample comprising cells with a collection of extracellular matrix (ECM) components under conditions and for a time sufficient for a set of interactions to occur between particular cells in the sample and ECM components in the collection sufficient to isolate the cells, wherein the particular cells are human embryonic stem cells, human induced pluripotent stem cells, hepatocytes, mesenchymal stem cells, or cancer cells. In some embodiments, the mesenchymal stem cells are derived from bone marrow, adipose tissue, umbilical cord blood or umbilical cord. In some embodiments, the cells are cells in a certain stage of development. In some embodiments, the cancer cells are from a primary tumor, lymph nodes, or metastases at organ sites. In some embodiments, the cancer cells are from a primary tumor, lymph nodes, metastases at organ sites, or metastatic tissue. In some embodiments, the cancer cells are non-small cell lung cancer cells. In some embodiments, the cancer cells are breast cancer cells.

The invention also provides for a method of determining the effects on cells of interacting with extracellular matrix components, the method comprising steps of: exposing a first population of cells to a first set of conditions that includes contacting with a collection of extracellular matrix components, exposing a second population of cells, which second population of cells is comparable to the first population of cells, to a second set of conditions, which second set of conditions is comparable to the first set of conditions except that some or all of the extracellular matrix components are absent from the contacting; and determining one or more cell population features that differs between the first and second populations of cells after the exposing has occurred.

The invention also provides for a method for culturing a cell type of interest comprising contacting a sample comprising cells of a cell type of interest with a collection of extracellular matrix (ECM) components appropriate to promote growth and/or replication of cells of the cell type of interest as compared with cells of one or more different cell types. In some embodiments, the collection of ECM components is suspended in media. In some embodiments, the collection of ECM components is attached to a solid phase. In some embodiments, the method of culturing a cell type of interest further comprises isolating cells of the cell type of interest from the solid phase. In some embodiments, the collection of ECM components comprises ECM components that participate in interactions defining an adhesion signature characteristic of the cell type of interest in that it distinguishes cells of the cell type of interest from otherwise comparable cells of a different cell type. In some embodiments, the cell type of interest is cells in a developmental stage of interest and the different cell type is otherwise comparable cells in a different developmental stage.

In some embodiments, the invention provides for any of the disclosed methods wherein the cell type of interest is human embryonic stem cells or human induced pluripotent stem cells, and wherein the collection of ECM components comprises at least two ECM components selected from collagen II and galectin 4, collagen IV and galectin 8, collagen I and Laminin, or functional fragments thereof.

In some embodiments, the invention provides for any of the disclosed methods wherein the cell type of interest is human mesenchymal stem cells. In some embodiments, the mesenchymal stem cells are derived from bone marrow, adipose tissue, umbilical cord blood or umbilical cord.

The invention further provides for a method for culturing a cell type of interest comprising contacting a sample comprising cells of a cell type of interest with a collection of extracellular matrix (ECM) components appropriate to promote growth and/or replication of cells of the cell type of interest as compared with cells of one or more different cell types, wherein the collection of ECM components comprises at least two ECM components selected from biglycan and collagen IV, biglycan and galectin 4, brevican and collagen I, brevican and collagen IV, brevican and galectin 3c, collagen I and galectin 1, collagen I and galectin 3, collagen I and galectin 3c, collagen I and galectin 8, collagen I and nidogen-2, collagen I and SPARC, collagen I and tenascin-C, collagen I and testican 1, collagen I and vitronectin, collagen II and galectin 3, collagen II and galectin 8, collagen II and nidogen-1, collagen II and nidogen-2, collagen IV and decorin, collagen IV and galectin 8, collagen IV and nidogen-1, collagen IV and nidogen-2, collagen IV and testican 1, collagen IV and testican 2, collagen VI and f-spondin, collagen VI and galectin 3, collagen VI and galectin 8, collagen VI and tenascin-C, collagen VI and testican 2, collagen VI and thrombospondin-4, f-spondin and vitronectin, fibrin and galectin 4, fibronectin and galectin 4, fibronectin and nidogen-1, fibronectin and tenascin-C, fibronectin and testican 1, fibronectin and testican 2, galectin 3 and vitronectin, galectin 3c and merosin, galectin 3c and superfibronectin, galectin 4 and superfibronectin, galectin 8 and superfibronectin, galectin 8 and vitronectin, laminin and vitronectin, SPARC and testican 1, superfibronectin and vitronectin and/or functional fragments thereof. In some embodiments, the cell type of interest is human embryonic stem cells, mouse embryonic stem cells and/or human induced pluripotent stem cells. In some embodiments, the collection of ECM components comprises fibronectin and merosin.

In some embodiments, the invention further provides for a method for culturing hepatocytes comprising contacting a sample comprising hepatocytes with a collection of extracellular matrix (ECM) components appropriate to promote growth and/or replication of the hepatocytes. In some embodiments, the collection of ECM components comprises at least two ECM components selected from agrin and collagen I, collagen I and laminin, collagen I and merosin, collagen II and galectin 8, collagen II and SPARC, and/or collagen IV and nidogen-1.

The invention further provides for any of the disclosed methods herein wherein each of the disclosed steps is performed in a serum-free environment. The invention also provides for any of the disclosed methods herein comprising cells of a cell type of interest that are isolated from serum-free media or fully defined synthetic media.

In some embodiments, the invention provides for a kit for cell isolation and growth comprising: a substrate coated with a collection of ECM components characterized in that, when a sample containing cells of a plurality of different cell types, which plurality of different cell types includes at least one cell type of interest, is contacted with the substrate, cells of the cell type of interest form a set of interactions with ECM components in the collection sufficient to isolate the cells of the cell type of interest from other cells in the sample. In some embodiments, the isolation comprises growth of the cells of the cell type of interest. In some embodiments, the growth of cells comprises proliferation of the cell type of interest. In some embodiments, the growth is sufficient to overpopulate the sample with the cell type of interest as compared with other cell types.

The invention further provides for a kit comprising: a substrate coated with a collection of ECM components characterized in that, when a sample containing cells of a plurality of different cell types, which plurality of different cell types includes at least one cell type of interest, is contacted with the substrate, cells of the cell type of interest form a set of interactions with ECM components in the collection sufficient to isolate the cells of the cell type of interest from other cells in the sample. In some embodiments, the kit further comprises cell media. In some embodiments, the kit further comprises serum-free media or fully defined synthetic cell media. In some embodiments, the kit further comprises cells of the cell type of interest. In some embodiments, the kit further comprises cells of the cell type of interest, wherein the cell type of interest is a stem cell, cancer cell, or hepatocyte. In some embodiments, the substrate is coated with an array of ECM components. In some embodiments, the substrate is coated with an array of any pair of ECM components disclosed herein.

The invention further provides for a system for culturing cells comprising: a substrate coated with a collection of ECM components characterized in that, when a sample containing cells of a plurality of different cell types, which plurality of different cell types includes at least one cell type of interest, is contacted with the substrate, cells of the cell type of interest form a set of interactions with ECM components in the collection sufficient to isolate the cells of the cell type of interest from other cells in the sample. In some embodiments, the isolation comprises growth of the cells of the cell type of interest. In some embodiments, the growth comprises proliferation. In some embodiments, the growth is sufficient to overpopulate the sample with the cell type of interest as compared with other cell types. In some embodiments, the system comprises a substrate comprised of polystyrene or polypropylene. In some embodiments, the cell type of interest is human mesenchymal stem cells. In some embodiments, the cell type of interest is mesenchymal stem cells are derived from bone marrow, adipose tissue, umbilical cord blood or umbilical cord. In some embodiments, the cell type of interest is human mesenchymal stem cells are derived from bone marrow, adipose tissue, umbilical cord blood or umbilical cord.

The invention further provides for a system for culturing cells comprising: a substrate coated with a collection of ECM components wherein the collection of ECM components comprises at least two ECM components selected from biglycan and collagen IV, biglycan and galectin 4, brevican and collagen I, brevican and collagen IV, brevican and galectin 3c, collagen I and galectin 1, collagen I and galectin 3, collagen I and galectin 3c, collagen I and galectin 8, collagen I and nidogen-2, collagen I and SPARC, collagen I and tenascin-C, collagen I and testican 1, collagen I and vitronectin, collagen II and galectin 3, collagen II and galectin 8, collagen II and nidogen-1, collagen II and nidogen-2, collagen IV and decorin, collagen IV and galectin 8, collagen IV and nidogen-1, collagen IV and nidogen-2, collagen IV and testican 1, collagen IV and testican 2, collagen VI and f-spondin, collagen VI and galectin 3, collagen VI and galectin 8, collagen VI and tenascin-C, collagen VI and testican 2, collagen VI and thrombospondin-4, f-spondin and vitronectin, fibrin and galectin 4, fibronectin and galectin 4, fibronectin and nidogen-1, fibronectin and tenascin-C, fibronectin and testican 1, fibronectin and testican 2, galectin 3 and vitronectin, galectin 3c and merosin, galectin 3c and superfibronectin, galectin 4 and superfibronectin, galectin 8 and superfibronectin, galectin 8 and vitronectin, laminin and vitronectin, SPARC and testican 1, and/or superfibronectin, vitronectin, or functional fragments thereof. In some embodiments, the cell type of interest is human embryonic stem cells or human induced pluripotent stem cells and the collection of ECM components comprises at least two ECM components selected from collagen II and galectin 4, collagen IV and galectin 8, or collagen I and Laminin. In some embodiments, the cell type of interest comprises hepatocytes.

The invention further provides for a system for culturing cells comprising: a substrate coated with a collection of ECM components wherein the collection of ECM components comprises at least two ECM components selected from agrin and collagen I, collagen I and laminin, collagen I and merosin, collagen II and galectin 8, collagen II and SPARC, and/or collagen IV and nidogen-1.

The invention provides for a kit for cancer stage diagnosis comprising: a substrate coated with a collection of ECM components characterized in that, when a sample containing cells of a plurality of different cell types, which plurality of different cell types includes at least one cell type of interest is contacted with the substrate, cells of the cell type of interest form a set of interactions with ECM components in the collection sufficient to isolate the cells of the cell type of interest from other cells in the sample. In some embodiments, the cell type of interest is cancer cells of a particular stage of metastasis. In some embodiments, the kit comprises a substrate coated with a collection of ECM components characterized in that, when a sample containing cells of a plurality of different cell types, which plurality of different cell types includes at least one cell type of interest is contacted with the substrate, cancer cells form a set of interactions with ECM components in the collection sufficient to isolate the growth of the cancer cells of a particular stage. In some embodiments, the growth comprises proliferation. In some embodiments, the growth is sufficient to overpopulate the sample with the cancer cells of a particular stage as compared with other cell types. In some embodiments, wherein the cancer cells at a particular stage of metastasis are from a primary tumor, lymph nodes, or metastases at organ sites or are non-small cell lung cancer cells. In some embodiments, the kit further comprises cell media.

In some embodiments, the kit further comprises a means for assessing abundance of the cancer cells of a particular stage. In some embodiments, the cancer cells at a particular stage of metastasis are breast cancer cells.

The invention further provides a kit for cancer stage diagnosis comprising: a substrate coated with a collection of ECM components characterized in that, when a sample containing cells of a plurality of different cell types, which plurality of different cell types includes at least one cell type of interest is contacted with the substrate, cells of the cell type of interest form a set of interactions with ECM components in the collection sufficient to isolate the cells of the cell type of interest from other cells in the sample, wherein the collection of extracellular matrix components comprises at least two ECM components selected from agrin and collagen IV, agrin and fibrin, biglycan and collagen II, biglycan and fibrin, collagen I and thrombospondin-4, collagen II and decorin, collagen II and tenascin-C, collagen II and testican 2, collagen III and collagen VI, collagen III and thrombospondin-4, collagen IV and galectin 4, collagen IV and SPARC, collagen IV and vitronectin, collagen V and galectin 1, collagen VI and galectin 3, fibrin and galectin 3c, fibrin and galectin 4, fibrin and keratin, fibrin and osteopontin, fibrin and SPARC, f-spondin and fibronectin, fibronectin and galectin 3, fibronectin and galectin 8, fibronectin and laminin, and/or fibronectin and testican 1.

In some embodiments, the collection of extracellular matrix components comprises at least two ECM components selected from agrin and collagen II, agrin and laminin, biglycan and collagen II, brevican and fibronectin, collagen I and testican 2, collagen II and collagen IV, collagen II and laminin, collagen II and nidogen-1, collagen II and testican 2, collagen III and galectin 8, collagen III and superfibronectin, collagen V and fibronectin, collagen V and galectin 1, collagen VI and fibronectin, collagen VI and nidogen-1, collagen VI and tenascin-C, decorin and fibronectin, decorin and galectin 8, decorin and laminin, elastin and galectin 4, fibrin and galectin 3, fibronectin and galectin 1, fibronectin and galectin 3, fibronectin and galectin 4, fibronectin and mucin, fibronectin and SPARC, fibronectin and testican 2, galectin 1 and galectin 3, galectin 1 and keratin, galectin 3 and heparan sulfate, galectin 3 and superfibronectin, galectin 4 and nidogen-1, galectin 8 and tenascin-C, keratin and laminin, laminin and merosin, laminin and thrombospondin-4, SPARC and superfibronectin, and/or superfibronectin and testican 1.

### BRIEF DESCRIPTION OF THE DRAWINGS

FIGS. 1A-1E illustrate an extracellular matrix microarray platform. (1A) An exemplary embodiment of the present invention, in which ECM arrays are generated by spotting nearly 800 different combinations of ECM components on glass slides coated with polyacrylamide followed by seeding of cells onto the slides. (1B) Polyacrylamide acts to entrap molecules of a large range of molecular weights. (1C) Verification of measurement of fluorescence as protein is captured with increasing molecular weight. (1D) Verification of presentation of all molecules by immunolabeling or NHS-fluorescein labeling. (1E) Representative images of cells adhered to ECM spots demonstrating selective adhesion in the locations of ECM stained on the left panel for phase of cell cycle and cell count by nuclear staining.

FIGS. 2A through 2C demonstrate exemplary ECM arrays identifying key adhesive changes in metastatic progression. (2A) Unsupervised hierarchical clustering of exemplary adhesion profiles generated using ECM arrays. In FIGS. 2B and 2C tumor adhesion is plotted as a function of tumor progression. Vertical axis represents different ECM component combinations shown. Horizontal axis represents different cell lines. Light grey bars indicate primary tumors. Dark grey bars indicate nodal or distant metastases. Adhesion of tumor cell lines from each of four stages of development to individual ECM components; the cell adhesion appears magnified in FIG. 2B left panel), and further magnified in 2B (or as depicted in right panel). FIG. 2C depicts all combinations of ECM components with one polypeptide of the adhesion set represented on the y axis of the slide, and a second polypeptide of the adhesion set on the x axis of the slide. Combinations with greatest increase or decrease in adhesion across tumor progression (determined by linear regression) are shown in FIG. 2C (as depicted in three panels on right-hand side). FIG. 2C (as depicted 2Bd in three panels on right-hand side) depicts average adhesion of metastatic cell lines to each combination compared to those of metastatic primary tumor cell lines.

FIG. 3 illustrates adhesion signatures from mouse lung adenocarcinoma cells from cell samples that are derived from a primary lineage and those cell samples noted to be of more metastatic in character. FIG. 3 A depicts ECM arrays spotted and seeded similar to the above Example 2 were then used to analyze cell lines from each of the four classes of cell lines. FIGS. 3B and 3C depict normalized adhesion values of ECM components alone (left column), in combinations (middle column), and with the top combinations of adhesion sets (right column) in terms of cell lines related to tumor progression (x-axis). Higher bars indicate a cell type with more adhesion to the ECM components listed (y-axis) versus a lower bar indicating a low level of adhesion.

FIG. 3D depicts the validation of adhesion to ECM components base dupon wild-type vs. metastatic cell lineages with the three ECm adhesion sets (light grey or open circles) demonstrating high adhesion values. FIG. 3E depicts the trend towards increased binding to fibronectin/galectin-3, fibronectin/laminin and fibronectin/galectin-8 combinations was consistent across tumor progression when we compared the average adhesion of all TnonMet, TMet, N and M cell lines.

FIG. 4A depicts trichrome staining of lungs with extensive tumor burden revealed a significant presence of ECM deposition in the tumor-bearing lung. FIG. 4B depicts a summary of the immunohistological data is presented in FIG. 4A showing ECM component staging across primary tumor types, tumor metastased to the lymph node, and metastases migrated to distant organ sites.

FIGS. 5A, 5B, and 5C depict RNA transcription of cognate integrins as compared to adhesion signatures of various cells lines responding to the ECM component listed. FIG. 6A and FIG. 6B depict flow cytometry of integrin surface expression in 393T5 (TMet) and 393M1 (M) cell lines. FIG. 6C depicts metastasis-associated integrins in mice bearing autochthonous tumours with spontaneous metastases to the liver and lymph nodes. Scale barsare 100 μm.

FIG. 7A depicts a metastasis protein network of a lung cell. FIG. 7B depicts a protein network of a adenocarcinoma cell of a primary tumor.

FIGS. 8A and 8B depict a knockdown experiment of both the α3 and β1 subunits (Itga3 and Itgb1, respectively) using short-hairpin mediated RNA-interference. FIG. 8C depicts another knowndown experiment that relates to adhesion. FIG. 8C shows reduced adhesion to metastasis-associated molecules in vitro when compared with the control hairpin targeting the firefly luciferase gene. FIG. 8D depicts liver metasis seeding in mice treated with short hairpin mediated RNA interference of α3 integrin as compared to the control knockdown by measuring number of surface tumors on the surface of the liver of treated animals. FIG. 8E depicts liver samples of mice injected with the 393M1-shα3 cells.

FIG. 9A depicts the relative intensity of ECM components in human lung samples. FIG. 9B depicts the staining of levels of ECM components in human lung adenocarcinoma lines across malignant lymph, distant malignant, and malignant lung samples.

FIG. 10 shows adhesion profiles of wild-type mammary epithelial cells as compared to mammary epithelial cells with metastatic character. FIG. 10A depicts the adhesion profiles of wild-type mammary epithelial cells. FIG. 10A depicts the adhesion profiles of metastatic mammary epithelial cells expressing twist (having undergone EMT as defined below). FIG. 10C depicts the ECM components that exhibit the highest differential adhesion in the wild-type as compared to the metastatic mammary epithelial cells.

FIG. 11A depicts the ECM components responsible for the highest levels of metastatic mammary epithelial cell proliferation. FIG. 11B depicts the ECM components responsible for the highest levels of normal mammary epithelial cell proliferation. FIG. 11C depicts the ECM components with the greatest differential for stimulating proliferation in wild-type versus metastatic mammary epithelial cells.

FIG. 12A depicts the ECM components responsible for the stimulation of E-Cadherin (as signal that metastatic cells that undergo EMT have switched phenotypes to colony-forming metastases in distant organs). FIG. 12B depicts the top adhesion sets responsible for conversion from a mobile metastatic human epithelial cell phenotype to colony-forming metastatic human epithelial cell.

FIGS. 13A, 13B, 13C, and 13D show an exemplary ECM array identifying key adhesive changes related to cell differentiation. In FIGS. 13A, 13, B, and 13C (representing three portions of the same experimental dataset broken into three different parts), the horizontal axis represents different ECM component combinations. Vertical axis represents different cell lines. In FIGS. 13A, 13, B, and 13C depict unsupervised hierarchical clustering of adhesion profiles generated by ECM arrays during osteogenic and adipogenic differentiation of Mesenchymal Stem Cells (MSCs). In FIG. 13D, unsupervised hierarchical clustering of adhesion profiles generated by ECM arrays during hepatic differentiation of human induced Pluripotent Stem Cells (iPSCs).

FIGS. 14A and 14B show bar graphs of differentiation profiles of mouse Embryonic Stem (ES) cells towards hepatic and pancreatic lineages on the ECM array. Nuclei and differentiation marker expression on different ECM component combinations is shown.

FIGS. 15A though 15F illustrate an exemplary ECM arrays identifying key adhesive molecules for Mesenchymal Stem Cells culture and proliferation.

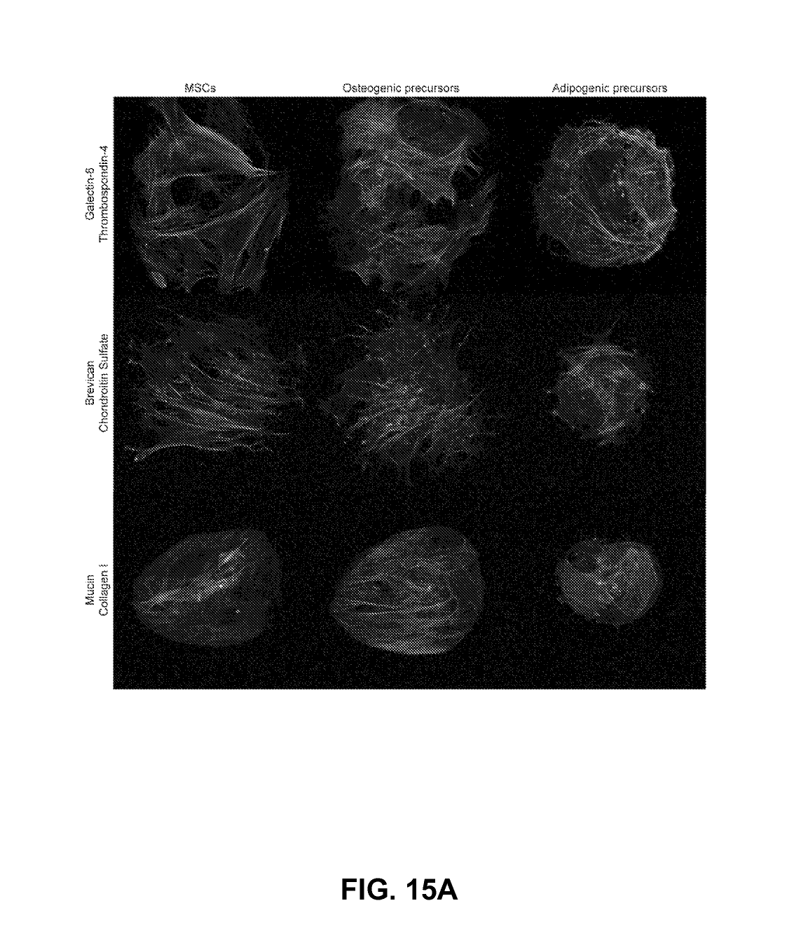

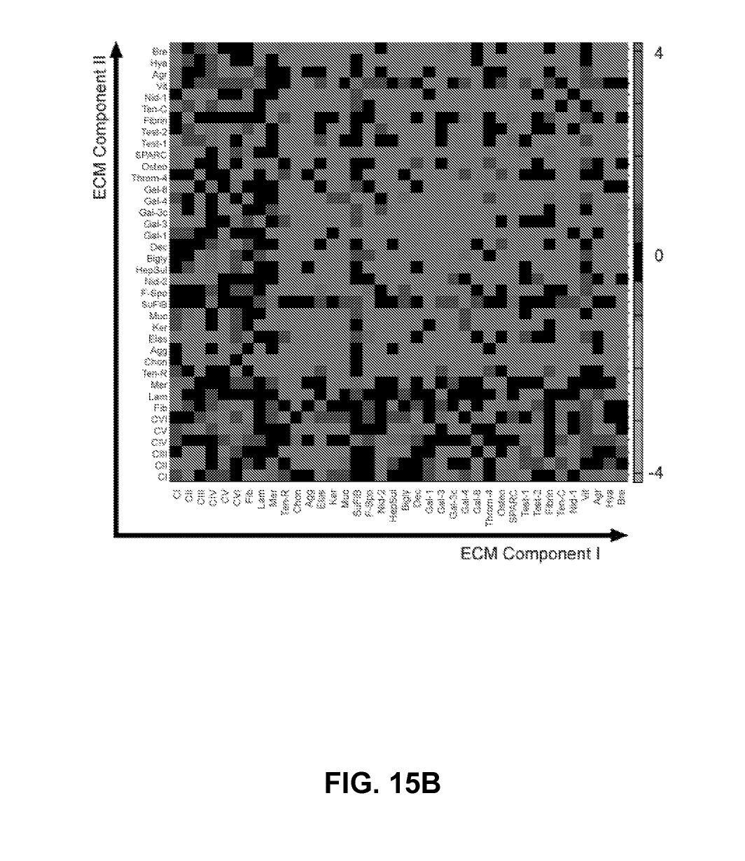

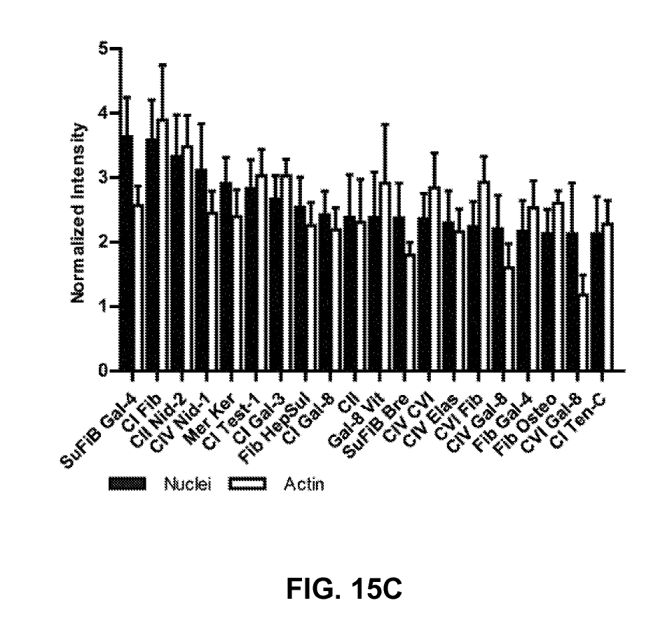

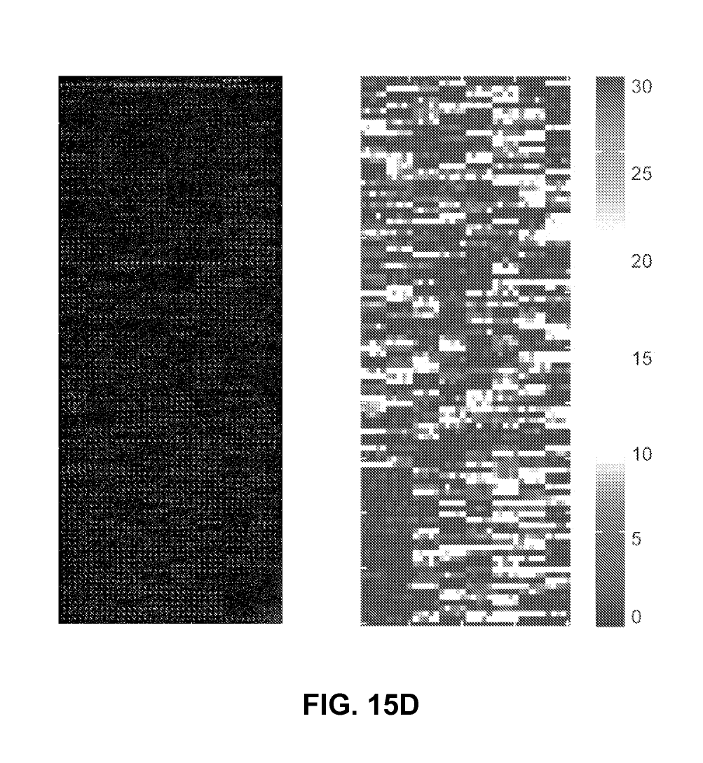

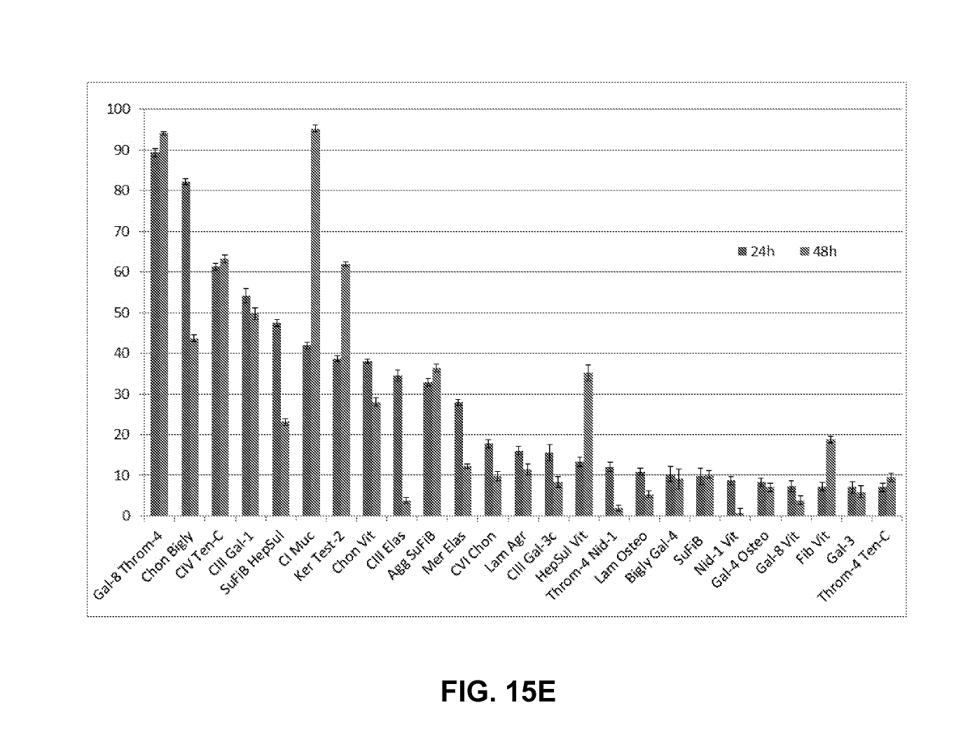

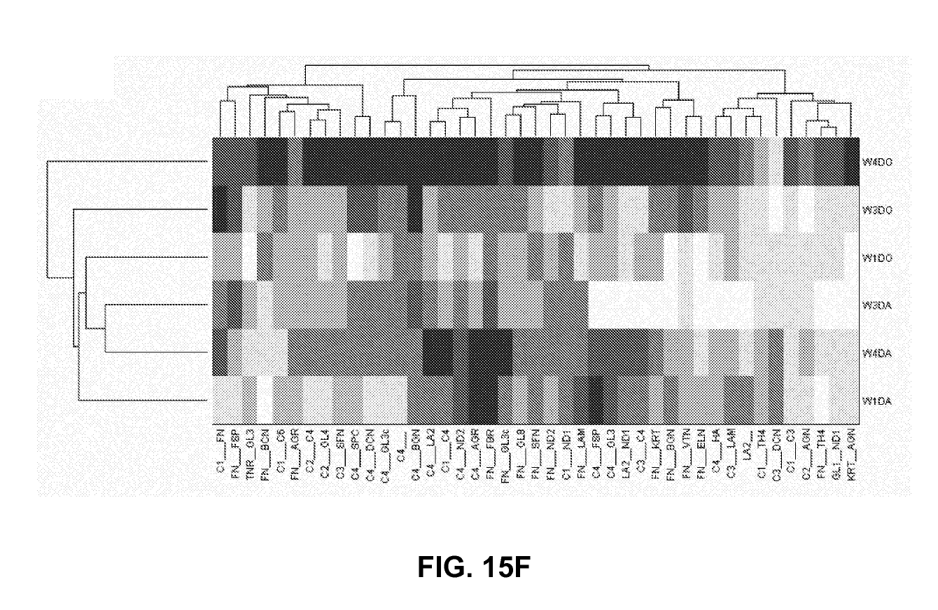

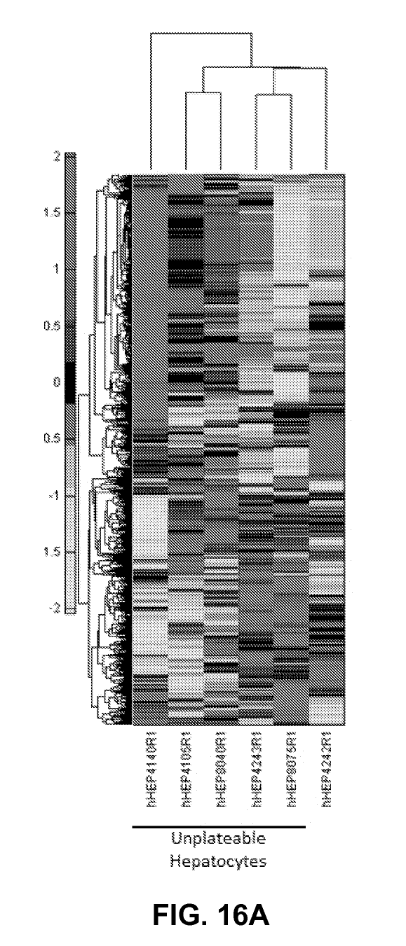

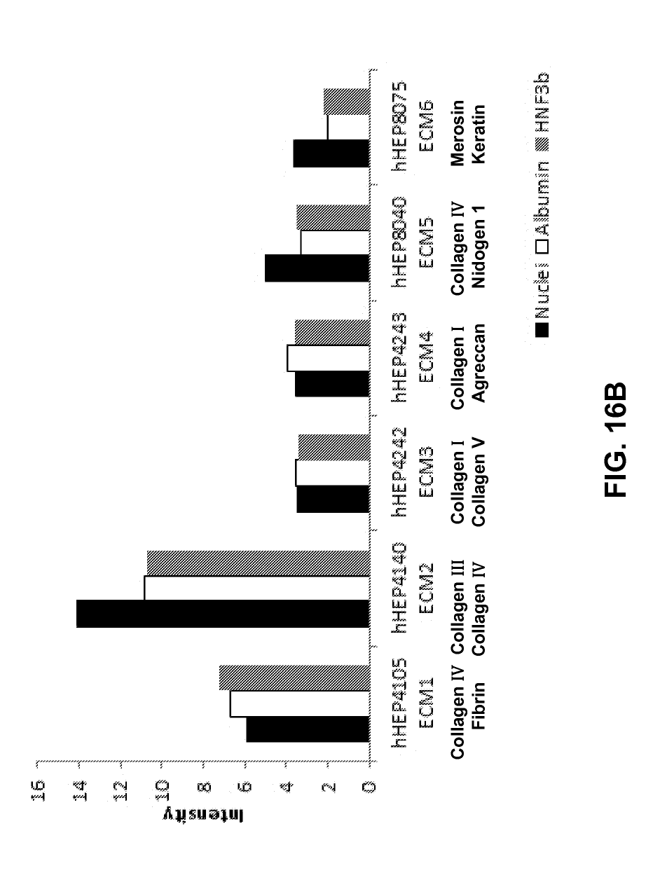

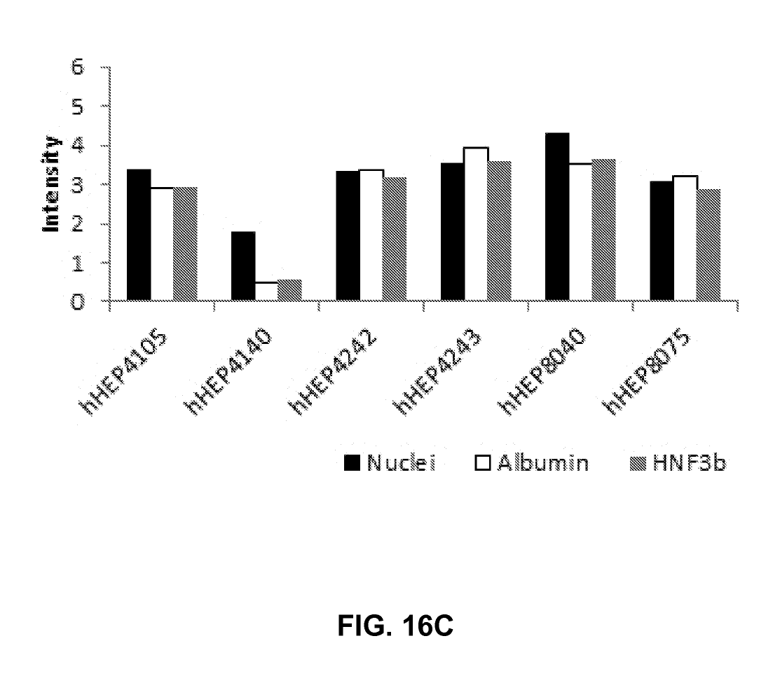

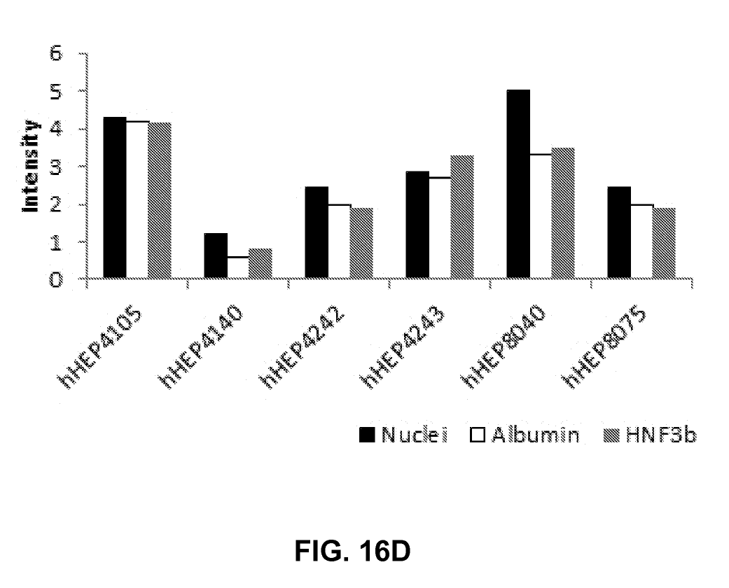

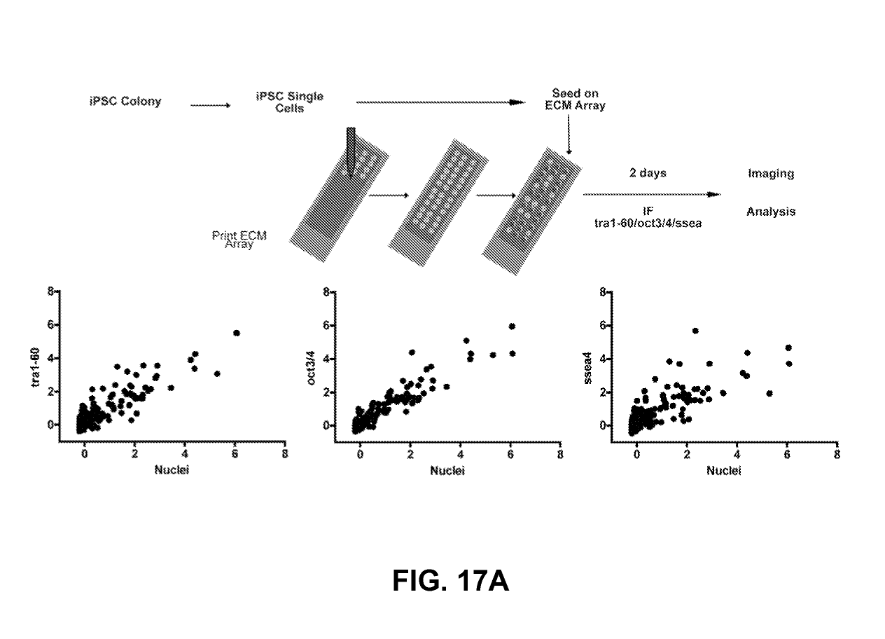

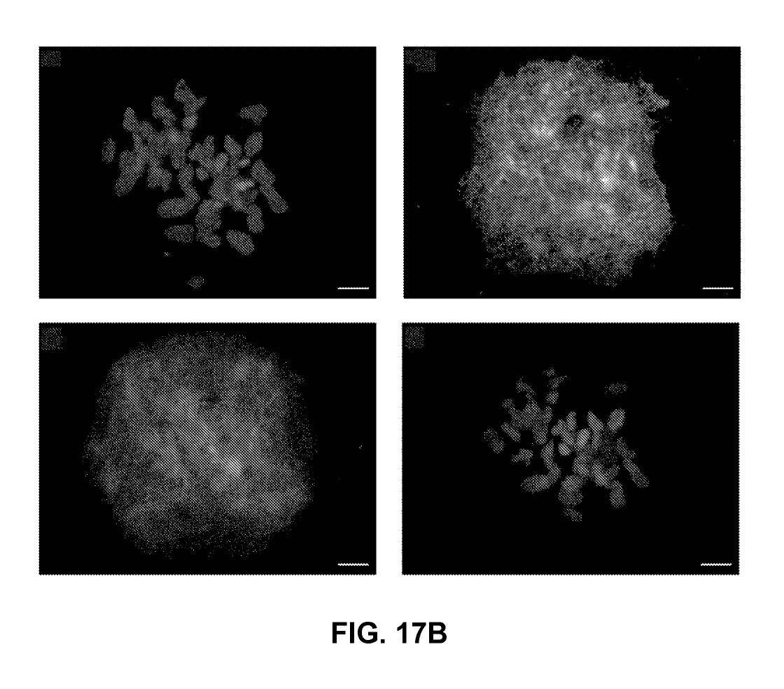

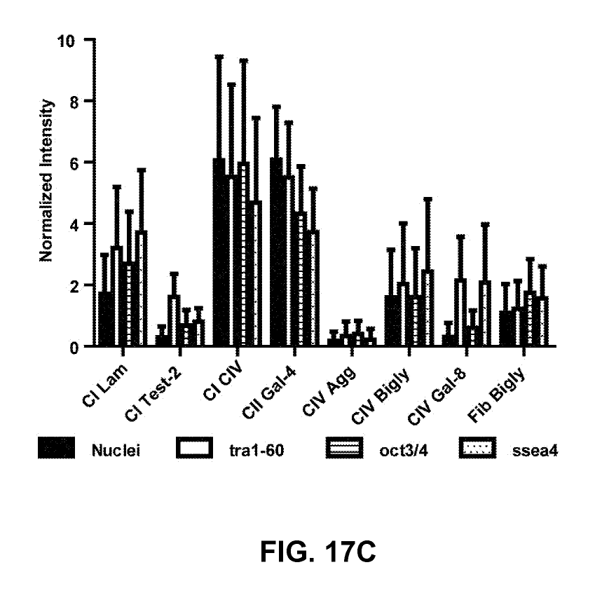

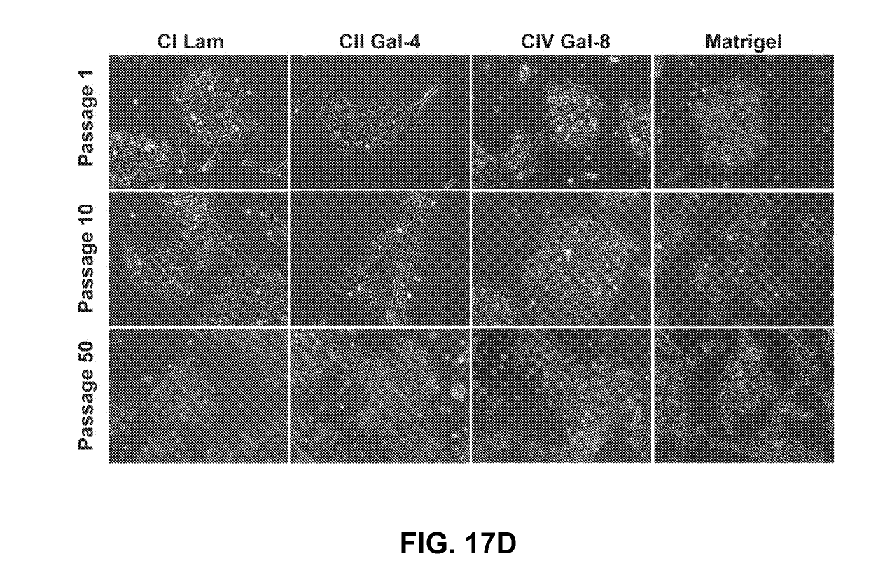

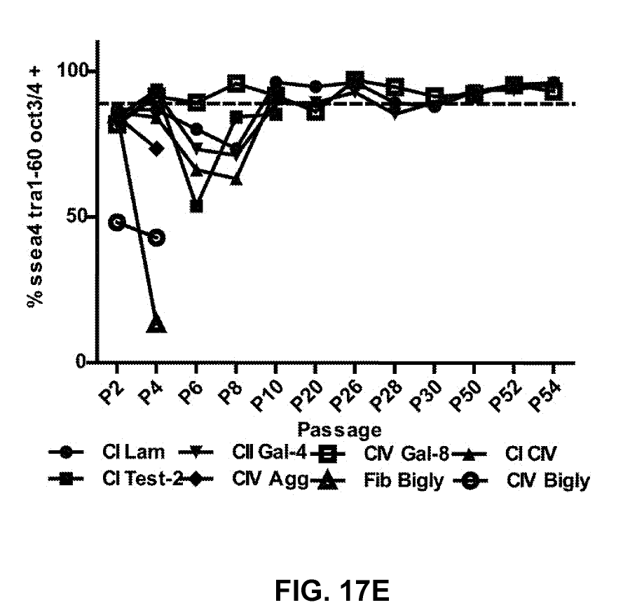

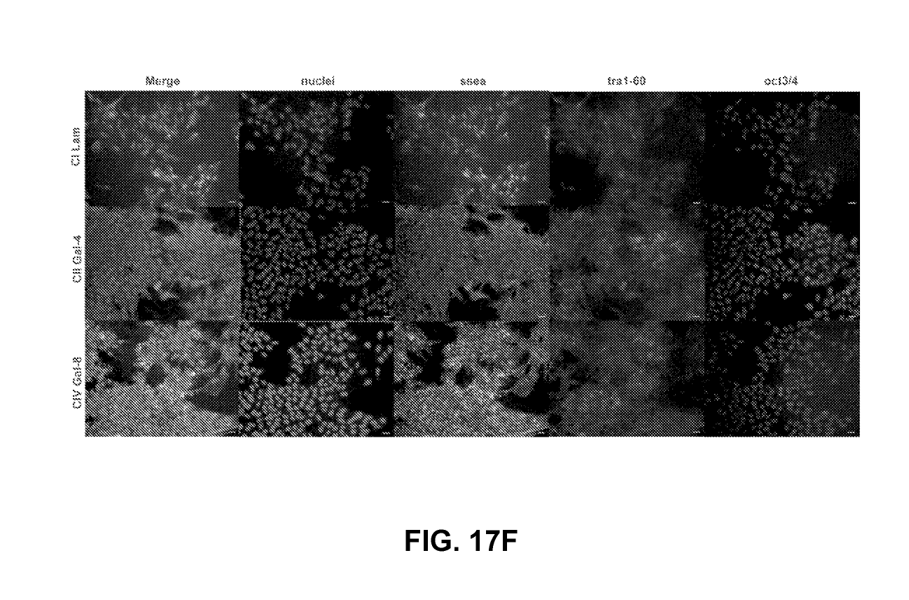

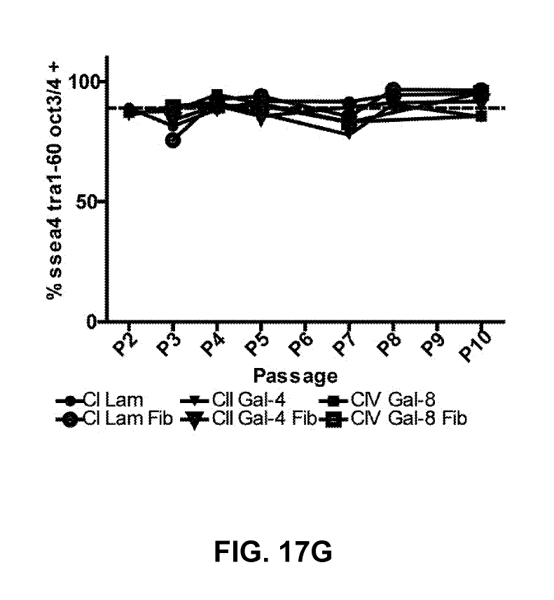

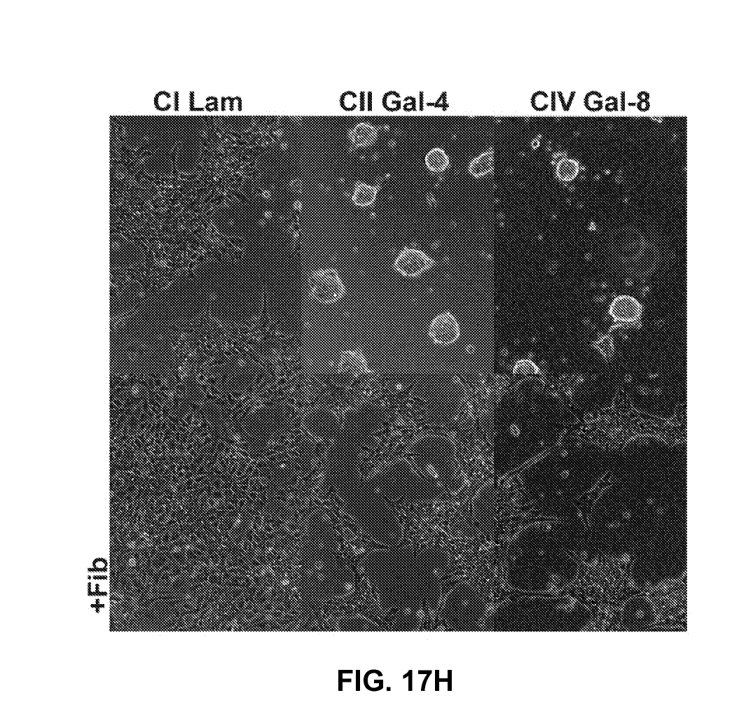

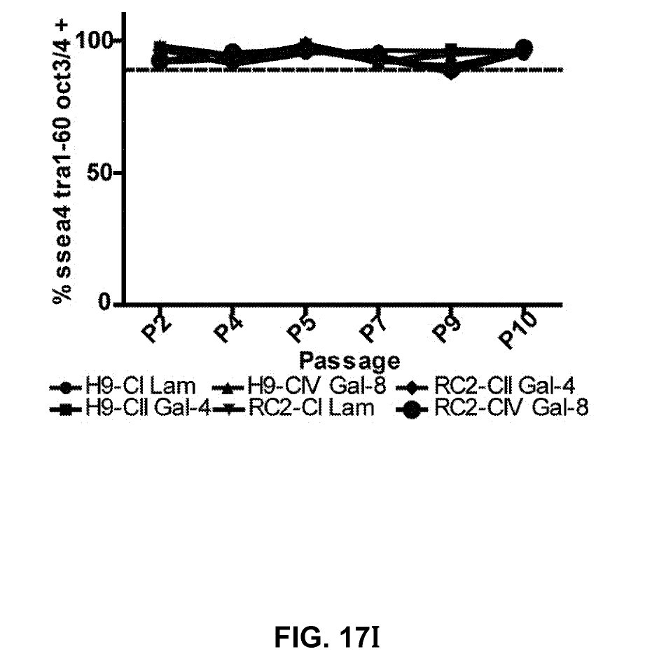

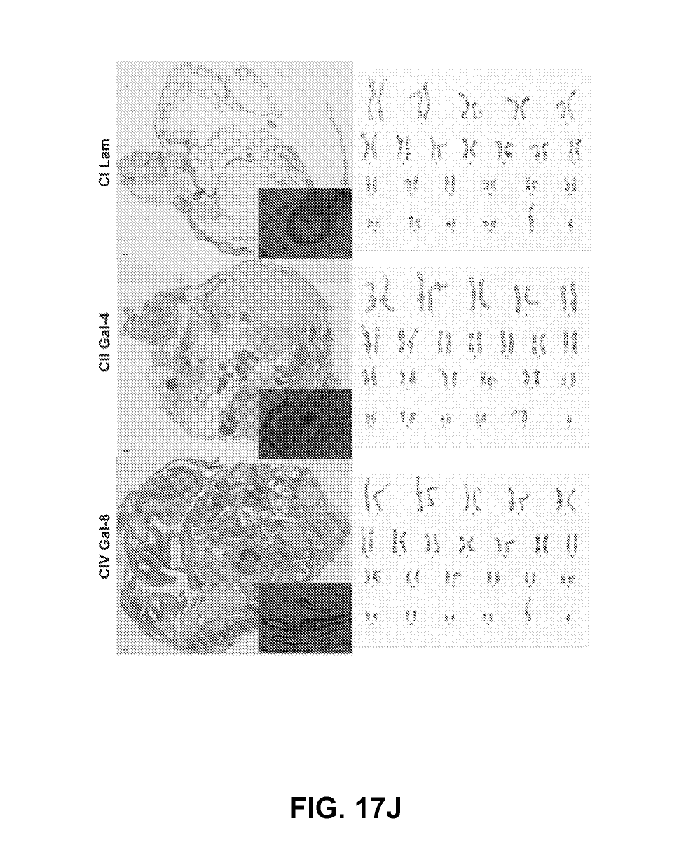

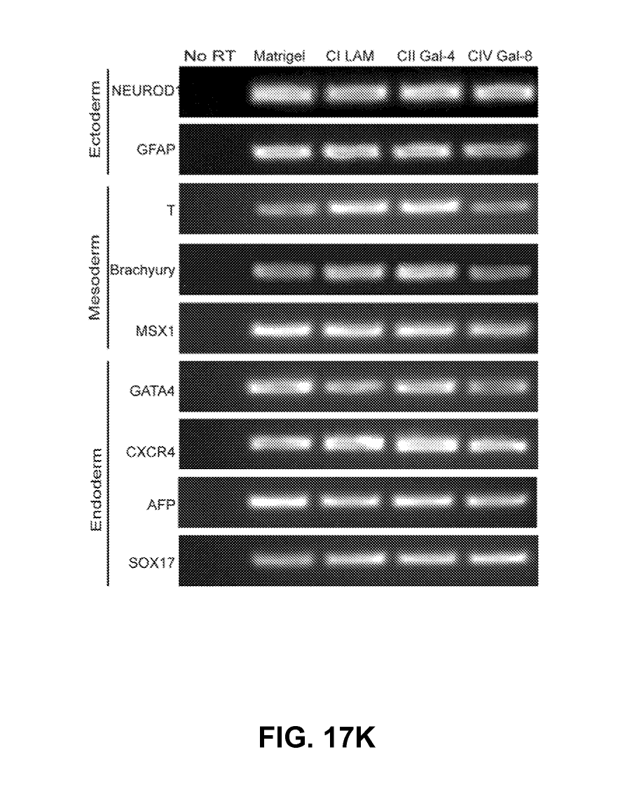

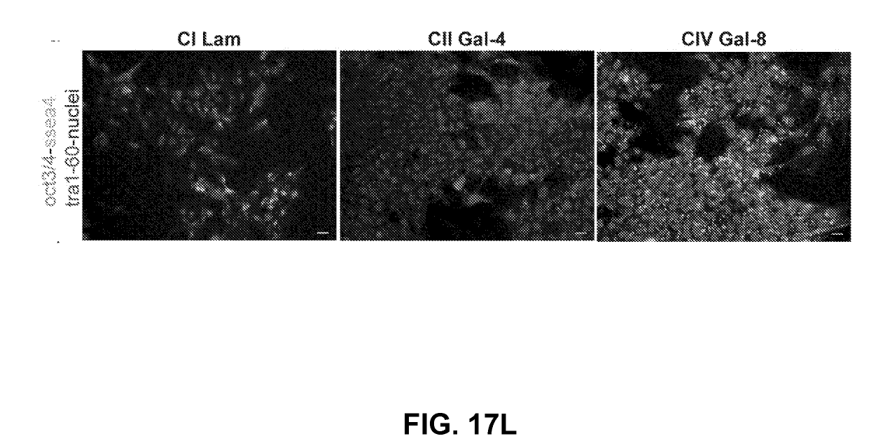

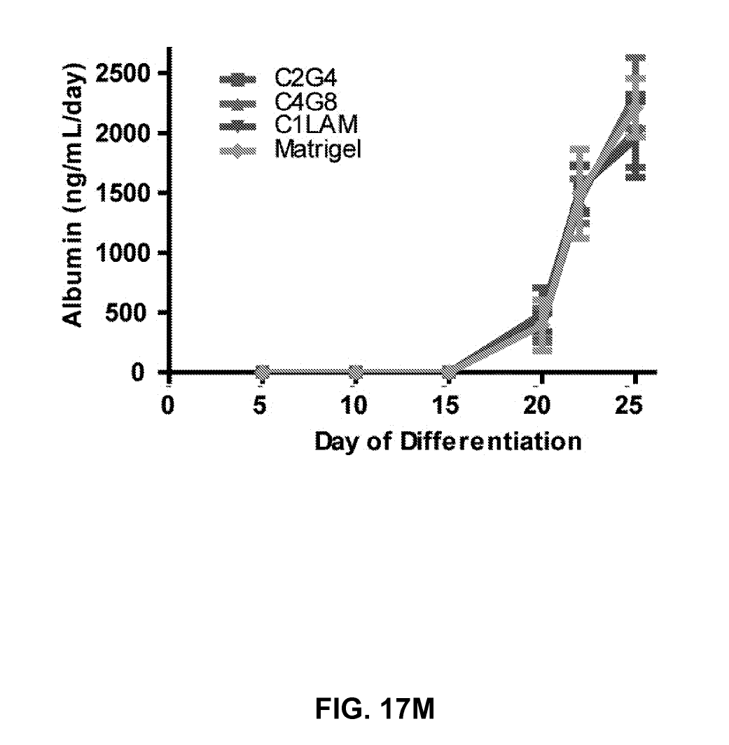

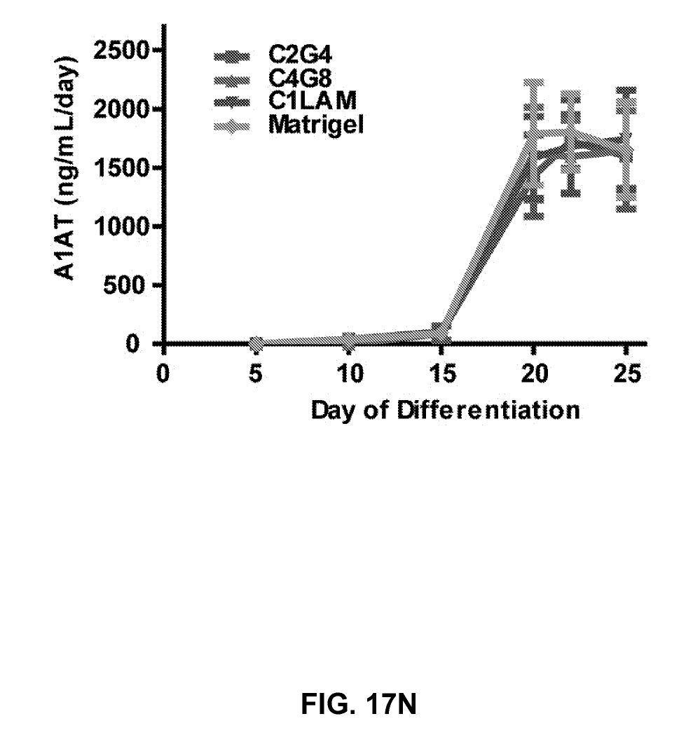

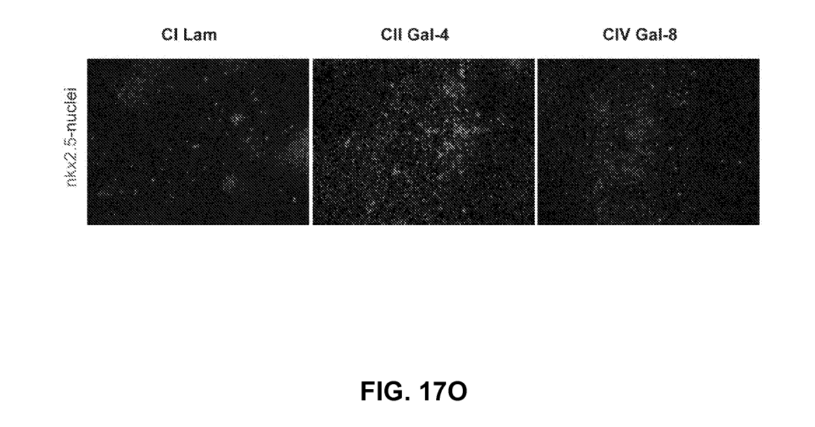

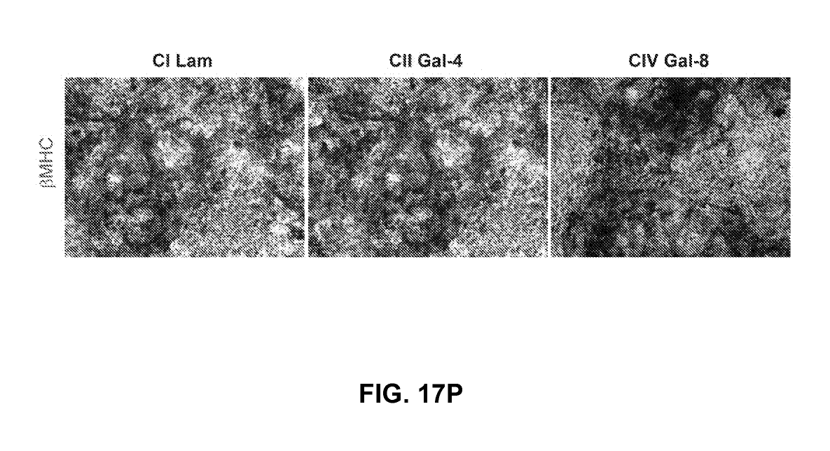

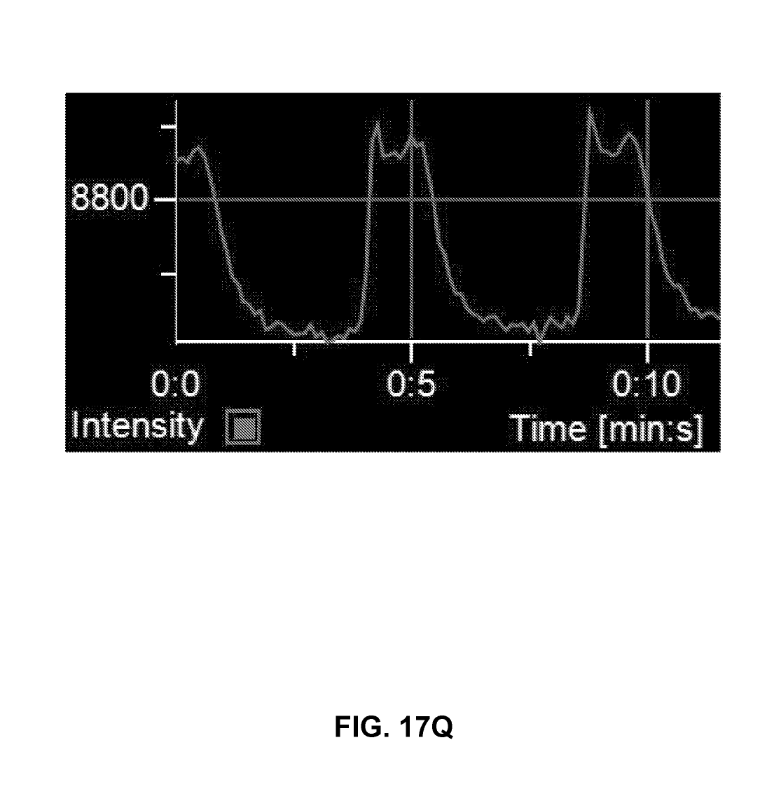

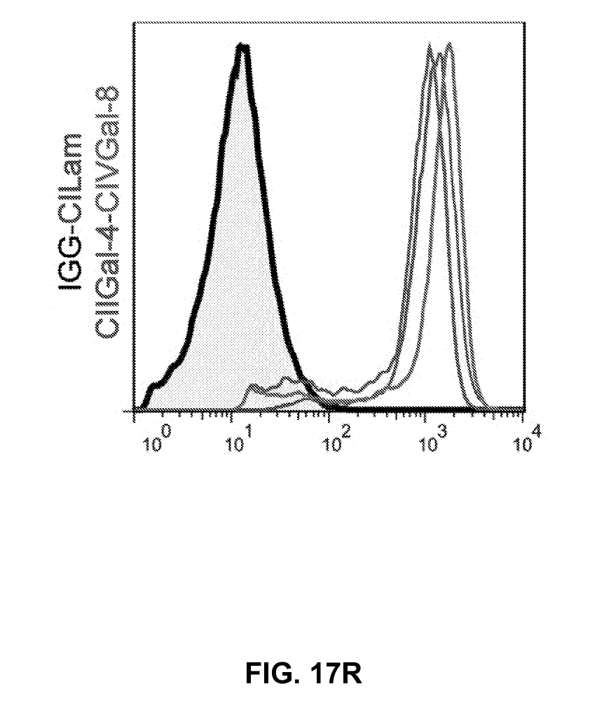

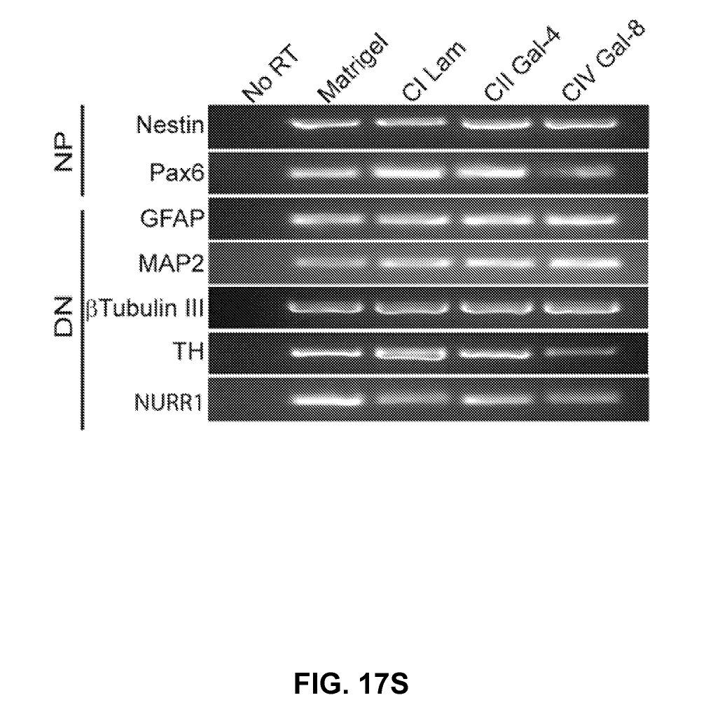

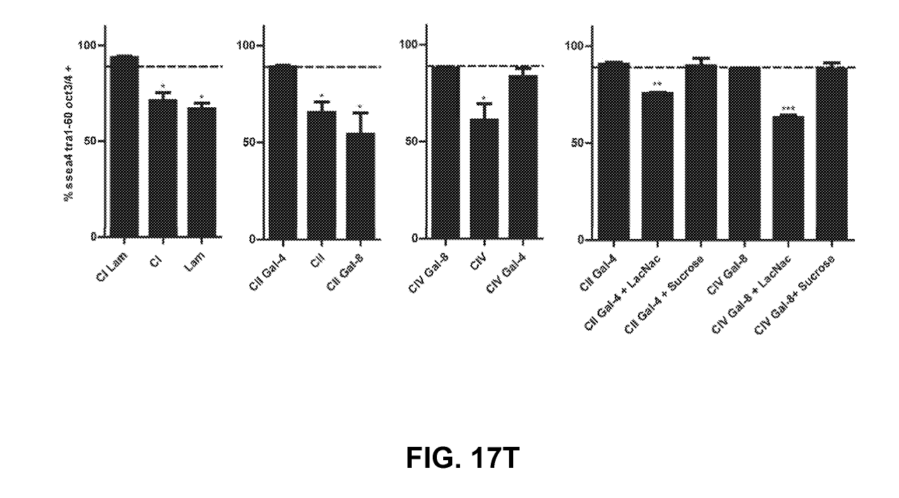

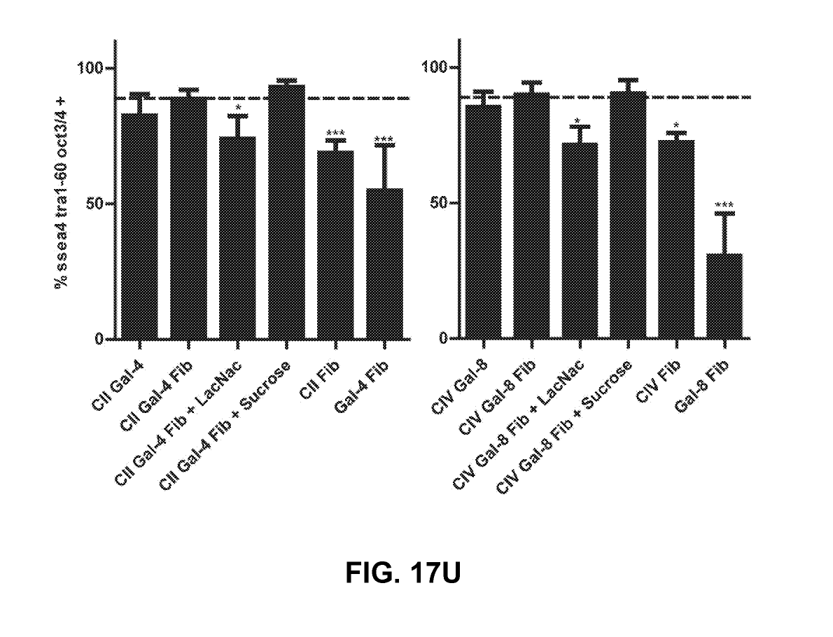

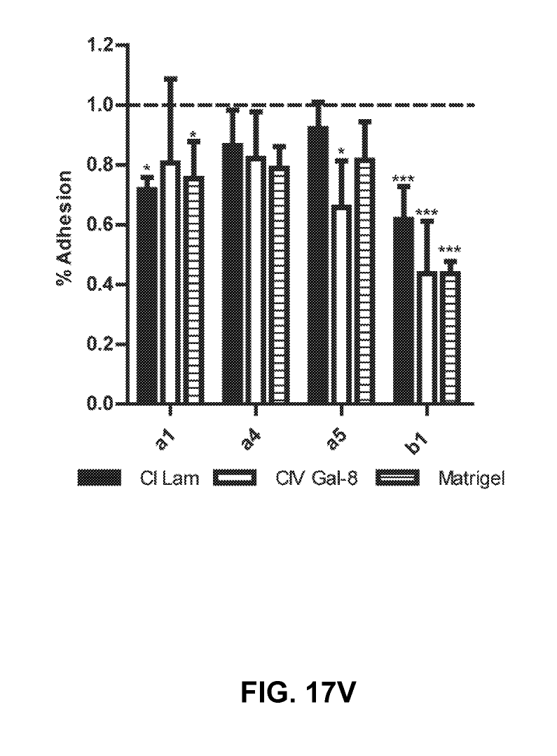

Cell isolation and expansion. FIG. 15A depicts representative ECM islands with different cell populations. MSCs or MSC-derived osteogenic and adipogenic precursors obtained by in vitro differentiation of MSCs before seeding in the array. Cells were stained for nuclei (dark grey) and actin (light grey). FIGS. 15B and 15C depict an MSC adhesion profile. FIG. 15B depicts a heatmap of MSC adhesion to an ECM array. Each axis represents ECM components and intersections are the ECM combinations present in the array. FIG. 15C depicts the top 20 adhesion combinations for MSCs. FIG. 15C shows how different ECM combinations induce different cytoskeleton organization. The left panel of-FIG. 15D shows immunofluorescence image of MSCs on ECM array after 2 days in culture. The right panel of FIG. 15D depicts an adhesion profile of MSCs on an ECM array. Heatmap represents cell number per spot. FIG. 15E depicts graphical expansion of MSCs on ECM array and depicts fold increase over day 0 for specific ECM combinations on x-axis. FIG. 15F shows a adhesion profile of MSCs during adipogenic and osteogenic differentiation of MSCs adhesion profiles of differentiating cells change over time.

FIGS. 16A through 16D illustrate an exemplary ECM arrays identifying key adhesive molecules for plating unplateable hepatocytes. (FIG. 16A) Unsupervised hierarchical clustering of adhesion profiles generated by ECM arrays for different lots of unplateable hepatocytes. Horizontal axis represents different ECM combinations. Vertical axis represents different cell lines. (FIG. 16B) Top ECM combination for each lot of unplateable hepatocytes. (16C) Collagen I and Aggrecan promote adhesion for all unplateable hepatocyte lots. (16D) Collagen IV and Nidogen-1 promote adhesion for all unplateable hepatocyte lots.

FIGS. 17A through 17V illustrate use of exemplary ECM arrays to identify key adhesive molecules for expansion, self-renewal and differentiation of human ES/iPSC cells. FIGS. 17A though 17C depict ECM component combinations promote adhesion of human ES/iPS cells and maintain expression of pluripotency markers tra1-60, ssea4 and oct3/4.

FIGS. 17D though 17I demonstrate that adsorbed ECM combinations on polystyrene plates maintain the pluripotent phenotype similar to spotted high-throughout slides and as compared to typical Matrigel culture growth. FIG. 17D depicts phase images of hIPSC cultured on selected ECM combinations over 50 passages. FIG. 17E depicts the percent of oct3/4-ssea4-tra1-60 positive hIPSC culture on ECM combinations over 50 passages. FIG. 17F depicts immunofluorescence images for oct3/4-ssea4-tra1-60 of hIPSC cultured on ECM combinations at passage 10. FIG. 17G ECM combinations support hIPSC self-renewal on defined media conditions as shown by the expression of oct3/4-ssea4-tra1-60 for at least 10 passages. FIG. 17H depicts phase images of hIPSC cells on ECM combinations in defined media conditions. FIG. 17I depicts ECM combinations support self-renewal of hESC and different hIPSC lines ECM component combinations support long term expansion of human ES/iPS cells. ECM component combinations support long term expansion of human ES/iPS cells in defined media.

FIGS. 17J and 17K hIPSC cultured on ECM combinations maintain pluripotency and multilineage differentiation potential. The left hand panel of FIG. 17J depicts how hIPSCs are able to form teratomas in vivo after being cultured for 10 passages on ECM combinations. The right hand panel of FIG. 17J depicts how the same hIPSCs maintain normal karyotype after expansion for 10 passages on ECM combinations. FIG. 17K depicts how hIPSC are able to form Embryoid Bodies and generate cells from the three germ layers after culture on ECM combinations.

FIGS. 17L through 17S depict how ECM component combinations support differentiation of human ES/iPS cells (hiPSCs) towards the hepatic lineage, cardiac, and neuronal lineages. Differentiations occurs toward multiple lineages after 10 passages on EMC combinations (FIG. 17L) hIPSC differentiate towards the hepatic lineage and produce albumin (FIG. 17M) and α1 antitrypsin (FIG. 17N); the cardiac lineage shown by the expression of nkx2.5 (FIG. 17O); and beta myosin heavy chain) (FIG. 17P) and the responsiveness to calcium signals (FIG. 17Q); and differentiation towards the neuronal lineage is confirmed by the expression of β-tubulin (FIG. 17S).

FIGS. 17T, 17U, and 17V depict Specific ECM combinations are important for the maintenance of the pluripotent phenotype. Collagen I and Laminin alone are unable to maintain pluripotency (left panel of FIG. 17T). Collagen II alone or Collagen II with Galectin-8 do not support hIPSC self-renewal (middle left panel of FIG. 17T). Collagen IV alone does not support hIPSC self-renewal (right middle panel of FIG. 17T). Blocking the galectin carbohydrate domain with LacNac induces loss of pluripotency (right panel of FIG. 17T). Specific ECM combinations are also important under defined media condition (FIG. 17U). Blocking integrin subunits induces a reduction of cell adhesion to specific ECM combinations (FIG. 17V).

### DETAILED DESCRIPTION OF THE INVENTION

Various terms relating to the methods and other aspects of the present invention are used throughout the specification and claims. Such terms are to be given their ordinary meaning in the art unless otherwise indicated. Other specifically defined terms are to be construed in a manner consistent with the definition provided herein.

As used in this specification and the appended claims, the singular forms “a,” “an,” and “the” include plural referents unless the content clearly dictates otherwise.

The term “about” as used herein when referring to a measurable value such as an amount, a temporal duration, and the like, is meant to encompass variations of ±20%, ±10%, ±5%, ±1%, or ±0.1% from the specified value, as such variations are appropriate to perform the disclosed methods.

The term “addressable location” as used herein means a discrete surface area or position on a solid support onto which one or a plurality of adhesion sets are immobilized or absorbed such that exposure of the one or plurality of adhesion sets to a sample comprising a biomaterial or cell for a sufficient time period results in contact between the cell or biomaterial and the adhesion set. In some embodiments, the invention relates to an array comprising one or a plurality of addressable locations of the array with a width or diameter of about 10 nanometers. In some embodiments, the invention relates to an array comprising one or a plurality of addressable locations of the array with a width or diameter of about 20 nanometers. In some embodiments, the invention relates to an array comprising one or a plurality of addressable locations of the array with a width or diameter of about 30 nanometers. In some embodiments, the invention relates to an array comprising one or a plurality of addressable locations of the array with a width or diameter of about 40 nanometers. In some embodiments, the invention relates to an array comprising one or a plurality of addressable locations of the array with a width or diameter of about 50 nanometers. In some embodiments, the invention relates to an array comprising one or a plurality of addressable locations of the array with a width or diameter of about 60 nanometers. In some embodiments, the invention relates to an array comprising one or a plurality of addressable locations of the array with a width or diameter of about 70 nanometers. In some embodiments, the invention relates to an array comprising one or a plurality of addressable locations of the array with a width or diameter of about 80 nanometers. In some embodiments, the invention relates to an array comprising one or a plurality of addressable locations of the array with a width or diameter of about 90 nanometers. In some embodiments, the invention relates to an array comprising one or a plurality of addressable locations of the array with a width or diameter of about 100 nanometers. In some embodiments, the one or a plurality of addressable locations of the array is no more than 250 nanometers in diameter. In some embodiments, the one or a plurality of addressable locations of the array is no more than 120 nanometers in diameter or width. In some embodiments, the one or a plurality of addressable locations of the array is no more than 80 nanometers in diameter or width. In some embodiments, the one or a plurality of addressable locations of the array is no more than 70 nanometers in diameter or width. In some embodiments, the one or plurality of addressable locations of the array is no more than 60 nanometers in diameter or width. In some embodiments, the one or plurality of addressable locations of the array is no more than 50 nanometers in diameter or width. In some embodiments, the one or plurality of addressable locations of the array is no more than 40 nanometers in diameter or width. In some embodiments, the one or plurality of addressable locations of the array is no more than 30 nanometers in diameter or width. In some embodiments, the one or plurality of addressable locations of the array is no more than 20 nanometers in diameter or width. In some embodiments, the one or plurality of addressable locations of the array is no more than 10 nanometers in diameter or width. In some embodiments, the one or plurality of addressable locations of the array is from about 10 nanometers in diameter or width to about 100 nanometers in diameter or width. In some embodiments, the one or plurality of addressable locations of the array is spotted manually by a pipet or automatically by a robotic device.

As used herein, the terms “attach,” “attachment,” “adhere,” “adhered,” “adherent,” or like terms generally refer to immobilizing or fixing, for example, a group, a compound or adhesion set, to a surface, such as by physical absorption, chemical bonding, and like processes, or combinations thereof.

The term “adhesion set” or “adhesion sets” as used herein means at least two polypeptides comprising a protein or functional fragment of a protein that are covalently or non-covalently immobilized to a surface at a discrete, addressable location. In some embodiments, the adhesion set comprises a pair of polypeptides or functional fragments thereof. In some embodiments, the adhesion set comprises a plurality of polypeptides or functional fragments thereof covalently or non-covalently bound to a surface at a discrete location. In some embodiments, the adhesion set comprises three or more of polypeptides or functional fragments thereof covalently or non-covalently bound to a surface at a discrete location.

As used herein, the terms “animal-derived ECM material” mean any macromolecule component of an extracellular matrix or biomaterial derived therefrom, including a protein, polysaccharide, polypeptide modified with a polysaccharide, or group of the same that is produced by, originated from, or sourced from an animal species, including a human.

The terms “adhesion value” as used herein means a single quantitative value that can be used as a criterion for whether a particular cell or cell sample expresses or does not express a particular quantity of protein such that, when normalized against a quantitative value calculated for a control tissue, the adhesion value can be used in a predictive model for the diagnosis, prognosis, or clinical treatment plan of a subject. In some embodiments, the adhesion value means a single quantitative value that can be used as a criterion for how tightly or how readily a particular cell or cell sample does or does not associate (or bind) to a particular quantity of protein such that, when normalized against a calculated quantitative value for a reference or control sample, the adhesion value can be used in a predictive model for the diagnosis, prognosis, or clinical treatment plan of a subject. In some embodiments, the quantitative value is calculated by combining quantitative data regarding the association of a cell or cell sample to one or a plurality of adhesion sets through an interpretation function or algorithm described herein. In some embodiments, the subject is suspected of having, is at risk of developing, or has been diagnosed with a metastatic cancer. In some embodiments, the subject is suspected of having, is at risk of developing, or has been diagnosed with a metastatic lung or metastatic breast cancer.

As used herein, the terms “biopsy” means a cell sample, collection of cells, or tissue removed from a subject or patient for analysis. In some embodiments, the biopsy is a bone marrow biopsy, punch biopsy, endoscopic biopsy, needle biopsy, shave biopsy, incisional biopsy, excisional biopsy, or surgical resection.

As used herein the terms “electronic medium” mean any physical storage employing electronic technology for access, including a hard disk, ROM, EEPROM, RAM, flash memory, nonvolatile memory, or any substantially and functionally equivalent medium. In some embodiments, the software storage may be co-located with the processor implementing an embodiment of the invention, or at least a portion of the software storage may be remotely located but accessible when needed.

As used herein, the term “hyperproliferative diseases” is meant to refer to those diseases and disorders characterized by hyperproliferation of cells. Examples of hyperproliferative diseases include all forms of cancer, psoriasis, neoplasia, and hyperplasia.

As used herein, “sequence identity” is determined by using the stand-alone executable BLAST engine program for blasting two sequences (bl2seq), which can be retrieved from the National Center for Biotechnology Information (NCBI) ftp site, using the default parameters (Tatusova and Madden, FEMS Microbiol Lett., 1999, 174, 247-250; which is incorporated herein by reference in its entirety).

The term “subject” is used throughout the specification to describe an animal from which a cell sample is taken. In some embodiment, the animal is a human. For diagnosis of those conditions which are specific for a specific subject, such as a human being, the term “patient” may be interchangeably used. In some instances in the description of the present invention, the term “patient” will refer to human patients suffering from a particular disease or disorder. In some embodiments, the subject may be a human suspected of having or being identified as at risk to develop a hyperproliferative disease. In some embodiments, the subject may be diagnosed as having malignant cancer and of having or being identified as at risk to develop a metastatic hyperproliferative disease. In some embodiments, the subject is suspected of having or has been diagnosed with breast cancer or lung cancer. In some embodiments, the subject may be a human suspected of having or being identified as at risk to develop lung cancer or breast cancer. In some embodiments, the subject may be a mammal which functions as a source of the isolated cell sample. In some embodiments, the subject may be a non-human animal from which a cell sample is isolated or provided. The term “mammal” encompasses both humans and non-humans and includes but is not limited to humans, non-human primates, canines, felines, murines, bovines, equines, and porcines.

As used herein, “conservative” amino acid substitutions may be defined as set out in Tables A, B, or C below. Hyperactive transposases include those wherein conservative substitutions have been introduced by modification of polynucleotides encoding polypeptides of the invention. Amino acids can be classified according to physical properties and contribution to secondary and tertiary protein structure. A conservative substitution is recognized in the art as a substitution of one amino acid for another amino acid that has similar properties. Exemplary conservative substitutions are set out in Table A.

TABLE A
Conservative Substitutions I

| Side Chain | |
|---|---|
| Characteristics | Amino Acid |
| Aliphatic | |
| Non-polar | G A P I L V F |
| Polar - uncharged | C S T M N Q |
| Polar - charged | D E K R |
| Aromatic | H F W Y |
| Other | N Q D E |

Alternately, conservative amino acids can be grouped as described in Lehninger, (Biochemistry, Second Edition; Worth Publishers, Inc. NY, N.Y. (1975), pp. 71-77) as set forth in Table B.

TABLE B
Conservative Substitutions II

| Side Chain | |
|---|---|
| Characteristic | Amino Acid |
| Non-polar (hydrophobic) | |
| Aliphatic: | A L I V P. |
| Aromatic: | F W Y |
| Sulfur-containing: | M |
| Borderline: | G Y |
| Uncharged-polar | |
| Hydroxyl: | S T Y |
| Amides: | N Q |
| Sulfhydryl: | C |
| Borderline: | G Y |
| Positively Charged (Basic): | K R H |
| Negatively Charged (Acidic): | D E |

Alternately, exemplary conservative substitutions are set out in Table C.

TABLE C
Conservative Substitutions III

| Original Residue | Exemplary Substitution |
|---|---|
| Ala (A) | Val Leu Ile Met |
| Arg (R) | Lys His |
| Asn (N) | Gln |
| Asp (D) | Glu |
| Cys (C) | Ser Thr |
| Gln (Q) | Asn |
| Glu (E) | Asp |
| Gly (G) | Ala Val Leu Pro |
| His (H) | Lys Arg |
| Ile (I) | Leu Val Met Ala Phe |
| Leu (L) | Ile Val Met Ala Phe |
| Lys (K) | Arg His |
| Met (M) | Leu Ile Val Ala |
| Phe (F) | Trp Tyr Ile |
| Pro (P) | Gly Ala Val Leu Ile |
| Ser (S) | Thr |
| Thr (T) | Ser |
| Trp (W) | Tyr Phe Ile |
| Tyr (Y) | Trp Phe Thr Ser |
| Val (V) | Ile Leu Met Ala |

It should be understood that the polypeptides comprising polypeptide sequences associated with the extracellular matrix described herein are intended to include polypeptides bearing one or more insertions, deletions, or substitutions, or any combination thereof, of amino acid residues as well as modifications other than insertions, deletions, or substitutions of amino acid residues.

As used herein, the term “prognosing” means determining the probable course and outcome of a disease.

As used herein, the term “functional fragment” means any portion of a polypeptide that is of a sufficient length to retain at least partial biological function that is similar to or substantially similar to the wild-type polypeptide upon which the fragment is based. In some embodiments, a functional fragment of a polypeptide associated with the extracellular matrix is a polypeptide that comprises 80, 85, 90, 95, 96, 97, 98, or 99% sequence identity of any polypeptide disclosed in Table 1 and has sufficient length to retain at least partial binding affinity to one or a plurality of ligands that bind to the polypeptide in Table 1. In some embodiments, the fragment is a fragment of any polypeptide disclosed in Table 1 and has a length of at least about 10, about 20, about 30, about 40, about 50, about 60, about 70, about 80, about 90, or about 100 contiguous amino acids. In some embodiments, the fragment is a fragment of any polypeptide disclosed in Table 1 and has a length of at least about 50 amino acids. In some embodiments, the fragment is a fragment of any polypeptide disclosed in Table 1 and has a length of at least about 100 amino acids. In some embodiments, the fragment is a fragment of any polypeptide disclosed in Table I and has a length of at least about 150 amino acids. In some embodiments, the fragment is a fragment of any polypeptide disclosed in Table 1 and has a length of at least about 200 amino acids. In some embodiments, the fragment is a fragment of any polypeptide disclosed in Table I and has a length of at least about 250 amino acids. In some embodiments, the fragment is a fragment of any polypeptide disclosed in Table 1 and has a length of at least about 300 amino acids. In some embodiments, the fragment is a fragment of any polypeptide disclosed in Table 1 and has a length of at least about 350 amino acids. In some embodiments, the fragment is a fragment of any polypeptide disclosed in Table 1 and has a length of at least about 400 amino acids. In some embodiments, the fragment is a fragment of any polypeptide disclosed in Table 1 and has a length of at least about 450 amino acids. In some embodiments, the fragment is a fragment of any polypeptide disclosed in Table 1 and has a length of at least about 500 amino acids. In some embodiments, the fragment is a fragment of any polypeptide disclosed in Table 1 and has a length of at least about 550 amino acids. In some embodiments, the fragment is a fragment of any polypeptide disclosed in Table 1 and has a length of at least about 600 amino acids. In some embodiments, the fragment is a fragment of any polypeptide disclosed in Table 1 and has a length of at least about 650 amino acids. In some embodiments, the fragment is a fragment of any polypeptide disclosed in Table 1 and has a length of at least about 700 amino acids. In some embodiments, the fragment is a fragment of any polypeptide disclosed in Table 1 and has a length of at least about 750 amino acids. In some embodiments, the fragment is a fragment of any polypeptide disclosed in Table 1 and has a length of at least about 800 amino acids. In some embodiments, the fragment is a fragment of any polypeptide disclosed in Table 1 and has a length of at least about 850 amino acids. In some embodiments, the fragment is a fragment of any polypeptide disclosed in Table 1 and has a length of at least about 900 amino acids. In some embodiments, the fragment is a fragment of any polypeptide disclosed in Table 1 and has a length of at least about 950 amino acids. In some embodiments, the fragment is a fragment of any polypeptide disclosed in Table 1 and has a length of at least about 1000 amino acids. In some embodiments, the fragment is a fragment of any polypeptide disclosed in Table 1 and has a length of at least about 1050 amino acids. In some embodiments, the fragment is a fragment of any polypeptide disclosed in Table 1 and has a length of at least about 1250 amino acids. In some embodiments, the fragment is a fragment of any polypeptide disclosed in Table 1 and has a length of at least about 1500 amino acids. In some embodiments, the fragment is a fragment of any polypeptide disclosed in Table 1 and has a length of at least about 1750 amino acids. In some embodiments, the fragment is a fragment of any polypeptide disclosed in Table 1 and has a length of at least about 2000 amino acids. In some embodiments, the fragment is a fragment of any polypeptide disclosed in Table 1 and has a length of at least about 2250 amino acids. In some embodiments, the fragment is a fragment of any polypeptide disclosed in Table 1 and has a length of at least about 2500 amino acids. In some embodiments, the fragment is a fragment of any polypeptide disclosed in Table 1 and has a length of at least about 2750 amino acids. In some embodiments, the fragment is a fragment of any polypeptide disclosed in Table 1 and has a length of at least about 3000 amino acids.

As used herein, the terms “polypeptide sequence associated with the extracellular matrix” means any polypeptide or fragment thereof, modified or unmodified by any macromolecule (such as a sugar molecule or macromolecule), that is produced naturally by cells in any multicellular organism and is an ECM component or whose structure is based upon an ECM component. In some embodiments, a polypeptide sequence associated with the extracellular matrix is any polypeptide that polypeptide sequence comprising any of the polypeptides disclosed in Table 1. In some embodiments, a polypeptide sequence associated with the extracellular matrix is any polypeptide sequence comprising any of the polypeptides disclosed in Table 1 or a sequence that shares 85, 90, 95, 96, 97, 98, or 99% sequence identity with the polypeptides disclosed in Table 1 or a functional fragment thereof. In some embodiments, a polypeptide sequence associated with the extracellular matrix consists of any of the polypeptides disclosed in Table 1 or a sequence that shares 85, 90, 95, 96, 97, 98, or 99% sequence identity with the polypeptides disclosed in Table 1.

As used herein, the terms “xeno-free” media mean cell culture media free of animal serum or animal-derived components or macromolecules, except those proteins or other macromolecules derived and/or isolated from human tissue or human samples. In some embodiments, the arrays, the systems, kits or the composition described herein comprise xeno-free media. In some embodiments, the methods described herein comprise a step of culturing or contacting cells (such as stem cells) in the presence of xeno-free media. In some embodiments, the array or system does not comprise animal-derived ECM material. In some embodiments, the array or system or kit comprises xeno-free media. In some embodiments, the array or system or kit comprises media free of animal-derived components. In some embodiments, the system or array is free of any macromolecule derived from an animal, except a human. In some embodiments, the system or array is free of any macromolecule derived from an animal.

As used herein, the terms “media free of animal-derived components” mean any cell media that is free of any macromolecule component of an extracellular matrix or biomaterial derived therefrom, including a protein, polysaccharide, polypeptide modified with a polysaccharide, or group of the same that is produced by, originated from, or sourced from an animal species, including a human. In some embodiments, media free of animal-derived components comprises vegetable-derived components. In some embodiments, the media free of animal-derived components comprises only synthetic ECM components. In some embodiments, media free of animal-derived components does not comprise vegetable-derived components or macromolecules. In some embodiments, media free of animal-derived components does not comprise any human-derived ECM material or components. In some embodiments, the arrays, the systems, kits or the composition described herein comprise media free of animal-derived components. In some embodiments, the methods described herein comprise a step of culturing or contacting cells (such as stem cells) in the presence of media free of animal-derived components.

Adhesion signature: An “adhesion signature”, as that term is used herein, refers to a set of ECM binding affinity values (or range(s) of values) sufficient to characterize or distinguish a particular cell or cell type of interest from one or more different cells or cell types. In some embodiments, an adhesion signature includes a binding affinity value or range for at least one ECM component; in some embodiments, an adhesion signature includes binding affinity values or ranges for at least 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40 or more different ECM components and/combinations thereof. In some embodiments, an adhesion signature is a collection of data collected by a user of an array or system disclosed herein related to the quantity, intensity, presence, absence of cellular binding of a cell in a cell sample to one or more adhesion sets relative to the quantity, intensity, presence, absence of binding of a reference, or control, cell or reference cell sample. In some embodiments, the adhesion signature is a collection of data collected by a user of an array or system disclosed herein related to the quantity or proportion of cells that bind one or more adhesion sets as compared to the quantity or proportion of reference cells or control cells that bind the same one or more adhesion sets. In some embodiments, adhesion values are quantified by measuring the number of cells bound to one or more adhesion sets through fluorescent microscopy after staining the cells in a cell sample with fluorescent dye or other fluorescent marker.

“Cell type” means the organism, organ, and/or tissue type from which the cell is derived or sourced, state of development, phenotype or any other categorization of a particular cell that appropriately forms the basis for defining it as “similar to” or “different from” another cell or cells.

Affinity: As is known in the art, “affinity” is a measure of the tightness with which a particular ligand binds to (e.g., associates non-covalently with) and/or the rate or frequency with which it dissociates from, its partner. As is known in the art, any of a variety of technologies can be utilized to determine affinity. In many embodiments, affinity represents a measure of specific binding. In some embodiments a binding affinity is a measure of binding between a cell and an ECM component or collection of ECM components. In some embodiments, a binding affinity of cells to ECM components is expressed relative to binding affinities of cells to other ECM components. In some embodiments, a relative binding affinity of cells to an ECM component or collection of ECM components is expressed as a fold change relative to an average of all binding affinities of cells to ECM components or collection of ECM components assayed. In some embodiments, a relative binding affinity is 0. In some embodiments, a relative binding affinity is between 0 and 1. In some embodiments, a relative binding affinity is 1, 2, 3, 4, 5, 6, 7, 8, 9, 10 or more fold. In some embodiments, a relative binding affinity is between 0 and −1. In some embodiments, a relative binding affinity is −1, −2, −3, −4, −5, −6, −7, −8, −9, −10 or more fold.

Aggrecan polypeptide: In accordance with the present invention, the term “aggrecan polypeptide” is used to refer to a polypeptide that 1) shares an overall level of sequence identity and/or 2) shares at least one characteristic sequence element with an aggrecan protein, for example as set forth in Table 1 of the Appendix.

Agrin polypeptide: In accordance with the present invention, the term “agrin polypeptide” is used to refer to a polypeptide that 1) shares an overall level of sequence identity and/or 2) shares at least one characteristic sequence element with an agrin protein, for example as set forth in Table 1 of the Appendix.

Antibody: As used herein, the term “antibody” refers to any immunoglobulin, whether natural or wholly or partially synthetically produced. In some embodiments, an antibody is a complex comprised of 4 full-length polypeptide chains, each of which includes a variable region and a constant region, e.g., substantially of the structure of an antibody produced in nature by a B cell. In some embodiments, an antibody is a single chain. In some embodiments, an antibody is cameloid. In some embodiments, an antibody is an antibody fragment. In some embodiments, an antibody is chimeric. In some embodiments, an antibody is bi-specific. In some embodiments, an antibody is multi-specific. In some embodiments, an antibody is monoclonal. In some embodiments, an antibody is polyclonal. In some embodiments, an antibody is conjugated (i.e., antibodies conjugated or fused to other proteins, radiolabels, cytotoxins). In some embodiments, an antibody is a human antibody. In some embodiments, an antibody is a mouse antibody. In some embodiments, an antibody is a rabbit antibody. In some embodiments, an antibody is a rat antibody. In some embodiments, an antibody is a donkey antibody.

Array: An “array”, as that term is used herein, typically refers to an arrangement of entities (e.g., ECM components) in spatially discrete locations with respect to one another, and usually in a format that permits simultaneous exposure of the arranged entities to potential interaction partners (e.g., cells) or other reagents, substrates, etc. In some embodiments, an array comprises entities arranged in spatially discrete locations on a solid support. In some embodiments, spatially discrete locations on an array are termed “spots” (regardless of their shape). In some embodiments, spatially discrete locations on an array are arranged in a regular pattern with respect to one another (e.g., in a grid).

Biglycan polypeptide: In accordance with the present invention, the term “biglycan polypeptide” is used to refer to a polypeptide that 1) shares an overall level of sequence identity and/or 2) shares at least one characteristic sequence element with a biglycan protein, for example as set forth in Table 1 of the Appendix.

Binding partners: In general, the term “binding partner” is used herein to refer to any two entities that specifically bind with each other in a given context. In some embodiments, binding is specific in that a binding agent has a greater affinity for its target binding partner than for other potential binding partners in its environment. Binding partners may be of any chemical type. In some embodiments, binding partners are polypeptides. In some embodiments, binding partners are integrins, syndecans, proteoglycans, glycosaminoglycans, and/or lectins. In some embodiments, binding partners are carbohydrates.

Brevican polypeptide: In accordance with the present invention, the term “brevican polypeptide” is used to refer to a polypeptide that 1) shares an overall level of sequence identity and/or 2) shares at least one characteristic sequence element with a brevican protein, for example as set forth in Table 1 of the Appendix.

Collagen I polypeptide: In accordance with the present invention, the term “collagen I polypeptide” is used to refer to a polypeptide that 1) shares an overall level of sequence identity and/or 2) shares at least one characteristic sequence element with a collagen I protein, for example as set forth in Table 1 of the Appendix.

Collagen II polypeptide: In accordance with the present invention, the term “collagen II polypeptide” is used to refer to a polypeptide that 1) shares an overall level of sequence identity and/or 2) shares at least one characteristic sequence element with a collagen II protein, for example as set forth in Table 1 of the Appendix.

Collagen III polypeptide: In accordance with the present invention, the term “collagen III polypeptide” is used to refer to a polypeptide that 1) shares an overall level of sequence identity and/or 2) shares at least one characteristic sequence element with a collagen III protein, for example as set forth in Table 1 of the Appendix.

Collagen IV polypeptide: In accordance with the present invention, the term “collagen IV polypeptide” is used to refer to a polypeptide that 1) shares an overall level of sequence identity and/or 2) shares at least one characteristic sequence element with a collagen IV protein, for example as set forth in Table 1 of the Appendix.

Collagen V polypeptide: In accordance with the present invention, the term “collagen V polypeptide” is used to refer to a polypeptide that 1) shares an overall level of sequence identity and/or 2) shares at least one characteristic sequence element with a collagen V protein, for example as set forth in Table 1 of the Appendix.

Collagen VI polypeptide: In accordance with the present invention, the term “collagen VI polypeptide” is used to refer to a polypeptide that 1) shares an overall level of sequence identity and/or 2) shares at least one characteristic sequence element with a collagen VI protein, for example as set forth in Table 1 of the Appendix.

Characteristic: As is used herein, the term “characteristic” refers to any detectable feature of a cell type that allows it to be distinguished from a comparable cell type. In some embodiments, a characteristic is an amount or sequence of a gene. In some embodiments, a characteristic is an amount or sequence of a gene transcript. In some embodiments, a characteristic is an amount, sequence of, or modification of a protein. In some embodiments a characteristic is an amount of a carbohydrate. In some embodiments, a characteristic is an amount of a small molecule. In some embodiments, a characteristic is an amount of an ECM component.

Comparable: As is used herein, the term “comparable” is used to refer to two entities that are sufficiently similar to permit comparison, but differing in at least one feature.

Decorin polypeptide: In accordance with the present invention, the term “decorin polypeptide” is used to refer to a polypeptide that 1) shares an overall level of sequence identity and/or 2) shares at least one characteristic sequence element with a decorin protein, for example as set forth in Table 1 of the Appendix.

ECM component: In accordance with the present invention, the term “ECM component” is used to refer to any molecule or molecular complex that is part of an ECM of a cell and that has contributes to one or more adhesion signatures for a cell. In some embodiments, an ECM component is or comprises a polypeptide. In some embodiments, an ECM component is or comprises a polysaccharide. In some embodiments, an ECM component is or comprises a glycosaminoglycan. In some embodiments, an ECM component is or comprises a proteoglycan. In some embodiments an ECM component comprises a carbohydrate. In some embodiments, the ECM component is any fragment of a polypeptide, glycosaminoglycan, proteoglycan, or carbohydrate disclosed herein. In some embodiments, the ECM component is a polypeptide that shares at least 85%, 90%, 95%, 96%, 97%, 98%, or 99% sequence identity to any of the polypeptides disclosed in Table 1.

Elastin polypeptide: In accordance with the present invention, the term “elastin polypeptide” is used to refer to a polypeptide that 1) shares an overall level of sequence identity and/or 2) shares at least one characteristic sequence element with an elastin protein, for example as set forth in Table 1 of the Appendix.

F-Spondin polypeptide: In accordance with the present invention, the term “F-spondin polypeptide” is used to refer to a polypeptide that 1) shares an overall level of sequence identity and/or 2) shares at least one characteristic sequence element with an F-spondin protein, for example as set forth in Table 1 of the Appendix.

Fibrin polypeptide: In accordance with the present invention, the term “fibrin polypeptide” is used to refer to a polypeptide that 1) shares an overall level of sequence identity and/or 2) shares at least one characteristic sequence element with a fibrin protein, for example as set forth in Table 1 of the Appendix.

Fibronectin polypeptide: In accordance with the present invention, the term “fibronectin polypeptide” is used to refer to a polypeptide that 1) shares an overall level of sequence identity and/or 2) shares at least one characteristic sequence element with a fibronectin protein, for example as set forth in Table 1 of the Appendix.

Galectin 1 polypeptide: In accordance with the present invention, the term “galectin 1 polypeptide” is used to refer to a polypeptide that 1) shares an overall level of sequence identity and/or 2) shares at least one characteristic sequence element with a galectin 1 protein, for example as set forth in Table 1 of the Appendix.

Galectin 3 polypeptide: In accordance with the present invention, the term “galectin 3 polypeptide” is used to refer to a polypeptide that 1) shares an overall level of sequence identity and/or 2) shares at least one characteristic sequence element with a galectin 3 protein, for example as set forth in Table 1 of the Appendix.

Galectin 3c polypeptide: In accordance with the present invention, the term “galectin 3c polypeptide” is used to refer to a polypeptide that 1) shares an overall level of sequence identity and/or 2) shares at least one characteristic sequence element with a galectin 3c protein, for example as set forth in Table 1 of the Appendix.

Galectin 4 polypeptide: In accordance with the present invention, the term “galectin 4 polypeptide” is used to refer to a polypeptide that 1) shares an overall level of sequence identity and/or 2) shares at least one characteristic sequence element with a galectin 4 protein, for example as set forth in Table 1 of the Appendix.

Galectin 8 polypeptide: In accordance with the present invention, the term “galectin 8 polypeptide” is used to refer to a polypeptide that 1) shares an overall level of sequence identity and/or 2) shares at least one characteristic sequence element with a galectin 8 protein, for example as set forth in Table 1 of the Appendix.

Glycosaminoglycan: In accordance with the present invention, the term “glycosaminoglycan” is used to refer to an unbranched polysaccharides consisting of a repeating disaccharide unit. The repeating unit consists of a hexose or a hexuronic acid, linked to a hexosamine.

Keratin polypeptide: In accordance with the present invention, the term “keratin polypeptide” is used to refer to a polypeptide that 1) shares an overall level of sequence identity and/or 2) shares at least one characteristic sequence element with a keratin protein, for example as set forth in Table 1 of the Appendix.

Kit: As used herein, the term “kit” refers to a set of components provided in the context of a delivery system for delivering materials. Such delivery systems may include, for example, systems that allow for storage, transport, or delivery of various diagnostic or therapeutic reagents (e.g., oligonucleotides, enzymes, extracellular matrix components etc. in appropriate containers) and/or supporting materials (e.g., buffers, media, cells, written instructions for performing the assay etc.) from one location to another. For example, in some embodiments, kits include one or more enclosures (e.g., boxes) containing relevant reaction reagents and/or supporting materials. As used herein, the term “fragmented kit” refers to delivery systems comprising two or more separate containers that each contain a subportion of total kit components. Containers may be delivered to an intended recipient together or separately. For example, a first container may contain a petri dish or polysterence plate for use in a cell culture assay, while a second container may contain cells. The term “fragmented kit” is intended to encompass kits containing Analyte Specific Reagents (ASR's) regulated under section 520(e) of the Federal Food, Drug, and Cosmetic Act, but are not limited thereto. Indeed, any delivery system comprising two or more separate containers that each contain a subportion of total kit components are included in the term “fragmented kit.” In contrast, a “combined kit” refers to a delivery system containing all components in a single container (e.g., in a single box housing each of the desired components). The term “kit” includes both fragmented and combined kits.

Laminin polypeptide: In accordance with the present invention, the term “laminin polypeptide” is used to refer to a polypeptide that 1) shares an overall level of sequence identity and/or 2) shares at least one characteristic sequence element with a laminin protein, for example as set forth in Table 1 of the Appendix.

Lineage: In accordance with the present invention, the term “lineage” encompasses cells at any point in a developmental process from undifferentiated cells to fully differentiated cells of a specific cell type.

Merosin polypeptide: In accordance with the present invention, the term “merosin polypeptide” is used to refer to a polypeptide that 1) shares an overall level of sequence identity and/or 2) shares at least one characteristic sequence element with a merosin protein, for example as set forth in Table 1 of the Appendix.

Mucin polypeptide: In accordance with the present invention, the term “mucin polypeptide” is used to refer to a polypeptide that 1) shares an overall level of sequence identity and/or 2) shares at least one characteristic sequence element with a mucin protein, for example as set forth in Table 1 of the Appendix.

Nidogen-1 polypeptide: In accordance with the present invention, the term “nidogen-1 polypeptide” is used to refer to a polypeptide that 1) shares an overall level of sequence identity and/or 2) shares at least one characteristic sequence element with a nidogen-1 protein, for example as set forth in Table 1 of the Appendix.

Nidogen-2 polypeptide: In accordance with the present invention, the term “nidogen-2 polypeptide” is used to refer to a polypeptide that 1) shares an overall level of sequence identity and/or 2) shares at least one characteristic sequence element with a nidogen-2 protein, for example as set forth in Table 1 of the Appendix.

Osteopontin polypeptide: In accordance with the present invention, the term “osteopontin polypeptide” is used to refer to a polypeptide that 1) shares an overall level of sequence identity and/or 2) shares at least one characteristic sequence element with an osteopontin protein, for example as set forth in Table 1 of the Appendix.

Polypeptide: The term “polypeptide”, as used herein, generally has its art-recognized meaning of a polymer of at least three amino acids. Those of ordinary skill in the art will appreciate that the term “polypeptide” is intended to be sufficiently general as to encompass not only polypeptides having the complete sequence recited herein, but also to encompass polypeptides that represent functional fragments (i.e., fragments retaining at least one activity) of such complete polypeptides. Moreover, those of ordinary skill in the art understand that protein sequences generally tolerate some substitution without destroying activity. Thus, any polypeptide that retains activity and shares at least about 30-40% overall sequence identity, often greater than about 50%, 60%, 70%, or 80%, and further usually including at least one region of much higher identity, often greater than 90% or even 95%, 96%, 97%, 98%, or 99% in one or more highly conserved regions, usually encompassing at least 3-4 and often up to 20 or more amino acids, with another polypeptide of the same class, is encompassed within the relevant term “polypeptide” as used herein.

Reference cell: As will be understood from context, a reference cell or cell type is one that is sufficiently similar to a particular cell or cell type of interest to permit a relevant comparison. In some embodiments, information about a reference cell or cell type is obtained simultaneously with information about the particular cell or cell type. In some embodiments, information about a reference cell or cell type is historical. In some embodiments, information about a reference cell or cell type is stored for example in a computer-readable medium. In some embodiments, comparison of a particular cell or cell type of interest with a reference cell or cell type establishes identity with, similarity to, or difference of the particular cell or cell type of interest relative to the reference.

Sample: As used herein, the term “sample” refers to a biological sample obtained or derived from a source of interest, as described herein. In some embodiments, a source of interest comprises an organism, such as an animal or human. In some embodiments, a biological sample comprises biological tissue or fluid. In some embodiments, a biological sample may be or comprise bone marrow; blood; blood cells; ascites; tissue or fine needle biopsy samples; cell-containing body fluids; free floating nucleic acids; sputum; saliva; urine; cerebrospinal fluid, peritoneal fluid; pleural fluid; feces; lymph; gynecological fluids; skin swabs; vaginal swabs; oral swabs; nasal swabs; washings or lavages such as a ductal lavages or bronchioalveolar lavages; aspirates; scrapings; bone marrow specimens; tissue biopsy specimens; surgical specimens; feces, other body fluids, secretions, and/or excretions; and/or cells therefrom, etc. In some embodiments, a biological sample is or comprises cells obtained from an individual. In some embodiments, a sample is a “primary sample” obtained directly from a source of interest by any appropriate means. For example, in some embodiments, a primary biological sample is obtained by methods selected from the group consisting of biopsy (e.g., fine needle aspiration or tissue biopsy), surgery, collection of body fluid (e.g., blood, lymph, feces etc.), etc. In some embodiments, as will be clear from context, the term “sample” refers to a preparation that is obtained by processing (e.g., by removing one or more components of and/or by adding one or more agents to) a primary sample. For example, filtering using a semi-permeable membrane. Such a “processed sample” may comprise, for example nucleic acids or proteins extracted from a sample or obtained by subjecting a primary sample to techniques such as amplification or reverse transcription of mRNA, isolation and/or purification of certain components, etc.

Superfibronectin polypeptide: In accordance with the present invention, the term “superfibronectin polypeptide” is used to refer to a polypeptide that 1) shares an overall level of sequence identity and/or 2) shares at least one characteristic sequence element with a superfibronectin protein, for example as set forth in Table 1 of the Appendix.

SPARC/Osteonectin polypeptide: In accordance with the present invention, the term “SPARC/osteonectin polypeptide” is used to refer to a polypeptide that 1) shares an overall level of sequence identity and/or 2) shares at least one characteristic sequence element with a SPARC/osteonectin protein, for example as set forth in Table 1 of the Appendix.

Tenascin-C polypeptide: In accordance with the present invention, the term “tenascin-C polypeptide” is used to refer to a polypeptide that 1) shares an overall level of sequence identity and/or 2) shares at least one characteristic sequence element with a tenascin-C protein, for example as set forth in Table 1 of the Appendix.

Tenascin-R polypeptide: In accordance with the present invention, the term “tenascin-R polypeptide” is used to refer to a polypeptide that 1) shares an overall level of sequence identity and/or 2) shares at least one characteristic sequence element with a tenascin-R protein, for example as set forth in Table 1 of the Appendix.

Testican 1/SPOCK1 polypeptide: In accordance with the present invention, the term “testican 1/SPOCK1 polypeptide” is used to refer to a polypeptide that 1) shares an overall level of sequence identity and/or 2) shares at least one characteristic sequence element with a testican 1/SPOCK1 protein, for example as set forth in Table 1 of the Appendix.

Testican 2/SPOCK2 polypeptide: In accordance with the present invention, the term “testican 2/SPOCK2 polypeptide” is used to refer to a polypeptide that 1) shares an overall level of sequence identity and/or 2) shares at least one characteristic sequence element with a testican 2/SPOCK2 protein, for example as set forth in Table 1 of the Appendix.

Thrombospondin-4 polypeptide: In accordance with the present invention, the term “thrombospondin-4 polypeptide” is used to refer to a polypeptide that 1) shares an overall level of sequence identity and/or 2) shares at least one characteristic sequence element with a thrombospondin-4 protein, for example as set forth in Table 1 of the Appendix.

Vitronectin polypeptide: In accordance with the present invention, the term “vitronectin polypeptide” is used to refer to a polypeptide that 1) shares an overall level of sequence identity and/or 2) shares at least one characteristic sequence element with a vitronectin protein, for example as set forth in Table 1 of the Appendix.

### Extracellular Matrix (ECM)

Many significant cellular components reside within the extracellular matrix (ECM), a gelatinous layer on the exterior surface of cells. The ECM plays a variety of important roles, including serving as scaffolding for cellular components and providing biochemical and mechanical cues involved in intracellular communication and tissue differentiation. The ECM includes proteoglycan and fibrous protein, typically produced within cells and then secreted to form the ECM.

ECMs of different cell types are highly variable. For example, differing ECM compositions of different types of fibroblasts determine properties of connective tissue. Chondrocytes secrete an ECM composed primarily of collagen II, which forms cartilage, whereas osteoplasts secrete an ECM composed primarily of osteoid, a progenitor of bone tissue. This variability is created during development by an interaction of cells with the microenvironments in which they are located.

In addition to providing structural support, another important role of the ECM is intracellular communication, in particular through integrins. Integrins are cell surface receptors that regulate attachment of a cell to the ECM, and also transduce intracellular signals from the ECM to the interior of a cell. In addition to having a unique ECM composition, each respective cell type also has an individualized profile of cell surface integrins and other receptors for best interacting with its specific ECM. Thus, the ECM composition and the affinity for ECM components of a cell represents a potentially useful way of distinguishing between genetically identical cell types.

### Extracellular Matrix Components

The present invention relates generally to definition and/or use of adhesion signatures that embody or characterize a cell's affinity for of Extracellular Matrix (ECM) components.

In some embodiments, an ECM component is or comprises any polypeptide present in the ECM. In some embodiments, an ECM component is or comprises an aggrecan polypeptide, an agrin polypeptide, a biglycan polypeptide, a brevican polypeptide, a collagen I polypeptide, a collagen II polypeptide, a collagen III polypeptide, a collagen IV polypeptide, a collagen V polypeptide, a collagen VI polypeptide, a decorin polypeptide, an elastin polypeptide, an f-spondin polypeptide, a fibrin polypeptide, a fibronectin polypeptide, a galectin 1 polypeptide, a galectin 3 polypeptide, a galectin 3c polypeptide, a galectin 4 polypeptide, a galectin 8 polypeptide, a keratin polypeptide, a laminin polypeptide, a merosin polypeptide, a mucin polypeptide, nidogen-1 polypeptide, a nidogen-2 polypeptide, an osteopontin polypeptide, a SPARC/osteonectin superfibronectin polypeptide, a tenascin-C polypeptide, a tenascin-R polypeptide, a testican 1/SPOCK1 polypeptide, a testican 2/SPOCK2 polypeptide, a thrombospondin-4 polypeptide, a vitronectin polypeptide and/or combinations thereof.

In some embodiments, an ECM component is or comprises one or more carbohydrate moieties. In some embodiments, an ECM component is or comprises a carbohydrate moiety that is naturally found in ECM produced by cells (e.g., on an ECM polypeptide). Representative such carbohydrate moieties include, for example, ECM components chondroitin sulfate glycosaminoglycans, heparan sulfate glycosaminoglycans, hyaluronic acid glycosaminoglycans or other glycosaminoglycans, and/or combinations thereof.

In some embodiments, an ECM component is or comprises a protein, peptide, glycoprotein, proteoglycans, glycosaminoglycans, and/or carbohydrate that is secreted by cells into the extracellular environment. In some embodiments, the secreted protein, peptide, glycoprotein, proteoglycans, glycosaminoglycans, and/or carbohydrate, or structures composed thereof can be bound to by cells as a means of immobilizing the cell permanently or transiently (as in cases of providing a means for directional motility).

ECM components interact with cells, typically through non-covalent binding interactions with one or more entities on or near cell surfaces. In some embodiments, cell components that interact or bind with ECM components include entities selected from groups consisting of cell membranes, cell surface entities (e.g., proteins, proteoglycans, glycoproteins, etc.), secreted entities (e.g., cell signaling molecules), laminins, integrins, syndecans, and actin.

### Cells

In various embodiments, the present invention is useful in the identification, characterization, detection, isolation, and/or culturing of cells. In general, teachings of the invention are relevant to any cell that has, produces, and/or interacts with an ECM or ECM component(s).

In some embodiments, cells utilized in accordance with the present invention are cells that retain viability, and optionally growth capabilities, when suspended in solution. In some embodiments, cells are eukaryotic cells. In certain embodiments, cells are human cells. In some embodiments, cells are mouse cells. In certain embodiments, cells are obtained from cell culture. In some embodiments, cells are obtained from a living organism. In some embodiments, cells are hepatic cells. In some embodiments, cells are immune cells. In certain embodiments, cells are blood cells. In some embodiments, cells are nerve cells. In certain embodiments, cells are epithelial cells. In certain embodiments, cells are reproductive cells. In some embodiments, cells are stem cells. In some embodiments, cells are cancer cells. In some embodiments, the cell sample comprises an individual cell. In some embodiments, the cell sample is a composition comprising a plurality of cells. In some embodiments, the cell sample is a tissue sample taken from a subject suspected of having cancer or being diagnosed as having cancer. In some embodiments, the cell sample is a tissue sample taken from a subject with lung cancer or breast cancer. In some embodiments, the cell sample comprises a plurality of cells from the adrenal gland, bladder, blood, bone, bone marrow, brain, spine, breast, cervix, gall bladder, ganglia, gastrointestinal tract, stomach, colon, heart, kidney, liver, lung, lymphnodes, muscle, overay, pancreas, parathyroid, penis, prostate, salivary glands, skin, spleen, testis, thymus, thyroid, or uterus. In some embodiment, the cell sample comprises a plurality of cells derived from the lung. In some embodiments, the cell sample comprises a plurality of cells derived from the breast.

In various embodiments, the present invention is useful in identification, characterization, detection, isolation, and/or culturing of stem cells at particular states of differentiation. As is commonly understood in the art, stem cells are cells with a capacity to differentiate into diverse specialized cell types. Different types of stem cells are at different stages of differentiation, ranging from completely undifferentiated (totipotent) to mostly differentiated (multipotent). In some embodiments stem cells are totipotent stem cells (e.g., undifferentiated cells having an ability to differentiate into any mature cell type). Types of totipotent stem cells include, for example, embryonic stem cells. In some embodiments, stem cells are pluripotent stem cells (e.g., having an ability to differentiate into most mature cell types). Types of pluripotent stem cells include, for example, induced pluripotent stem cells. In some embodiments, stem cells are multipotent stem cells (e.g., having an ability to differentiate into several related types of cells). Types of multipotent stem cells include, for example, mesenchymal stem cells. In some embodiments, mesenchymal stem cells are derived from bone marrow, adipose tissue, umbilical cord blood and/or umbilical cord. In certain embodiments, cells utilized in accordance with the present invention are cells differentiated from stem cells.

In various embodiments, the present invention is useful in identification, characterization, detection, isolation, and/or culturing of cancer cells generally and specifically cancer cells at particular states of metastasis. As is commonly understood in the art, metastasis is a process of cancer spreading from an initial tumor site and is correlated with a poor prognosis for cancer patients. Metastatic cells are characterized by an altered gene expression profile that directly correlates with ability to metastasize (Ramaswamy S. et al. “A molecular signature of metastasis in primary solid tumors”. Nature Genetics 33 (1): 49-54, 2003). Types of cancer cells include but are not limited to lung adenocarcinoma cells, non-metastatic primary tumor cells, metastatic primary tumor cells, metastatic lymph node cells, metastatic liver cells, breast cancer cells, colon cancer cells, prostate cancer cells, ovarian cancer cells, testicular cancer cells and/or leukemia cells.

In accordance with certain embodiments of the present invention, cells are contacted with ECM components, under conditions and for a time sufficient to allow cells to bind to ECM components. In certain embodiments, contacted cells are suspended in a solution. In some embodiments, cells are suspended at a concentration ranging from 100 to 10,000,000 cells/ml, from 1,000 to 1,000,000 cells/ml, or from 10,000 to 100,000 cells/ml. In one exemplary embodiment, cells are suspended at a concentration of 80,000 cells/ml. In certain embodiments, cells and ECM components are contacted in the presence of culture media. Any of a variety of cell culture media, including complex media and/or serum-free culture media, that support survival and/or growth of the one or more cell types or cell lines may be used in accordance with the present disclosure. Typically, a cell culture medium contains a buffer, salts, energy source, amino acids, vitamins and/or trace elements. Cell culture media may optionally contain a variety of other ingredients, including but not limited to, carbon sources, cofactors, lipids, sugars, nucleosides, animal-derived components, hydrolysates, hormones/growth factors, surfactants, indicators, minerals, activators/inhibitors of specific enzymes, and organics, and/or small molecule metabolites.

In certain embodiments, cell culture media utilized in accordance with the present invention is or comprises serum-free cell culture media. In certain embodiments, utilized cell culture media is fully defined synthetic cell culture media. In certain embodiments, utilized cell culture media is Dulbecco's Modified Eagle Medium (DMEM). In certain embodiments, utilized cell culture media is RPMI, Ham's F-12, or Mammary Epithelial Cell Growth Media (MEGM). In some embodiments, the cell culture media comprises additional components including Fetal Bovine Serum (FBS), Bovine Serum (BS), and/or Glutamine or combinations thereof. In some embodiments, utilized media are supplemented with an antibiotic to prevent contamination. Useful antibiotics in such circumstances include, for example, penicillin, streptomycin, and/or gentamicin and combinations thereof. Those of skill in the art are familiar with parameters relevant to selection of appropriate cell culture media.

### System and Arrays

In many embodiments, an array comprises a solid support to whose surface(s) ECM components are affixed in spatially discrete locations. Such an array can be prepared using ECM components from any source (e.g., recombinantly produced, biochemically isolated, commercially purchased, etc). Moreover, identity and relative amounts of individual ECM components may be determined or adjusted in accordance with requirements of a particular project or interests of a particular researcher.

For example, in many embodiments, it will be desirable to design, prepare and/or utilize an ECM array that includes as many different ECM components as is feasible. Alternatively or additionally, in some embodiments, it may be desirable to design, prepare, and/or utilize an ECM array that includes only ECM components known to be associated with (or not associated with) a particular cell or cell type. To give a few particular examples, in some embodiments, an ECM array is utilized that contains at least 1, 2, 3, 4, 5, 6, 7, 8, 9, 10, 11, 12, 13, 14, 15, 16, 17, 18, 19, 20 or more different “spots” (physically discrete locations) containing different ECM components. In some embodiments, an ECM array is utilized that contains between about 1 and about 100,000 spots, between about 100 and about 10,000, or between about 1,000 and about 5,000 spots.

In some embodiments, spots on an array show spatial organization. In some embodiments, spots on an array are arranged in a grid.

In some embodiments, a variety of ECM components and combinations thereof are represented in spots of an ECM array with each spot corresponding to both a known location on the ECM array and a known composition of ECM components. In certain embodiments, at least one ECM component is spotted upon the ECM array. In certain embodiments, the ECM components are spotted individually. In some embodiments, mixtures of several ECM components are contained within a single spot. In some embodiments, an ECM array for use in accordance with the present invention includes both spots of single ECM components and spots of combinations of ECM components. In some embodiments, ECM components are spotted multiple times in the same array, so that the array includes replicate spots. In some embodiments, an ECM array for use in accordance with the present invention contains spots that lack an ECM component, and therefore for example may be utilized as negative controls in addition to spots containing ECM components. In certain embodiments, rhodamine dextran is included in a negative control spot.

An ECM array for use in accordance with the present invention may be prepared on any suitable substrate material. In many embodiments, the material will support viability and/or growth of cells, e.g., mammalian cells. In some embodiments, an ECM arrays utilizes a substrate material selected from the group consisting of polyamides, polyesters, polystyrene, polypropylene, polyacrylates, polyvinyl compounds (e.g. polyvinylchloride), polycarbonate, polytetrafluoroethylene (PTFE), nitrocellulose, cotton, polyglycolic acid (PGA), cellulose, dextran, gelatin, glass, fluoropolymers, fluorinated ethylene propylene, polyvinylidene, polydimethylsiloxane, polystyrene, silicon substrates (such as fused silica, polysilicon, or single silicon crystals), and the like, or combinations thereof. Alternatively or additionally, metals (gold, silver, titanium films) can be used. In a some embodiments, acrylic slides coated with polyacrylamide are used.

In some embodiments, the present invention provides ECM arrays for use in culturing cells. In some embodiments the ECM arrays for use in culturing cells are provided with medium. In some embodiments the ECM arrays for use in culturing cells are provided with a sufficient volume of medium to support cell culture for 1, 2, 3, 4, 5 or more days.

In some embodiments, the present invention provides ECM arrays for use as diagnostic assays. In some embodiments the ECM arrays are provided as part of a diagnostic or detection kit. In some embodiments the ECM arrays are provided as part of a detection kit. In certain embodiments, kits for use in accordance with the present invention may include one or more reference samples; instructions (e.g., for processing samples, for performing tests, for interpreting results, etc.); media; and/or other reagents necessary for performing tests.

The invention provides an array of polypeptides, the array comprising: a solid support and a plurality of adhesion sets, wherein each adhesion set comprises two or more different polypeptides comprising a polypeptide sequence associated with the extracellular matrix or a functional fragment thereof, and wherein the adhesion sets are attached to the solid support at an addressable location of the array. In some embodiments, the solid support is a slide optionally coated with a polymer. In some embodiments, the solid support is coated with a polymer. In some embodiments, the polymer is polyacrylamide. In some embodiments, the solid support is a material chosen from: polystyrene (TCPS), glass, quarts, quartz glass, poly(ethylene terephthalate) (PET), polyethylene, polyvinyl difluoride (PVDF), polydimethylsiloxane (PDMS), polytetrafluoroethylene (PTFE), polymethylmethacrylate (PMMA), polycarbonate, polyolefin, ethylene vinyl acetate, polypropylene, polysulfone, polytetrafluoroethylene, silicones, poly(meth)acrylic acid, polyamides, polyvinyl chloride, polyvinylphenol, and copolymers and mixtures thereof. In some embodiments, the at least one adhesion set comprises two different polypeptides attached to a solid support.

The invention further relates to a system comprising one or a plurality of arrays, wherein the one or plurality of arrays comprises: a solid support and a plurality of adhesion sets, wherein each adhesion set comprises two or more different polypeptides comprising a polypeptide sequence associated with the extracellular matrix or a functional fragment thereof, and wherein the adhesion sets are attached to the solid support at an addressable location of the array. In some embodiments, the one or plurality of arrays comprises a solid support is a slide optionally coated with a polymer. In some embodiments, the solid support is coated with a polymer. In some embodiments, the one or plurality of arrays comprises a solid support coated with a polymer that is polyacrylamide. In some embodiments, the one or plurality of arrays comprises a solid support comprising a material chosen from: polystyrene (TCPS), glass, quarts, quartz glass, poly(ethylene terephthalate) (PET), polyethylene, polyvinyl difluoride (PVDF), polydimethylsiloxane (PDMS), polytetrafluoroethylene (PTFE), polymethylmethacrylate (PMMA), polycarbonate, polyolefin, ethylene vinyl acetate, polypropylene, polysulfone, polytetrafluoroethylene, silicones, poly(meth)acrylic acid, polyamides, polyvinyl chloride, polyvinylphenol, and copolymers and mixtures thereof. In some embodiments, the at least one adhesion set comprises two different polypeptides attached to a solid support. In some embodiments, the system comprises a horizontally positioned or substantially horizontally positioned divide comprising at least one receptacle within which one or a plurality of solid supports is mounted. In some embodiments, the system comprises a horizontally positioned or substantially horizontally positioned divide comprising at least one receptacle and at least one gasket, such that the gasket is mounted between the one or a plurality of arrays and the divide. In some embodiments, the system comprises a horizontally positioned or substantially horizontally positioned divide defining an upper portion and a lower portion of the system wherein the divide comprises at least one receptacle and at least one gasket within which one or a plurality of arrays are mounted such that the gasket is positioned between the array and the divide. In some embodiments, the system comprises: (i) a horizontally positioned or substantially horizontally positioned divide defining an upper portion and a lower portion of the system wherein the divide comprises at least one receptacle and at least one gasket within which one or a plurality of arrays are mounted such that the gasket is positioned between the array and the divide; and (ii) a pair of side walls positioned orthogonally to the divide; and (iii) a base comprising an air inlet positioned between the pair of side walls such that the divide, the pair of side walls, and the base define a cavity; wherein the air inlet is adapted to receive a connector through which a vacuum is drawn, the vacuum capable of drawing fluid from the upper portion of the system to the lower portion. In some embodiments, the system comprises: (i) a horizontally positioned or substantially horizontally positioned divide defining an upper portion and a lower portion of the system wherein the divide comprises at least one receptacle and at least one gasket within which one or a plurality of arrays are mounted such that the gasket is positioned between the array and the divide; and (ii) a pair of side walls positioned orthogonally to the divide; and (iii) a base comprising an air inlet positioned between the pair of side walls such that the divide, the pair of side walls, and the base define a cavity; wherein the air inlet is adapted to receive a connector through which a vacuum is drawn, the vacuum capable of drawing fluid from the upper portion of the system to the lower portion. In some embodiments, the system comprises a vacuum pump operably connected to the base via a tube adapted to fit the air inlet.

The invention relates to a system comprising at least one, two, three, or four arrays as described herein. The invention also relates to a system comprising at least one array comprising at least 50, 60, 70, 80, 90, 100, 200, 300, 400, 500, 600, 700, 710, 720, 730, 740, 750, 760, 770, or 780 adhesion sets positioned at separate addressable locations on the at least one array. In some embodiments, the system is free of animal-derived ECM material, embryonic fibroblasts, material deposited from Engelbreth-Holm-Swarm (EHS) mouse sarcoma cells, or any combination thereof. In some embodiments, the array is free of serum derived or sourced from any animal species. In some embodiments, the system comprises at least one array wherein the at least one array comprises no less than 30, 40, 50, 60, 70, 80, 90, 100, 125, 150, 175, 200, 225, 250, 275, 300, 325, 350, 375, 400, 425, 450, 475, 500, 525, 550, 575, 600, 625, 650, 675, 700, 725, 750, 775, 800, 825, or more adhesion sets comprising at least one polypeptide sequence associated with the extracellular matrix chosen from the polypeptides of Table 1 or functional fragments thereof.

In some embodiments, the system comprises at least one array, prepared by the step comprising: affixing no fewer than 10, 20, 30, 40, 50, 60, 70, 80, 90, 100, 125, 150, 175, 200, 225, 250, 275, 300, 325, 350, 375, 400, 425, 450, 475, 500, 525, 550, 575, 600, 625, 650, 675, 700, 725, 750, 775, 800, or 825 adhesion sets to discrete addressable locations on a solid support.

In some embodiments, the system comprises at least one array, prepared by the steps comprising: affixing no fewer than 10, 20, 30, 40, 50, 60, 70, 80, 90, 100, 125, 150, 175, 200, 225, 250, 275, 300, 325, 350, 375, 400, 425, 450, 475, 500, 525, 550, 575, 600, 625, 650, 675, 700, 725, 750, 775, 800, or 825 adhesion sets to discrete addressable locations on a solid support; wherein the adhesion sets comprise at least two or more polypeptides each of which comprising a polypeptide sequence associated with the extracellular matrix or a functional fragment thereof chosen from the polypeptides of Table 1. In some embodiments, the system comprises at least one array for the diagnosis or prognosis of a disorder of a patient, prepared by the steps comprising: (i) affixing no fewer than 10, 20, 30, 40, 50, 60, 70, 80, 90, 100, 125, 150, 175, 200, 225, 250, 275, 300, 325, 350, 375, 400, 425, 450, 475, 500, 525, 550, 575, 600, 625, 650, 675, 700, 725, 750, 775, 800, or 825 adhesion sets to discrete addressable locations on a solid support. In some embodiments, the system comprises at least one array for the diagnosis or the prognosis of a disorder of a patient, prepared by the steps comprising: (i) affixing no fewer than 10, 20, 30, 40, 50, 60, 70, 80, 90, 100, 125, 150, 175, 200, 225, 250, 275, 300, 325, 350, 375, 400, 425, 450, 475, 500, 525, 550, 575, 600, 625, 650, 675, 700, 725, 750, 775, 800, or 825 adhesion sets to discrete addressable locations on a solid support; wherein the adhesion sets comprise at least two or more polypeptides and wherein each of the two or more polypeptides comprises a polypeptide sequence associated with the extracellular matrix or a functional fragment thereof chosen from the polypeptides of Table 1. In some embodiments, the system comprises at least one array comprising a solid support, prepared by the steps comprising: (i) coating a solid support with a polymer; (ii) affixing no fewer than 10, 20, 30, 40, 50, 60, 70, 80, 90, 100, 125, 150, 175, 200, 225, 250, 275, 300, 325, 350, 375, 400, 425, 450, 475, 500, 525, 550, 575, 600, 625, 650, 675, 700, 725, 750, 775, 800, or 825 adhesion sets to discrete, addressable locations on the polymer; wherein the adhesion sets comprise at least two or more polypeptides each of which comprising a polypeptide sequence associated with the extracellular matrix or a functional fragment thereof chosen from the polypeptides of Table 1.

In some embodiments, the system comprises at least one array comprising a solid support, prepared by the steps comprising: affixing at least one adhesion set to the solid support; wherein the adhesion set comprises at least two or more polypeptides each comprising a polypeptide sequence associated with the extracellular matrix or a functional fragment thereof chosen from the polypeptides of Table 1. In some embodiments, the system comprises at least one array comprising a solid support, prepared by the steps comprising: affixing at least one adhesion set to the solid support; wherein the adhesion set comprises at least two or more polypeptides each comprising a polypeptide sequence associated with the extracellular matrix or a functional fragment thereof chosen from the polypeptides of Table 1; wherein the solid support comprises a material chosen from: polystyrene (TCPS), glass, quarts, quartz glass, poly(ethylene terephthalate) (PET), polyethylene, polyvinyl difluoride (PVDF), polydimethylsiloxane (PDMS), polytetrafluoroethylene (PTFE), polymethylmethacrylate (PMMA), polycarbonate, polyolefin, ethylene vinyl acetate, polypropylene, polysulfone, polytetrafluoroethylene, silicones, poly(meth)acrylic acid, polyamides, polyvinyl chloride, polyvinylphenol, and copolymers mixtures thereof.

In some embodiments, the system comprises at least one array comprising a solid support, prepared by the steps comprising: (i) preparing a first and second solution, each first and second solution comprising a known concentration of a polypeptide comprising a polypeptide sequence associated with the extracellular matrix or a functional fragment thereof; (ii) contacting the first and second solution with the solid support for a sufficient time period to adsorb polypeptide comprising a polypeptide sequence associated with the extracellular matrix or a functional fragment thereof to the solid support; wherein the polypeptide sequence associated with the extracellular matrix or a functional fragment thereof is chosen from the polypeptides of Table 1. In some embodiments, the system comprises at least one array comprising a solid support, prepared by the steps comprising: (i) preparing a first and second solution, each first and second solution comprising a known concentration of a polypeptide comprising a polypeptide sequence associated with the extracellular matrix or a functional fragment thereof; (ii) contacting the first and second solution with the solid support for a sufficient time period to adsorb polypeptide comprising a polypeptide sequence associated with the extracellular matrix or a functional fragment thereof to the solid support; wherein the polypeptide sequence associated with the extracellular matrix or a functional fragment thereof is chosen from the polypeptides of Table 1; wherein the solid support comprises a material chosen from: polystyrene (TCPS), glass, quarts, quartz glass, poly(ethylene terephthalate) (PET), polyethylene, polyvinyl difluoride (PVDF), polydimethylsiloxane (PDMS), polytetrafluoroethylene (PTFE), polymethylmethacrylate (PMMA), polycarbonate, polyolefin, ethylene vinyl acetate, polypropylene, polysulfone, polytetrafluoroethylene, silicones, poly(meth)acrylic acid, polyamides, polyvinyl chloride, polyvinylphenol, and copolymers mixtures thereof.

In some embodiments, the system comprises at least one array comprising a solid support, prepared by the steps comprising: (i) preparing a first and second solution, each first and second solution comprising a known concentration of a polypeptide comprising a polypeptide sequence associated with the extracellular matrix or a functional fragment thereof; (ii) contacting the first and second solution with the solid support for a sufficient time period absorb polypeptide comprising a polypeptide sequence associated with the extracellular matrix or a functional fragment thereof to the solid support; wherein the polypeptide sequence associated with the extracellular matrix or a functional fragment thereof is chosen from the polypeptides of Table 1; and wherein the steps of preparing a solution and contacting the solution with the solid support is repeated at least 700 times corresponding to the number of adhesion sets present on the at least one array. In some embodiments, the one or more repeated steps of contacting the first and second solution with the solid support is performed by an automated device such that each polypeptide comprising a polypeptide sequence associated with the extracellular matrix or fragment thereof is absorbed at discrete addressable locations on the at least one array.

### Adhesion Signatures

The present invention encompasses the recognition that cells can be identified and/or characterized by “adhesion signatures” that embody a cell's affinity for one or more Extracellular Matrix (ECM) components. In some embodiments, an adhesion signature includes binding information sufficient to compare a particular cell or cell type of interest with a reference cell or cell type and/or to identify, characterize, and/or distinguish a particular cell or cell type with respect to other cells or cell types.

In some embodiments, an adhesion signature comprises information respecting absence, presence and/or level of binding interactions with one or more ECM components selected from the group consisting of aggrecan, agrin, biglycan, brevican, chondroitin sulfate, collagen I, collagen II, collagen III, collagen IV, collagen V, collagen VI, decorin, elastin, f-spondin, fibrin, fibronectin, galectin 1, galectin 3, galectin 3c, galectin 4, galectin 8, heparan sulfate, hyaluronic acid, keratin, laminin, merosin, mucin, nidogen-1, nidogen-2, osteopontin, SPARC/osteonectin, superfibronectin, tenascin-C, tenascin-R, testican 1/SPOCKI, testican 2/SPOCK2, thrombospondin-4, vitronectin and combinations thereof.

In some embodiments, an adhesion signature distinguishes a cell or cell type from comparable cells or cell types of other tissue origin. In some embodiments, an adhesion signature distinguishes a cell or cell type from comparable cells or cell types of a different developmental stage (or point in development). In some embodiments, an adhesion signature distinguishes a cell or cell type from comparable cells or cell types that differ in presence of and/or susceptibility to one or more disease states, disorders, or conditions. In some embodiments, an adhesion signature distinguishes a cell or cell type from comparable cells or cell types that differ in physiologic state. In some embodiments, an adhesion signature distinguishes a cell or cell type from comparable cells or cell types that differ with respect to extent, degree, or type of exposure to one or more environmental factors (including drugs, toxins, etc).

In some embodiments, detection or determination of an adhesion signature reveals information about identity, extent, and or nature of one or more components of ECM produced by a cell, and/or of one or more factors present on (e.g., expressed or captured on) a cell surface. To give but one example, existence and/or level of particular binding interactions in an adhesion signature of a cell can reveal identity, extent, and or nature of a cell surface component such as, for example, an integrin that participates in binding interaction(s).

In some embodiments, adhesion signatures are determined by contacting a cell or cell sample with an array or system disclosed herein; quantifying one or more adhesion values; and compiling the one or more adhesion values to create or determine one or more adhesion signatures, or profiles. In some embodiments, the step of quantifying one or more adhesion values comprises detecting a quantitative signal or signals relative to the cell or cell sample binding to one or a plurality of adhesion sets, normalizing the quantitative signals as compared to a control or reference cell or cell sample, and applying an algorithm or interpretation function disclosed herein to the quantitative signal or signals such that the output of the algorithm or interpretation function disclosed herein is one or a plurality of adhesion values. In some embodiments, the step of applying the algorithm or interpretation function disclosed herein is performed by a non-transitory computer program product. In some embodiments, one or more steps of the methods disclosed herein are performed by a non-transitory computer implemented method. In some embodiments, the algorithm or interpretation function for quantifying one or more adhesion values is performed using CellProfiler software (Carpenter A E, J. T., Lamprecht M R, Clarke C, Kang I H, Friman O, Guertin D A, Chang J H, Lindquist R A, Moffat J, Golland P, Sabatini D M (2006). CellProfiler: image analysis software for identifying and quantifying cell phenotypes. Genome Biology 7, R100; which is herein incorporated by reference in its entirety). Nuclei are identified using the “IdentifyPrimaryObjects” module of the CellProfiler software with the Otsu Global thresholding method. Clumped objects are distinguished using “Intensity”. In some embodiments, adhesion values for a given cell line are determined by computing the average of replicate slides run for that given cell line. In some embodiments, the step of normalizing the adhesion values as compared to a control or reference cell or cell sample is accomplished by hierarchical clustering using Spotfire software, a hierarchical agglomerative method. For row clustering, the cluster analysis begins with each row placed in a separate cluster. Then the distance between all possible combinations of two rows is calculated using the Euclidean distance measure. The two most similar clusters are then grouped together and form a new cluster. In subsequent steps, the distance between the new cluster and all remaining clusters is recalculated using the UPGMA (Unweighted Pair-Group Method with Arithmetic mean) method. The number of clusters is thereby reduced by one in each iteration step. Eventually, all rows are grouped into one large cluster. The order of the rows in a dendrogram are defined by the average value weight. In some embodiments, no column clustering was performed.

Once the one or plurality of adhesion values are calculated using the algorithm or interpretation function, one can create or determine an adhesion signature for the cell or cell sample which, in some embodiments, is a quantitative binding profile (collection of adhesion values) of a cell or cell sample relative to the one or plurality of adhesion sets to which a reference cell or reference cell sample has been contacted. A user of the array or system disclosed herein can subsequently compare the adhesion signature of the cell or cell sample to one or a plurality of adhesion control or reference cells. In some embodiments, the adhesion signatures of the one or plurality of control samples is predetermined and/or catalogued so that the user of the array or system disclosed herein can compare the signatures of the cell or cell sample to the predetermined and/or catalogued control signature to identify or characterize the phenotype of the cell or cell sample. In some embodiments, the adhesion signatures of the one or plurality of control is predetermined and/or catalogued so that the user of the array or system disclosed herein can compare the signatures of the cell or cell sample to the predetermined and/or catalogued control adhesion signature to qualitatively assess the cell or cell sample as having physical characteristics more or less similar to the control adhesion signature. In some embodiments, the user of the array or system disclosed herein and generate a profile related to similarities or dissimilarities as between the cell or cell sample adhesion signature and the control adhesion signature. In some embodiments, the control adhesion signature is adhesion signature that quantitatively describes a set of adhesion values from a metastatic tissue. In some embodiments, the control adhesion signature is an adhesion signature that quantitatively describes a set of adhesion values from cancerous tissue. In some embodiments, the control adhesion signature is adhesion signature that quantitatively describes a set of adhesion values from a pre-cancerous tissue. In some embodiments, the control adhesion signature is adhesion signature that quantitatively describes a set of adhesion values from a stem cell. In some embodiments, the control adhesion signature is adhesion signature that quantitatively describes a set of adhesion values from an embryonic stem cell. In some embodiments, the control adhesion signature is adhesion signature that quantitatively describes a set of adhesion values from a mesenchymal stem cell. In some embodiments, the control adhesion signature is adhesion signature that quantitatively describes a set of adhesion values from an induced pluripotent stem cell. In some embodiments, the control adhesion signature is adhesion signature that quantitatively describes a set of adhesion values from a primary lineage of hepatocytes. In some embodiments, the control adhesion signature is adhesion signature that quantitatively describes a set of adhesion values from a cellular stage of development in respect to any of the cells disclosed herein. In some embodiments, the control adhesion signature is adhesion signature that quantitatively describes a set of adhesion values from one or various stages of tumor growth. In some embodiments, the control adhesion signature is adhesion signature that quantitatively describes a set of adhesion values from one or more induced pluripotent stem cells. In some embodiments, the control adhesion signature is adhesion signature that quantitatively describes a set of adhesion values from one or more mesenchymal stem cells. In some embodiments, the control adhesion signature is adhesion signature that quantitatively describes a set of adhesion values from one or more bone-derived stem cells. In some embodiments, the control adhesion signature is adhesion signature that quantitatively describes a set of adhesion values from one or more embryonic stem cells. In some embodiments, the control adhesion signature is adhesion signature that quantitatively describes a set of adhesion values from one or more adipose-derived stem cells. In some embodiments, the control adhesion signature is adhesion signature that quantitatively describes a set of adhesion values from one or more stem cells.

According to some embodiments, the invention provides a software component or other non-transitory computer program product that is encoded on a computer-readable storage medium, and which optionally includes instructions (such as a programmed script or the like) that, when executed, cause operations related to the calculation of adhesion values and/or adhesion signatures. In some embodiments, the computer program product is encoded on a computer-readable storage medium that, when executed: quantifies one or more adhesion values; normalizes the one or more adhesion values over a control set of data; creates an adhesion profile or signature; and displays the adhesion profile or signature to a user of the computer program product. In some embodiments, the computer program product is encoded on a computer-readable storage medium that, when executed: calculates one or more adhesion values, normalizes the one or more adhesion values, and creates an adhesion signature, wherein the computer program product optionally displays the adhesion signature and/or adhesion values on a display operated by a user. In some embodiments, the invention relates to a non-transitory computer program product encoded on a computer-readable storage medium comprising instructions for: quantifying one or more adhesion values; and displaying the one or more adhesion values to a user of the computer program product. In some embodiments, the invention provides a non-transitory computer program product encoded on a computer-readable storage medium comprising instructions for: quantifying one or more adhesion values; normalizing the one or more adhesion values to a control set of data; creating an adhesion signature; and displaying the adhesion profile to a user of the computer program product. In some embodiments, the step of calculating one or more adhesion values comprises quantifying an average and standard deviation of counts on replicate spots. In some embodiments, the step of calculating one or more adhesion values comprises discarding the spots for which the count is greater or less than one standard deviation above or below the mean, respectively, and computing an average of the remaining counts (such average denoted as “x”). In some embodiments, the step of normalizing the one or more adhesion values over a control set of data is performed by first computing the average count across all ECM combinations on the slide for which the count is greater than zero (X). In some embodiments, the normalized adhesion value for each combination is then computed by dividing the average of the raw counts for the combination by the average of the non-zero counts for the slide (x/X).

### Uses

In general, one challenge faced by researchers and medical professionals is a need to identify cell types, differentiation states, and phenotypes, and to adequately isolate and grow specific cell populations. For example, because of interplay between genetic and environmental factors, two sub-populations of cells may be genetically identical and differ detectably only in composition of or adhesion to ECM components. Thus, one advantage of determining adhesion signature of cells as provided herein is that it can permit researchers to distinguish between cell populations that have not previously been distinguishable. Alternatively or additionally, provided methods and compositions allow characterization and/or classification of cells in ways not previously available or appreciated. Provided methods and compositions also provide basis for isolation or separation of cells from one another and/or from other components, materials, or entities.

The arrays, compositions, kits and systems disclosed herein allow the performance of methods to isolate, expand, differentiate, and maintain culture of mesenchymal stem cells and/or induced pluripotent stem cells. In some embodiments, the invention relates to a method of expanding of mesenchymal stem cells and/or induced pluripotent stem cells comprising the step of contacting mesenchymal stem cells and/or induced pluripotent stem cells to an array, composition, kit and/or system disclosed herein comprising at least one adhesion set. In some embodiments, the adhesion set comprises a polypeptide comprising a polypeptide sequence associated with the extracellular matrix that is chosen from one or a combination of: collagen I, laminin, collagen II, collagen IV, galectins-4, galectin-8, and/or fibronectin. In some embodiments, the adhesion set consists of collagen I and laminin. In some embodiments, the adhesion set consists of collagen II and galectin-4. In some embodiments, the adhesion set consists of collagen IV and galectin-4. In some embodiments, the adhesion set consists of collagen IV and galectin-8. In some embodiments, the adhesion set consists of collagen I and laminins and fibronectin. In some embodiments, the adhesion set consists of collagen II and galectin-4 and fibronectin. In some embodiments, the adhesion set consists of collagen IV and galectin-4 and fibronectin. In some embodiments, the adhesion set consists of collagen IV and galectin-8 and fibronectin. In some embodiments, the arrays, compositions, kits and systems disclosed herein are free of any polypeptide sequence associated with the extracellular matrix except collagen I, laminin, collagen II, collagen IV, galectins-4, galectin-8, and/or fibronectin. In some embodiments, the arrays, compositions, kits and systems disclosed herein are free of any media comprising inhibitors or antagonists of integrins.

In some embodiments, the invention relates to a method of isolating, expanding, differentiating, and/or maintaining a culture of mesenchymal stem cells and/or induced pluripotent stem cells by contacting a cell sample with one or more adhesion sets described herein in the presence of xeno-free media. In some embodiments, the invention relates to a method of isolating, expanding, differentiating, and/or maintaining a culture of mesenchymal stem cells and/or induced pluripotent stem cells by contacting a cell sample with one or more adhesion sets described herein in the presence of media free of animal-derived components. In some embodiments, the invention relates to a method of isolating, expanding, differentiating, and/or maintaining a culture of mesenchymal stem cells and/or induced pluripotent stem cells by contacting a cell sample with one or more adhesion sets described herein in the presence of media free of any inhibitors of any integrins.

In some embodiments, the invention relates to a method of maintaining or culturing hepatocytes in culture derived from primary lineages of cells comprising the step of contacting any of the arrays or systems disclosed herein to a primary hepatocyte.

In some embodiments, the invention relates to a method of culturing mesenchymal stem cells comprising the step of contacting any of the arrays or systems disclosed herein to a MSC.

In some embodiments, the invention relates to a method of differentiating an MSC comprising the step of contacting any of the arrays or systems disclosed herein to a MSC.

In some embodiments, the invention relates to a method of differentiating an iPSC into a cardiac lineage, liver lineage, or neural lineage comprising the step of contacting any of the arrays or systems disclosed herein to iPSC.

In some embodiments, the invention relates to a method of culturing a iPSCs comprising the step of contacting any of the arrays or systems disclosed herein to a iPSC.

In some embodiments, the invention relates to a method of culturing normal mammary epithelial cells in culture comprising the step of contacting any of the arrays or systems disclosed herein to a cell sample comprising a mammary epithelial cell.

In some embodiments, the invention relates to a method of culturing metastatic mammary epithelial cells in culture comprising the step of contacting any of the arrays or systems disclosed herein to a cell sample comprising a metastatic mammary epithelial cell.

In some embodiments, the invention relates to a method of proliferating normal mammary epithelial cells in culture comprising the step of contacting any of the arrays or systems disclosed herein to a cell sample comprising a mammary epithelial cell.

In some embodiments, the invention relates to a method of proliferating metastatic mammary epithelial cells in culture comprising the step of contacting any of the arrays or systems disclosed herein to a cell sample comprising a metastatic mammary epithelial cell.

In some embodiments, the invention relates to a array or system, or kit consisting of any one or plurality of adhesion sets disclosed herein adsorbed to solid support comprising polystyrene.

In some embodiments, the invention relates to a pharmaceutical composition comprising: a therapeutically effective amount or prophylactically effective amount of a nucleic acid molecule that interferes with the expression of any of the cognate integrins disclosed herein; and a pharmaceutical acceptable carrier. In some embodiments, the invention relates to a pharmaceutical composition comprising: a therapeutically effective amount of a nucleic acid molecule that interferes with the expression of any of the cognate integrins disclosed herein; and a pharmaceutical acceptable carrier; wherein the therapeutically effective amount of a nucleic acid molecule that interferes with the expression of any of the cognate integrins disclosed herein inhibits migration of cancer cells from the lymph node to distant organs.

In some embodiments, the invention relates to a pharmaceutical composition comprising: a therapeutically effective amount or prophylactically effective amount of a polypeptide or functional fragment thereof that interferes with the expression or binding of any of the cognate integrins disclosed herein; and a pharmaceutical acceptable carrier. In some embodiments, the invention relates to a pharmaceutical composition comprising: a therapeutically effective or prophylactically effective amount of a polypeptide or functional fragment thereof that interferes with the expression or binding of any of the cognate integrins disclosed herein; and a pharmaceutical acceptable carrier; wherein the therapeutically effective amount of a polypeptide or functional fragment thereof that interferes with the expression of any of the cognate integrins disclosed herein inhibits migration of cancer cells from the lymph node to distant organs. In some embodiments, the invention relates to a pharmaceutical composition comprising: a therapeutically effective or prophylactically effective amount of a polypeptide or functional fragment thereof that interferes with the expression of any of the cognate integrins disclosed herein; and a pharmaceutical acceptable carrier; wherein the therapeutically effective amount of a polypeptide or functional fragment thereof that interferes with the expression of any of the cognate integrins disclosed herein inhibits migration of cancer cells from the lymph node to distant organs. In some embodiments, the polypeptide or functional fragment thereof that interferes with the expression or binding of any of the cognate integrins disclosed herein is an antibody or antibody fragment. In some embodiments, the composition comprises a polypeptide or nucleic acid sequence that inhibits migration of cancer cells from the tissue from which the cancer cell originates to a lymph node.

The present invention provides for the use of an antibody or binding composition which specifically binds to a specified cognate binding pair to an ECM components disclosed herein or to an integrin disclosed herein. In some embodiments the antibody specifically binds the integrin from a mammalian polypeptide, e.g., a polypeptide derived from a primate, human, cat, dog, rat, or mouse. Antibodies can be raised to various integrins, including individual, polymorphic, allelic, strain, or species variants, and fragments thereof, both in their naturally occurring (full-length) forms or in their synthetic forms. Additionally, antibodies can be raised to the analogs in their inactive state or active state. Anti-idiotypic antibodies may also be used.

A number of immunogens may be selected to produce antibodies specifically reactive with ligand or receptor proteins. Synthetic integrins disclosed herein may serve as an immunogen for the production of monoclonal or polyclonal antibodies. Such antibodies may be used as antagonists or agonists for their targets modulating the disease state associated with the naturally occurring integrins or cognate integrins disclosed herein. Synthetic polypeptides of the claimed invention may also be used either in pure or impure form. Synthetic peptides, made using the appropriate protein sequences, may also be used as an immunogen for the production of antibodies. Naturally folded or denatured material can be used, as appropriate, for producing antibodies. Either monoclonal or polyclonal antibodies may be generated, e.g., for subsequent use in immunoassays to measure the protein, or for immunopurification methods. Methods of producing polyclonal antibodies are well known to those of skill in the art.

Typically, an immunogen, such as a purified integrin disclosed herein of the invention, is mixed with an adjuvant and animals are immunized with the mixture. The animal's immune response to the immunogen preparation is monitored by taking test bleeds and determining the titer of reactivity to the protein of interest. For example, when appropriately high titers of antibody to the immunogen are obtained, usually after repeated immunizations, blood is collected from the animal and antisera are prepared. Further fractionation of the antisera to enrich for antibodies reactive to the protein can be performed if desired. See, e.g., Harlow and Lane; or Coligan. Immunization can also be performed through other methods, e.g., DNA vector immunization. See, e.g., Wang, et al. (1997) Virology 228:278-284.

Monoclonal antibodies may be obtained by various techniques familiar to researchers skilled in the art. Typically, spleen cells from an animal immunized with a desired integrin disclosed herein are immortalized, commonly by fusion with a myeloma cell. See, Kohler and Milstein (1976) Eur. J. Immunol. 6:511-519. Alternative methods of immortalization include transformation with Epstein Barr Virus, oncogenes, or retroviruses, or other methods known in the art. See, e.g., Doyle, et al. (eds. 1994 and periodic supplements) Cell and Tissue Culture: Laboratory Procedures, John Wiley and Sons, New York, N.Y. Colonies arising from single immortalized cells are screened for production of antibodies of the desired specificity and affinity for the antigen, and yield of the monoclonal antibodies produced by such cells may be enhanced by various techniques, including injection into the peritoneal cavity of a vertebrate host. Alternatively, one may isolate DNA sequences which encode a monoclonal antibody or a binding fragment thereof by screening a DNA library from human B cells according, e.g., to the general protocol outlined by Huse, et al. (1989) Science 246:1275-1281.

Antibodies or binding compositions, including binding fragments, single chain antibodies, Fᵥ, F_{ab}, single domain V_{H}, disulfide-bridged Fᵥ, single-chain Fᵥ or F(_{ab}′)₂ fragments of antibodies, diabodies, and triabodies against predetermined fragments of the integrins disclosed herein can be raised by immunization of animals with integrins disclosed herein or conjugates of integrins disclosed herein. Monoclonal antibodies are prepared from cells secreting the desired antibody. These antibodies can be screened for binding to integrins disclosed herein. These monoclonal antibodies will usually bind with at least a K_{D} of about 1 mM, usually at least about 300 μM, typically at least about 10 μM, at least about 30 μM, at least about 10 μM, and at least about 3 μM or more. These antibodies can be screened for binding to the naturally occurring polypeptides upon which the antibodies bind.

In some instances, it is desirable to prepare monoclonal antibodies (mAbs) from various mammalian hosts, such as mice, rodents, primates, humans, etc. Description of techniques for preparing such monoclonal antibodies may be found in, e.g., Stites, et al. (eds.) Basic and Clinical Immunology, 4th ed., Lange Medical Publications, Los Altos, Calif., and references cited therein; Harlow and Lane (1988) Antibodies: A Laboratory Manual CSH Press; Goding (1986) Monoclonal Antibodies: Principles and Practice, 2nd ed., Academic Press, New York, N.Y.; and particularly in Kohler and Milstein (1975) Nature 256:495-497, which discusses one method of generating monoclonal antibodies. Summarized briefly, this method involves injecting an animal with an polypeptide that binds an integrin disclosed herein. The animal is then sacrificed and cells taken from its spleen, which are then fused with myeloma cells.

### Pharmaceutical Compositions

The elucidation of the role played by the integrins associated metastatic cancer and adhesion profiles described herein in adhesion to ECM of a subject facilitates the development of pharmaceutical compositions useful for treatment and diagnosis of metastatic cancer. In some embodiments, the elucidation of the role played by the integrins associated metastatic cancer and adhesion profiles described herein in adhesion to ECM of a subject facilitates the development of pharmaceutical compositions useful for treatment and diagnosis of metastatic breast or lung cancer. These compositions may comprise, in addition to one of the above substances, a pharmaceutically acceptable excipient, carrier, buffer, stabilizer or other materials well known to those skilled in the art. Such materials should be non-toxic and should not interfere with the efficacy of the active ingredient. The precise nature of the carrier or other material may depend on the route of administration, e.g. oral, intravenous, cutaneous or subcutaneous, nasal, intramuscular, intraperitoneal routes. Whether it is a polypeptide, antibody, peptide, nucleic acid molecule, small molecule or other pharmaceutically useful compound according to the present invention that is to be given to an individual, administration is preferably in a “prophylactically effective amount” or a “therapeutically effective amount” (as the case may be, although prophylaxis may be considered therapy), this being sufficient to show benefit to the individual.

### Characterizing Cells

The present invention encompasses the recognition that adhesion signatures characteristic of particular cells of interest are useful in a variety of contexts, for example to identify, characterize, detect, and/or isolate cells of interest.

The present invention provides systems for determining adhesion signatures characteristic of cells. In certain embodiments, the system comprises contacting a sample comprising cells with a collection of extracellular matrix (ECM) components and detecting presence or level of interactions between cells in the sample and ECM components in the collection.

In some embodiments, the system comprises contacting cells with ECM components to allow the cells to adhere to the ECM components. In many embodiments, the interaction between ECM components and particular cells will be higher for cells that interact with higher affinity to a given collection of ECM components. In some embodiments, higher overall affinity may be achieved through individual high affinity interactions. In some embodiments, higher overall affinity may be achieved through a larger number of interactions, whether or not all are particularly high affinity. In some embodiments, overall affinity is affected or determined by multiple interactions between a plurality of distinct pairs of interacting entities. Alternatively or additionally overall affinity is affected or determined by copy number of individual interacting entities; as is understood in the art, a higher concentration of interacting entities can result in a higher number of interactions, which can achieve a higher overall affinity even when individual interactions are relatively modest affinity.

In some embodiments, the systems described herein comprise contacting a sample comprising cells with a collection of extracellular matrix (ECM) components. In some embodiments, a collection of ECM components comprises a single ECM component. In some embodiments, a collection of ECM components comprises 2 ECM components. In some embodiments, a collection of ECM components comprises 3, 4, 5, 6, 7, 8, 9, 10 up to 4,000 or more ECM components.

In some embodiments collections of ECM components for identifying, characterizing, detecting, and/or isolating cancer cells including non-small cell lung cancer cells and cells from primary tumors, lymph nodes, or metastases at organ sites comprises at least two ECM components selected from agrin and collagen IV, agrin and fibrin, biglycan and collagen II, biglycan and fibrin, collagen I and thrombospondin-4, collagen II and decorin, collagen II and tenascin-C, collagen II and testican 2, collagen III and collagen VI, collagen III and thrombospondin-4, collagen IV and galectin 4, collagen IV and SPARC, collagen IV and vitronectin, collagen V and galectin 1, collagen VI and galectin 3, fibrin and galectin 3c, fibrin and galectin 4, fibrin and keratin, fibrin and osteopontin, fibrin and SPARC, f-spondin and fibronectin, fibronectin and galectin 3, fibronectin and galectin 8, fibronectin and laminin, and/or fibronectin and testican 1.

In some embodiments collections of ECM components for identifying, characterizing, detecting, and/or isolating breast cancer cells comprise at least two ECM components selected from agrin and collagen II, agrin and laminin, biglycan and collagen II, brevican and fibronectin, collagen I and testican 2, collagen II and collagen IV, collagen II and laminin, collagen II and nidogen-1, collagen II and testican 2, collagen III and galectin 8, collagen III and superfibronectin, collagen V and fibronectin, collagen V and galectin 1, collagen VI and fibronectin, collagen VI and nidogen-1, collagen VI and tenascin-C, decorin and fibronectin, decorin and galectin 8, decorin and laminin, elastin and galectin 4, fibrin and galectin 3, fibronectin and galectin 1, fibronectin and galectin 3, fibronectin and galectin 4, fibronectin and mucin, fibronectin and SPARC, fibronectin and testican 2, galectin 1 and galectin 3, galectin 1 and keratin, galectin 3 and heparan sulfate, galectin 3 and superfibronectin, galectin 4 and nidogen-1, galectin 8 and tenascin-C, keratin and laminin, laminin and merosin, laminin and thrombospondin-4, SPARC and superfibronectin, and/or superfibronectin and testican 1.

In some embodiments, ECM components are attached to a solid phase. In some embodiments, a solid phase comprises any solid or semi-solid surface. In some embodiments, a solid phase comprises any traditional laboratory material for growing or maintaining cells including petri dishes, beakers, flasks, test tubes, microtitre plates, and/or culture slides. In some embodiments, a solid phase comprises a glass slide.

In some embodiments, ECM components in the collection are attached to discrete sites on a solid phase. In some embodiments the collection of ECM components are attached to a plurality of discrete sites on the solid phase. In some embodiments, a plurality of discrete sites comprises individual site containing only one ECM component. In some embodiments, a plurality of discrete sites comprises individual site containing two or more different ECM components. In some embodiments, a plurality of discrete sites comprises individual sites containing only one ECM component and individual sites containing two or more different ECM components. In some embodiments, different sites within the plurality of sites contain same component(s). In some embodiments, different sites within the plurality of sites contain different component(s). In some embodiments, the plurality of sites comprises sites comprising the same component(s) as other sites within the plurality of sites and sites comprising different component(s) from other sites within the plurality of sites. In some embodiments, the ECM components in the collection attached to discrete sites on a solid phase comprises an array.

In some embodiments, the solid or semi-solid surface comprising a solid phase is comprised of any material on which ECM components can be attached. In some embodiments, a solid phase comprises polyamides, polyesters, polystyrene, polypropylene, polyacrylates, polyvinyl compounds (e.g. polyvinylchloride), polycarbonate, polytetrafluoroethylene (PTFE), nitrocellulose, cotton, polyglycolic acid (PGA), cellulose, dextran, gelatin, glass, fluoropolymers, fluorinated ethylene propylene, polyvinylidene, polydimethylsiloxane, polystyrene, silicon substrates (such as fused silica, polysilicon, or single silicon crystals) or combinations thereof.

In some embodiments, contacting cells with a collection of ECM components in accordance with systems of the present invention comprises mixing cells with a collection of ECM components. In some embodiments, contacting cells with a collection of ECM components comprises overlaying cells on a collection of ECM components on a solid support. In some embodiments, contacting cells with a collection of ECM components comprises submerging ECM components on a solid support in cells. In some embodiments, contacting cells with a collection of ECM components comprises seeding cells onto ECM components on a solid support. In some embodiments, contacting cells with a collection of ECM components comprises seeding from 0.1 to 100 ml or from 1 to 10 ml of cells onto ECM components on a solid support. Alternatively, cells can be brought into contact with ECM components using any other means of transporting liquid.

In some embodiments, cells are contacted with ECM components under conditions and for a time sufficient to allow cells to interact with ECM components. In some embodiments, cells are contacted with ECM components for from 10 minutes to 48 hours, from 30 minutes to 24 hours, or from 1 hour to 12 hours. In a specific exemplary embodiment, cells are contacted to ECM components for 2 hours.

In some embodiments, contacting is performed at a temperature within a range consistent with cell viability and/or metabolic function. In some embodiments, contacting is performed at a temperature of between 10 to 70, of between 20 to 60, or of between 25 to 40 degrees Celsius. In a specific exemplary embodiment, the temperature is 37 degrees Celsius.

In some embodiments, contacting cells with a collection of ECM components further comprises washing ECM components. In some embodiments, ECM components are washed to remove excess cells. In some embodiments, ECM components are washed to remove non-interacting cells. In some embodiments, ECM components are washed in any solution that will not damage the cells or ECM components. In certain embodiments, ECM components are washed in the same cell culture media that is used to contact the cells to the ECM components. Alternatively, ECM components can be washed with PBS.

In certain embodiments, ECM components are washed in any arrangement that allows the cells interacting with ECM components to maintain their interaction with the ECM components. In certain embodiments, ECM components are washed in a stationary arrangement. In certain embodiments, ECM components, are mechanically agitated during washing. Methods for agitating cells in culture are well known in the art and include but are not limited to use of nutators, rockers, rotators, and shakers.

In some embodiments, the level of interactions between cells in the sample and ECM components in the collection is detected. In some embodiments, interactions between cells and ECM components is detected using any technology that allows cells interacting with ECM components to be quantified. In some embodiments interactions between cells and ECM components is detected by microscopy. In some embodiments interactions between cells and ECM components is detected by confocal microscopy. In some embodiments interactions between cells and ECM components is detected by fluorescence microscopy. In some embodiments interactions between cells and ECM components is detected by microscopy on live cells. In some embodiments interactions between cells and ECM components is detected by microscopy on fixed cells. Appropriate fixatives are well known in the art and include but are not limited to formaldehyde, glutaraldehyde, and formalin. In some embodiments interactions between cells and ECM components is detected by microscopy on stained cells. Appropriate stains for counting cells via microscopy are well known in the art. Examples include but are not limited to Hoechst, 4′,6-diamidino-2-phenylindole (DAPI), and acridine orange. In some embodiments interactions between cells and ECM components is detected by immunocytochemistry.

In some embodiments, detecting interactions between cells and ECM components comprises quantifying the interactions. In some embodiments interactions between cells and ECM components is/are quantified by any means that allows quantification of cells interacting with ECM components. In some embodiments interactions between cells and ECM components detected by microscopy is/are quantified visually. In some embodiments interaction between cells and ECM components detected by microscopy is/are quantified with the aid of a computer program or other computational device. Computer programs for quantifying cell number from microscopic images are well known in the art. One exemplary mathematical programs for quantification of cells visualized by microscopy includes CellProfiler (Carpenter, A. E., et al. CellProfiler: image analysis software for identifying and quantifying cell phenotypes. Genome Biology, 7: R100, 2006, which is incorporated by reference in its entirety).

In some embodiments cluster analysis is performed on quantified interactions between cells and ECM components. Analyzing array data is a technique that is well known in the art. Computer programs for analyzing array data include but are not limited to Spotfire (Tibco) and Genespring (Agilent).

In some embodiments, methods in accordance with the disclosure may be used as a diagnostic tool to distinguish between cell types by detecting adhesion signatures characteristic of particular cells that distinguish those cells from other cells in the sample or from reference cells. For example, Example 4 of the present application demonstrates two metastatic cancer cell lines that cluster more closely to each other than to parental tumor cells from which they are derived. When the same cells are characterized by microarray analysis, however, each metastatic cell line clusters with the parental line from which it is derived. These data suggests that adhesion signatures can be used to detect metastatic changes in cancer cells that are undetectable by microarrays.

In certain embodiments, cells differing in one or more characteristic are distinguished by adhesion signatures. In certain embodiments, cells are distinguished from reference cells by adhesion signatures. In certain embodiments, cells at different stages of disease progression are distinguished by adhesion signatures. In certain embodiments, cancer cells at different stages of metastasis are distinguished by adhesion signatures. In some embodiments, cells at different stages of development are distinguished by adhesion signatures. In some embodiments, stem cells at differing stages of differentiation are distinguished by adhesion signatures. In some embodiments, stem cells at differing stages of differentiation include mesenchymal stem cells at different stages of differentiation towards osteogenic, chondrogenic or adipogenic lineages. In some embodiments, stem cells at differing stages of differentiation include human induced pluripotent stem cells or embryonic stem cells at different stages of differentiation towards any cell lineage of circulatory, nervous, or immune systems.

The present invention also provides systems for determining effects on cells of interacting with extracellular matrix components comprising exposing a first population of cells to a first set of conditions that includes contacting with a collection of extracellular matrix components, exposing a second population of cells, which second population of cells is comparable to the first population of cells, to a second set of conditions, which second set of conditions is comparable to the first set of conditions except that some or all of the extracellular matrix components are absent from the contacting, and determining one or more cell population features that differs between the first and second populations of cells after the exposing has occurred. In certain embodiments, information about cells or cell types, including information that characterizes the particular cell or cell type as compared with a different cell type, may be obtained while cells are in contact with ECM components. In certain embodiments effects on cells that result from exposure to and/or interaction with one or more ECM components are determined in accordance with the present invention, for example by determining features that differ in cells that are exposed to different ECM components. In some embodiments, presence or degree of features is determined to correlate with presence or level of one or more ECM components and/or with one or more adhesion signatures. In certain embodiments, cells are probed.

In certain embodiments, a population of cells comprises any collection of cells. In certain embodiments, a population of cells comprises cells of a certain type, wherein the cell type is unknown. In certain embodiments, a population of cells comprises cells of a known cell type. In some embodiments, a population of cells comprises a mixture of known or unknown cell types. In some embodiments, a population of cells comprises cells of a biological sample.

In some embodiments, cells are probed with antibodies that allow cells with different characteristics to be distinguished. It will be appreciated that the use of antibodies as probes is well known to those in the art. Antibodies are available to detect cell lineages or disease states. For example, anti-AFP antibodies can be used to distinguish undifferentiated stem cells from those differentiated towards hepatic lineages and anti-Pdx1 antibodies can be used to distinguish undifferentiated stem cells from those differentiated towards pancreatic lineages. Degree of antibody staining can be detected by techniques well known in the art using fluorescently or chemiluminescently labeled antibodies or by probing with a fluorescently or chemiluminescently labeled secondary antibodies.

In some embodiments, cells are probed with any sort of DNA probe. In some embodiments, cells are probed with DNA probes that allow cells with different characteristics to be distinguished by genotype. In some embodiments, cells are probed with DNA probes that allow cells with different characteristics to be distinguished by RNA transcripts. In some embodiments, cells are probed with any sort of labeled substrate. In some embodiments, cells are probed with labeled substrate that allows cells with different characteristics to be distinguished by enzymatic activity. In some embodiments, cells probed with any sort of protein. In some embodiments, cells are probed with a protein that allow cells with different characteristics to be distinguished by affinity for proteins other than ECM components.

### Diagnosis

As described above, certain embodiments of the present invention may be used to distinguish between cells at different states of cancer progression, making it a promising tool for diagnosing disease. This system is potentially useful, for example, when testing cells of a patient to determine whether disease is present. Diagnosing a patient using adhesion signatures would include, for example, comparing an adhesion signature of a sample from a patient with and adhesion signature of reference cells.

In certain embodiments, adhesion signatures are used to diagnose and/or prognose a patient suspected of having any condition causing his or her cells to have a distinguishing characteristic from reference cells as a result of the condition. In certain embodiments, adhesion signatures are used to diagnose and/or prognose a patient suspected of having any disease that affects adhesion signatures of his or her cells. In certain embodiments, adhesion signatures are used to diagnose and/or prognose a patient suspected of having any form of cancer. In certain embodiments, adhesion signatures are used to diagnose and/or prognose a patient suspected of having lung cancer. In certain embodiments, adhesion signatures are used to diagnose and/or prognose a patient suspected of having metastatic cancer. In certain embodiments, adhesion signatures are used to diagnose and/or prognose a patient suspected of having breast cancer. In certain embodiments, adhesion signatures are used to diagnose and/or prognose a patient suspected of having colon cancer. In certain embodiments, adhesion signatures are used to diagnose and/or prognose a patient suspected of having prostate cancer. In certain embodiments, adhesion signatures are used to diagnose and/or prognose a patient suspected of having testicular cancer. In certain embodiments, adhesion signatures are used to diagnose and/or prognose a patient suspected of having brain cancer. In certain embodiments, adhesion signatures are used to diagnose and/or prognose a patient suspected of having leukemia.

In some embodiments, kits in accordance with the disclosure provide a means of diagnosing cancer stage. Providing tools for diagnosis and/or prognosis via adhesion signatures in kit form brings adhesion signature technology to clinical settings. In some embodiments, kits for cancer stage diagnosis comprise a substrate coated with a collection of ECM components characterized in that, when a sample containing cells of a plurality of different cell types, which plurality of different cell types include cancer cells of a particular stage of metastasis, is contacted with the substrate, cancer cells of a particular stage of metastasis form a set of interactions with ECM components in the collection sufficient to isolate the cells of the cell type of interest from other cells in the sample. In some embodiments, the kit further comprises medium. In some embodiments, kits in accordance with the disclosure provide a means of detecting non-small cell lung cancer cells and cells from primary tumors, lymph nodes, or metastases at organ sites and comprise at least two ECM components as disclosed herein.

In some embodiments, presence of cancer cells of a particular stage of metastasis is detected by growth of those cells. In some embodiments, the kit further comprises a means for assessing growth or abundance of the cells. Methods for detecting and/or assessing cell growth and/or abundance are well known in the art and include but are not limited to spectrophotometry, FACS, microscopy, and/or plating. In some embodiments, a means for assessing growth or abundance of the cells comprises a container for sending the kit to a facility where growth and/or abundance is assessed.

### Cell Type Isolation

In some embodiments, methods in accordance with the disclosure may be used as a tool to isolate cells of interest. This system is useful, for example, when trying to isolate certain types of cells out of a mixed cell population. When given a complex mixture of cells, for example partially differentiated stem cells, a patient biopsy, or a bone marrow sample, deconvolving this mixture using traditional methods can be difficult. In general, it is thought that one of the easiest ways to achieve this result is by flow cytometry, but flow cytometry requires an initial prediction of what might be present in a sample to establish a panel of markers that would represent that population. In some embodiments of the present invention, the use of adhesion signatures simplifies this process. Example 7, for instance, demonstrates that mesenchymal stem cells, which are normally isolated out of bone marrow, have high affinity for a combination of galectin-8 and thrombospondin-4. Stem cells can be human or derived from any other type of animal.

In some embodiments, the steps of isolating a particular cell type comprises contacting a sample comprising cells with a collection of extracellular matrix (ECM) components under conditions and for a time sufficient for a set of interactions to occur between particular cells in the sample and ECM components in the collection sufficient to isolate the cells from other components of the sample. In some embodiments, ECM components are used to separate cells from other cells. In some embodiments, ECM components are used to separate cells from other cells that make a different set of interactions with the ECM components than do the isolated cells. In some embodiments, cells are isolated using ECM components attached to a solid phase. In some embodiments, cells are isolated using ECM components attached to a solid phase by separating the solid phase from the sample.

### Cell Culture

In some embodiments, methods in accordance with the disclosure may be used to identify suitable culture conditions for and/or to propagate cells or cell types of interest. Any type of cell grown in culture that originates from a tissue requires a solid surface on which to attach and proliferate. It is generally understood that ECM components facilitate attachment to surfaces. There exist many cell types for which ideal culturing conditions remain unknown and ECM arrays could potentially provide this information.

This system is particularly useful for culturing stem cells because current methods to grow induced pluripotent stem cells require mitotically inactivated feeder cells (MEFs) or undefined extracellular matrix (ECM) mixes (i.e. Matrigel) and thus introduce animal factors and lot variability. Use of defined ECM components, particularly combinations of collagen II and galectin 4, collagen IV and galectin 8, or collagen I and laminin in combination with a defined media offer the potential to generate and maintain pluripotent stem cells without contamination by animal products and may therefore have translational implications for treatment of human disease.

This system is also particularly useful for cell types that are difficult to culture because it allows testing a wide variety of conditions simultaneously. One example is culturing of hepatocytes—the main hepatic cell types. In general, it is thought that only around 10% of donor cells are plateable after isolation. As described in example 8, for all of 6 different lots of unplateable hepatocytes, several ECM matrix combinations were identified that promoted cell adhesion. Combinations of collagen I with aggrecan and of collagen IV with nidogen-1 seem to have a universal effect.

In certain embodiments, culturing a cell type of interest comprises contacting a sample comprising cells of a cell type of interest with a collection of extracellular matrix (ECM) components appropriate to promote growth and/or replication of cells of the cell type of interest as compared with cells of one or more different cell types.

In certain embodiments, a cell type of interest in accordance with the present disclosure comprises human embryonic stem cells or human induced pluripotent stem cells; in some such embodiments, the collection of ECM components comprises at least two ECM components selected from collagen II and galectin 4, collagen IV and galectin 8, or collagen I and Laminin.

In certain embodiments, a cell type of interest in accordance with the present disclosure comprises hepatocytes; in some such embodiments, the collection of ECM components comprises at least two ECM components selected from agrin and collagen I, collagen I and laminin, collagen I and merosin, collagen II and galectin 8, collagen II and SPARC, and/or collagen IV and nidogen-1.

In certain embodiments, a cell type of interest in accordance with the present disclosure comprises mesenchymal stem cells; in some such embodiments, the collection of ECM components comprises at least two ECM components selected from biglycan and collagen IV, biglycan and galectin 4, brevican and collagen I, brevican and collagen IV, brevican and galectin 3c, collagen I and galectin 1, collagen I and galectin 3, collagen I and galectin 3c, collagen I and galectin 8, collagen I and nidogen-2, collagen I and SPARC, collagen I and tenascin-C, collagen I and testican 1, collagen I and vitronectin, collagen II and galectin 3, collagen II and galectin 8, collagen II and nidogen-1, collagen II and nidogen-2, collagen IV and decorin, collagen IV and galectin 8, collagen IV and nidogen-1, collagen IV and nidogen-2, collagen IV and testican 1, collagen IV and testican 2, collagen VI and f-spondin, collagen VI and galectin 3, collagen VI and galectin 8, collagen VI and tenascin-C, collagen VI and testican 2, collagen VI and thrombospondin-4, f-spondin and vitronectin, fibrin and galectin 4, fibronectin and galectin 4, fibronectin and nidogen-1, fibronectin and tenascin-C, fibronectin and testican 1, fibronectin and testican 2, galectin 3 and vitronectin, galectin 3c and merosin, galectin 3c and superfibronectin, galectin 4 and superfibronectin, galectin 8 and superfibronectin, galectin 8 and vitronectin, laminin and vitronectin, SPARC and testican 1, and/or superfibronectin and vitronectin. In some embodiments, the mesenchymal stem cells are derived from bone marrow, adipose tissue, umbilical cord blood or umbilical cord.

In certain embodiments, culturing a cell type of interest comprises culturing cells in any type of media that is capable of supporting growth of the cell type of interest. In certain embodiments, media comprises cell culture media. In certain embodiments, media comprises complex media. In certain embodiments, media comprises serum-free media. The selection of appropriate cell culture media appropriate for various cell types is well known in the art.

In some embodiments, the cells are cultured at a temperature within a range consistent with cell viability and/or metabolic function. In some embodiments, the cells are cultured a temperature of between from 10 to 70, of between 20 to 60, or of between 25 to 40 degrees Celsius.

In some embodiments, systems in accordance with the present disclosure may be used to culture and/or to propagate cells or cell types of interest. In some embodiments, systems for culturing cells comprise a substrate coated with a collection of ECM components characterized in that, when a sample containing cells of a plurality of different cell types, which plurality of different cell types includes at least one cell type of interest is contacted with the substrate, cells of the cell type of interest form a set of interactions with ECM components in the collection sufficient to isolate the cells of the cell type of interest from other cells in the sample by promoting growth of the cell type of interest. In some embodiments, systems in accordance with the present disclosure may be used to culture and/or to propagate mesenchymal stem cells, hepatocytes, human induced pluripotent stem cells or embryonic stem cells and comprise at least two ECM components as described herein.

### Kits

In some embodiments, kits in accordance with the present disclosure may be used to culture and/or to propagate cells or cell types of interest. In some embodiments, kits for culturing cells comprise the substrate described above and optionally further comprise medium and a cell type of interest. Any array, system, or component thereof disclosed may be arranged in a kit either individually or in combination with any other array, system, or component thereof. The invention provides a kit to perform any of the methods described herein. In some embodiments, the kit comprises at least one container comprising one or a plurality of polypeptides comprising a polypeptide sequence associated with the extracellular matrix or functional fragments thereof. In some embodiments, the kit comprises at least one container comprising any of the polypeptides or functional fragments described herein. In some embodiments, the polypeptides are in solution (such as a buffer with adequate pH and/or other necessary additive to minimize degradation of the polypeptides during prolonged storage). In some embodiments, the polypeptide are lyophilized for the purposes of resuspension after prolonged storage. In some embodiments, the kit comprises: at least one container comprising one or a plurality of polypeptides comprising a polypeptide sequence associated with the extracellular matrix (or functional fragments thereof); and a solid support upon which the polypeptides or fragments may be affixed. In some embodiments, the kit optionally comprises instructions to perform any or all steps of any method described herein. In some embodiments, the kit comprises an array or system described herein and instructions for implementing one or a plurality of steps using a computer program product disclosed herein. It is understood that one or a plurality of the steps from any of the methods described herein can be performed by accessing a computer program product encoded on computer storage medium directly through one or more computer processors or remotely through one or more computer processors via an internet connection or other virtual connection to the one or more computer processors. In some embodiments, the kit comprises a computer-program product described herein or requisite information to access a computer processor comprising the computer program product encoded on computer storage medium remotely. In some embodiments, the computer program product, when executed by a user, calculates one or more adhesion values, normalizes the one or more adhesion values, generates one or more adhesion signatures or one or more adhesion profiles, and/or displays any of the adhesion values, adhesion signatures, adhesion profiles to a user. In some embodiments, the kit comprises a computer program product encoded on a computer-readable storage medium that comprises instructions for performing any of the steps of the methods described herein. In some embodiments, the invention relates to a kit comprising instructions for providing one or more adhesion values, one or more normalized adhesion values, one or more adhesion profiles, one or more adhesion signatures, or any combination thereof. In some embodiments, the kit comprises a computer program product encoded on a computer storage medium that when, executed on one or a plurality of computer processors, quantifies an adhesion value, determines an adhesion signature or adhesion profile, and/or displays an adhesion signature, adhesion value, adhesion signature, and/or any combination thereof. In some embodiments, the kit comprises a computer program product encoded on a computer storage medium that, when executed by one or a plurality of computer processors, quantifies adhesion values of one or more cells samples and determines an adhesion signature based at least partially upon the adhesion values. In some embodiments, kit comprises instructions for accessing the computer storage medium, quantifying adhesion values, normalizing adhesion values, determining an adhesion signature of a cell type, and/or any combination of steps thereof. In some embodiments, the computer-readable storage medium comprises instructions for performing any of the methods described herein. In some embodiments, the kit comprises an array or system disclosed herein and a computer program product encoded on computer storage medium that, when executed, performs any of the method steps disclosed herein individually or in combination and provides instructions for performing any of the same steps. In some embodiments, the instructions comprise an instruction to adhere any one or plurality of polypeptides disclosed herein to a solid support.

The invention further provides for a kit comprising one or a plurality of containers that comprise one or a plurality of the polypeptides or fragments disclosed herein. In some embodiments, the kit comprises cell media free of serum, or any animal-based derivative of serum that enhances the culture or proliferation of cells. In some embodiments, the kit comprises: an array disclosed herein, any cell media disclosed herein, and a computer program product disclosed herein optionally comprising instructions to perform any one or more steps of any method disclosed herein. In some embodiments, the kit does not comprise cell media. In some embodiments, the kit comprises a solid support free of any one individual pair of polypeptides disclosed herein. In some embodiments, the kit comprises a device for affixing one or more adhesion sets to a solid support.

The kit may contain two or more containers, packs, or dispensers together with instructions for preparation of an array. In some embodiments, the kit comprises at least one container comprising the array or system described herein and a second container comprising a means for maintenance, use, and/or storage of the array such as storage buffer. In some embodiments, the kit comprises a composition comprising any polypeptide disclosed herein in solution or lyophilized or dried and accompanied by a rehydration mixture. In some embodiments, the polypeptides and rehydration mixture may be in one or more additional containers.

The compositions included in the kit may be supplied in containers of any sort such that the shelf-life of the different components are preserved, and are not adsorbed or altered by the materials of the container. For example, suitable containers include simple bottles that may be fabricated from glass, organic polymers, such as polycarbonate, polystyrene, polypropylene, polyethylene, ceramic, metal or any other material typically employed to hold reagents or food; envelopes, that may consist of foil-lined interiors, such as aluminum or an alloy. Other containers include test tubes, vials, flasks, and syringes. The containers may have two compartments that are separated by a readily removable membrane that upon removal permits the components of the compositions to mix. Removable membranes may be glass, plastic, rubber, or other inert material.

Kits may also be supplied with instructional materials. Instructions may be printed on paper or other substrates, and/or may be supplied as an electronic-readable medium, such as a floppy disc, CD-ROM, DVD-ROM, zip disc, videotape, audio tape, or other readable memory storage device. Detailed instructions may not be physically associated with the kit; instead, a user may be directed to an internet web site specified by the manufacturer or distributor of the kit, or supplied as electronic mail.

The invention also provides a kit comprising: an array of polypeptides, the array comprising: a solid support and a plurality of adhesion sets, wherein each adhesion set comprises two or more different polypeptides comprising a polypeptide sequence associated with the extracellular matrix or a functional fragment thereof; and optionally comprising a cell culture vessel. In some embodiments, the kit further comprises at least one of the following: cell media, a volume of fluorescent stain or dye, a cell sample, and a set of instructions, optionally accessible remotely through an electronic medium.

Any and all journal articles, patent applications, issued patents, or other cited references disclosed herein are incorporated by reference in their respective entireties.

### EXAMPLES

### Example 1

### ECM Array Fabrication

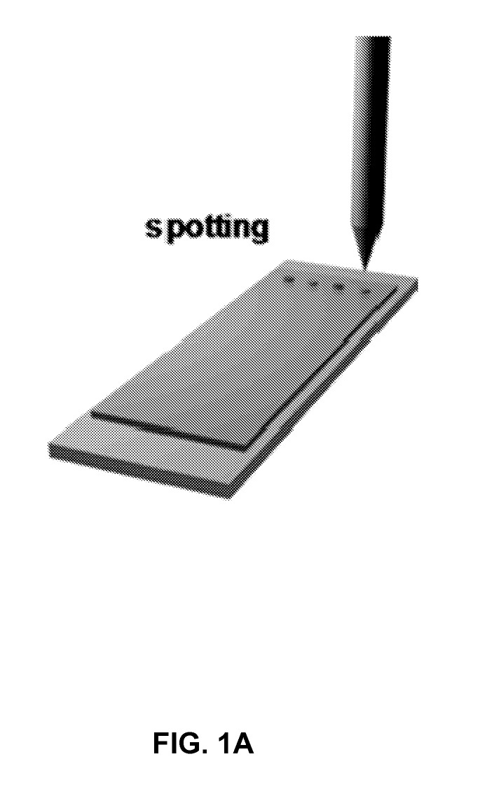

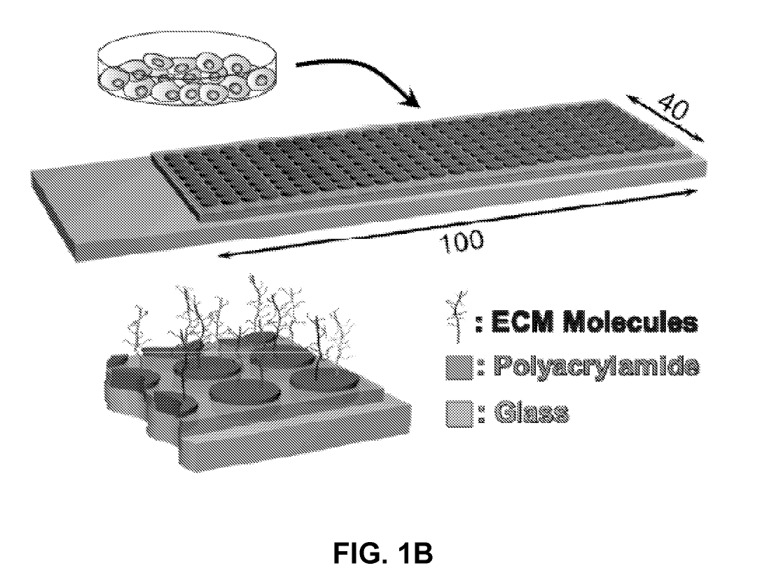

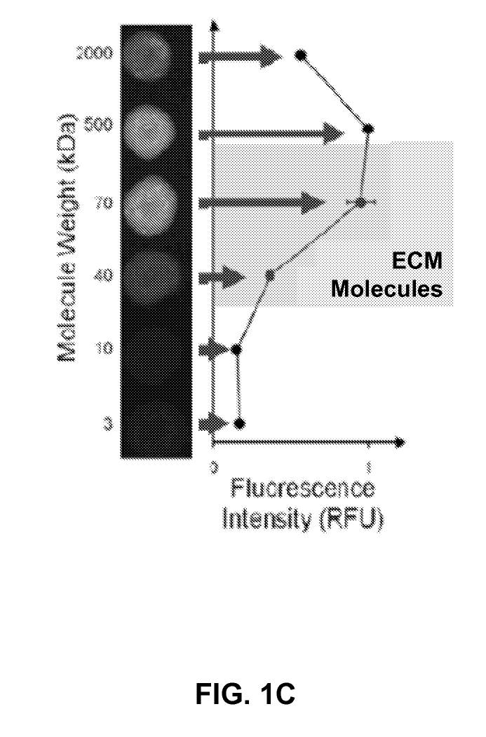

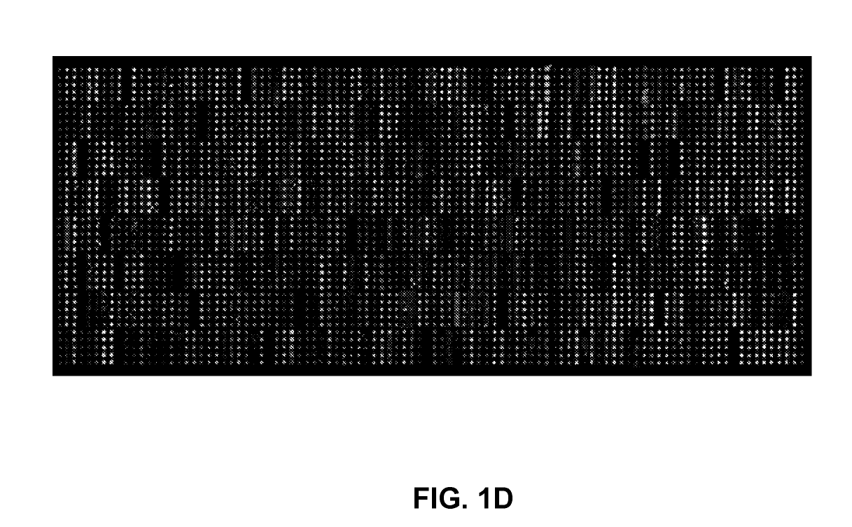

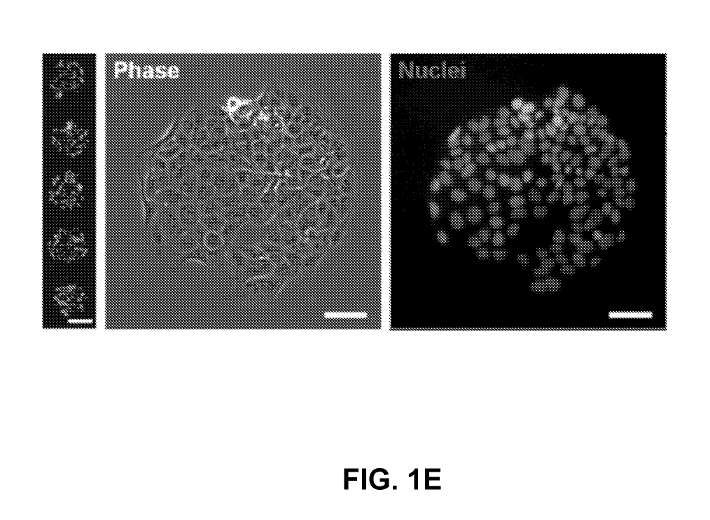

The following example describes an ECM array. Among other things, the present invention provides a collection of ECM components attached to a solid surface useful in accordance with the present invention to define, detect, or utilize one or more features of an adhesion signature of a cell or cell type. In some embodiments, this collection can be defined as an ECM array. In the following example, an expanded Extracellular Matrix (ECM) array is developed for the purpose of identifying different cell types via their adhesion signature. The array described in US Published Patent Application 2006/0160066 A1 was adapted to incorporate all single and pair-wise combinations of 38 different ECM components (Table 1) for a total of 768 combinations presented in quintuplicate in the ECM array resulting in an overall number of 4000 spots per microscope slide (FIG. 1B).

TABLE D
ECM components

Aggrecan
Agrin
Biglycan
Brevican
Chondroitin Sulfate
Collagen I
Collagen II
Collagen III
Collagen IV
Collagen V
Collagen VI
Decorin
Elastin
F-Spondin
Fibrin
Fibronectin
Galectin 1
Galectin 3
Galectin 3c
Galectin 4
Galectin 8
Heparan Sulfate
Hyaluronic Acid
Keratin
Laminin

Briefly, vantage acrylic slides (CEL Associates VACR-25C) were coated with polyacrylamide as previously described (Flaim, C. J. et al. An extracellular matrix microarray for probing cellular differentiation. Nat Meth, 2(2): 119-125, 2005). Before deposition of the molecules, slides are coated with a polyacrylamide hydrogel that is allowed to dry after soaking to remove any unpolymerized monomer. The dehydrated hydrogel acts to entrap molecules without requiring their chemical modification. Slides were then spotted using a DNA Microarray spotter (Cartesian Technologies Pixsys Microarray Spotter and ArrayIt 946 Pins) from source plates prepared using a Tecan liquid handler. Molecules were prepared at a concentration of 200 μg/ml using a buffer described previously (Flaim et al.). 768 pairwise combinations were spotted in replicates of five (FIG. 1A). Rhodamine dextran (Invitrogen) was spotted as negative controls and for use in image alignment. The following molecules were used: Collagen I (Millipore), Collagen II (Millipore), Collagen III (Millipore), Collagen IV (Millipore), Collagen V (BD Biosciences), Collagen VI (BD Biosciences), Fibronectin (Millipore), Laminin (Millipore), Merosin (Millipore), Tenascin-R (R&D Systems), Chondroitin Sulfate (Millipore), Aggrecan (Sigma), Elastin (Sigma), Keratin (Sigma), Mucin (Sigma), Superfibronectin (Sigma), F-Spondin (R&D Systems), Nidogen-2 (R&D Systems), Heparan Sulfate (Sigma), Biglycan (R&D Systems), Decorin (R&D Systems), Galectin 1 (R&D Systems), Galectin 3 (R&D Systems), Galectin 3c (EMD Biosciences), Galectin 4 (R&D Systems), Galectin 8 (R&D Systems), Thrombospondin-4 (R&D Systems), Osteopontin (R&D Systems), Osteonectin (R&D Systems), Testican 1 (R&D Systems), Testican 2 (R&D Systems), Fibrin (Sigma), Tenascin-C(R&D Systems), Nidogen-1 (R&D Systems), Vitronectin (R&D Systems), Rat Agrin (R&D Systems), Hyaluronan (R&D Systems), Brevican (R&D Systems). Slides were then stored in a humidity chamber at 4° C. To quantify cells bound to each spot, nuclei are stained according to conventional fluorescence staining protocols, and the slides are imaged using an automated inverted epifluorescent microscope with NIS Elements software. Our data indicate that molecules larger than ˜10 kDa can be robustly entrapped in the hydrogel (FIG. 1C). We verified their entrapment using NHS-Fluorescein labelling or antibody-mediated detection after entrapment (FIG. 1D). Of the 38 molecules that we tested by these methods, all showed excellent reproducibility and uniformity within the expected region of printing (FIG. 1D). Representative images of cells adhered to ECM spots demonstrating selective adhesion in the locations of ECM (FIG. 1E). Scale bar on five-spot image is 200 μm. Scale bars on single-spot images are 50 μm.

### Example 2

### Using the ECM Array to Assay Cell Adhesion Signatures

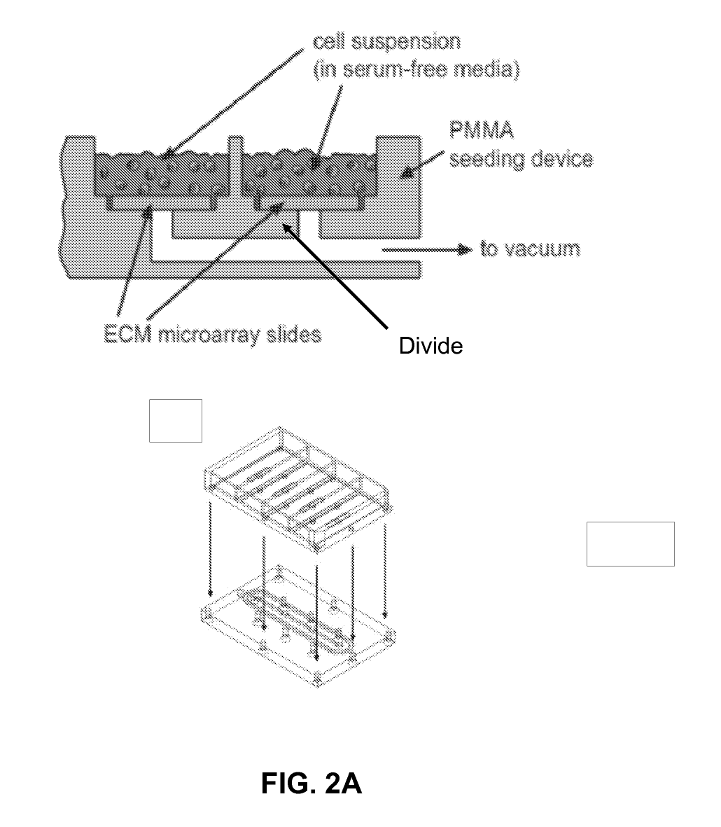

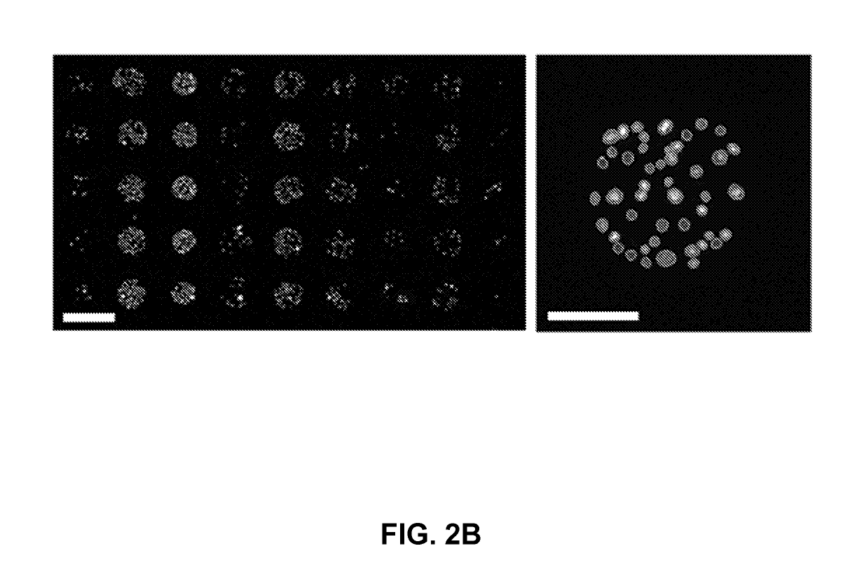

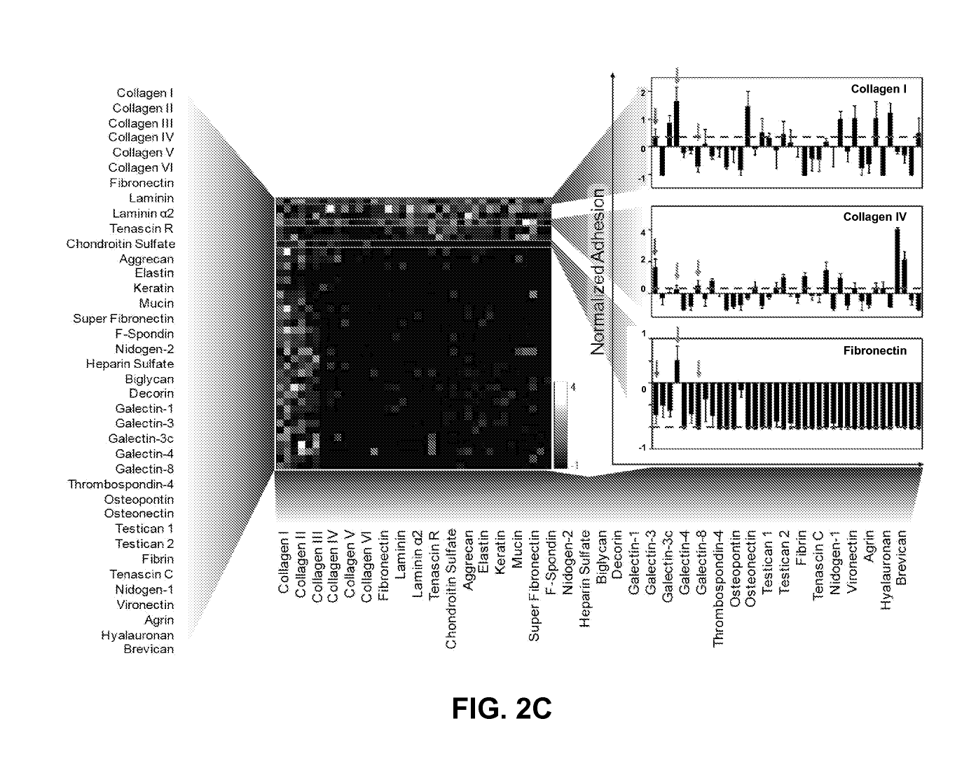

In this example, a protocol for detection of adhesion signatures using ECM arrays is described. Extracellular matrix microarrays preparation. Vantage acrylic slides (CEL Associates VACR-25C) were coated with polyacrylamide by depositing prepolymer containing Irgacure 2959 photoinitiator (Ciba) between the slide and a glass coverslip22. Following polymerization, slides were soaked in ddH2O and the coverslips were removed. Slides were allowed to dry before molecule deposition. Slides were spotted using a DNA Microarray spotter (Cartesian Technologies Pixsys Microarray Spotter and ArrayIt 946 Pins). 768 combinations were spotted in replicates of five. Rhodamine dextran (Invitrogen) was spotted as negative controls and for use in image alignment. The following molecules were used: Collagen I (Millipore), Collagen II (Millipore), Collagen III (Millipore), Collagen IV (Millipore), Collagen V (BD Biosciences), Collagen VI (BD Biosciences), Fibronectin (Millipore), Laminin (Millipore), Merosin (Millipore), Tenascin-R(R&D Systems), Chondroitin Sulphate (Millipore), Aggrecan (Sigma), Elastin (Sigma), Keratin (Sigma), Mucin (Sigma), Superfibronectin (Sigma), F-Spondin (R&D Systems), Nidogen-2 (R&D Systems), Heparan Sulphate (Sigma), Biglycan (R&D Systems), Decorin (R&D Systems), Galectin 1 (R&D Systems), Galectin 3 (R&D Systems), Galectin 3c (EMD Biosciences), Galectin 4 (R&D Systems), Galectin 8 (R&D Systems), Thrombospondin-4 (R&D Systems), Osteopontin (R&D Systems), Osteonectin (R&D Systems), Testican 1 (R&D Systems), Testican 2 (R&D Systems), Fibrin (Sigma), Tenascin-C(R&D Systems), Nidogen-1 (R&D Systems), Vitronectin (R&D Systems), Rat Agrin (R&D Systems), Hyaluronan (R&D Systems), Brevican (R&D Systems). The laminin used is Millipore catalogue no. AG56P, and is a mixture of human laminins that contain the beta1 chain. Source plates used in the spotter were prepared using a Tecan liquid handler. Molecules were prepared at a concentration of 200 μg ml-1 using a buffer described previously22. Slides were stored in a humidity chamber at 4° C. before use. Extracellular matrix microarray seeding and analysis. Slides were washed in PBS and treated with UV before seeding cells. Slides were washed in PBS and treated with UV prior to seeding cells. To measure cell-ECM interactions, cells are seeded onto the arrays in serum-free media and allowed to adhere for 1.5 h at 37° C. To ensure uniform seeding, the slides are agitated every 15 minutes. Furthermore, the top surfaces of the slides are held flush with the bottom of the plate through the use of a custom-designed seeding device that employs a vacuum seal (FIG. 2A). This device minimizes seeding variability between experiments and avoids cell loss by preventing cells from settling below the slide surface or on the backs of the slides. Uniformity of seeding across individual arrays and between replicate arrays was confirmed using test slides composed of only one matrix molecule.

Adhesion signatures of mouse tumor cells were characterized. To determine whether metastatic progression is characterized by discrete changes in the ability of cancer cells to adhere to ECM components a panel of cell lines derived from a genetically-engineered model of lung adenocarcinoma was used. Cell lines have been described in Winslow, M. M. et al. Suppression of lung adenocarcinoma progression by Nkx2-1; Nature 473, 101-104 (2011). Lung adenocarcinoma cells in people often contain an activating mutation in the KRAS oncogene and an inactivating mutation in the p53-tumor suppressor pathway. In this mouse model, inhalation of lentiviral Cre-recombinase by genetically-engineered mice G12D containing a loxP-Stop-loxP Kras knock-in allele and both p53 alleles flanked by loxP sites (KrasLSL-G12D/+; p53flox/flox) initiates lung adenocarcinoma development (DuPage, M. et al. Conditional mouse lung cancer models using adenoviral or lentiviral delivery of Cre recombinase. Nat. Protocols, 4(8): 10641072, 2009, Jackson, E. L., et al., Analysis of lung tumor initiation and progression using conditional expression of oncogenic K-ras. Genes & Development, 15(24): 3243-3248, 2001). Distal metastases form over months in lymph nodes as well as many secondary organs (kidneys, adrenal glands, liver, etc.). Tumors can be resected from the lung and metastatic sites and cultured in vitro as cell lines. Metastatic populations can be correlated to their primary tumor of origin through the use of linker-mediated polymerase chain reaction (LMPCR) and specific PCR for the integration site, since the lentiviruses integrate stably into the genome (Winslow, M. M., et al. Suppression of lung adenocarcinoma progression by Nkx2-1. Nature, 473(7345): 101-104, 2011). Thus, cell lines were generated from primary tumors that did not form detectable metastases (T_{nonMet}), primaries that did form metastases (T_{Met}), lymph node metastases (LN), and metastases to other sites (Met) (FIGS. 2B and 2C). These lines were used in combination with a novel high-throughput ECM array screening platform to determine how phenotypic changes in adhesion correlate with tumor progression. Briefly, tumor initiation was achieved using intratracheal injection of lentiviral Cre recombinase. Tumors were resected, digested and plated onto tissue culture treated plastic to generate cell lines. Cell lines were subsequently cultured in Dulbecco's modified Eagle's medium (DMEM), 10% foetal bovine serum, penicillin/streptomycin and glutamine. These lines were derived from both primary lung tumors and their metastases.

They were placed in a seeding device that holds the top surface of the slides flush with bottom of the well (FIG. 2A). In all, 400,000 cells were seeded on each slide in 6 ml of serum-free medium (DMEM and penicillin/streptomycin). Cells were allowed to attach for two hours at 37° C. After attachment, slides were washed three times, transferred to quadriperm plates (NUNC, 167063), and new medium was added (DMEM, 10% foetal bovine serum, penicillin/streptomycin and glutamine). Slides were left at 37° C. for two additional hours before removal for staining. Slides were washed twice with PBS and fixed with 4% paraformaldehyde. Nuclei were stained using Hoechst (Invitrogen) in combination with 0.1% Triton-X and PBS. Slides were mounted with Fluoromount-G (Southern Biotech 0100-01) and stored at 4° C. before imaging. Slides were imaged using a Nikon Ti-E inverted fluorescence microscope and NIS Elements Software (Nikon). The entire slide was scanned and images stitched using that software. Image manipulation and analysis was performed in MATLAB (Mathworks) and quantification of nuclei was performed using CellProfiler software disclosed in Example 2. Clustering analysis was performed using Spotfire (Tibco). Replicate spots on each slide were averaged and those whose values were >1 s.d. above or below the mean of the replicates were excluded. Slides were normalized to the mean of their non-zero adhesion values. Clustering was performed based on Euclidean distances using Spotfire with the Hierarchical Clustering algorithm (normalized adhesion >0.01). In vitro adhesion seeding. In vitro ECM adhesion tests were performed using 96-well-plates (Corning 3603). Plates were coated with 20 μg/ml of fibronectin alone or 20 μg/ml of fibronectin and 20 μg/ml of the second molecule in PBS overnight at 4° C. Plates were then blocked with 1 wt % BSA at room temperature for 1 h. Plates were allowed to dry before adding 2×10⁴ cells per well in warm serum-free DMEM. Cells were allowed to adhere for 1 h at 37° C. and shaken every 15 min to ensure uniform seeding. Cells were washed with PBS, fixed with 4% paraformaldehyde and stained with Hoechst (Invitrogen). Wells were imaged using a Nikon Ti-E inverted epifluorescent microscope and analyzed with Nikon elements software.

To uncover changes in global adhesion signatures of cancer cells during progression and metastatic spread, a panel of murine lung adenocarcinoma cell lines derived from nonmetastatic primary tumors (T_{nonMet}), metastatic primary tumors (T_{Met}), and metastases from the lymph node (LN) and liver (Met) were analyzed. Technically, analysis of these cell lines showed very highly reproducible adhesion between replicate spots confirming the overall high quality of the ECM spotting and quantitative nature of the assay. Analysis of the adhesion signatures of these cell lines highlighted the diverse adhesion of each cell line to different ECM combinations (FIG. 2B and FIG. 2C).

Whether cells had greater or lesser adhesion to combinations of ECM components than to the molecules in isolation was assessed. FIG. 2C depicts adhesion profiles for three molecules: Collagen I (top), Collagen IV (middle) and Fibronectin (bottom) in combination with all other molecules. Dashed grey lines represent adhesion to that molecule alone. Arrows denote combinations with either of the other two molecules or alone. Error bars are s.e.m. of three replicate slides. The data presented herein suggest that within the checkerboard of pairwise-combinations, different partner molecules had additive, synergistic, and antagonistic effects on adhesion. For example FIG. 2C depicts that, for this T_{nonMet} cell line, many molecules improve adhesion to Collagen I, while others reduce its adhesion in comparison to the molecule in isolation (FIG. 2C, right, top panel). The same was true for the other molecules including Collagen IV and fibronectin (FIG. 2C middle and bottom panels, respectively). These types of combinatorial effects were present for many molecules and across all cell lines tested. For instance, the bottom panel of FIG. 2B depicts a comparison of three replicate slides for two representative cell lines. The repeatability of the assay is evident from the conserved profiles between replicate slides of the same cell line, Scale bars in (left panel) and (right panel) are 450 μm and 100 μm, respectively.

### Example 3

### Adhesion Signatures Across Lung Cancer Cell Lines

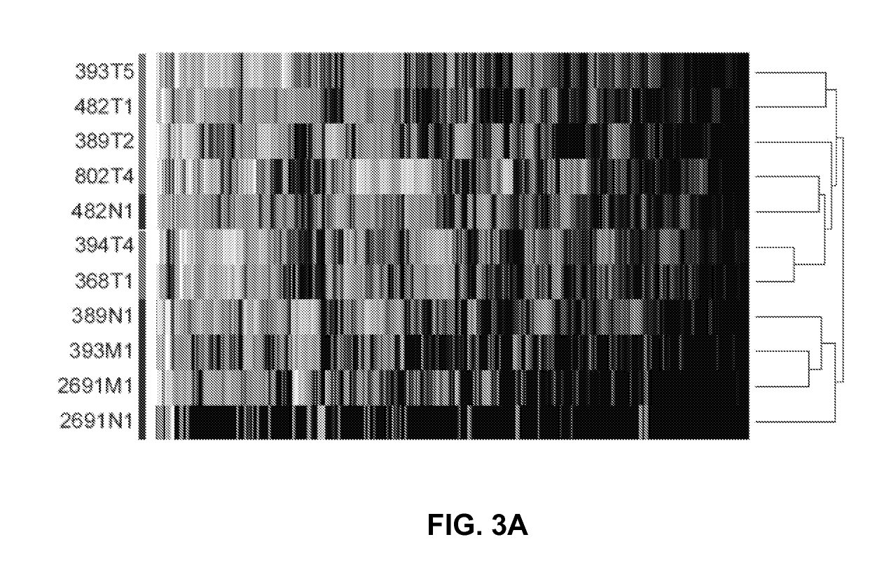

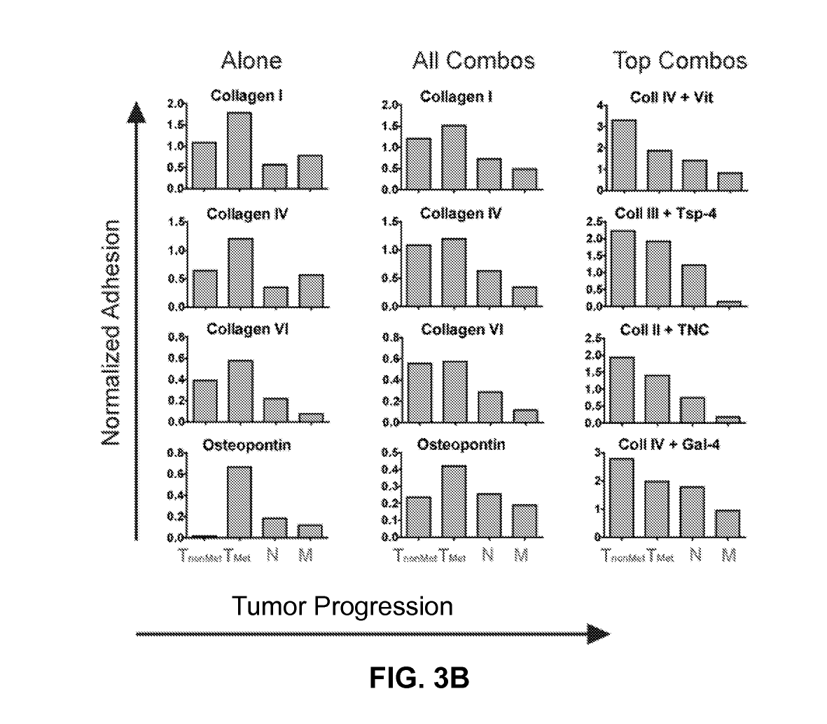

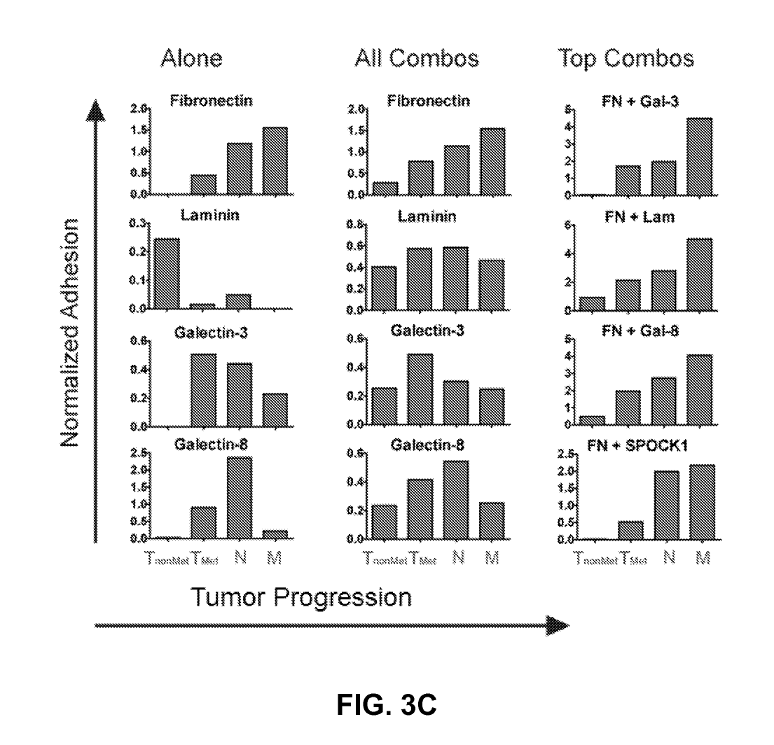

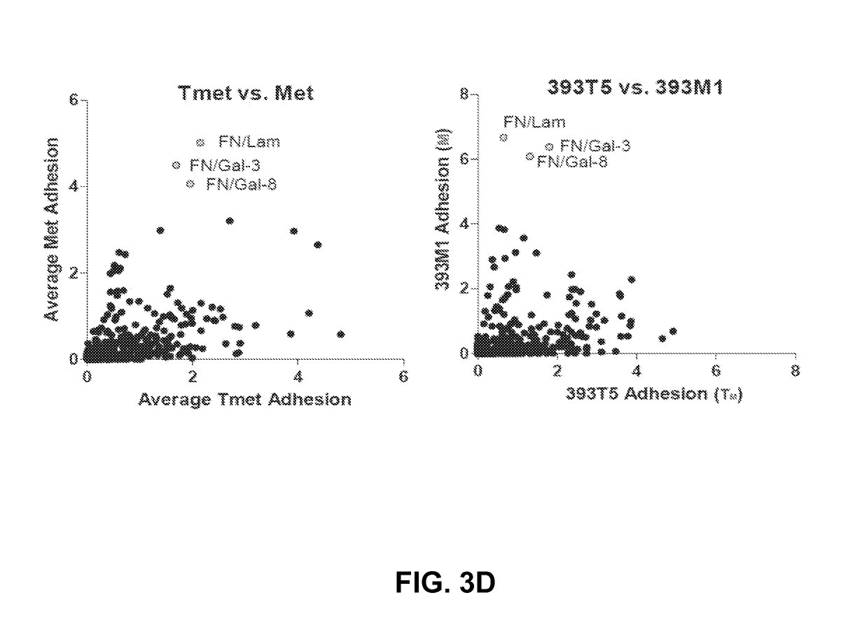

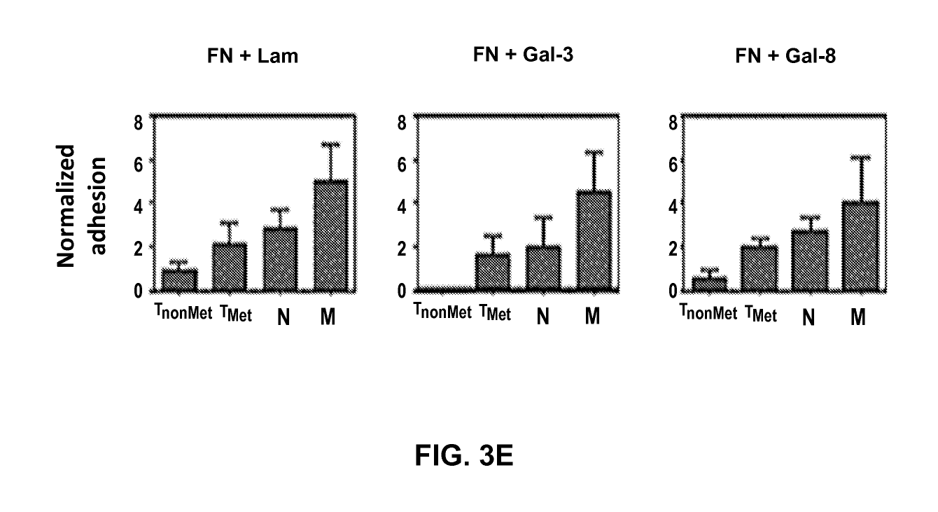

ECM arrays spotted and seeded similar to the above Example 2 were then used to analyze cell lines from each of the four classes of cell lines (FIG. 3A). To compare the adhesion signatures of the different lines, unsupervised hierarchical clustering analysis of the adhesion values in a manner analogous to clustering of data applied above. The vertical axis represents different ECM combinations. The horizontal axis represents different cell lines. Cell lines identified as 393T5, 482T1, 389T2, 394T4, and 368T1 are primary tumours (TnonMet and TMet lines). The other remaining cell lines are those derived from nodal (N) or distant metastases (M). The data presented herein demonstrate that all cell lines derived from metastases, save for one lymph node line, clustered separately from cell lines derived from primary lung tumors (see adhesion pattern in FIG. 3A).

This result is particularly surprising since two of these metastatic lines (389N1 and 393M1) were from tumors that directly disseminated from two of the primary lines screened (389T2 and 393T5, respectively), and yet clustered more closely with the other metastases than to their parental lines. This finding suggests that there is a conserved phenotypic change in the ECM adhesion signature of cancer cells from a metastatic site versus those that remain in the primary tumor. Interestingly, this differential clustering was not evident from unsupervised hierarchical clustering of gene expression of these lines (Winslow, M. M. et al. Suppression of lung adenocarcinoma progression by Nkx2-1. Nature 473, 101-104 (2011)). The present disclosure therefore indicates that this phenotype, which may influence metastatic progression, is undetectable by examining specific mRNA or protein expression of specific genes.

To determine whether phenotype-based adhesion screening using an ECM array uncovered characteristics of tumor progression that could not have been detected by gene expression studies, whether changes in adhesion found could be explained by changes in expression of related molecules was assessed. Gene expression profiling was performed on cell-lysate harvested from cells at the time the ECM arrays were run.

Expression data for each of the ECM genes was compared to adhesion to those molecules for each of the eleven cell lines. While some expression data correlated well with adhesion (low adhesion/low expression, high adhesion/high expression), many molecules exhibited either high expression with little adhesion or high adhesion with little expression. There was no statistically significant difference in adhesion between ECM genes expressed at a low, medium, or high level (p>0.6). The present disclosure therefore indicates that there is likely a complex interplay with other parenchymal or stromal cells that either act to provide molecules necessary for adhesion of the tumor cells or react to ECM components produced by tumor cells, perhaps in a manner that promotes tumorigenesis.

In light of the hierarchical clustering results, we asked whether there were particular combinations of molecules that are favored by metastatic cells rather than by cells from primary tumours. Thus, we compared the average adhesion of the liver metastasis-derived cell lines (M) for each ECM combination to the average adhesion of the TMet lines (FIG. 3D) FIG. 3D depicts the average adhesion of metastatic cell lines (M) to each combination compared with those of the metastatic primary tumour cell lines (TMet) (on the left). FIG. 3D also depicts a comparison of 393M1 adhesion for each combination to its matching primary tumour line, 393T5 (on the right). Light grey dots indicate top ECM combinations exhibiting preferential adhesion by metastatic lines over the metastatic primary tumour lines. Although many of the M lines exhibit elevated binding to combinations containing fibronectin, pairings that combined fibronectin with any of galectin-3, galectin-8 or laminin had the highest differential adhesion between line classes. To explore changes in adhesion that specifically correlated with changes in metastatic progression, we compared the TMet cell line 393T5 and the clonally related liver metastasis-derived cell line 393M1. This pair of lines was derived from a primary tumour and a metastasis that disseminated from that tumour, as confirmed by examination of the lentiviral integration site.

Furthermore, the differential adhesion to the aforementioned ECM combinations was clear in both the group-wise comparison (FIG. 3A) and in the direct comparison with this primary tumor-liver metastasis pair (FIG. 3D). Collectively, the patterns observed suggest that combinations of molecules may have a more significant role in the adhesion profile of a given population than the tendency to bind to any of the ECM molecules alone. Interestingly, the trend towards increased binding to fibronectin/galectin-3, fibronectin/laminin and fibronectin/galectin-8 combinations was consistent across tumor progression when we compared the average adhesion of all TnonMet, TMet, N and M cell lines (FIG. 3E). Binding to these molecules, when presented alone, showed minimal (fibronectin) or no trend (laminin, galectin-3 and galectin-8) across the four groups of cell lines (FIG. 3B and FIG. 3C). When in combination, however, these pairs demonstrate enhanced effects that exceed the additive values of their individual adhesion. In contrast, other combinations demonstrated a reduced adhesion trend in relatively more metastatic populations, including a variety of collagens and osteopontin (FIG. 3B and FIG. 3C). Taken together, these data suggest that adhesion to fibronectin in combination with any of galectin-3, galectin-8 or laminin is highly associated with tumor progression in this model system.

Overall, the present disclosure demonstrates that adhesion signatures allow one to determine a cellular state that is predictive of disease state and that is otherwise unpredictable using available techniques. This signature can act as a diagnostic test for metastatic disease, predicting the TNM stage of a clinical sample and potentially identifying which distal organs the disease will most readily metastasize to. This finding is of major significance to the diagnosis of cancer.

### Example 4

### Adhesion Signatures Across Lung Cancer Cells from Mouse to Human Samples

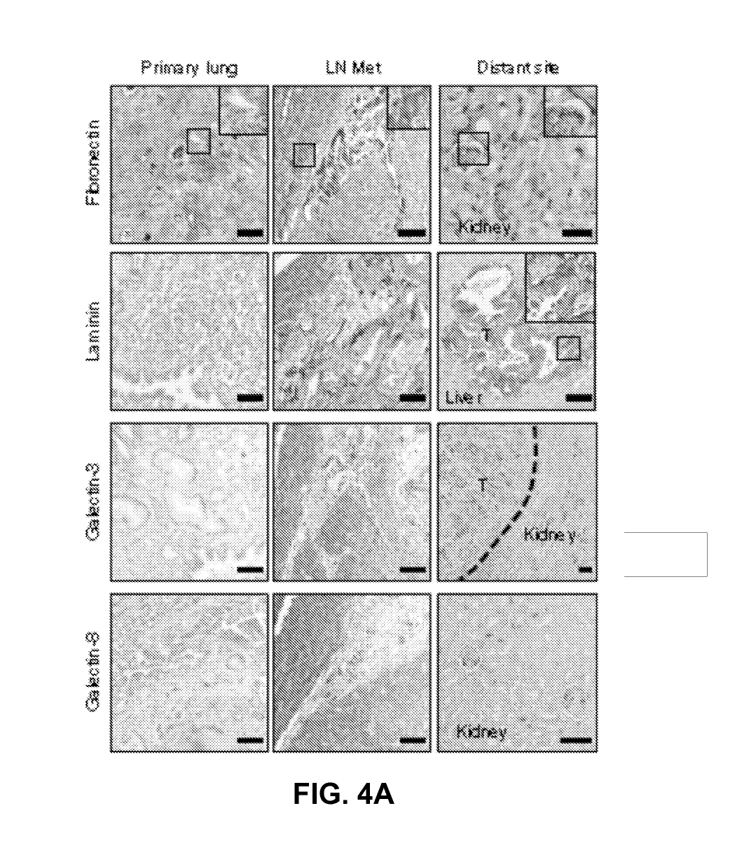

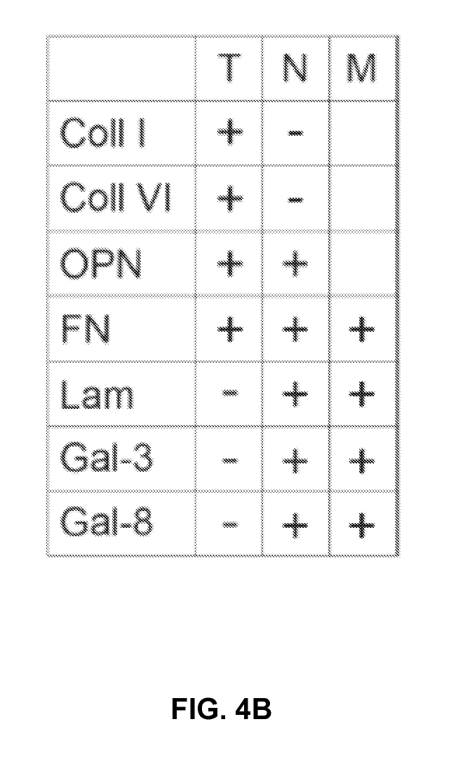

Next we sought to correlate our in vitro adhesion profiles with ECM expression in vivo. To investigate whether the identified ECM molecules may be important in natural tumorigenesis, organs containing primary autochthonous tumors and their metastases were resected from KrasLSL-G12D/+; p53flox/flox mice and stained. Trichrome staining of lungs with extensive tumor burden revealed a significant presence of ECM deposition in the tumor-bearing lung (FIG. 4A). Previously, we found that primary tumors that have acquired the ability to metastasize (TMet tumors) upregulate the chromatin-associated protein Hmga2. Therefore, we used Hmga2 immunohistochemistry in addition to histological characteristics to identify areas of highly aggressive cancer cells. As anticipated, primary lung tumors were positive for collagen I, collagen VI, and osteopontin with the most intense staining overlapping with the high-grade tumor areas (data not shown). In particular, osteopontin staining strongly co-localized with Hmga2pos regions, suggesting that increased osteopontin production is associated with metastatic primary lung tumors. Furthermore, little to no laminin, galectin-3 or galectin-8 staining was detected in the primary tumors (FIG. 4A). Interestingly, fibronectin staining in the tumor was strong, revealing a correlation between increasingly metastatic populations and the presence of fibronectin early in the metastatic cascade (FIG. 4A).

We next asked whether the lymph node and distant organ metastases contained the metastasis-associated ECM molecules. Again, trichrome staining revealed the presence of significant matrix deposition within the lymph nodes (Data not shown). As expected, the entirety of the lymph node tumors was histologically high-grade and was Hmga2pos. There was also clear expression of all four of the metastasis-associated molecules (fibronectin, laminin, galectin-3 and galectin-8) within the lymph node metastases (FIG. 4A). Furthermore, there was essentially no collagen I or collagen VI (data not shown). Osteopontin, however, was present in the metastases (data not shown) and had its highest expression along the invasive front. A summary of the immunohistological data is presented in FIG. 4B. “T” denotes primary lung tumor; “N” denotes lymph node metastases; “M” denotes distant organ metastases; and stained ECM components are as follows: “Coll I” is collagen 1; “Coll IV” is collagen IV; “OPN” is osteopontin; “FN” is fibronectin; “Lam” is laminins; “Gal-3” is galectins-3; “Gal-8” is galectins-8.

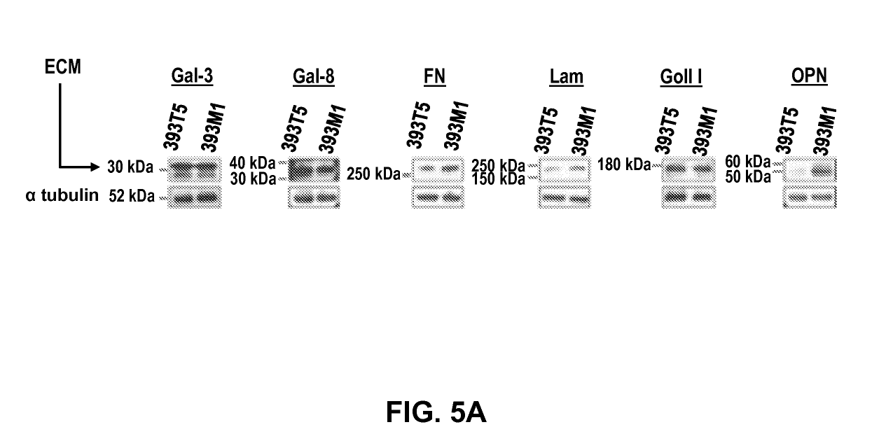

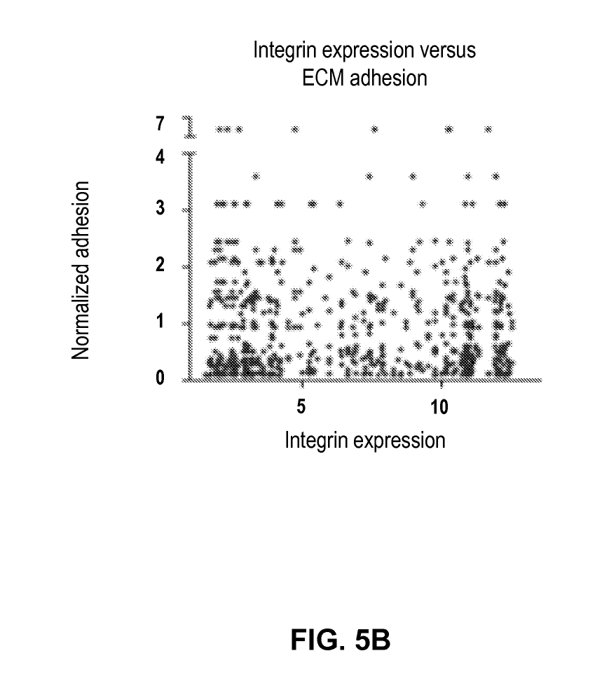

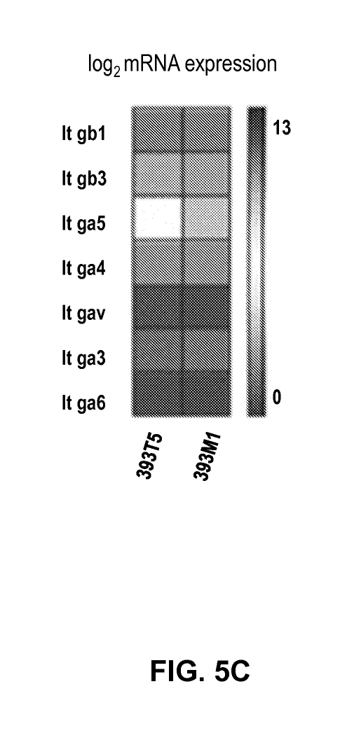

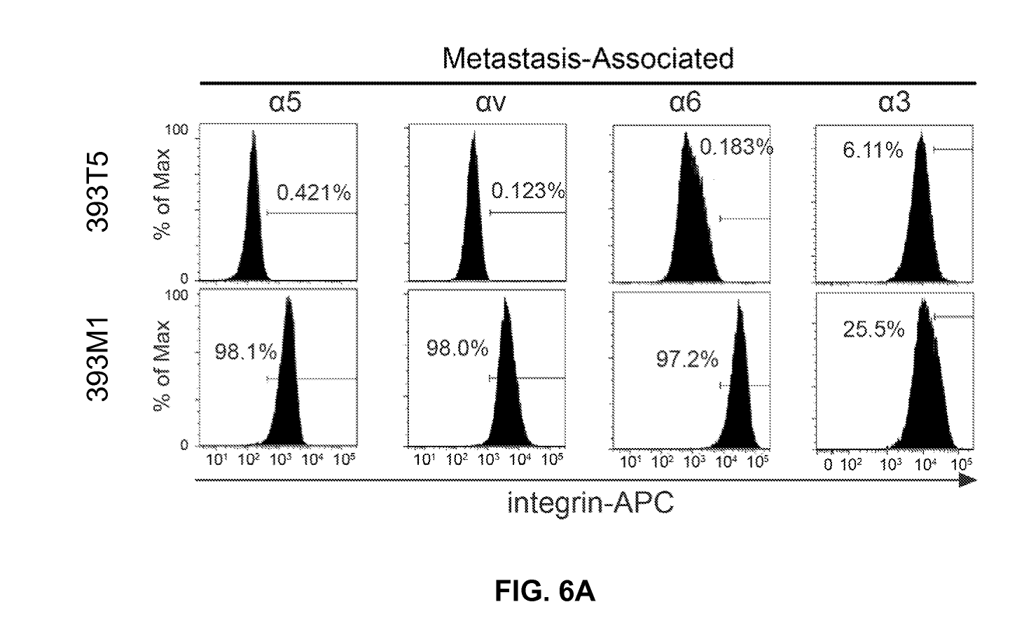

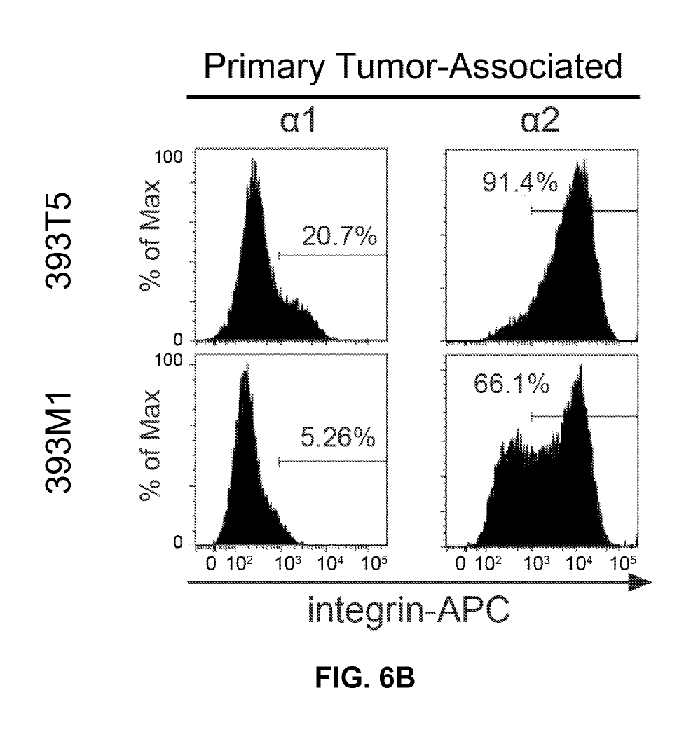

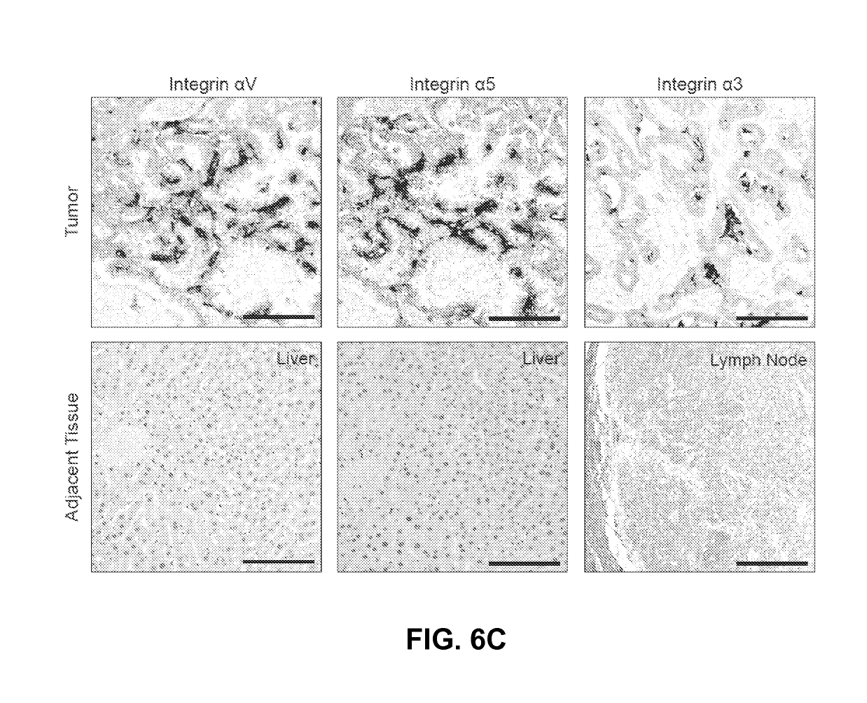

We also examined common metastatic sites for the presence of the metastasis-associated molecules (FIG. 4A). Both galectin-3 and galectin-8 were distinctly visible in these sites. Laminin and fibronectin both appeared to line the sinusoids of the livers of the mice and were also present in the metastases formed there. To determine whether these differences between the primary and metastatic sites were due to altered matrix production by the tumor cells, we performed immunoblots on the 393T5 and 393M1 TMet and M cell lines. Although the M line showed slight increases in fibronectin and laminin production compared with the TMet line, production of both galectins was constant (FIG. 5A). Furthermore, collagen I production was constant, and osteopontin production was actually increased in the M line. Taken together, these data suggest that the ECM microarrays identified molecules that were found within the physiologically relevant sites of mice bearing autochthonous tumors, and that production of these molecules is not solely performed by the tumor cells present in those sites.

Integrin Surface Expression Correlates with ECM-Binding Profiles.

Additionally, whether adhesion to specific ECM components correlated with expression of their cognate integrins was assessed. As was the case with the ECM component expression, expression of integrins often did not correlate with adhesion to their known ligands. This finding suggests that small alterations in expression of many integrins may result large changes in adhesion to molecules that they interact with or that more complex mechanisms, such as ECM or integrin post-processing, contribute dramatically to adhesion.

Whether presentation of molecules of interest at the protein level correlated with adhesion to those molecules was assessed. Western blots of lysate from the characteristic primary line 393T5 were compared to that of metastatic line 393M1. The data presented herein demonstrate that expression of both galectins was unchanged between the lines despite an increase in adhesion to them in the 393M1s (FIGS. 5A, 5B, and 5C). The present disclosure therefore indicates that protein levels of either galectin-3 or galectin-8 may not be sufficient markers of disease progression or surrogate metrics for adhesion to those molecules. Furthermore, increased Osteopontin expression was detected in the 393M1 line despite having a lower adhesion score for that line. This finding suggests a role for signaling by Osteopontin from the metastatic population despite their lack of adhesion to the molecule. The present disclosure therefore indicates that, while Osteopontin may promote development of the metastatic niche, loss of adhesion to it may be necessary for tumor cells to invade and progress through the metastatic cascade.

We noted that comparisons of adhesion trends on our ECM arrays did not necessarily correlate with transcriptional profiles of the cognate integrins (FIG. 5B). Thus, to correlate our findings with the presence of receptors for these metastasis-associated ECM molecules, we examined the clonally related pair of representative TMet and M cell lines for surface expression of their cognate integrins. Although the mRNA expression patterns did not show significant upregulation of the metastasis-associated integrins in the M line by gene expression microarray (FIG. 5C), flow cytometry analysis of the integrin subunits corresponding with either the primary tumor-associated molecules or metastasis-associated molecules revealed that the receptor expression trends were consistent with the observed binding patterns. Specifically, integrin subunits known to bind fibronectin (α5 and αv), laminin (α6 and α3) and galectins (α3) were all more prevalent on the metastasis-derived line, while those associated with collagens (α1 and α2) were relatively higher on the primary tumor-derived line (FIG. 6A and FIG. 6B). FIG. 6A and FIG. 6B depict flow cytometry of integrin surface expression in 393T5 (TMet) and 393M1 (M) cell lines. Integrin subunits that bind to metastasis-associated molecules show increased surface presentation in the metastatic line (α5, αv, α6, α3), while those that bind to primary tumour-associated molecules show decreased presentation (α1 and α2).

Nonetheless, the surface expression trends were consistent for the other TMet and M lines as well (data not shown). Furthermore, within a given cell line, we observed relatively homogeneous surface expression of the metastasis-associated integrins as measured by flow cytometry (data not shown), suggesting that variations in adhesion between lines are due to global increases in surface receptor expression rather than binding patterns of select subpopulations. Immunohistochemistry revealed that these integrins were also present in the metastases of mice bearing autochthonous tumors, but not the adjacent tissue (FIG. 6C). FIG. 6C depicts metastasis-associated integrins in mice bearing autochthonous tumours with spontaneous metastases to the liver and lymph nodes. Scale barsare 100 μm.

The finding that the transcriptional levels of the integrins do not agree with the adhesion trends suggests that post-transcriptional regulation, post-translational modifications such as altered glycosylation or alterations in activation state of the integrins are likely responsible for the changes in adhesion. Thus, by utilizing our platform that investigates specific ECM binding rather than receptor gene or protein expression, we are able to identify candidate ECM interactions that might otherwise have been overlooked.

Integrin α3β1 Mediates Adhesion and Seeding In Vitro and In Vivo.

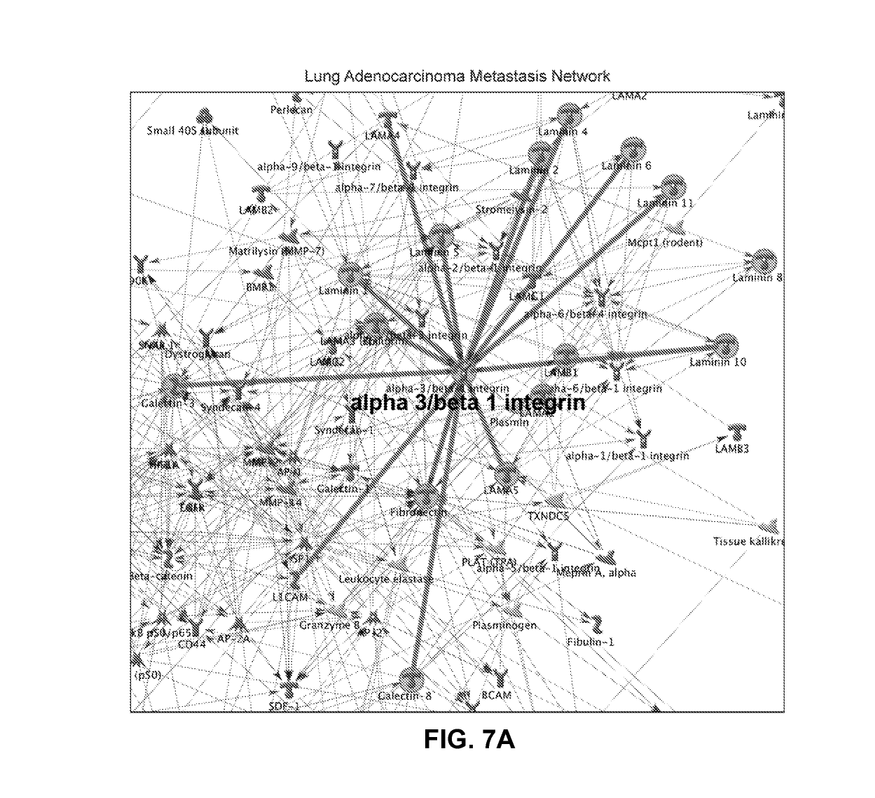

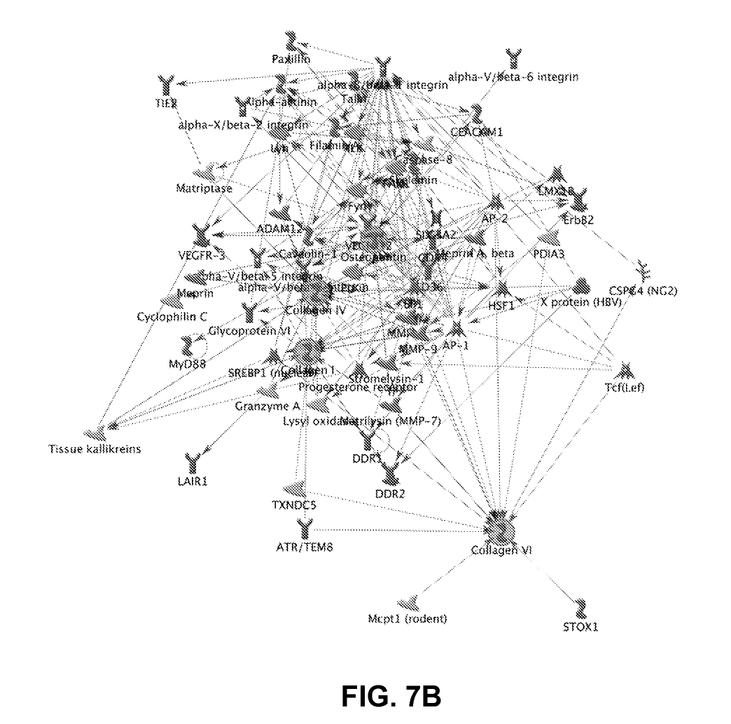

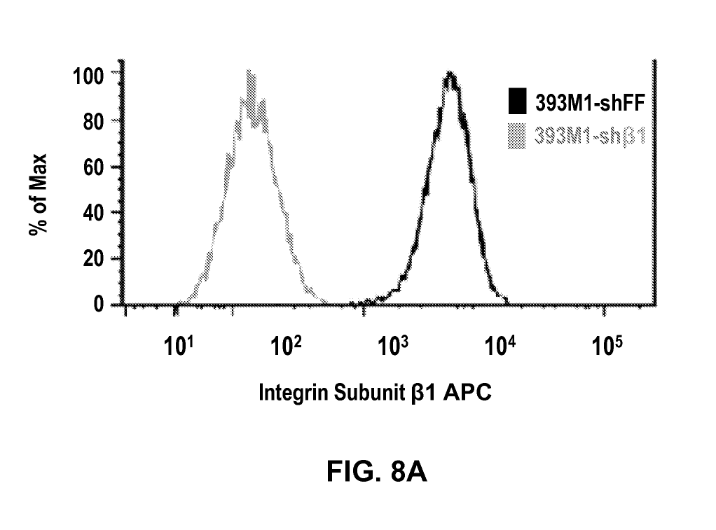

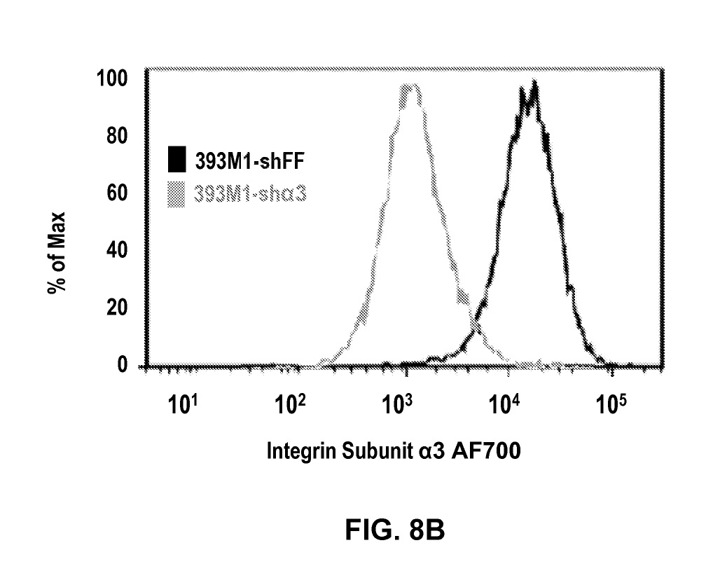

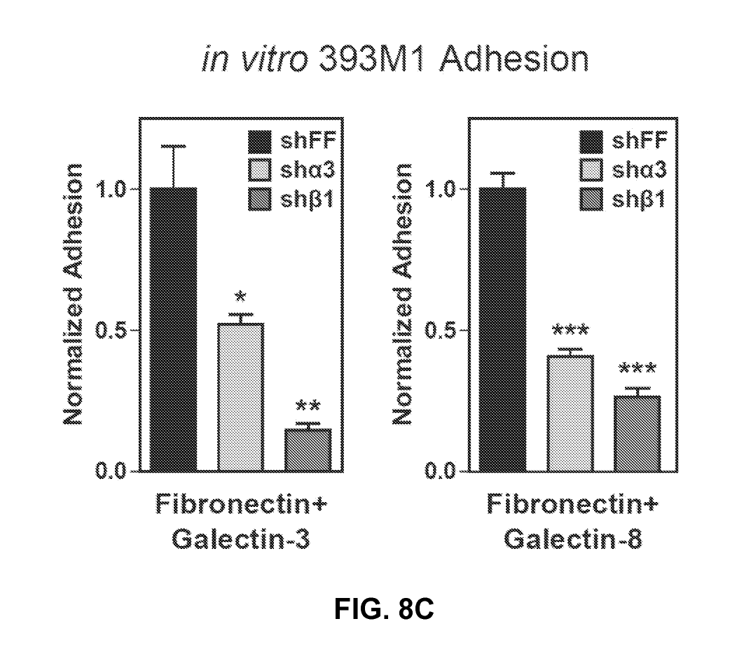

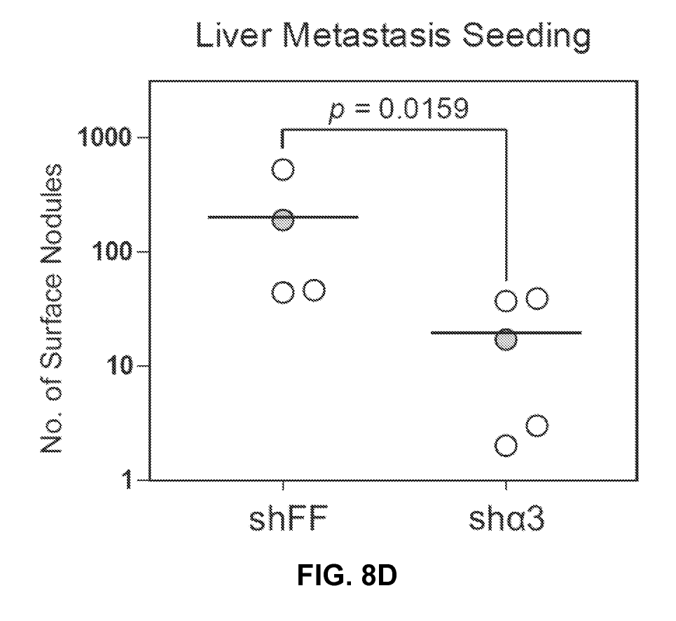

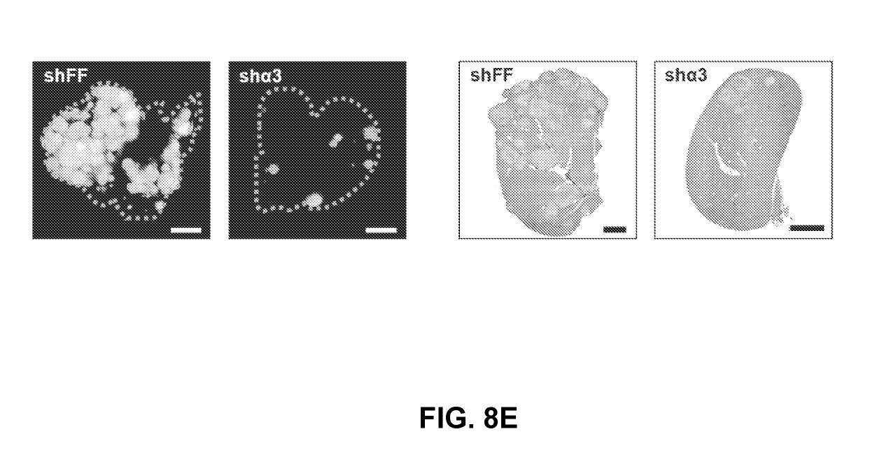

To examine which candidate receptor/ECM interactions may participate in the observed binding patterns, we performed in silico network mapping of the metastasis-associated ECM molecules using GeneGO software (Metacore) of manually curated molecular interactions. We generated a network map that we termed the lung adenocarcinoma metastasis network that has a greatest disease association with ‘Neoplasm Metastasis’ (P=1.094×10−45, hypergeometric test, FIG. 7A). A network generated using the same parameters but with the primary tumor-associated molecules did not exhibit any disease association with metastasis (FIG. 7B). Analysis of the lung adenocarcinoma metastasis network identified integrin α3β1 as the surface receptor with the greatest number of edges (FIG. 7A). The results were generated in GeneGO (Metacore) generated using an autoexpand algorithm and initiated with the metastasis-associated ECM molecules (laminins, fibronectin, galectin-3, and galectin-8). The lower panel of FIG. 7A depicts disease association rank of the lung adenocarcinoma network. P-values determined by hypergeometric test. On the basis of this finding, we performed a knockdown of both the α3 and β1 subunits (Itga3 and Itgb1, respectively) using short-hairpin mediated RNA-interference (FIG. 8A and FIG. 8B). FIG. 8A depicts flow cytometry analysis of integrin surface expression with knockdown of ITGB1; and knockdown of ITGA3 (FIG. 8B). Curve on the right side of the graph represents control hairpin against firefly luciferase; while the curves on the left hand side of the graphs represent hairpin against integrin subunits. Knockdown of both α3 and β1 integrin subunits by shRNA also reduced adhesion to metastasis-associated molecules in vitro when compared with the control hairpin targeting the firefly luciferase gene. shFF is the control hairpin targeting firefly luciferase. (FIG. 8C). Error bars in FIG. 8C represent s.e. (n=3). One-way ANOVA with Tukey's Multiple Comparison Test was used to analyse the data in FIG. 8C.

We next assessed whether this integrin dimer has a role in metastatic seeding in vivo. Thus, we conducted experimental metastasis assays by intrasplenic injection of 393M1-shα3 or 393M1-shFF cells into wild-type mice, and monitoring for liver tumor formation. We found that mice injected with the 393M1-shα3 cells formed fewer tumor nodules than the controls (FIGS. 8D and 8E). The number of liver tumor nodules of the surface of livers 2.5 weeks after intrasplenic injection were determined through analysis of surface fluorescence of ZSgreen⁺ cells or through histological evaluation following paraffin embedding, section, and staining with hematoxylin and eosin. Mann-Whitney (non-parametric) test was used to analyse significance. Taken together, these findings suggest that the α3β1 integrin dimer has a role in adhesion of metastatic cells to the metastasis-associated ECM molecules and in metastatic seeding.

Galectin-3/8 is Present in Human Lung Cancer Metastases.

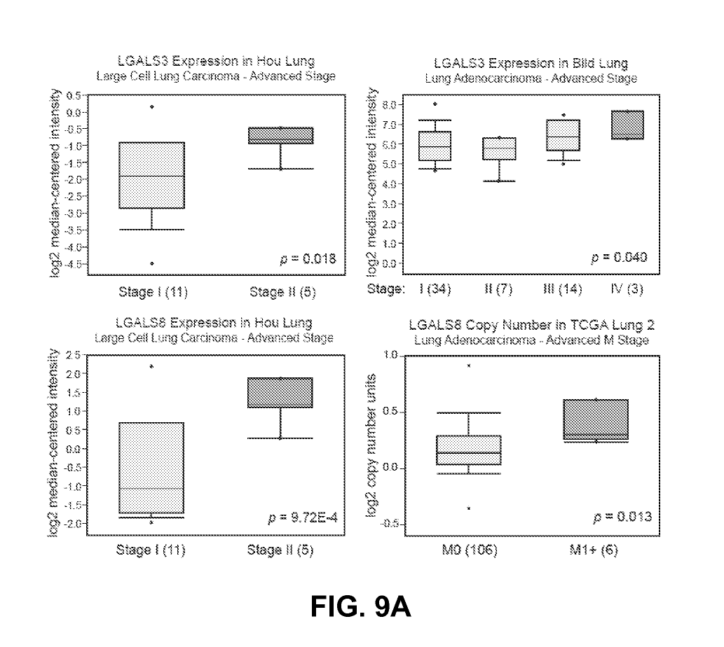

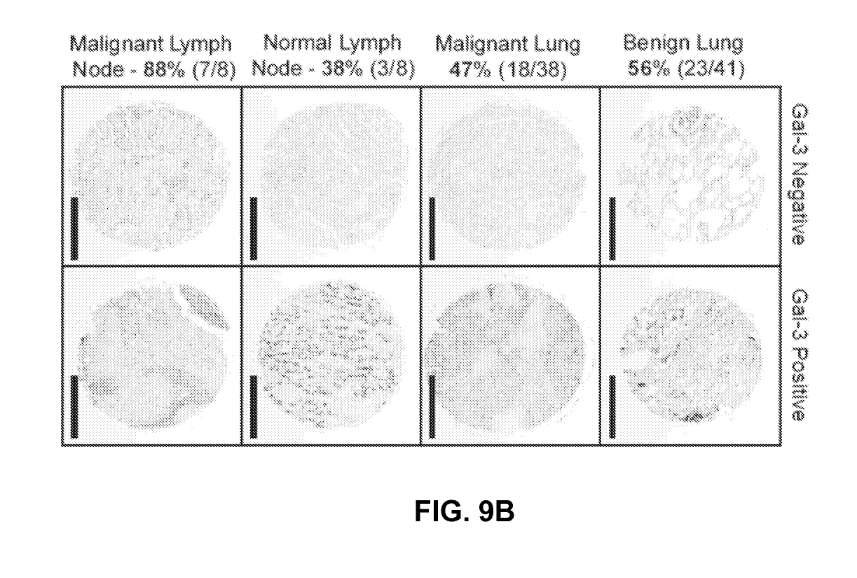

Based on the in vitro adhesion data and in vivo mouse findings, we sought to explore the role of the metastasis-associated ECM molecules in human samples. Using Oncomine-32, a human genetic dataset analysis tool, we examined the correlation of ECM gene expression and disease severity (for example, clinical stage or the presence of metastases). Results of these queries demonstrate that increased gene expression or copy number of LGALS3 or LGALS8 (galectin-3 and galectin-8, respectively) correlate with increased clinical stage or the presence of metastases (FIG. 9A). We next investigated whether galectin-3 protein is present at higher levels in malignant human lung tumors compared with benign non-neoplastic human lung tissue using samples taken from lungs and lymph nodes of patients. Staining for galectin-3 in human tissue microarrays revealed a higher presence of the molecule in lymph nodes of patients with malignant disease (88%) compared with those without cancer (38%) (FIG. 9B). Furthermore, there was a higher fraction of galectin-3-positive lymph nodes (88%) than positive primary lung tumor samples (47%), confirming its association with the metastatic site over the primary tumor (P<0.05, Fisher's exact test). Thus, the ECM microarrays were capable of identifying interactions associated with metastasis in human lung cancer.

### Methods

Protein analysis. Western blot analysis of ECM molecules was performed with the following antibodies: galectin-3 (Abcam, ab53082, 1:500), galectin-8 (Abcam, ab69631, 1:500), osteopontin (Abcam, ab8448, 1:2,000), fibronectin (Abcam, ab2413, 1:1,000), laminin (Abcam, b11575, 1:1,000), collagen I (Abcam, ab34710, 1:5,000) and α-tubulin (Cell Signaling, 2125, 1:1,000). Immunohistochemistry of ECM molecules was performed with the following antibodies: galectin-3, galectin-8 (1:75), osteopontin, laminin (Abcam, ab11575, 1:100), fibronectin (Millipore, AB2033, 1:80), Hmga2 (Biocheck, 59170AP, 1:1,000), collagen I (Abcam, ab34710, 1:500) and collagen VI (Abcam ab6588, 1:100). Integrin staining was performed using the following antibodies: integrin αv (Millipore AB1930, 1:200), integrin α5 (Chemicon AB1928, 1:200), integrin α3 antibody was prepared using known methods. Tissue microarrays were acquired from LifeSpan Biosciences (LS-SLUCA50), and were stained with the same galectin-3 antibody. Murine tissues were harvested from KrasLSL-G12D, p53flox/flox mice27-29. IHC was performed following resection from mice, fixation in formalin and embedding in paraffin. Flow cytometry analysis of integrin expression was performed using the following antibodies: integrin α5 (Abcam and BioLegend-clone 5H10-27, 1:100), integrin αv (BD-clone RMV-7, 1:100), integrin α6 (BD and BioLegend-clone GoH3, 1:100), integrin α3 (R&D, 1:100), integrin α1 (BD-clone Ha31/8 and BioLegend-clone HMα1, 1:100) and integrin α2 (BD-clone HMα2, 1:100).

RNA isolation and expression profiling. Cell lysates were harvested using Trizol (Sigma). Chloroform extraction was performed followed by RNA purification using Qiagen RNeasy spin columns. Lysates were analyzed for RNA integrity and prepared with Affymetrix GeneChip WT Sense Target Labelling and Control Reagents kit, followed by hybridization to Affymetrix Mouse 3′ Arrays (Mouse 430A 2.0) Lysates used for gene expression microarrays were harvested at the same time as the ECM microarrays were seeded to ensure minimal variability introduced by cell culture. R/Bioconductor software was used to process array images. Unsupervised hierarchical clustering analysis was performed in Spotfire (Tibco) for all probe sets with variance >0.5 and expression >3.0 using Euclidean distances. Data sets are publically available from NCBI under accession number GSE40222 Retroviral short hairpin RNA (shRNA) constructs. miR30-based shRNAs targeting integrins β1 (5′ TGCTGTTGACAGTGAGCGCGGCTCTCAAACTATAAAGAAATAGTGAAGCCACAGATGT ATTTCTTTATAGTTTGAGAGCCTTGCCTACTGCCTCGGA-3′), α3 (5′-TGCTGTTGACAGTGAGCGCCGGATGGACATTTCAGAG AAATAGTGAAGCCACAGATGTATTTCTCTGAAATGTCCATCCGTTGCCTACTGCCTCGG A-3′), or control firefly luciferase (5′-AAGGTATATTGCTGTTGACAGTGAGCGAGCTCCC GTGA ATTGGAATCCTAGTGAAGCCACAGATGTAGGATTCCAATTCAGCGGGAG CCTGCCTACTGCCTCG-3′) were designed using the shRNA retriever software available at the Katandin homepage (http://katandin.cshl.edu/homepage/siRNA/RNAi.cgi?type=shRNA), synthesized (IDT, Coralville, Iowa), and then cloned into the MSCV-ZSG-2A-Puro-miR30 vector. Packaging of retrovirus and transduction of cells was done as described previously.

All animal procedures were performed in accordance with the MIT Institutional Animal Care and Use Committee under protocol 0211-014-14. Cell injection studies were performed in B6129SF1/J mice (Jackson Laboratory, Stock Number 101043). Intrasplenic injections were performed using 5×10⁵ cells resuspended in 100 μl of phosphate-buffered saline (PBS) and injected into the tip of the spleen following existing protocols29. Animals were anaesthetized with avertin before surgery. Fur was removed from the animals and they were sterilized with Betadine and 70% ethanol. The spleen was exteriorized following incisions in the skin and body wall. Cells were injected into the end of the spleen with a 27-gauge syringe and allowed to travel into circulation for 2 min. Spleens were then excised from the animals following cauterization of the splenic vessels. The muscle wall was closed using 5-0 dissolvable sutures, and the skin was closed using 7 mm wound clips (Roboz). Mice were killed 2.5-4 weeks following injection, and their livers were excised. Quantification of surface nodules and imaging of livers was performed using a dissection microscope. Tissues were embedded in paraffin following fixation in 4% paraformaldehyde and stained using hematoxylin and eosin.

### Discussion

Our ECM microarrays provide a high-throughput multiplexed platform capable of measuring a variety of cellular responses to ECM. Here, we show they are capable of identifying adhesion patterns that differentiate metastatic populations from primary tumors. We found that metastatic lung cancer cells preferentially bind to fibronectin in combination with laminin, galectin-3 or galectin-8 compared with cells derived from primary tumors. These changes in adhesion correlate with changes in surface presentation of various integrins. In particular, α3β1 mediates adhesion to these molecules in vitro and permits metastatic seeding in vivo. Furthermore, metastases derived from both a genetically engineered mouse lung cancer model and from human lung cancers express the metastasis-associated ECM molecules. It is worth noting that the combinations of these ECM components elicited the strongest effects, highlighting the importance of using a platform that is capable of measuring responses to more than individual molecules.

Galectins are a class of lectins that bind β-galactosides and can associate with other ECM molecules such as fibronectin. Galectin-3 is associated with metastasis in a variety of cancers and can bind to the oncofetal Thomsen-Friedenreich antigen, a carbohydrate antigen overexpressed by many carcinomas. Our platform confirmed its importance in lung adenocarcinoma, and also identified galectin-8 as having similar importance. Although galectin-8 is known to affect adhesion of cells to other matrix molecules, its role in cancer and metastasis has been less clear as it has been found to have both a positive and negative association with adhesion and tumorigenesis. Using the ECM microarrays, we showed that binding to galectin-8 in combination with fibronectin is strongly associated with metastatic progression in lung adenocarcinoma.

Furthermore, in addition to many collagens, we found that loss of adhesion to osteopontin accompanied metastatic progression. Osteopontin levels correlate with prognosis in patients with metastatic disease, and secretion of osteopontin by primary tumors results in mobilization of bone marrow-derived stromal precursors that help establish the metastatic niche. In addition to confirming the presence of the metastatic molecules at the sites of metastases, we found that the invasive portions of primary tumors and the invasive front of the metastases secrete osteopontin (FIG. 4B). A metastatic tumor line also produces more osteopontin than its corresponding primary. These findings suggest that while some primary tumors may activate bone marrow cells by secreting osteopontin, in our model, metastatic cells may contribute to this recruitment at a comparable or higher level than the instigating primaries, despite their own loss of adhesion to the immobilized molecule. The use of gene expression signatures for patient stratification in the clinic has become more widespread, but while genomic approaches have been beneficial for identifying candidate genes, the diversity of findings makes the development of broad therapeutic options seem nearly impossible. By assaying for conserved mechanisms at the phenotypic level, however, relevant targets can be identified and therapeutics can be developed for a broad spectrum of patients. Our results highlight the utility of phenotypic screening approaches for identifying clinical biomarkers. Although we identify α3β1 integrin as a therapeutic target, we also demonstrate that the adhesion signatures generated by the ECM microarrays are capable of differentiating between genetically similar populations with varying metastatic potential. Furthermore, no increase in the mRNA levels of the galectins or their receptors was observed by gene expression microarrays in the M lines (FIGS. 5A, 5B, and 5C), despite the association of these molecules with metastasis. The presence of galectin-3 and galectin-8 in human samples (FIG. 9) demonstrates the relevance of this platform to human disease, and thus, we envision that these arrays may be a useful clinical tool for stratification of cancer patients beyond traditional TNM staging.

The value of the ECM microarray platform extends beyond the specific application of cancer metastasis. Although this study documents the ability to profile adhesion patterns, cells bound to the arrays can be kept in culture for multiple days to monitor longterm responses to ECM such as cell death, proliferation and alterations in gene or protein expression. Toward that end, one could use multiplexed antibody staining to probe the effects of ECM on stem cell differentiation or activation. Orthogonal screens can be performed to look at the effects of growth factors, small molecules or RNAinterference agents in the context of ECM. Reduction of requisite cell numbers can be achieved using miniaturized arrays to screen rare cell populations such as circulating tumor cells or cancer stem cells and to help expand those populations in vitro for further biological studies.

### Example 6

### Differing Adhesion Signatures of Human Mammary Epithelial Cells at Different Stages of Cancer Progression

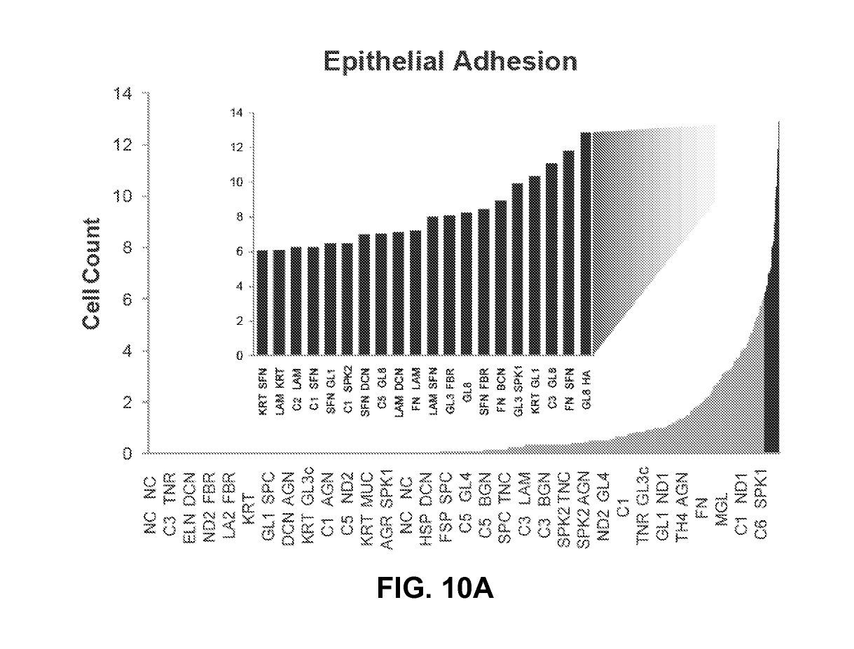

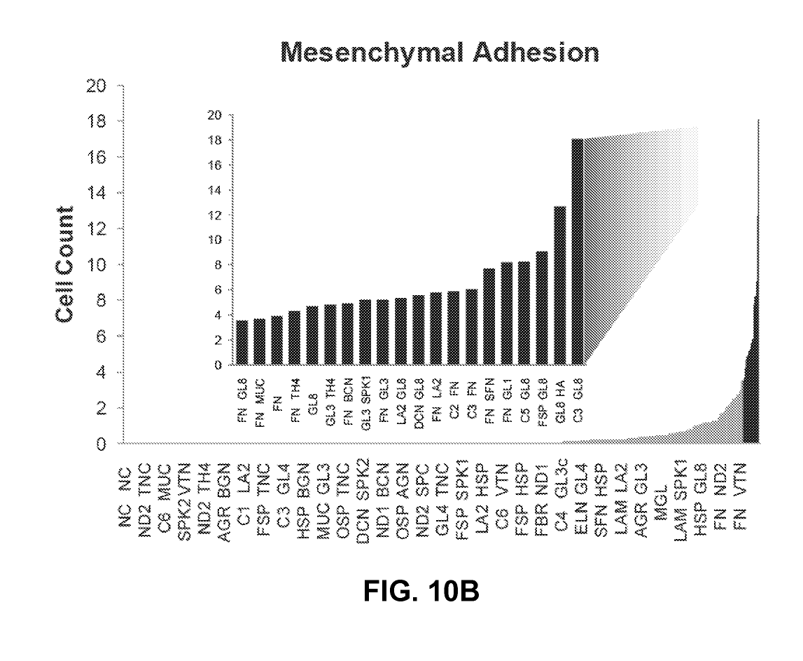

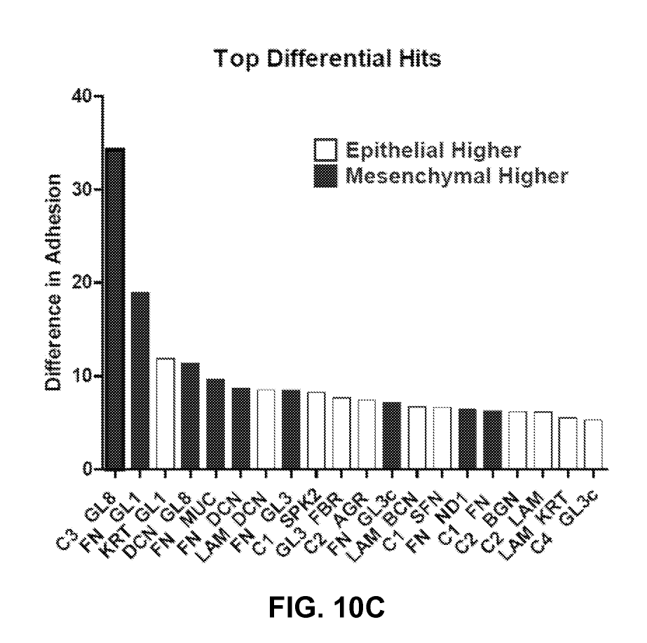

The findings and utility of the array to identify characterizing protein expression information of lung cancer metastases can be applied to other cancer types, such as breast cancer. The Epithelial-Mesenchymal Transition (EMT) describes a process by which epithelial cells that are typically tightly bound to each other and a basement membrane undergo a transition to a mesenchymal state in which they exhibit enhanced migratory capabilities. While this process occurs naturally during embryogenesis and wound healing, recent studies have implicated its role in a variety of pathologies. In particular, it is now appreciated that, in at least some instances, it is the driving force behind the acquisition of metastatic potential by neoplasms. Carcinomas that turn on this embryonic program known as EMT are capable of breaking free from the cells and extracellular matrix (ECM) around them and can invade through tissue, blood vessels, lymphatics, and eventually reach distant sites in the body. In order to form a secondary tumor at these distant sites, however, it is thought that the cells must undergo the reverse transition, known as mesenchymal to epithelial transition (MET), in order to colonize that distant site and grow into clinically detectable overt metastases. While a variety of extracellular signaling molecules such as TGFbeta are known to induce EMT, the factors driving MET are still poorly understood. Perhaps, the most well-studied tissue in the field of EMT as it relates to cancer, is the breast. Others have developed model systems to characterize breast cancer metastasis in the context of EMT (Genes Dev. Jan. 1, 2001 15: 50-65; Yang, J., et al. (2004); Twist, a master regulator of morphogenesis, plays an essential role in tumor metastasis. Cell 117, 927-939). A variety of transcription factors are known to turn on this program. In particular, Twist, Snail, and Slug are potent inducers of the EMT phenotype. Thus, in this work we have used a pair of cell lines that represent the two states: the epithelial cells (wild-type) and those that have undergone EMT (mesenchymal) (See FIGS. 10A and 10B, respectively). To achieve this, their lab immortalized normal mammary epithelial cells and subsequently made them oncogenic through the incorporation of the Ras oncogene. To make a mesenchymal version of the cells, they overexpressed the Twist transcription factor, inducing an EMT.

The ECM array was created using the techniques described above resulting in an ECM array comprising more than 700 different pairs of ECM components to determine how this process affects the interactions of cells with the ECM. We ran both the Epithelial (wt) and Mesenchymal (Twist+) cells (HMLERs) on the array. Alterations in their interactions may likely be representative of changes that occur to confer greater metastatic potential and be representative of more advanced stages of malignancy. Algorithms used in determining adhesion values and adhesion signature of the particular cells were previously described herein. The results from arrays are shown in the FIGS. 10, 11, and 12.

FIG. 10 describes differences in adhesion between the Epithelial and Mesenchymal cells. More specifically, FIG. 10A shows the adhesion of the wild-type mammary epithelial cells to all of the ECM combinations in the aforementioned array. The inset graph shows the twenty combinations of ECM components to which the epithelial cells have the greatest affinity according to their normalized adhesion values. Hyaluronic Acid and Galectin-8. No filtering based on the twist+ cell line has been done here. Adhesion signals were collected based upon quantification of nuclei (stained with Hoeschst, as before) on individual spots.

FIG. 10B describes the same method performed in the preparation and exposure of the cells to the array described in FIG. 10A. The inset graph depicts the twenty adhesion sets to which the twist+ cells exhibited the highest affinity.

[FIG. 10D represents a Differential Adhesion Heatmap based upon the comparative analysis of adhesion signatures of the cells contacted with the array described in FIGS. 10A and 10B. This heatmap depicts the top differentially adhered to combinations between the two cell types. The combinations listed under column “1” are the ECM combinations to which the epithelial cells preferentially adhere, whereas those under column “2” are the combinations of ECM components to which the mesenchymal cells preferentially adhere. Those cells that lose adhesion of the adhesion sets under column 1 have a likelihood of becoming more metastatic, whereas those cells that exhibit more affinity to the adhesion sets of column 2 combos have a greater likelihood of exhibiting more metastatic characteristics. Furthermore, these adhesion signatures are potentially indicative of disease state.]

FIG. 10C shows the top 20 ECM combinations that exhibit the greatest differences in adhesion between the two cell states. Dark grey bars represent adhesion sets to which mesenchymal (metastatic) cells more frequently bind. Light grey bars illustrate those adhesion sets to which non-metastatic epithelial cells frequently bind. The data allow a user of the array or system to characterize cells from a cell sample as having metastatic character or non-metastatic character.

To identify whether certain adhesion sets can stimulate proliferation and not simply adhesion of certain cell populations, experiments were performed on the arrays to measure cell doubling times with normal epithelial cells (labeled “wild-type” or “wt” in FIGS. 10-12) or metastatic epithelial cells derived from a mammary lineage (labeled “twist+” or “tw+” in FIGS. 10-12). One application of this experiment is to identify adhesion sets that are capable of stimulating the proliferation of normal mammary epithelial cells from primary lineages of mammary cells as well as creating a cell culture which can be used as a system to observe cellular response to stimuli while such cells are in a proliferative state. Cells were seeded in accordance with the previously disclosed protocols above.

FIG. 10A illustrates the adhesion sets that stimulate the top wild type doubling times after 48 hours of exposure to the system disclosed herein. FIG. 10B illustrates the adhesion sets that encourage proliferation of metastatic mammary epithelial cells after exposure to the system or array after 48 hours of exposure. It is probably worth noting that many of these combinations contain galectin-3. The results demonstrate that galectin-3, among other ECM components stimulates proliferation of mammary cells lineages with both wild-type and metastatic character. This data suggests that arrays or system comprising galectins-3 with another ECM binding partner should encourage growth of mammary cells in culture. Thus, it appears that galectin-3 promotes proliferation of both epithelial and mesenchymal mammary carcinomas. Differential mammary cell adhesion on array over both of the cell lines tested appears in FIG. 10C. Adhesion sets that preferentially bind normal epithelial cells are highlighted by open or white bars. Adhesion sets that preferentially bind metastatic cells are highlighted by black bars.

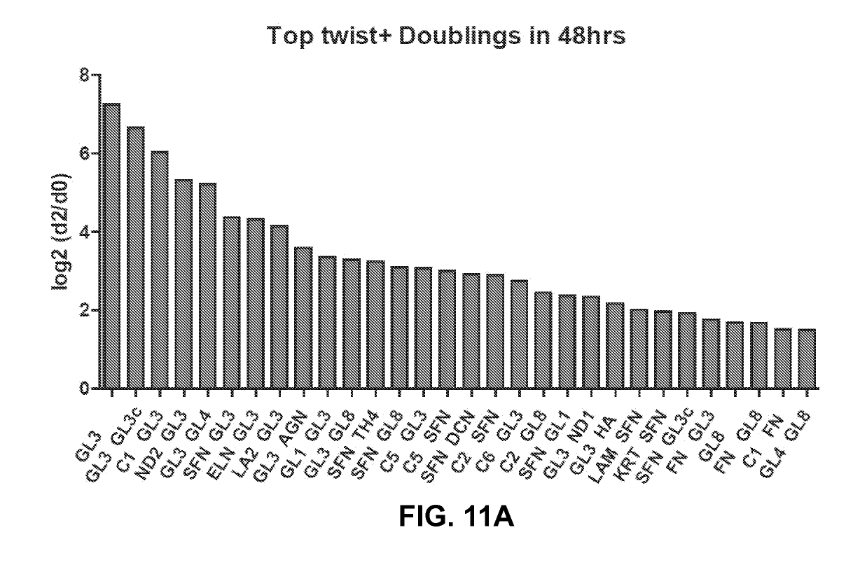

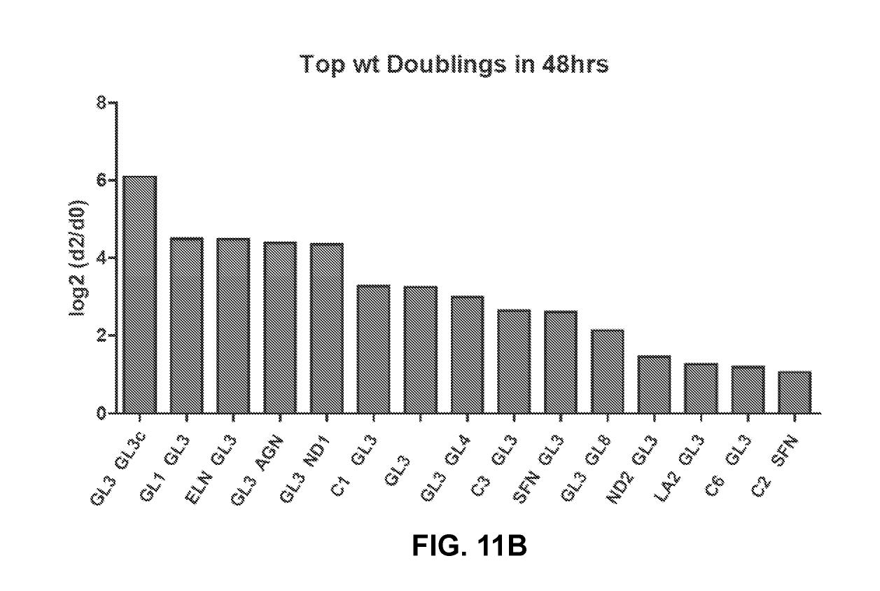

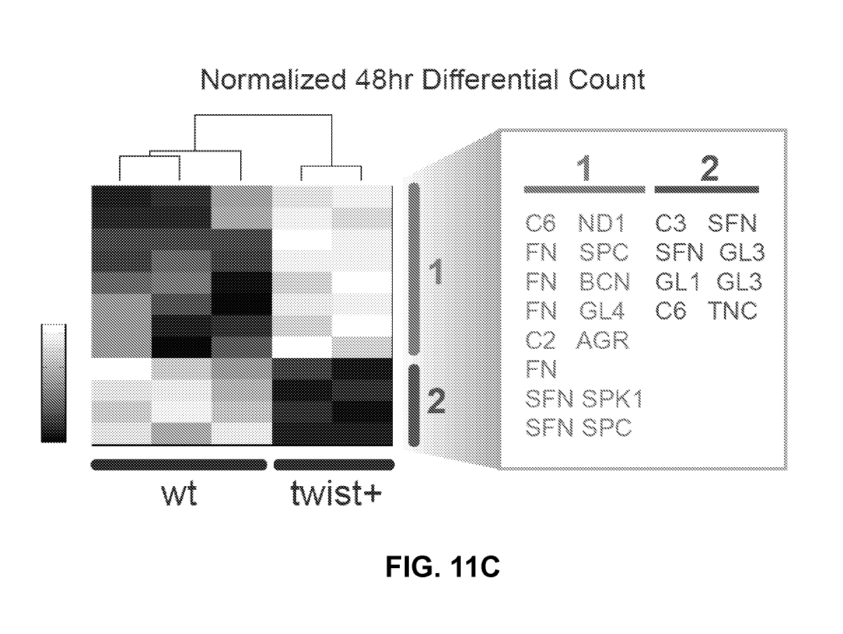

To study differential or selective proliferation capabilities of the array or system in respect to the both wild type mammary epithelial cells or metastatic mammary epithelial cells, adhesion signatures were collected for both cell types at 48 hours after seeding. FIG. 11A depicts the adhesion sets that preferentially proliferate normal mammary epithelial cells. FIG. 11B depicts the adhesion sets that preferentially bound to metastatic epithelial cells. All raw adhesion values were then normalized by the following protocol: each combination has five replicates per slide. Any replicate that is one standard deviation above or below the mean of the replicates is discarded and the new mean is calculated. After the mean of each combination is computed, the mean of all the combinations where the count is greater than zero is computer for each slide. All combinations on the slide are then normalized to this mean. This normalization scheme is useful particularly for analysis of adhesion as it removes biases from the following two sources. FIG. 11C depicts a heatmap representative of the adhesion sets to which there was a greater magnitude of differential binding counts between the two, depicted cell types. Here, “counts” refers to the normalized number of cells on a given combination at 48 hours after initial seeding of cells. The adhesion sets labeled “1” are those adhesion sets that stimulated mesenchymal cells proliferation more robustly than wild-type mammary epithelial cells. The adhesion sets under the column labeled “2”, are those adhesion sets that stimulated proliferation of wild type mammary epithelial cells more robustly than those cells with metastatic character.

A heatmap was generated to illustrate raw differential adhesion values (data not shown). Normalization is as follows: variations in cell numbers put on each slide due to error from pipetting, counting, or both; and global adhesive changes that are not representative in changes to particular ECM combinations (i.e. one cell type being generally more “sticky” than another) The latter normalization step is particularly relevant as metastatic cells that still exhibit the same relative adhesion to a particular combination as their primary tumor counterparts will not appear to have reduced adhesion to it simply because those cells tend to be globally less adhesive.

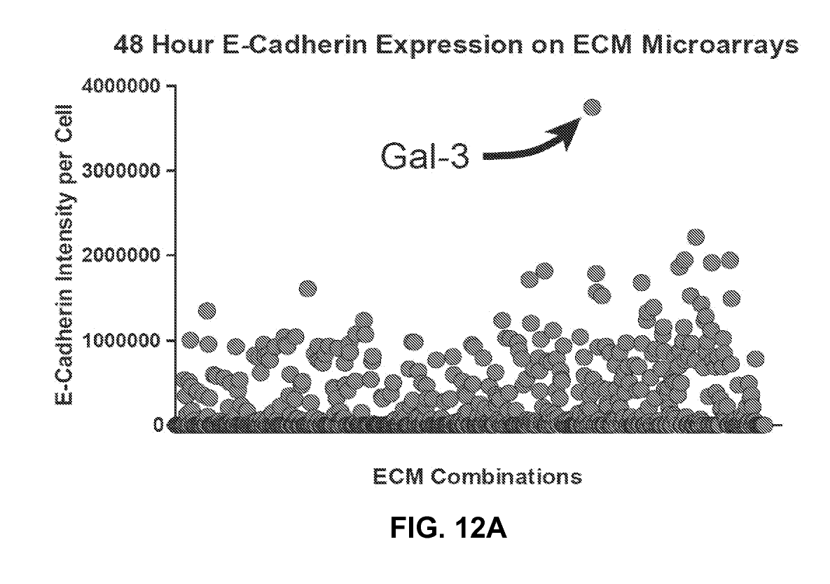

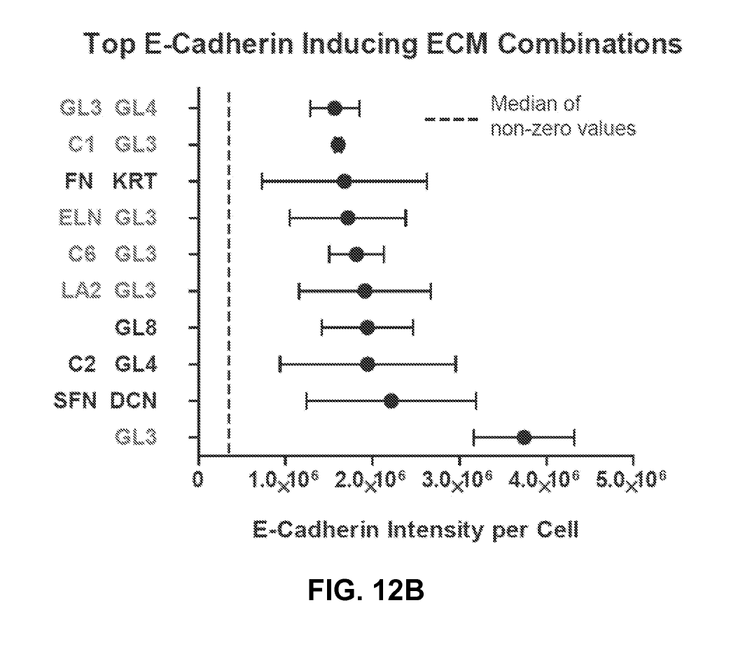

FIG. 12A-FIG. 12B relate to induction of the reverse transition known as MET. The goal of the study is to determine if any ECM combinations are capable of inducing this transition as it would likely confer the ability of a cell (or cluster of cells) that has reached a distant site to actually form a tumor at that site. One of the strongest markers of epithelial states (in comparison to mesenchymal) is the presence of E-Cadherin (a cell-cell junction protein). Thus the experiment measures E-Cadherin staining (on a per cell basis) of cells on all of different adhesion sets listed.

48 Hour E-Cadherin Expression on ECM Microarrays: FIG. 12A shows E-Cad protein expression (by cell staining) on the epithelial (wt) cells on each adhesion plotted as a single dot on our arrays. 48 hours after seeding cells, the slides were fixed and stained using a murine anti-human monoclonal antibody (BD Transduction Laboratories, clone 34/E-Cadherin). A TexasRed goat anti-mouse secondary antibody (Jackson Laboratories) was used. Imaging was performed as before. For each ECM combination, the staining intensity was determined for the spot and normalized by dividing by the number of cells on the spot in order to determine the intensity of staining per cell. Each dot on this plot represents the staining intensity of a unique ECM combination for the epithelial cells. The adhesion set that stimulated expression of E-Cadherin cells (i.e. the strongest activation of the epithelial programs) was galectin-3 without a pair-wise partner.

FIG. 12B depicts the adhesion sets that induce E-Cadherin expression and colonization capacity of cells with metastatic mammary character. Combinations highlighted in gray contain galectin-3.

48 Hour TWIST+E-Cadherin Expression on ECM Microarrays: This graph is the same as the first but with the mesenchymal (twist+) cells instead of the epithelial cells. Here, black dots depict combinations containing galectin-3. It is worth noting that E-Cadherin intensity (even of the top combinations) is much lower than the epithelial cells. This is due to their mesenchymal state (which should be lacking E-Cadherin expression). We expect that galectin-3 would induce a potent upregulation of epithelial markers such as E-Cadherin in this case. Nonetheless, the strong evidence for increased E-Cadherin expression in the context of the epithelial cells is quite convincing for its role in inducing an epithelial phenotype and likely conferring the ability of metastatic tumors to colonize distant sites.

Taken together, FIGS. 10 through 12 suggest that galectin-3 and galectins-8 alone and in combination with other ECM components induce an MET and subsequent proliferation once metastatic cells have reached their secondary site.

### Example 7

### Differing Adhesion Signatures of Differentiated Human Stem Cells

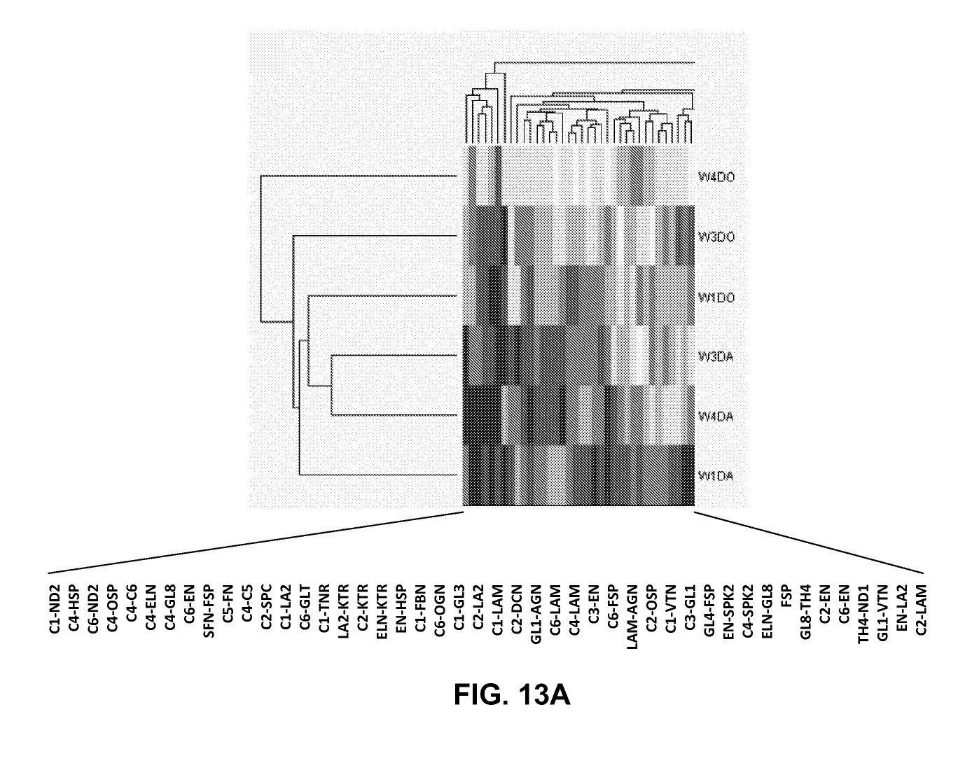

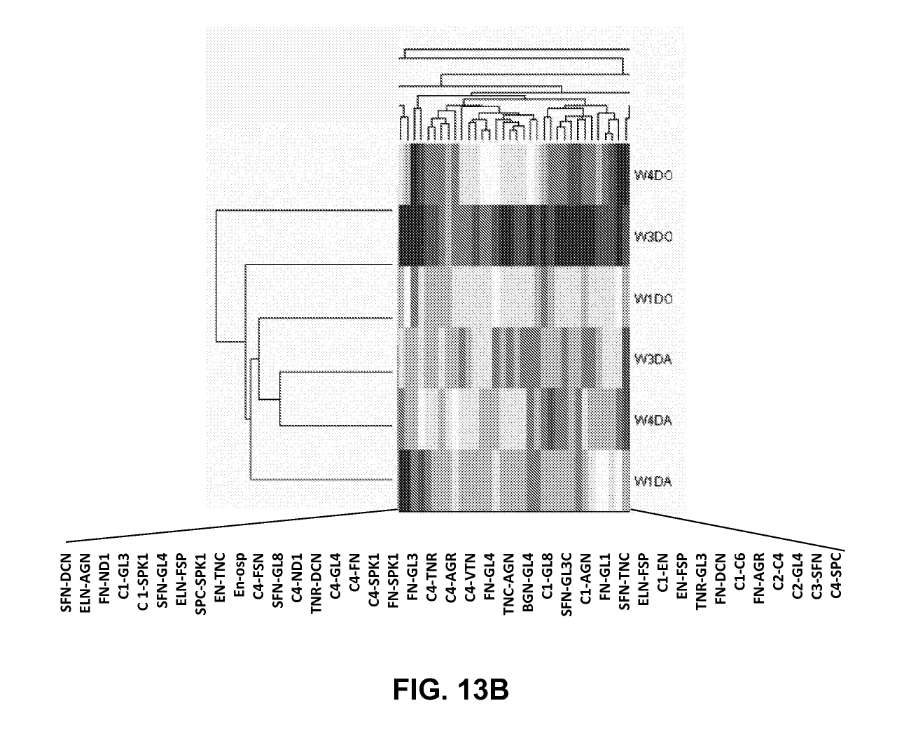

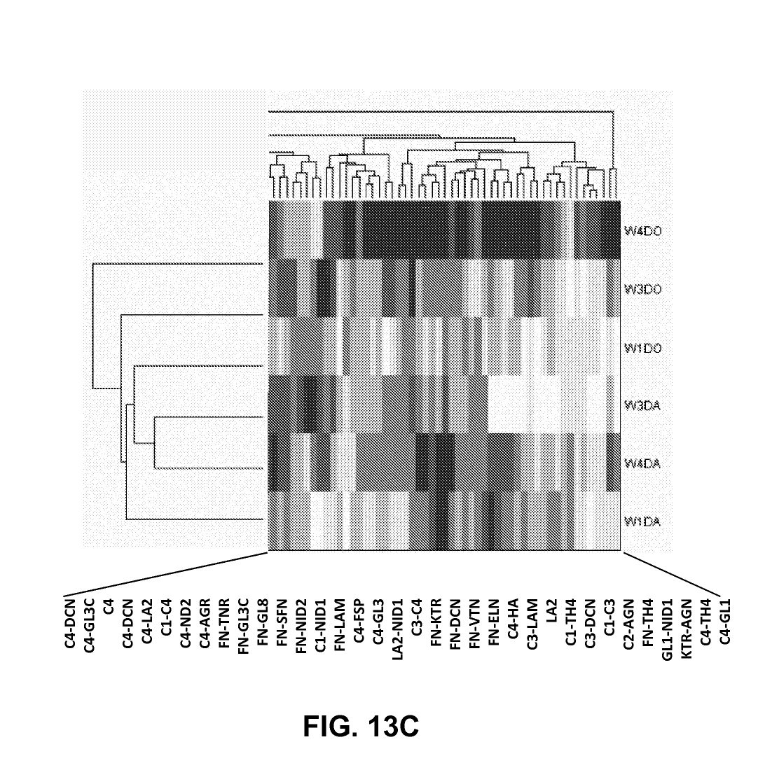

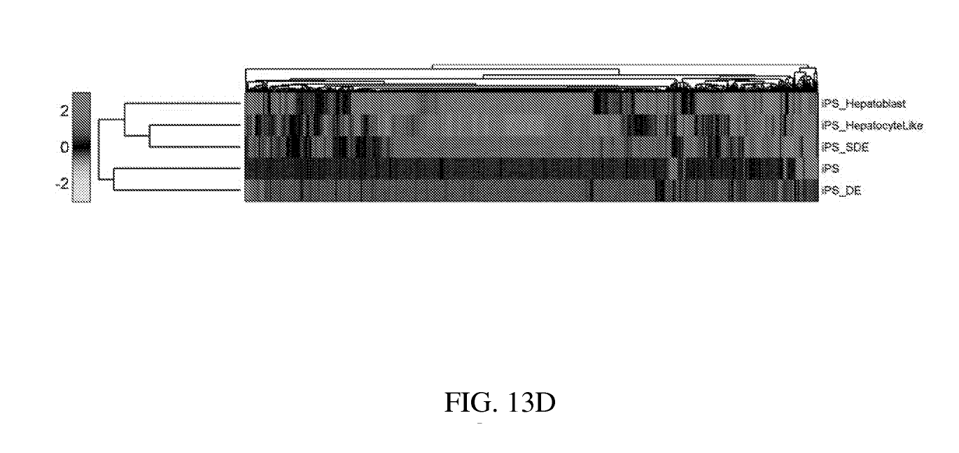

The following example demonstrates the use of ECM arrays to characterize differentiation states of stem cells. Stem cells are a promising approach to treatment of human disease due to their inherent ability to proliferate and differentiate to all cell types in the human body. These proprieties make stem cells an ideal cell source for cellular therapy, but so far there is no available method to access and identify if a differentiated stem cell indeed resembles a native cell and to track the differentiation status of the cell. To address this issue, adhesion signatures generated by ECM arrays of human mesenchymal stem cells during differentiation towards osteogenic and adipogenic lineages (FIGS. 13A, 13B, and 13C) and of human induced pluripotent stem cells towards the hepatic lineage (FIG. 13D) were tracked and this signature was benchmarked against native cells for the specific tissues.

Adhesion signatures generated by ECM arrays are able to distinguish between differentiation states of stem cells from different sources and towards different lineages, enabling the clear identification of a differentiation status of a given cell sample and to compare it to the native tissue. Out of these signatures it is also possible, in accordance with the present disclosure, to select an appropriate ECM component to isolate and culture cells in specific states of differentiation out of a mixed culture. For instance during hepatic differentiation vitronectin in combination with galectin-3 restricts cells in the endoderm stage.

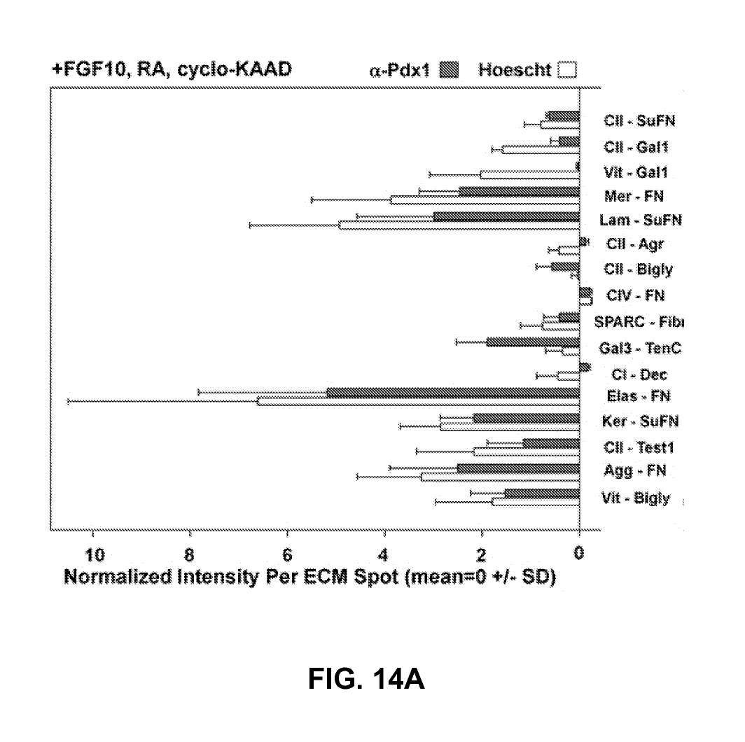

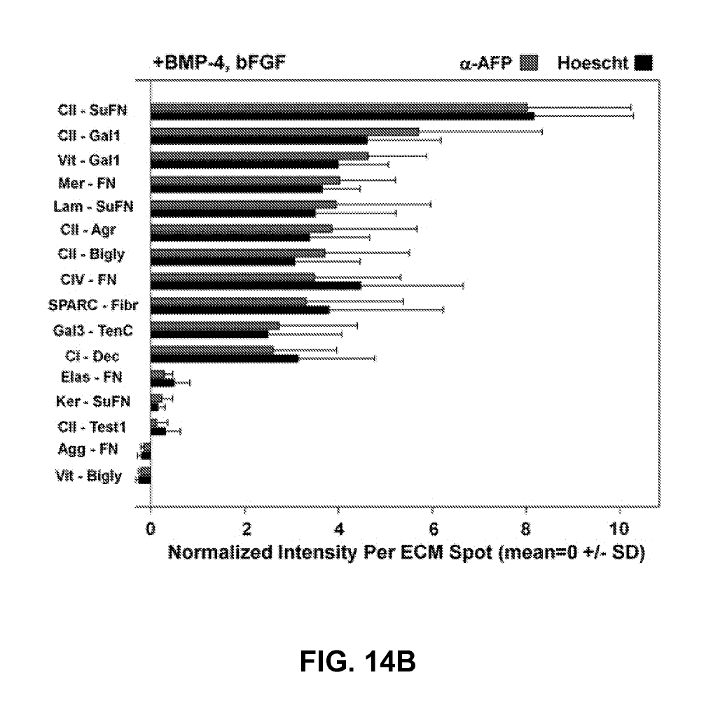

In addition to the ability to isolate and expand these cells, induction of differentiation is an area of active research. Definition of specific cell fates is still unclear for the majority of cellular fates, and the extra cellular signaling component has been mostly ignored in these efforts. The ability of ECM to support and induce differentiation of stem cells towards a specific lineage was investigated, using the liver-pancreatic fate switch as a model system (FIGS. 14 A and 14B). The present disclosure suggests that ECM play an important role in the fate choice of stem cells during the differentiation process, and that determination of ECM adhesion signatures, as described herein, usefully permits characterization of cells at various stages of differentiation.

### Example 8

### Determining Growth Rate as a Function of Adhesion Signature

In the present example, determining growth rate as a function of adhesion signature is described. Growth rate of the cells on different ECM components or combinations thereof can be determined. This system allows selection of the ECM composition that supports both the highest adhesion to the ECM array and the greatest growth rate of attached cells. In preferred embodiments, the steps of culturing a cell type of interest would additionally include determining or obtaining a second adhesion signature of cells that had been incubated with ECM components, and thus allowed to multiply, and comparing this adhesion signature to the adhesion signature of the cells without the added incubation step to determine the growth rate of the attached cells. In certain embodiments, both adhesion signatures are obtained using ECM arrays. In further embodiments, the second ECM array is incubated for between 30 minutes and 5 days. In preferred embodiments, the second ECM array is incubated for between 12 and 48 hours.

### Example 9

### Isolation of Mesenchymal Stem Cells with ECM Arrays

The following example describes the use of ECM arrays to identify growth conditions for mesenchymal stem cells. Adult stem cells, in particular mesenchymal stem cells (MSCs), are actively being explored in the clinic for their immune-modulatory proprieties. Currently there are around 200 clinical trials ongoing using these cells. A major bottleneck to the use of these cells in a clinical setting is their isolations and culture in xeno-free conditions. Currently, there are xeno-free medias available, but they all rely on complex, non-characterized animal derived matrices for isolation and culture of these cells. The FDA has mandated that clinical trials for efficacy require a xeno-free culture system for these cells. Thus, an alternative is needed to animal derived extracellular matrices that are currently being used.

Arrays were fabricated using vantage acrylic slides (CEL-1 Associates VACR-25C) coated with polyacrylamide gel pads (60×22 mm) as described previously 30. ECM arrays were spotted using a DNA Microarray spotter (Cartesian Technologies Pixsys Microarray Spotter and ArrayIt 946 Pins) from 384 well source plates containing the ECM combinations previously prepared using a Tecan EVO 150 liquid handler. Molecules were prepared to a final concentration of 200 g/ml in a buffer described previously. 741 combinations were spotted in replicates of five and rhodamine dextran (invitrogen) was spotted as negative controls and alignment reference for analysis. ECM arrays were stored in a humidified chamber at 4° C., until later use. The following ECM molecules were incorporated in the array: Collagen I, Collagen II, Collagen III, Collagen IV, Fibronectin, Laminin, Chondroitin Sulfate, Merosin (Millipore), Collagen V, Collagen VI (BD Biosciences), Aggrecan, Elastin, Keratin, Mucin, Heparan Sulfate, Superfibronectin, Fibrin, Hyaluronan (Sigma), Tenascin-R, F-Spondin, Nidogen-2, Biglycan, Decorin, Galectin 1, Galectin 3, Galectin 4, Galectin 8, Thrombospondin-4, Osteopontin, Osteonectin, Testican 1, Testican 2, Tenascin-C, Nidogen-1, Vitronectin, Rat, Agrin, Brevican (R&D Systems) and Galectin 3c (EMD Biosciences).

Before cell seeding slides were washed in PBS and sterilized with UV light. Cell seeding occurs in specially designed devices that hold the top surface of the slides flush with bottom of the well and secure the slides under vacuum. Cell were seeded on ECM arrays in serum free conditions and cultured in appropriate conditions. After seeding, slides were transferred to quadriperm plates (NUNC, 167063), and fresh media was added. Cells grew for different periods under these conditions and fed daily in longer studies. Slides were then stained for nuclei and marker expression. Briefly, slides were washed three times with PBS and fixed with 4% paraformaldehyde. At the same time nuclei were stained using Hoechst (Invitrogen) in combination with 0.1% Triton-X and PBS. Slides were then washed again and blocked using a blocking solution containing the anti-sera from the animal where secondary antibodies were raised for one hour. After blocking slides were incubated with primary overnight at 4° C. Secondary antibody (Invitrogen) incubation for 45 minutes followed after PBS washes. Slides were finally washed and mounted with Fluoromount-G (Southern Biotech) and stored at 4° C. until imaging. The entire slide was imaged using a Nikon Ti-Eclipse inverted fluorescence microscope and NIS Elements Software (Nikon). Image processing and analysis was performed in MATLAB (Mathworks) and nuclei and marker intensity quantification using CellProfiler (Carpenter, et al., “CellProfiler: image analysis software for identifying and quantifying cell phenotypes. Genome Biology 7, R100 (2006)). Replicate spots on each slide were averaged and those whose values were greater than one standard deviation above or below the mean of the replicates were excluded. Data was then normalized to allow averaging independent experiment data points.

### Cell Culture and Differentiation

MSCs were isolated from the mononuclear fraction of bone marrow cells of healthy donors, via adhesion in DMEM supplemented with fetal bovine serum and pen/strep (Invitrogen). Differentiation of MSCs towards the osteogenic and adipogenic lineages was carried out using the Invitrogen adipogenic and osteogenic differentiation kits and following the manufacturer's recommendations.

### Immunostaining

Immunostaining of ECM arrays for the presence ECM molecules was done by a 2 step immunofluorescence protocol. First slides were blocked for 1 hour with BSA and incubated overnight at 4 C with primary antibodies. After three washing steps slides were incubated with secondary antibodies labeled with near IR dies and imaged using the Licor System. Obtained images were colored and merged to form the 38 antibody stain ECM array representation. Cells were labeled for nuclear content using Hoechst stain and for actin with Alexa488 conjugated phalloidin (Invitrogen) according to supplier's instructions.

### Analysis

To quantify cell-ECM interactions in the ECM array we developed and automated an image acquisition and analysis process that combines both publicly available and in house developed software. After nuclear and specific marker staining according to conventional fluorescent staining protocols slides are imaged and ECM effects quantified. First, slides are imaged using an automated inverted epifluorescent microscope with NIS Elements software. This generates a multi-channel image of the entire ECM array (20×40 mm) at 4× magnification. Large images are then imported to MATLAB, individual channels isolated, and individual spots are cropped and indexed. Individual images are then fed to CellProfiler where specific cell parameters can be quantified 497. Typically, parameters as nuclei number, nuclei occupied area, nuclei intensity, and specific marker intensity and area are calculated. CellProfiler output data is then imported back to MATLAB where array data is extracted. The first step is to transform the output data in a 40 by 100 matrix that represents each individual island in the ECM array. Replicate ECM combinations are then averaged to generate an 8 by 100 matrix comprising the average ECM score for each ECM combination in the array. Before further analysis, a statistic test is run to exclude outlier spots. Outliers are considered if the statistical distance between the spot score and the average of the five replicas present in the array is larger than the standard deviation in the five replicates. Analysis of multiple experiments revealed that less than 3% of the 4000 features per array are considered outlying points (data not shown). To allow comparison between independent experiments, the ECM array data is normalized. This step facilitates comparison of measurements between experimental batches and corrects for overall differences in the imaging process (mostly due to fluorescence lamp intensity fluctuations). The score, or adhesion value, for each ECM combination is normalized against the average and standard deviation of the array according to the following formula:

$$\frac{x - {μ}_{array}}{{σ}_{array}}$$

Obtained data is centered on 0 and individual ECM scores represent the distance in standard deviations from the mean of the slide creating a relative score for each ECM combination. ECM combinations close to the average value of the array have a score close to 0 whereas combinations with high or low scores have positive and negative values depending on the distance from the average of the slide.

Initial tests with MSCs focused on the multiple parameters that can be obtained during image quantification using CellProfiler for the selection of the best descriptors of cell-ECM interactions. In these experiments, MSCs were differentiated in vitro towards the adipogenic and osteogenic lineages and the cell-ECM interaction descriptors of both these differentiated cell types where compared with undifferentiated MSCs by analysis on ECM arrays. Specifically, arrays 12 hours post seeding were fixed and stained both for DNA content and actin (FIG. 15A). This double stain allows for quantification of cell numbers and cell spreading for a given ECM combination across multiple cell differentiation stages within the same genetic background.

FIG. 15A highlights ECM combinations with different effects on both cell adhesion and cell spreading. For example, Galectin-8/Thrombospondin-4 promotes high adhesion levels of both MSCs and their osteogenic progeny whereas adipogenic differentiated cells have significantly less tendency for adhesion. In addition to differences in cell numbers, the cell spreading phenotype is dramatically different on this combination: the overall occupied area of adipogenic cells does not change significantly, but actin organization seems to be different. For Mucin/Collagen I and Brevican/Chondroitin Sulfate the impact is different. There are no significant differences in the number of cells present across different cell types, but cell spreading seems to be reduced in adipogenic cells. Finally, actin organization is strongly dependent of ECM composition and even differentiation stage. Different ECM niches seem to induce different cytoskeleton organization profiles.

The quantification of adherent nuclei was used to generate MSC adhesion profiles (FIGS. 15B and 15C). Analysis of the MSC adhesion profile clearly shows that MSCs have a stronger tendency to attach to ECM combinations with at least one component that is characteristic of basement membranes like collagens or laminins MSCs have strong adhesion scores to more than 30% of ECM combinations in the array and top adhesion ECM combinations can be used in the future for the isolation and culture of MSCs in animal free conditions (FIGS. 15B and 15C). MSC proliferation can also be indirectly measured in the array via the comparison of adhesion profiles at early (6 h) and later time points (24 h). Therefore, combinations that represent promising candidates for animal free expansion of MSCs can be selected both from strong mediators of adhesion at early and late time points in culture.

To address FDA requirements, ECM arrays were used to identify xeno-free extracellular matrices for isolation and expansion of MSCs (FIGS. 15D and 15E). As demonstrated herein, several combinations have been identified as holding the potential to achieve this goal. For example, combination of Galectin-8 and Thrombospondin-4 (both recombinant molecules) permits the highest yield of adhesion and expansion in a xeno-free environment. Use of this combination ensures practitioners can successfully utilize fully synthetic and defined culture systems for MSC in a clinical setting, as required by regulatory organizations like the FDA.

Adhesion profiles can also be used to compare and distinguish different cell states. For instance, during adipogenic and osteogenic differentiation of MSCs adhesion profiles of differentiating cells change overtime (FIG. 15E). Unsupervised clustering of adhesion profiles at three distinct time points (weeks 1, 3 and 4) of adipogenic and osteogenic differentiation of MSCs shows that adipogenic and osteogenic adhesion profiles cluster apart from each other from the earliest time point (FIGS. 13A, 13B, and 13C). Osteogenic cells show a preference for ECM combinations that present ECM molecules known to form stiffer tissues like fibronectin or collagens (FIG. 15F). Notably, adhesion profiles have the potential to be used as cell signatures to distinguish differential phenotypes in cells of the same genetic background. Such analysis also has the potential to be utilized to isolate cells at specific stages of development from a differentiating culture based on positive and negative adhesion profiles. These profiles can also be used to identify culture conditions that allow robust expansion and differentiation of stem cells or to culture isolated primary cells.

Table 3 below is a list of the combination of adhesion sets useful for mesenchymal stem cell isolation, culture and differentiation:

TABLE 3

| ECM combination | |
|---|---|
| ID number | ECM combinations |
| 1 | Gal-8 + Vit |
| 2 | Gal-3 + Vit |
| 3 | Osteo + Vit |
| 4 | Ten-R + Bre |
| 5 | Bigly + Gal-4 |
| 6 | Test-2 + Vit |
| 7 | Dec + Osteo |
| 8 | Throm-4 + Osteo |
| 9 | Dec + Vit |
| 10 | Throm-4 + Vit |
| 11 | Test-1 + Vit |
| 12 | Nid-2 + Vit |
| 13 | Bigly + Vit |
| 14 | Vit + Agr |
| 15 | CII + Ker |
| 16 | CI + Fib |
| 17 | Gal-4 + Thom-4 |
| 18 | Gal3 |
| 19 | Test-1 |
| 20 | Gal-1 + Bre |
| 21 | Nid-2 + Bre |
| 22 | Dec + Gal-8 |
| 23 | Fib + Bre |
| 24 | Fib + Test-1 |
| 25 | Fib + Gal-4 |
| 26 | Fib + Osteo |
| 27 | Fib + Vit |
| 28 | Fib + Gal-3 |
| 29 | Col-1 + Test-1 |
| 30 | Col-1 + Gal-3 |
| 31 | Col-1 + Gal-8 |
| 32 | Col-1 + Osteo |
| 33 | Col-1 + Vit |
| 34 | Col-1 + Nid-2 |

### Example 10

### Identification of Adhesion Molecules to Enable Plating of Unplatable Hepatocytes with ECM Arrays

In the following example, the use of ECM arrays to identify growth conditions for hepatocytes is described. Hepatocytes are the main cell type in the liver. They are responsible for metabolism of the majority of drugs in the human body. Hepatic disease affects around 20 million Americans. The availability of cells to study liver disease is limited, mainly because only around 10% of donor cells are plateable after isolation. The definition of plateability is adhesion to collagen I, an ECM component traditionally used to culture hepatocytes. The complexity of ECM in the human body is significantly greater than that of a single ECM component, so we sought to look for an appropriate ECM that would enable plating unplateable hepatocytes (FIG. 16A through 16D). Our results, described herein, show that across 6 different lots of unplateable hepatocytes, all of them had several ECM matrix combinations that promoted cell adhesion and combinations of Collagen I with Aggrecan and of Collagen IV with Nidogen-1 seem to have a universal effect.

### Example 11

### Identification of Adhesion Molecules to Maintain Induced Pluripotent Stem Cells Pluripotency with ECM Arrays

In the following example, the use of ECM arrays to identify conditions for maintaining stem cells is described. Human embryonic and induced pluripotent stem cells (hESC/hiPSC) have the ability to differentiate into all cell lineages and thus hold great promise for the treatment of human disease. hESC and hIPSC have the ability to differentiate into all cell lineages and thus hold great promise for the treatment of human disease. However, current methods to grow hIPSC require mitotically inactivated feeder cells (MEFs) or undefined ECM mixes (i.e. Matrigel) and thus introduce animal factors and lot-to-lot variability. To identify human native or recombinant ECM and understand the role of ECM in the maintenance of pluripotency, we employed the ECM platform characterized in the disclosure herein.

To identify human native or recombinant ECM components capable of maintaining pluripotency, we looked for adhesion signatures of hESC/hiPSC on our ECM array (FIG. 17A through 17C). At least three ECM combinations that support iPSC self-renewal and pluripotency for more than 30 passages (>85% oct3/4+tra1-60+sseal+) were identified. ECM expanded iPSC maintained a normal karyotype, the potential to form embryoid bodies in vitro and teratomas in vivo and to differentiate towards the hepatic lineage in vitro.

Follow-up showed dependence of pluripotency on specific ECM combinations: (i) single matrix molecules are unable to maintain the pluripotency phenotype and (ii) blocking ECM components and signaling induces loss of pluripotency. ECM combinations are also able to support iPSC in defined media. These ECM combinations also support differentiation of cells to specific lineages. Based on these results we were also able to study the relationships between ECM and pluripotency signal cascades. We show that different ECM combinations induce different SMAD activation profiles and that SMAD levels are related to AKT levels. Maintenance of pluripotency requires an initial activation of SMAD2/3 and this protein appears to interact with AKT. Overall, by employing an unbiased and high-throughput approach we were able to identify ECM combinations that support pluripotency and to translate these results to a simple tissue culture system that revealed aspects of the molecular mechanisms responsible for this maintenance.

### ECM Array Fabrication and Analysis

Briefly, vantage acrylic slides (CEL-1 Associates VACR-25C) were coated with polyacrylamide gel pads (60×22 mm) as described previously 30. ECM arrays were spotted using a DNA Microarray spotter (Cartesian Technologies Pixsys Microarray Spotter and ArrayIt 946 Pins) from 384 well source plates containing the ECM combinations previously prepared using a Tecan EVO 150 liquid handler. Molecules were prepared to a final concentration of 200 μg/ml in a buffer described previously. 741 combinations were spotted in replicates of five and rhodamine dextran (invitrogen) was spotted as negative controls and alignment reference for analysis. ECM arrays were stored in a humidified chamber at 4° C., until later use. The following ECM molecules were incorporated in the array: Collagen I, Collagen II, Collagen III, Collagen IV, Fibronectin, Laminin, Chondroitin Sulfate, Merosin (Millipore), Collagen V, Collagen VI (BD Biosciences), Aggrecan, Elastin, Keratin, Mucin, Heparan Sulfate, Superfibronectin, Fibrin, Hyaluronan (Sigma), Tenascin-R, F-Spondin, Nidogen-2, Biglycan, Decorin, Galectin 1, Galectin 3, Galectin 4, Galectin 8, Thrombospondin-4, Osteopontin, Osteonectin, Testican 1, Testican 2, Tenascin-C, Nidogen-1, Vitronectin, Rat, Agrin, Brevican (R&D Systems) and Galectin 3c (EMD Biosciences).

Before cell seeding slides were washed in PBS and sterilized with UV light. Cell seeding occurs in specially designed devices that hold the top surface of the slides flush with bottom of the well and secure the slides under vacuum. One million cells were seeded on each slide in 5 mL of conditioned media 513(hESC media described elsewhere condition by mouse CF-1 embryonal fibroblasts) and seeded overnight at 37° C. After seeding, slides were transferred to quadriperm plates (NUNC, 167063), and fresh media was added. Cells grew for 48 hours under these conditions and fed daily. Slides were then stained for nuclei and marker expression. Briefly, slides were washed three times with PBS and fixed with 4% paraformaldehyde. At the same time nuclei were stained using Hoechst (Invitrogen) in combination with 0.1% Triton-X and PBS. Slides were then washed again and blocked using a blocking solution containing the anti-sera from the animal where secondary antibodies were raised for one hour. After blocking slides were incubated with primary antibodies oct3/4 (BD), tra1-60 and ssea4 (EBiosciences) overnight at 4° C. Secondary antibody (Invitrogen) incubation for 45 minutes followed after PBS washes. Slides were finally washed and mounted with Fluoromount-G (Southern Biotech) and stored at 4° C. until imaging. The entire slide was imaged using a Nikon Ti-Eclipse inverted fluorescence microscope and NIS Elements Software (Nikon). Image processing and analysis was performed in MATLAB (Mathworks) and nuclei and marker intensity quantification using CellProfiler as described herein. Replicate spots on each slide were averaged and those whose values were greater than one standard deviation above or below the mean of the replicates were excluded. Data was then normalized to allow averaging independent experiment data points.

hIPSC/hESC Culture

Undifferentiated iPSC and hESC were maintained as described 95. In short, Human H9 (WA09) ESC and iPSC (IPSC2A and RC2) were cultured in hESC cell media (DMEM F12 medium supplemented with 20% knockout serum replacement, non-essential amino acids, glutamine, penicillin/streptomycin and bFGF (4 ng/ml; Invitrogen)) on mitotically inactivated mouse embryonic fibroblasts (MEFs) or on Matrigel coated plates using MEF-conditioned medium. Alternatively mTESR1 media was used for studies with defined media compositions. hIPSC and hESC cultured on ECM combinations were dispersed as singe cells and seed on regular TCP plates with adsorbed ECM molecules. ECM molecules were adsorbed in diH2O at a concentration of 15 g/ml for at least six hours and then UV treated for sterilization. The regular culture conditions were otherwise maintained.

### ImmunoFluorescence and Flow Cytometry

ImmunoFluorescence (IF) on cultured cells followed the protocol previously described for ECM array slides, with the adequate adaptations. For flow cytometry analysis cells were incubated for 10 min with Accutase (Millipore) at 37° C. Cells were then fixed, permeabilized and blocked with the Cytofix/Cytoperm solution (BD) for 15 min. Cells were incubated for 30 min at 4° C. with primary antibodies diluted in perm/wash buffer (BD), washed and kept on ice until analysis. Flow cytometry analysis was performed using the FACScalibur or LSRII system (BD). For phosphorylated proteins analyzed via flow cytometry, cells were incubated with for 10 min with Accutase (Millipore) at 37° C. and then fixed, permeabilized and blocked as previously reported. In brief, after accutase treatment cells were immediately fixed with 1.6% paraformaldehyde for 10 minutes at room temperature in phosphatase inhibitor containing solution (Roche). Cells were then pelleted, washed and then permeabilized with ice cold methanol for 10 minutes at 4° C. Cells were incubated for 30 min at 4° C. with primary antibodies diluted in blocking buffer (PBS with 1% BSA and PHOSSTOP), washed and kept on ice until analysis. Flow cytometry analysis was performed using the LSRII system (BD).

Adhesion Blocking Assay

Adhesion blocking experiments were done using the α and β integrin investigator kits from Millipore. Cells were incubated with integrin blocking antibodies (2 μg/ml) for 30 minutes on ice. Cells where then incubated for one hour at 37° C., fixed with 4% paraformaldehyde containing Hoechst nuclear counterstains. A scan image of the entire well was acquired using a Nikon Ti-Eclipse inverted fluorescence microscope and cell counts where performed on the NIS Elements Software (Nikon).

### Teratomas

All animals were housed in the Koch Institute animal facility and the Committee for Animal Care in the Department of Comparative Medicine at Massachusetts Institute of Technology approved all animal procedures. To generate teratomas cells were retrieved using accutase followed by centrifugation and resuspension in 250 μl of Matrigel (2 mg/ml in DMEM-F12; BD Bioscience). Cells were injected into the dorsal flank of Nude male mice (Taconic) using a 27G needle, and teratomas were dissected 8 to 11 weeks after injection and processed for histology using hematoxylin and eosin. Sections were analyzed by a trained pathologist to determine the nature of the obtained tumors.

### Karyotyping

Karyotyping analysis was done by Cell Line Genetics (Madison, Wis.) using the high-resolution G-band standard protocols.

### Differentiations

To generate hepatocytes, monolayers of pluripotent cells harvested using Accutase (Millipore) were plated on 6 well plates pre-coated with 2 mg/ml Matrigel (Growth Factor Reduced; BD Bioscience) at a density of 5.0×105 cells per well in hESC cell media and 10 μM Y27632 and washed the following day. Once cells reached confluency, differentiations were initiated by culture for 5 days with 100 ng/ml Activin A (R&D Systems) in RPMI/B27 medium (Invitrogen) under ambient oxygen/5% CO2, followed by 5 days with 20 ng/ml BMP4 (Peprotech)/10 ng/ml FGF-2 (Invitrogen) in RPMI/B27 under 4% O2/5% CO2, then 5 days with 20 ng/ml HGF (Peprotech) in RPMI/B27 supplement under 4% O2/5% CO2, and finally for 5 days with 20 ng/ml Oncostatin-M (R&D Systems) in Hepatocyte Culture Media (Lonza) supplemented with SingleQuots (without EGF) in ambient oxygen/5% CO2.

To generate cardiomyocytes, monolayers of pluripotent cells harvested using Accutase (Millipore) were plated on 6 well plates pre-coated with 2 mg/ml Matrigel (Growth Factor Reduced; BD Bioscience) at a density of 1.0×105 cells per well in hESC cell media and 10 μM Y27632 and washed the following day. Once cells reached confluency around day 10, differentiations were initiated by culture for 5 days with 50 ng/ml Activin A (R&D Systems) and 20 ng/mL BMP4 in RPMI/B27 medium (Invitrogen) under ambient oxygen/5% CO2, followed by 10 days with 20 ng/ml BMP4 (Peprotech)/10 ng/ml FGF-2 (Invitrogen) in RPMI/B27 under 4% O2/5% CO2, and then finally 5 days in RPMI/B27 supplement under 4% O2/5% CO₂.

iPSC were differentiated according to previously established methods to the neuronal lineage. Briefly, iPSC were cultured on Matrigel-coated plates and with hES MEF conditioned media. Colonies were lifted off with dispase solution and cultured in suspension for 4 days with fresh hES media and 3 days with neural differentiation media (consisting of DMEM/F12, N2 supplement, and nonessential amino acid) (all from Invitrogen Corporation, Carlsbad, Calif.). Aggregates were then plated on a laminin coated surface (Sigma-Aldrich, St. Louis, Mo.). On day 10, 0.1 μM retinoic acid (Sigma-Aldrich) was added to the neural differentiation media. Neural tube-like rosettes formed at day 15 of differentiation and were then detached mechanically and cultured in suspension in neural induction medium containing B27, 0.1 μM retinoic acid, and 1 μM purmorphamine (Cayman Chemical). After 5 days, neurospheres were collected and split using accutase (Millipore) and passaged in suspension. A sample was collected and lyzed for PCR analysis. Neurospheres were cultured in neural induction medium with B27, FGF8b 50 ng/mL, SHH 100 ng/mL, and ascorbic acid (200 μM) for 7 days. Neurospheres were then treated with accutase/trypsin and seeded as single cells onto polyornithine/laminin coated tissue culture plates in neural differentiation medium with FGF8b 50 ng/mL, SHH 100 ng/mL, ascorbic acid (200 μM), cAMP (1.0 μM), TGFβ3 (1 ng/mL), BDNF (10 ng/mL), GDNF (10 ng/mL), IGF-1 (10 ng/mL), and WNT3A (10 ng/mL) for 21 days. All cytokines are from Peprotech except WNT3A (R&D systems) A sample was collected and lyzed for PCR analysis.

Western and Immune-Precipitation

Total protein was extracted with radioimmuneprecipitation assay lysis buffer with STOP protease inhibitors (Roche), and samples were separated by electrophoresis on 12% (wt/vol) polyacrylamide gels and electrophoretically transferred to a PVDF membrane (Bio-Rad Laboratories). Blots were probed with primary antibodies, followed by HRP-conjugated secondary antibodies, and were developed by SuperSignal West Pico substrate (Thermo Scientific).

RT-PCR

Total RNA was isolated with the RNeasy Plus Mini Kit (Qiagen). First-strand cDNA was synthesized using Moloney murine leukemia virus reverse transcriptase (Bio-Rad). Quantitative PCR was carried out with Taq polymerase and SYBR Green in the supplier's reaction buffer containing 1.5 mM MgCl2 (Bio-Rad). Oligonucleotide primer sequences are available by request. Amplicons were analyzed by both melt curve analysis on the Biorad MYIQ QRTPCR machine and confirmed by 2% (wt/vol) agarose gel electrophoresis (Sigma).

Albumin and α-1-Antitrypsin ELISA.

Spent medium was stored at −20° C. α-1-Antitrypsin and albumin media concentrations were measured using sandwich ELISA technique with HRP detection (Bethyl Laboratories) and 3,3,5,5-tetramethylbenzidine (Thermo Scientific) as a substrate.

FIG. 17A through 17C depicts how the ECM array reveals ECM combinations that support hIPSC self-renewal. We focused on the influence of ECM in the pluripotent state and screened for ECM combinations that would support PSC self-renewal. hIPSC were dispersed as single cells, seeded on the ECM array, and cultured for 48 hours. The array was then stained for nuclei and the pluripotency markers (oct3/4, ssea4 and tra1-60) and analyzed to identify ECM combinations that support PSC self-renewal (FIG. 17A through 17C). This analysis focused on the ability of ECM combinations to support expansion of hIPSC and to maintain their pluripotent phenotype. Each ECM island in the array was scored for cell numbers (via nuclear content) and the expression of each of the three pluripotency markers (FIG. 17B). The data presented is the average of the 5 quintuplicates present in one array that is normalized and averaged in two technical replicates and repeated in three independent experiments (FIG. 17A, scatter plots). ECM combinations that align on the vertical axis have robust expression of pluripotency markers, combinations on the horizontal axis have good cell numbers and the ones on the x=y axis combine cell number and expression of pluripotency markers. This means that as expected we were able to identify a range of ECM environments with different influences on cell fate: (i) ECM combinations that do not mediate attachment and growth of PSC, (ii) ECM combinations that promote PSC attachment but do not support the maintenance of pluripotency, (iii) ECM combinations that support cells with high expression of pluripotency markers, but reduced cellular proliferation and (iv) ECM combinations that support expansion of PSC maintaining their pluripotent phenotype (FIG. 17D through 17I).

To fully characterize these results we selected a set of ECM combinations to be validated in a system that would be easily translatable to a regular tissue culture strategy and that would enable studying the impact of ECM on pluripotency. We selected 8 ECM combinations from the four categories mentioned above (FIG. 17D through 17I) and translated the ECM array findings to a culture system by the simple adsorption of the ECM molecules to tissue culture plastic. hIPSC were seeded as single cells on adsorbed ECM molecules and their growth and expression of pluripotency markers was followed over time (FIG. 17D through 17I). From the eight ECM combinations selected, three (collagen I/laminin, collagen II/galectin-4 and collagen IV/galectin-8) were able to support long term, single cell, passaging of hIPSC and hESC in MEF-conditioned media (CM) and chemically defined media mTeSR1 515 (FIG. 17E) for at least 50 passages. hIPSC expanded on ECM combinations maintain the expression of the pluripotency markers oct3/4, ssea4 and tra1-60 (FIGS. 17F and 17G) and retain the characteristic cellular morphology (FIG. 17F). To ensure the relevance of this strategy we repeated the assay using two different hIPSC lines (IPSC2a and RC2) and a hESC line H9 (FIG. 17G). All three lines showed robust self-renewal on ECM combinations when passaged as single cells.

In defined media conditions (mTESR1), all ECM combinations were able to maintain pluripotency but only one matrix combination (collagen I and laminin) could maintain the robust expansion of pluripotent stem cells. Cells on collagen II/galectin-4 and on collagen IV/galectin-8 maintained the expression of oct3/4, ssea4 and tra1-60, but had a tendency to detach from the dish forming spheroid colonies instead of spreading in the surface (FIG. 17H). Analyzing the possible factors, we hypothesized that MEF conditioned media was providing an additional adhesive factor that was absent in the chemically defined media. Examination of MEF expression profiles identified the robust secretion of fibronectin, a ECM molecule that is known for its adhesive properties. Addition of fibronectin to the adsorbed ECM combinations enabled the robust expansion of PSCs and did not alter the capacity to support self-renewal (FIG. 17I). Pluripotency is a functional property of stem cells defined as the ability of a cell to form derivatives of all three embryonic layers. Oct3/4, SSEA4, and Tra1-60 are proxy markers of pluripotent cells, although SSEA4 and Tra1-60 are not considered immediate drivers of the pluripotency network 85. The ability of each identified ECM to support pluripotency was confirmed by testing the ability of cultured cells to form teratomas in vivo after injection on the dorsal flank of (Nude mice) (FIG. 17J, left) and to form embryonic bodies (EBs) in vitro with contributions to all three germ layers (FIG. 17K). Teratomas were characterized by the presence of tissues derived from the three germ layers and organized in organoid-like structures: respiratory epithelium, ductal structures, cartilage, bone, and neuroectodermal structures. EBs robustly expressed markers of the three germ layers by quantitative real-time PCR (QRT-PCR) (FIG. 17K). Genetic instability of expanded cells is a known issue during culture on matrigel and other chemically defined ECMs, and this tendency is one of the major limitations blocking the development of chemically defined methods for the long term expansion of PSC 517. hIPSC expanded on identified ECM combinations retain their normal karyotype after 10 passages (2 months) in culture (FIG. 17J, right panel) as revealed by G-band analysis.

The present disclosure therefore indicates that ECM molecules, when presented in specific combinations, are a reliable and defined platform to support iPSC, a potential alternative to MEFs and Matrigel.

Pluriptient stem cells (PSCs) maintain their pluripotent potential. PSCs have been widely explored as sources for cellular modeling or as replacement therapies for human disease. The adoption of long term culture systems for PSC requires that expanded cells are still able to be directly differentiated towards functional somatic cells. We differentiated hIPSC expanded on ECM combinations towards hepatocytes (endodermal lineage), cardiomyocytes (mesodermal lineage) and neurons (ectodermal lineage) following established protocols. ECM expanded hIPSC robustly generated hepatocyte-like cells after the stimulation with activin A, BMP4/bFGF, HGF and OSM. Differentiated cells secrete albumin and al antitrypsin to levels compared to matrigel expanded cells (FIG. 17L, 17M, 17N). The ability to generate cardiomyocytes was confirmed by the presence beating cells in culture that express alpha-myosin heavy chain and NKX2.5 (FIGS. 17O and 17P), markers characteristic of cardiomyocytes and are responsive to calcium signals (FIG. 17Q). After induction to the neuronal lineage cell express characteristic markers of the progenitor stage such as nestin and differentiated state like β-tubulin. Screening for ECM combinations identified unique environments that support self-renewal of PSCs that were easily translated to robust culture systems by the simple adsorption of ECM molecules to tissue culture plastic in contrast to the current state of art i.e. mitotically inactivated feeder layers or poorly-defined and animal derived ECM (FIGS. 17R and 17S). The adoption of the identified and fully characterized ECM combinations enables the production and differentiation of PSC in a defined and reproducible manner. The understanding of the role of specific ECM molecules in stem cell fate and the identification of the molecular pathways that are involved in this process are critical for the development of robust and clinically translatable culture and differentiation strategies for PSCs.

In vivo tissues are formed through the combination of several ECM molecules and the specificity of ECM in each tissue can be used as a signature of the tissue. The differences in ECM composition account for the structural differences in tissues although their role in regulating cell fate is unclear. The ECM array data (FIGS. 17T, 17U, and 17V) demonstrated that ECM combinations exert a stronger effect on the maintenance of pluripotency than do single molecules; the top 200 ECM niches as defined by this assay were all formed by two ECM molecules. Consequently, we evaluated the importance of single components in the ECM combinations that support pluripotency and the specificity of these identified combinations. Cells were seeded on adsorbed ECM molecules, grown to subconfluence in conditioned media (4-5 days) and analyzed for the expression of pluripotency markers. Individual ECM molecules did not support pluripotency as shown by the drastic drop in triple positive (oct3/4-ssea4-tra1-60+) cells in a single passage (FIG. 17T, left panel). Collagens I, II and IV and Laminin To evaluate the specificity of ECM molecules we exploited the fact that two of the three hit ECM combinations share molecules of the same family, collagen II/galectin-4, and collagen IV/galectin-8. By switching the ECM partners in the two combinations, we addressed the specificity issue given that the recombined pairs (collagen II with galectin-8, and collagen IV with galectin-4) included molecules with distinct, but closely related structure and function. Collagen II/Galectin 8 did not maintain the pluripotent state, whereas Collagen IV/Galectin 4 supported PSC self-renewal (FIG. 17T, middle left and middle right panels). This finding was consistent with the original data from the ECM array that revealed that specific ECM combinations were needed to maintain pluripotency. Galectins are a family of lectins that bind galactose via a specific carbohydrate recognition domain (CRD). This domain can be blocked by lactose or an analog like LacNac, a small disaccharide that has been shown to irreversibly bind the CRD region. Blocking the galectin CRD impairs the self-renewal support ability of ECM combinations containing galectins (FIG. 17T, right panel), demonstrating that galectins signal via the CRD in this context. The CRD domain of galectin has been shown to interact with the glycosylated domains of the highly glycosylated α1 integrin. Blocking α1 integrin results in a 50% reduction of cellular attachment to the hit ECM combinations (FIG. 17T, right panel) indicating that this integrin might be involved in these adhesion and signaling processes.

To promote adhesion in defined media conditions (such as mTESR1), fibronectin was added to our hit ECM combinations (FIG. 17U, left and right panels). However, the role played by fibronectin to mediate the maintenance of pluripotency in the context of the specific ECM combination hits was unknown. Cells grown on collagen I and Laminin did not need the addition of fibronectin, and were neither improved nor adversely affected by its addition (FIG. 17T, 17U, 17V). To further dissect the specificity, we employed a similar blocking strategy as described above: (i) combined fibronectin with the individual components on the ECM combinations and (ii) blocked galectin signaling with LacNac. Pairwise combinations of fibronectin and collagen II and IV or galectin 4 and 8 were unable to support pluripotency (FIG. 17T, right panel) and blocking the CRD domain of galectins using LacNac prevented pluripotency maintenance (FIG. 17T, right panel, 17U). Thus, we conclude based on these results that while fibronectin might mediate adhesion, its role in promoting pluripotency is secondary relative to the impact of the ECM combination hits. This outcome illustrates that robust self-renewal of PSC requires both a specific ECM combination signal related to pluripotency as well as sufficient adhesion signaling to retain proliferating cells. The specificity of the ECM combinations is critical for maintaining pluripotency signaling. This data highlights the importance of studying ECM in an unbiased fashion and examining the complex combinations of ECM molecules rather than focusing on individual components. Only by approaching the in vivo complexity are we able to have a closer picture to the cellular microenvironment and identify unique factors that are involved in pluripotent signaling.

### Example 12

### Cell Culture on ECM Components Absorbed on a Solid Substrate

In the following example, growth of cells on a solid substrate on which ECM components have been absorbed is described. Human H9 (WA09) embryonic stem cells and iPSC (IPSC2A and RC2) were cultured in hESC cell media (DMEM F12 medium supplemented with 20% knockout serum replacement, non-essential amino acids, glutamine, penicillin/streptomycin and bFGF (4 ng/ml; Invitrogen)) on mitotically inactivated mouse embryonic fibroblasts (MEFs) or on Matrigel coated plates using MEF-conditioned medium. Alternatively mTESR1 media was used for studies with defined media compositions. hIPSC and hESC cultured on ECM combinations were dispersed as single cells and seed on regular TCP plates with adsorbed ECM molecules. ECM molecules were adsorbed in diH2O at a concentration of 15 μg/ml or 8 μg/ml for at least six hours and then UV treated for sterilization. The culture conditions described in the previous Example were otherwise maintained. Selected ECM combinations for validation in a regular tissue culture approach (FIG. 17D through 17I and 17L through 17S, based upon Methods Section in above Example).

The example demonstrates that ECM component adsorption to TCP (polystyrene) can function in a similar fashion to multiplexed arrays of ECM components spotted to slides.

TABLE 1

| ECM component | Accession |
|---|---|
| Aggrecan (SEQ ID NO. 1) | NCBI Reference Sequence: NP_001126.3 |
| 1 | mttllwvfvt lrvitaavtv etsdhdnsls vsipqpsplr vllgtsltip cyfidpmhpv |
| 61 | ttapstapla prikwsrvsk ekevvllvat egrvrvnsay qdkvslpnyp aipsdatlev |
| 121 | qslrsndsgv yrcevmhgie dseatlevvv kgivfhyrai strytldfdr aqraclqnsa |
| 181 | iiatpeqlqa ayedgfhqcd agwladqtvr ypihtpregc ygdkdefpgv rtygirdtne |
| 241 | tydvycfaee megevfyats pekftfqeaa necrrlgarl attgqlylaw qagmdmcsag |
| 301 | wladrsvryp iskarpncgg nllgvrtvyv hanqtgypdp ssrydaicyt gedfvdipen |
| 361 | ffgvggeedi tvqtvtwpdm elplprnite geargsvilt vkpifevsps plepeepftf |
| 421 | apeigatafa evenetgeat rpwgfptpgl gpataftsed lvvqvtavpg qphlpggvvf |
| 481 | hyrpgptrys ltfeeaqqac lrtgaviasp eqlqaayeag yeqcdagwlr dqtvrypivs |
| 541 | prtpcvgdkd sspgvrtygv rpstetydvy cfvdrlegev ffatrleqft fqealefces |
| 601 | hnatlattgq lyaawsrgld kcyagwladg slrypivtpr pacggdkpgv rtvylypnqt |
| 661 | glpdplsrhh afcfrgisav pspgeeeggt ptspsgveew ivtqvvpgva avpveeetta |
| 721 | vpsgettail efttepenqt ewepaytpvg tsplpgilpt wpptgaatee stegpsatev |
| 781 | psaseepsps evpfpseeps pseepfpsvr pfpsvelfps eepfpskeps pseepsasee |
| 841 | pytpsppvps wtelpssgee sgapdvsgdf tgsgdvsghl dfsgqlsgdr asglpsgdld |
| 901 | ssgltstvgs glpvesglps gdeeriewps tptvgelpsg aeilegsasg vgdlsglpsg |
| 961 | evletsasgv gdlsglpsge vlettapgve disglpsgev lettapgved isglpsgevl |
| 1021 | ettapgvedi sglpsgevle ttapgvedis glpsgevlet tapgvedisg lpsgevlett |
| 1081 | apgvedisgl psgevletaa pgvedisglp sgevletaap gvedisglps gevletaapg |
| 1141 | vedisglpsg evletaapgv edisglpsge vletaapgve disglpsgev letaapgved |
| 1201 | isglpsgevl etaapgvedi sglpsgevle taapgvedis glpsgevlet aapgvedisg |
| 1261 | lpsgevleta apgvedisgl psgevletaa pgvedisglp sgevletaap gvedisglps |
| 1321 | gevletaapg vedisglpsg evletaapgv edisglpsge vletaapgve disglpsgev |
| 1381 | letaapgved isglpsgevl ettapgveei sglpsgevle ttapgvdeis glpsgevlet |
| 1441 | tapgveeisg lpsgevlets tsavgdlsgl psggevleis vsgvedisgl psgevvetsa |
| 1501 | sgiedvselp sgegletsas gvedlsrlps geevleisas gfgdlsglps ggegletsas |
| 1561 | evgtdlsglp sgregletsa sgaedlsglp sgkedlvgsa sgdldlgklp sgtlgsgqap |
| 1621 | etsglpsgfs geysgvdlgs gppsglpdfs glpsgfptvs lvdstlvevv tastaseleg |
| 1681 | rgtigisgag eisglpssel disgrasglp sgtelsgqas gspdvsgeip glfgvsgqps |
| 1741 | gfpdtsgets gvtelsglss gqpgisgeas gvlygtsqpf gitdlsgets gvpdlsgqps |
| 1801 | glpgfsgats gvpdlvsgtt sgsgessgit fvdtslveva pttfkeeegl gsvelsglps |
| 1861 | geadlsgksg mvdvsgqfsg tvdssgftsq tpefsglpsg iaevsgessr aeigsslpsg |
| 1921 | ayygsgtpss fptvslvdrt lvesvtqapt aqeagegpsg ilelsgahsg apdmsgehsg |
| 1981 | fldlsglqsg liepsgeppg tpyfsgdfas ttnvsgessv amgtsgeasg lpevtlitse |
| 2041 | fvegvtepti sqelgqrppv thtpqlfess gkvstagdis gatpvlpgsg vevssvpess |
| 2101 | setsaypeag fgasaapeas redsgspdls ettsafhean lerssglgvs gstltfqege |
| 2161 | asaapevsge stttsdvgte apglpsatpt asgdrteisg dlsghtsqlg vvistsipes |
| 2221 | ewtqqtqrpa ethleiesss llysgeetht vetatsptda sipaspewkr esestaadqe |
| 2281 | vceegwnkyq ghcyrhfpdr etwvdaerrc reqqshlssi vtpeeqefvn nnaqdyqwig |
| 2341 | lndrtiegdf rwsdghpmqf enwrpnqpdn ffaagedcvv miwhekgewn dvpcnyhlpf |
| 2401 | tckkgtatty krrlqkrssr hprrsrpsta h |
| Agrin (SEQ ID NO. 2) | GenBank: CAI15575.2 |
| 1 | magrshpgpl rpllpllvva acvlpgaggt cperalerre eeanvvltgt veeilnvdpv |
| 61 | qhtysckvrv wrylkgkdlv areslldggn kvvisgfgdp licdnqvstg dtriffvnpa |
| 121 | ppylwpahkn elmlnsslmr itlrnleeve fcvedkpgth ftpvpptppd acrgmlcgfg |
| 181 | avcepnaegp grascvckks pcpsvvapvc gsdastysne celqraqcsq qrrirllsrg |
| 241 | pcgsrdpcsn vtcsfgstca rsadgltasc lcpatcrgap egtvcgsdga dypgecqllr |
| 301 | racarqenvf kkfdgpcdpc qgalpdpsrs crvnprtrrp emllrpescp arqapvcgdd |
| 361 | gvtyendcvm grsgaargll lqkvrsgqcq grdqcpeper fnavclsrrg rprcscdrvt |
| 421 | cdgayrpvca qdgrtydsdc wrqqaecrqq raipskhqgp cdqapspclg vqcafgatca |
| 481 | vkngqaacec lqacsslydp vcgsdgvtyg saceleatac tlgreiqvar kgpcdrcgqc |
| 541 | rfgalceaet grcvcpsecv alaqpvcgsd ghtypsecml hvhacthqis lhvasagpce |
| 601 | tcgdavcafg avcsagqcvc prcehpppgp vcgsdgvtyg sacelreaac lqqtqieear |
| 661 | agpceqaecg sggsgsgedg dceqelcrqr ggiwdedsed gpcvcdfscq svpgspvcgs |
| 721 | dgvtystece lkkarcesqr glyvaaqgac rgptfaplpp vaplhcaqtp ygccqdnita |
| 781 | argvglagcp sacqcnphgs yggtcdpatg qcscrpgvgg lrcdrcepgf wnfrgivtdg |
| 841 | rsgctpcscd pqgavrddce qmtglcsckp gvagpkcgqc pdgralgpag ceadasapat |
| 901 | caemrcefga rcveesgsah cvcpmltcpe anatkvcgsd gvtygnecql ktiacrqglq |
| 961 | isiqslgpcq eavapsthpt sasvtvttpg lllsqalpap pgalplapss tahsqttppp |
| 1021 | ssrprttasv prttvwpvlt vpptapspap slvasafges gstdgssdee lsgdqeasgg |
| 1081 | gsgglepleg ssvatpgppv erascynsal gccsdgktps ldaegsncpa tkvfqgvlel |
| 1141 | egvegqelfy tpemadpkse lfgetarsie stlddlfrns dvkkdfrsvr lrdlgpgksv |
| 1201 | raivdvhfdp ttafrapdva rallrqiqvs rrrslgvrrp lqehvrfmdf dwfpafitga |
| 1261 | tsgaiaagat arattasrlp ssavtpraph pshtsqpvak ttaapttrrp pttapsrvpg |
| 1321 | rrppapqqpp kpcdsqpcfh ggtcqdwalg ggftcscpag rggavcekvl gapvpafegr |
| 1381 | sflafptlra yhtlrlalef ralepqglll yngnargkdf lalalldgrv qlrfdtgsgp |
| 1441 | avltsavpve pgqwhrlels rhwrrgtlsv dgetpvlges psgtdglnld tdlfvggvpe |
| 1501 | dqaavalert fvgaglrgci rlldvnnqrl elgigpgaat rgsgvgecgd hpclpnpchg |
| 1561 | gapcqnleag rfhcqcppgr vgptcadeks pcqpnpchga apcrvlpegg aqcecplgre |
| 1621 | gtfcqtasgq dgsgpfladf ngfshlelrg lhtfardlge kmalevvfla rgpsglllyn |
| 1681 | gqktdgkgdf vslalrdrrl efrydlgkga avirsrepvt lgawtrvsle rngrkgalrv |
| 1741 | gdgprvlges pvphtvlnlk eplyvggapd fsklaraaav ssgfdgaiql vslggrqllt |
| 1801 | pehvlrqvdv tsfaghpctr asghpclnga scvpreaayv clcpggfsgp hcekglveks |
| 1861 | agdvdtlafd grtfveylna vtesekalqs nhfelslrte atqglvlwsg kateradyva |
| 1921 | laivdghlql synlgsqpvv lrstvpvntn rwlrvvahre qregslqvgn eapvtgsspl |
| 1981 | gatqldtdga lwlgglpelp vgpalpkayg tgfvgclrdv vvgrhplhll edavtkpelr |
| 2041 | pcptp |
| Biglycan (SEQ ID NO. 3) | GenBank: AAA52287.1 |
| 1 | mwplwrlvsl lalsqalpfe qrgfwdftld dgpfmmndee asgadtsgvl dpdsvtptys |
| 61 | amcpfgchch lrvvqcsdlg lefmlvvgvg plglkfmlvm gvgplglksv pkeispdttl |
| 121 | ldlqnndise lrkddfkglq hlyalvlvnn kiskihekaf splrnvqkly isknhlveip |
| 181 | pnlpsslvel rihdnrirkv pkgvfsglrn mnciemggnp lensgfepga fdglklnylr |
| 241 | iseakltgip kdlpetlnel hldhnkiqai eledllrysk lyrlglghnq irmiengsls |
| 301 | flptlrelhl dnnklarvps glpdlkllqv vylhsnnitk vgvndfcpmg fgvkrayyng |
| 361 | islfnnpvpy wevqpatfrc vtdrlaiqfg nykk |
| Brevican(SEQ ID NO. 4) | GenBank: AAH27971.1 |
| 1 | maqlflplla alvlaqapaa ladvlegdss edrafrvria gdaplqgvlg galtipchvh |
| 61 | ylrpppsrra vlgsprvkwt flsrgreaev lvargvrvkv neayrfrval paypasltdv |
| 121 | slalselrpn dsgiyrcevq hgiddssdav evkvkgvvfl yregsaryaf sfsgaqeaca |
| 181 | rigahiatpe qlyaaylggy eqcdagwlsd qtvrypiqtp reacygdmdg fpgvrnygvv |
| 241 | dpddlydvyc yaedlngelf lgdppekltl eearaycqer gaeiattgql yaawdggldh |
| 301 | cspgwladgs vrypivtpsq rcggglpgvk tlflfpnqtg fpnkhsrfnv ycfrdsaqps |
| 361 | aipeasnpas npasdgleai vtvtetleel qlpqeatese srgaiysipi medggggsst |
| 421 | pedpaeaprt llefetqsmv pptgfseeeg kaleeeekye deeekeeeee eeevedealw |
| 481 | awpselsspg peaslptepa aqekslsqap aravlqpgas plpdgeseas rpprvhgppt |
| 541 | etlptprern laspspstlv earevgeatg gpelsgvprg eseetgsseg apsllpatra |
| 601 | pegtreleap sednsgrtap agtsvqaqpv lptdsasrgg vavvpasgdc vpspchnggt |
| 661 | cleeeegvrc lclpgyggdl cdvglrfcnp gwdafqgacy khfstrrswe eaetqcrmyg |
| 721 | ahlasistpe eqdfinnryr eyqwiglndr tiegdflwsd gvpllyenwn pgqpdsyfls |
| 781 | gencvvmvwh dqgqwsdvpc nyhlsytckm glvscgpppe lplaqvfgrp rlryevdtvl |
| 841 | ryrcreglaq rnlplircqe ngrweapqis cvprrparal hpeedpegrq grllgrwkal |
| 901 | lippsspmpg p |
| Collagen 1 (SEQ ID NO. 5) | NCBI Reference Sequence: NP_000079.2 |
| 1 | mfsfvdlrll lllaatallt hgqeegqveg qdedippitc vqnglryhdr dvwkpepcri |
| 61 | cvcdngkvlc ddvicdetkn cpgaevpege ccpvcpdgse sptdqettgv egpkgdtgpr |
| 121 | gprgpagppg rdgipgqpgl pgppgppgpp gppglggnfa pqlsygydek stggisvpgp |
| 181 | mgpsgprglp gppgapgpqg fqgppgepge pgasgpmgpr gppgppgkng ddgeagkpgr |
| 241 | pgergppgpq garglpgtag lpgmkghrgf sgldgakgda gpagpkgepg spgengapgq |
| 301 | mgprglpger grpgapgpag argndgatga agppgptgpa gppgfpgavg akgeagpqgp |
| 361 | rgsegpqgvr gepgppgpag aagpagnpga dgqpgakgan gapgiagapg fpgargpsgp |
| 421 | qgpggppgpk gnsgepgapg skgdtgakge pgpvgvqgpp gpageegkrg argepgptgl |
| 481 | pgppgerggp gsrgfpgadg vagpkgpage rgspgpagpk gspgeagrpg eaglpgakgl |
| 541 | tgspgspgpd gktgppgpag qdgrpgppgp pgargqagvm gfpgpkgaag epgkagergv |
| 601 | pgppgavgpa gkdgeagaqg ppgpagpage rgeqgpagsp gfqglpgpag ppgeagkpge |
| 661 | qgvpgdlgap gpsgargerg fpgergvqgp pgpagprgan gapgndgakg dagapgapgs |
| 721 | qgapglqgmp gergaaglpg pkgdrgdagp kgadgspgkd gvrgltgpig ppgpagapgd |
| 781 | kgesgpsgpa gptgargapg drgepgppgp agfagppgad gqpgakgepg dagakgdagp |
| 841 | pgpagpagpp gpignvgapg akgargsagp pgatgfpgaa grvgppgpsg nagppgppgp |
| 901 | agkeggkgpr getgpagrpg evgppgppgp agekgspgad gpagapgtpg pqgiagqrgv |
| 961 | vglpgqrger gfpglpgpsg epgkqgpsga sgergppgpm gppglagppg esgregapga |
| 1021 | egspgrdgsp gakgdrgetg pagppgapga pgapgpvgpa gksgdrgetg pagpagpvgp |
| 1081 | vgargpagpq gprgdkgetg eqgdrgikgh rgfsglqgpp gppgspgeqg psgasgpagp |
| 1141 | rgppgsagap gkdglnglpg pigppgprgr tgdagpvgpp gppgppgppg ppsagfdfsf |
| 1201 | lpqppqekah dggryyradd anvvrdrdle vdttlkslsq qienirspeg srknpartcr |
| 1261 | dlkmchsdwk sgeywidpnq gcnldaikvf cnmetgetcv yptqpsvaqk nwyisknpkd |
| 1321 | krhvwfgesm tdgfqfeygg qgsdpadvai qltflrlmst easqnityhc knsvaymdqq |
| 1381 | tgnlkkalll qgsneieira egnsrftysv tvdgctshtg awgktvieyk ttktsrlpii |
| 1441 | dvapldvgap dqefgfdvgp vcfl |
| Collagen II (SEQ ID NO. 6) | GenBank: CAA34683.1 |
| 1 | mirlgapqsl vlltllvaav lrcqgqdvrq pgpkgqkgep gdikdivgpk gppgpqgpag |
| 61 | eqgprgdrgd kgekgapgpr grdgepgtpg npgppgppgp pgppglggnf aaqmaggfde |
| 121 | kaggaqlgvm qgpmgpmgpr gppgpagapg pqgfqgnpge pgepgvsgpm gprgppgppg |
| 181 | kpgddgeagk pgkagergpp gpqgargfpg tpglpgvkgh rgypgldgak geagapgvkg |
| 241 | esgspgengs pgpmgprglp gergrtgpag aagargndgq pgpagppgpv gpaggpgfpg |
| 301 | apgakgeagp tgargpegaq gprgepgtpg spgpagasgn pgtdgipgak gsagapgiag |
| 361 | apgfpgprgp pgpqgatgpl gpkgqtgepg iagfkgeqgp kgepgpagpq gapgpageeg |
| 421 | krgargepgg vgpigppger gapgnrgfpg qdglagpkga pgergpsgla gpkgangdpg |
| 481 | rpgepglpga rgltgrpgda gpqgkvgpsg apgedgrpgp pgpqgargqp gvmgfpgpkg |
| 541 | angepgkage kglpgapglr glpgkdgetg aagppgpagp agergeqgap gpsgfqglpg |
| 601 | ppgppgeggk pgdqgvpgea gapglvgprg ergfpgergs pgaqglqgpr glpgtpgtdg |
| 661 | pkgasgpagp pgaqgppglq gmpgergaag iagpkgdrgd vgekgpegap gkdggrgltg |
| 721 | pigppgpaga ngekgevgpp gpagsagarg apgergetgp pgpagfagpp gadgqpgakg |
| 781 | eqgeagqkgd agapgpqgps gapgpqgptg vtgpkgarga qgppgatgfp gaagrvgppg |
| 841 | sngnpgppgp pgpsgkdgpk gargdsgppg ragepglqgp agppgekgep gddgpsgaeg |
| 901 | ppgpqglagq rgivglpgqr gergfpglpg psgepgkqga pgasgdrgpp gpvgppgltg |
| 961 | pagepgrqgs pgadgppgrd gaagvkgdrg etgavgapgt pgppgspgpa gptgkqgdrg |
| 1021 | eagaqgpmgp sgpagargiq gpqgprgdkg eagepgergl kghrgftglq glpgppgpsg |
| 1081 | dqgasgpagp sgprgppgpv gpsgkdgang ipgpigppgp rgrsgetgpa gppgnpgppg |
| 1141 | ppgppgpgid msafaglgpr |
| Collagen III (SEQ ID NO. 7) | NCBI Reference Sequence: NP_000081.1 |
| 1 | mmsfvqkgsw lllallhpti ilaqqeaveg gcshlgqsya drdvwkpepc qicvcdsgsv |
| 61 | lcddiicddq eldcpnpeip fgeccavcpq pptaptrppn gqgpqgpkgd pgppgipgrn |
| 121 | gdpgipgqpg spgspgppgi cescptgpqn yspqydsydv ksgvavggla gypgpagppg |
| 181 | ppgppgtsgh pgspgspgyq gppgepgqag psgppgppga igpsgpagkd gesgrpgrpg |
| 241 | erglpgppgi kgpagipgfp gmkghrgfdg rngekgetga pglkgenglp gengapgpmg |
| 301 | prgapgergr pglpgaagar gndgargsdg qpgppgppgt agfpgspgak gevgpagspg |
| 361 | sngapgqrge pgpqghagaq gppgppging spggkgemgp agipgapglm gargppgpag |
| 421 | angapglrgg agepgkngak gepgprgerg eagipgvpga kgedgkdgsp gepganglpg |
| 481 | aagergapgf rgpagpngip gekgpagerg apgpagprga agepgrdgvp ggpgmrgmpg |
| 541 | spggpgsdgk pgppgsqges grpgppgpsg prgqpgvmgf pgpkgndgap gkngerggpg |
| 601 | gpgpqgppgk ngetgpqgpp gptgpggdkg dtgppgpqgl qglpgtggpp gengkpgepg |
| 661 | pkgdagapga pggkgdagap gergppglag apglrggagp pgpeggkgaa gppgppgaag |
| 721 | tpglqgmpge rgglgspgpk gdkgepggpg adgvpgkdgp rgptgpigpp gpagqpgdkg |
| 781 | eggapglpgi agprgspger getgppgpag fpgapgqnge pggkgergap gekgeggppg |
| 841 | vagppggsgp agppgpqgvk gergspggpg aagfpgargl pgppgsngnp gppgpsgspg |
| 901 | kdgppgpagn tgapgspgvs gpkgdagqpg ekgspgaqgp pgapgplgia gitgarglag |
| 961 | ppgmpgprgs pgpqgvkges gkpganglsg ergppgpqgl pglagtagep grdgnpgsdg |
| 1021 | 1pgrdgspgg kgdrgengsp gapgapghpg ppgpvgpagk sgdrgesgpa gpagapgpag |
| 1081 | srgapgpqgp rgdkgetger gaagikghrg fpgnpgapgs pgpagqqgai gspgpagprg |
| 1141 | pvgpsgppgk dgtsghpgpi gppgprgnrg ergsegspgh pgqpgppgpp gapgpccggv |
| 1201 | gaaaiagigg ekaggfapyy gdepmdfkin tdeimtslks vngqieslis pdgsrknpar |
| 1261 | ncrdlkfchp elksgeywvd pnqgckldai kvfcnmetge tcisanpinv prkhwwtdss |
| 1321 | aekkhvwfge smdggfqfsy gnpelpedvl dvqlaflrll ssrasqnity hcknsiaymd |
| 1381 | qasgnvkkal klmgsnegef kaegnskfty tvledgctkh tgewsktvfe yrtrkavrlp |
| 1441 | ivdiapydig gpdqefgvdv gpvcfl |
| Collagen IV (SEQ ID NO. 8) | NCBI Reference Sequence: NP_001836.2 |
| 1 | mgprlsvwll llpaalllhe ehsraaakgg cagsgcgkcd chgvkgqkge rglpglqgvi |
| 61 | gfpgmqgpeg pqgppgqkgd tgepglpgtk gtrgppgasg ypgnpglpgi pgqdgppgpp |
| 121 | gipgcngtkg ergplgppgl pgfagnpgpp glpgmkgdpg eilghvpgml lkgergfpgi |
| 181 | pgtpgppglp glqgpvgppg ftgppgppgp pgppgekgqm glsfqgpkgd kgdqgvsgpp |
| 241 | gvpgqaqvqe kgdfatkgek gqkgepgfqg mpgvgekgep gkpgprgkpg kdgdkgekgs |
| 301 | pgfpgepgyp gligrqgpqg ekgeagppgp pgivigtgpl gekgergypg tpgprgepgp |
| 361 | kgfpglpgqp gppglpvpgq agapgfpger gekgdrgfpg tslpgpsgrd glpgppgspg |
| 421 | ppgqpgytng ivecqpgppg dqgppgipgq pgfigeigek gqkgesclic didgyrgppg |
| 481 | pqgppgeigf pgqpgakgdr glpgrdgvag vpgpqgtpgl igqpgakgep gefyfdlrlk |
| 541 | gdkgdpgfpg qpgmtgrags pgrdghpglp gpkgspgsvg lkgergppgg vgfpgsrgdt |
| 601 | gppgppgygp agpigdkgqa gfpggpgspg lpgpkgepgk ivplpgppga eglpgspgfp |
| 661 | gpqgdrgfpg tpgrpglpge kgavgqpgig fpgppgpkgv dglpgdmgpp gtpgrpgfng |
| 721 | lpgnpgvqgq kgepgvglpg lkglpglpgi pgtpgekgsi gvpgvpgehg aigppglqgi |
| 781 | rgepgppglp gsvgspgvpg igppgargpp ggqgppglsg ppgikgekgf pgfpgldmpg |
| 841 | pkgdkgaqgl pgitgqsglp glpgqqgapg ipgfpgskge mgvmgtpgqp gspgpvgapg |
| 901 | lpgekgdhgf pgssgprgdp glkgdkgdvg lpgkpgsmdk vdmgsmkgqk gdqgekgqig |
| 961 | pigekgsrgd pgtpgvpgkd gqagqpgqpg pkgdpgisgt pgapglpgpk gsvggmglpg |
| 1021 | tpgekgvpgi pgpqgspglp gdkgakgekg qagppgigip glrgekgdqg iagfpgspge |
| 1081 | kgekgsigip gmpgspglkg spgsvgypgs pglpgekgdk glpgldgipg vkgeaglpgt |
| 1141 | pgptgpagqk gepgsdgipg sagekgepgl pgrgfpgfpg akgdkgskge vgfpglagsp |
| 1201 | gipgskgeqg fmgppgpqgq pglpgspgha tegpkgdrgp qgqpglpglp gpmgppglpg |
| 1261 | idgvkgdkgn pgwpgapgvp gpkgdpgfqg mpgiggspgi tgskgdmgpp gvpgfqgpkg |
| 1321 | lpglqgikgd qgdqgvpgak glpgppgppg pydiikgepg lpgpegppgl kglqglpgpk |
| 1381 | gqqgvtglvg ipgppgipgf dgapgqkgem gpagptgprg fpgppgpdgl pgsmgppgtp |
| 1441 | svdhgflvtr hsqtiddpqc psgtkilyhg ysllyvqgne rahgqdlgta gsclrkfstm |
| 1501 | pflfcninnv cnfasrndys ywlstpepmp msmapitgen irpfisrcav ceapamvmav |
| 1561 | hsqtiqippc psgwsslwig ysfvmhtsag aegsgqalas pgscleefrs apfiechgrg |
| 1621 | tcnyyanays fwlatierse mfkkptpstl kagelrthvs rcqvcmrrt |
| Collagen V (SEQ ID NO. 9) | NCBI Reference Sequence: NP_000084.3 |
| 1 | mdvhtrwkar salrpgapll ppllllllwa pppsraaqpa dllkvldfhn lpdgitkttg |
| 61 | fcatrrsskg pdvayrvtkd aqlsaptkql ypasafpedf silttvkakk gsqaflvsiy |
| 121 | neqgiqqigl elgrspvfly edhtgkpgpe dyplfrginl sdgkwhrial svhkknvtli |
| 181 | ldckkkttkf ldrsdhpmid ingiivfgtr ildeevfegd iqqllfvsdh raaydycehy |
| 241 | spdcdtavpd tpqsqdpnpd eyytegdgeg etyyyeypyy edpedlgkep tpskkpveaa |
| 301 | kettevpeel tptpteaapm petsegagke edvgigdydy vpsedyytps pyddltygeg |
| 361 | eenpdqptdp gagaeiptst adtsnssnpa pppgegaddl egefteetir nldenyydpy |
| 421 | ydptsspsei gpgmpanqdt iyegiggprg ekgqkgepai iepgmliegp pgpegpaglp |
| 481 | gppgtmgptg qvgdpgergp pgrpglpgad glpgppgtml mlpfrfgggg dagskgpmvs |
| 541 | aqesqaqail qqarlalrgp agpmgltgrp gpvgppgsgg lkgepgdvgp qgprgvqgpp |
| 601 | gpagkpgrrg ragsdgargm pgqtgpkgdr gfdglaglpg ekghrgdpgp sgppgppgdd |
| 661 | gergddgevg prglpgepgp rgllgpkgpp gppgppgvtg mdgqpgpkgn vgpqgepgpp |
| 721 | gqqgnpgaqg lpgpqgaigp pgekgplgkp glpgmpgadg ppghpgkegp pgekggqgpp |
| 781 | gpqgpigypg prgvkgadgi rglkgtkgek gedgfpgfkg dmgikgdrge igppgprged |
| 841 | gpegpkgrgg pngdpgplgp pgekgklgvp glpgypgrqg pkgsigfpgf pgangekggr |
| 901 | gtpgkpgprg qrgptgprge rgprgitgkp gpkgnsggdg pagppgergp ngpqgptgfp |
| 961 | gpkgppgppg kdglpghpgq rgetgfqgkt gppgppgvvg pqgptgetgp mgerghpgpp |
| 1021 | gppgeqglpg lagkegtkgd pgpaglpgkd gppglrgfpg drglpgpvga lglkgnegpp |
| 1081 | gppgpagspg ergpagaagp igipgrpgpq gppgpagekg apgekgpqgp agrdglqgpv |
| 1141 | glpgpagpvg ppgedgdkge igepgqkgsk gdkgeqgppg ptgpqgpigq pgpsgadgep |
| 1201 | gprgqqglfg qkgdegprgf pgppgpvglq glpgppgekg etgdvgqmgp pgppgprgps |
| 1261 | gapgadgpqg ppggignpga vgekgepgea gepglpgegg ppgpkgerge kgesgpsgaa |
| 1321 | gppgpkgppg ddgpkgspgp vgfpgdpgpp gepgpagqdg ppgdkgddge pgqtgspgpt |
| 1381 | gepgpsgppg krgppgpagp egrqgekgak geaglegppg ktgpigpqga pgkpgpdglr |
| 1441 | gipgpvgeqg lpgspgpdgp pgpmgppglp glkgdsgpkg ekghpgligl igppgeqgek |
| 1501 | gdrglpgpqg ssgpkgeqgi tgpsgpigpp gppglpgppg pkgakgssgp tgpkgeaghp |
| 1561 | gppgppgppg eviqplpiqa srtrrnidas qllddgngen yvdyadgmee ifgslnslkl |
| 1621 | eieqmkrplg tqqnpartck dlqlchpdfp dgeywvdpnq gcsrdsfkvy cnftaggstc |
| 1681 | vfpdkksega ritswpkenp gswfsefkrg kllsyvdaeg npvgvvqmtf lrllsasahq |
| 1741 | nvtyhcyqsv awqdaatgsy dkalrflgsn deemsydnnp yiralvdgca tkkgyqktvl |
| 1801 | eidtpkveqv pivdimfndf geasqkfgfe vgpacfmg |
| Collagen VI (SEQ ID NO. 10) | NCBI Reference Sequence: NP_001839.2 |
| 1 | mraarallpl llqacwtaaq depetprava fqdcpvdlff vldtsesval rlkpygalvd |
| 61 | kvksftkrfi dnlrdryyrc drnlvwnaga lhysdeveii qgltrmpggr dalkssvdav |
| 121 | kyfgkgtytd caikkgleql lvggshlken kylivvtdgh plegykepcg gledavneak |
| 181 | hlgvkvfsva itpdhleprl siiatdhtyr rnftaadwgq srdaeeaisq tidtivdmik |
| 241 | nnveqvccsf ecqpargppg lrgdpgfege rgkpglpgek geagdpgrpg dlgpvgyqgm |
| 301 | kgekgsrgek gsrgpkgykg ekgkrgidgv dgvkgemgyp glpgckgspg fdgiqgppgp |
| 361 | kgdpgafglk gekgepgadg eagrpgssgp sgdegqpgep gppgekgeag degnpgpdga |
| 421 | pgerggpger gprgtpgtrg prgdpgeagp qgdqgregpv gvpgdpgeag pigpkgyrgd |
| 481 | egppgsegar gapgpagppg dpglmgerge dgpagngteg fpgfpgypgn rgapgingtk |
| 541 | gypglkgdeg eagdpgddnn diaprgvkga kgyrgpegpq gppghqgppg pdeceildii |
| 601 | mkmcscceck cgpidllfvl dssesiglqn feiakdfvvk vidrlsrdel vkfepgqsya |
| 661 | gvvqyshsqm qehvslrsps irnvqelkea ikslqwmagg tftgealqyt rdqllppspn |
| 721 | nrialvitdg rsdtqrdttp lnvlcspgiq vvsvgikdvf dflpgsdqln viscqglaps |
| 781 | qgrpglslvk enyaelleda flknvtaqic idkkcpdytc pitfsspadi tilldgsasv |
| 841 | gshnfdttkr fakrlaerfl tagrtdpahd vrvavvqysg tgqqrperas lqflqnytal |
| 901 | asavdamdfi ndatdvndal gyvtrfyrea ssgaakkrll lfsdgnsqga tpaaiekavq |
| 961 | eaqragieif vvvvgrqvne phirvlvtgk taeydvayge shlfrvpsyq allrgvfhqt |
| 1021 | vsrkvalg |
| Decorin (SEQ ID NO. 11) | GenBank: AAA52301.1 |
| 1 | mkatiillll aqvswagpfq qrglfdfmle deasgigpev pddrdfepsl gpvcpfrcqc |
| 61 | hlrvvqcsdl gldkvpkdlp pdttlldlqn nkiteikdgd fknlknlhal ilvnnkiskv |
| 121 | spgaftplvk lerlylsknq lkelpekmpk tlqelrahen eitkvrkvtf nglnqmivie |
| 181 | lgtnplkssg iengafqgmk klsyiriadt nitsipqglp psltelhldg nkisrvdaas |
| 241 | lkglnnlakl glsfnsisav dngslantph lrelhldnnk ltrvvylhnn nisvvgssdf |
| 301 | cppghntkka sysgvslfsn pvqyweiqps tfrcvyvrsa iqlgnyk |
| Elastin (SEQ ID NO. 12) | GenBank: AAC98395.1 |
| 1 | magltaaapr pgvlllllsi lhpsrpggvp gaipggvpgg vfypgaglga lgggalgpgg |
| 61 | kplkpvpggl agaglgaglg afpavtfpga lvpggvadaa aaykaakaga glggvpgvgg |
| 121 | lgvsagavvp qpgagvkpgk vpgvglpgvy pggvlpgarf pgvgvlpgvp tgagvkpkap |
| 181 | gvggafagip gvgpfggpqp gvplgypika pklpggyglp yttgklpygy gpggvagaag |
| 241 | kagyptgtgv gpqaaaaaaa kaaakfgaga agvlpgvgga gvpgvpgaip giggiagvgt |
| 301 | paaaaaaaaa akaakygaaa glvpggpgfg pgvvgvpgag vpgvgvpgag ipvvpgagip |
| 361 | gaavpgvvsp eaaakaaaka akygarpgvg vggiptygvg aggfpgfgvg vggipgvagv |
| 421 | pgvggvpgvg gvpgvgispe aqaaaaakaa kygvgtpaaa aakaaakaaq fglvpgvgva |
| 481 | pgvgvapgvg vapgvglapg vgvapgvgva pgvgvapgig pggvaaaaks aakvaakaql |
| 541 | raaaglgagi pglgvgvgvp glgvgagvpg lgvgagvpgf gavpgalaaa kaakygaavp |
| 601 | gvlgglgalg gvgipggvvg agpaaaaaaa kaaakaaqfg lvgaaglggl gvgglgvpgv |
| 661 | gglggippaa aakaakygaa glggvlggag qfplggvaar pgfglspifp ggaclgkacg |
| 721 | rkrk |
| F-Spondin (SEQ ID NO. 13) | NCBI Reference Sequence: NP_006099.2 |
| 1 | mrlspaplkl srtpallala lplaaalafs detldkvpks egycsrilra qgtrregyte |
| 61 | fslrvegdpd fykpgtsyry tlsaappsyf rgftlialre nregdkeedh agtfqiidee |
| 121 | etqfmsncpv avtestprrr triqvfwiap pagtgcvilk asivqkriiy fqdegsltkk |
| 181 | lceqdstfdg vtdkpildcc acgtakyrlt fygnwsekth pkdyprranh wsaiiggshs |
| 241 | knyvlweygg yasegvkqva elgspvkmee eirqqsdevl tvikakaqwp awqplnvraa |
| 301 | psaefsvdrt rhlmsfltmm gpspdwnvgl saedlctkec gwvqkvvqdl ipwdagtdsg |
| 361 | vtyespnkpt ipqekirplt sldhpqspfy dpeggsitqv arvvieriar kgeqcnivpd |
| 421 | nvddivadla peekdeddtp etciysnwsp wsacssstcd kgkrmrqrml kaqldlsvpc |
| 481 | pdtqdfqpcm gpgcsdedgs tctmsewitw spcsiscgmg mrsreryvkq fpedgsvctl |
| 541 | pteetekctv neecspsscl mtewgewdec satcgmgmkk rhrmikmnpa dgsmckaets |
| 601 | qaekcmmpec htipcllspw sewsdcsvtc gkgmrtrqrm lkslaelgdc nedleqvekc |
| 661 | mlpecpidce ltewsqwsec nkscgkghvi rtrmiqmepq fggapcpetv qrkkcrirkc |
| 721 | lrnpsiqklr wrearesrrs eqlkeesege qfpgcrmrpw tawsectklc gggiqerymt |
| 781 | vkkrfkssqf tsckdkkeir acnvhpc |
| Fibrin (SEQ ID NO. 14) | NCBI Reference Sequence: NP_000499.1 |
| 1 | mfsmrivclv lsvvgtawta dsgegdflae gggvrgprvv erhqsackds dwpfcsdedw |
| 61 | nykcpsgcrm kglidevnqd ftnrinklkn slfeyqknnk dshslttnim eilrgdfssa |
| 121 | nnrdntynrv sedlrsriev lkrkviekvq hiqllqknvr aqlvdmkrle vdidikirsc |
| 181 | rgscsralar evdlkdyedq qkqleqviak dllpsrdrqh lplikmkpvp dlvpgnfksq |
| 241 | lqkvppewka ltdmpqmrme lerpggneit rggstsygtg setesprnps sagswnsgss |
| 301 | gpgstgnrnp gssgtggtat wkpgssgpgs tgswnsgssg tgstgnqnpg sprpgstgtw |
| 361 | npgssergsa ghwtsessys gstgqwhses gsfrpdspgs gnarpnnpdw gtfeevsgnv |
| 421 | spgtrreyht eklvtskgdk elrtgkekvt sgsttttrrs csktvtktvi gpdghkevtk |
| 481 | evvtsedgsd cpeamdlgtl sgigtldgfr hrhpdeaaff dtastgktfp gffspmlgef |
| 541 | vsetesrgse sgiftntkes sshhpgiaef psrgksssys kqftsstsyn rgdstfesks |
| 601 | ykmadeagse adhegthstk rghaksrpvr dcddvlqthp sgtqsgitni klpgsskifs |
| 661 | vycdqetslg gwlliqqrmd gslnfnrtwq dykrgfgsln degegefwlg ndylhlltqr |
| 721 | gsvlrveled wagneayaey hfrvgseaeg yalqvssyeg tagdaliegs veegaeytsh |
| 781 | nnmqfstfdr dadqweenca evygggwwyn ncqaanlngi yypggsydpr nnspyeieng |
| 841 | vvwvsfrgad yslravrmki rplvtq |
| Fibronectin (SEQ ID NO. 15) | NCBI Reference Sequence: NP_002017.1 |
| 1 | mlrgpgpgll llavqclgta vpstgasksk rqaqqmvqpq spvaysqskp gcydngkhyq |
| 61 | inqqwertyl gnalvctcyg gsrgfncesk peaeetcfdk ytgntyrvgd tyerpkdsmi |
| 121 | wdctcigagr grisctianr cheggqsyki gdtwrrphet ggymlecvcl gngkgewtck |
| 181 | piaekcfdha agtsyvvget wekpyqgwmm vdctclgegs gritctsrnr cndqdtrtsy |
| 241 | rigdtwskkd nrgnllqcic tgngrgewkc erhtsvqtts sgsgpftdvr aavyqpqphp |
| 301 | qpppyghcvt dsgvvysvgm qwlktqgnkq mlctclgngv scqetavtqt yggnsngepc |
| 361 | vlpftyngrt fyscttegrq dghlwcstts nyeqdqkysf ctdhtvlvqt rggnsngalc |
| 421 | hfpflynnhn ytdctsegrr dnmkwcgttq nydadqkfgf cpmaaheeic ttnegvmyri |
| 481 | gdqwdkqhdm ghmmrctcvg ngrgewtcia ysqlrdqciv dditynvndt fhkrheeghm |
| 541 | lnctcfgqgr grwkcdpvdq cqdsetgtfy qigdswekyv hgvryqcycy grgigewhcq |
| 601 | plqtypsssg pvevfitetp sqpnshpiqw napqpshisk yilrwrpkns vgrwkeatip |
| 661 | ghlnsytikg lkpgvvyegq lisiqqyghq evtrfdfttt ststpvtsnt vtgettpfsp |
| 721 | lvatsesvte itassfvvsw vsasdtvsgf rveyelseeg depqyldlps tatsvnipdl |
| 781 | lpgrkyivnv yqisedgeqs lilstsqtta pdappdptvd qvddtsivvr wsrpqapitg |
| 841 | yrivyspsve gsstelnlpe tansvtlsdl qpgvqyniti yaveenqest pvviqqettg |
| 901 | tprsdtvpsp rdlqfvevtd vkvtimwtpp esavtgyrvd vipvnlpgeh gqrlpisrnt |
| 961 | faevtglspg vtyyfkvfav shgreskplt aqqttkldap tnlqfvnetd stvlvrwtpp |
| 1021 | raqitgyrlt vgltrrgqpr qynvgpsysk yplrnlqpas eytvslvaik gnqespkatg |
| 1081 | vfttlqpgss ippyntevte ttivitwtpa prigfklgvr psqggeapre vtsdsgsivv |
| 1141 | sgltpgveyv ytiqvlrdgq erdapivnkv vtplspptnl hleanpdtgv ltvswerstt |
| 1201 | pditgyritt tptngqqgns leevvhadqs sctfdnlspg leynvsvytv kddkesvpis |
| 1261 | dtiipavppp tdlrftnigp dtmrvtwapp psidltnflv ryspvkneed vaelsispsd |
| 1321 | navvltnllp gteyvvsyss vyeqhestpl rgrqktglds ptgidfsdit ansftvhwia |
| 1381 | pratitgyri rhhpehfsgr predrvphsr nsitltnitp gteyvvsiva lngreespll |
| 1441 | igqqstvsdv prdlevvaat ptslliswda pavtvryyri tygetggnsp vqeftvpgsk |
| 1501 | statisglkp gvdytitvya vtgrgdspas skpisinyrt eidkpsqmqv tdvqdnsisv |
| 1561 | kwlpssspvt gyrvtttpkn gpgptktkta gpdqtemtie glqptveyvv svyaqnpsge |
| 1621 | sqplvqtavt nidrpkglaf tdvdvdsiki awespqgqvs ryrvtysspe dgihelfpap |
| 1681 | dgeedtaelq glrpgseytv svvalhddme sqpligtqst aipaptdlkf tqvtptslsa |
| 1741 | qwtppnvqlt gyrvrvtpke ktgpmkeinl apdsssvvvs glmvatkyev svyalkdtlt |
| 1801 | srpaqgvvtt lenvspprra rvtdatetti tiswrtktet itgfqvdavp angqtpiqrt |
| 1861 | ikpdvrsyti tglqpgtdyk iylytlndna rsspvvidas taidapsnlr flattpnsll |
| 1921 | vswqpprari tgyiikyekp gspprevvpr prpgvteati tglepgteyt iyvialknnq |
| 1981 | ksepligrkk tdelpqlvtl phpnlhgpei ldvpstvqkt pfvthpgydt gngiqlpgts |
| 2041 | gqqpsvgqqm ifeehgfrrt tppttatpir hrprpyppnv gqealsqtti swapfqdtse |
| 2101 | yiischpvgt deeplqfrvp gtstsatltg ltrgatynii vealkdqqrh kvreevvtvg |
| 2161 | nsvneglnqp tddscfdpyt vshyavgdew ermsesgfkl lcqclgfgsg hfrcdssrwc |
| 2221 | hdngvnykig ekwdrqgeng qmmsctclgn gkgefkcdph eatcyddgkt yhvgeqwqke |
| 2281 | ylgaicsctc fggqrgwrcd ncrrpggeps pegttgqsyn qysqryhqrt ntnvncpiec |
| 2341 | fmpldvqadr edsre |
| Galectin 1 (SEQ ID NO. 16) | NCBI Reference Sequence: NP_002296.1 |
| 1 | macglvasnl nlkpgeclrv rgevapdaks fvlnlgkdsn nlclhfnprf nahgdantiv |
| 61 | cnskdggawg teqreavfpf qpgsvaevci tfdqanltvk lpdgyefkfp nrlnleainy |
| 121 | maadgdfkik cvafd |
| Galectin 3 (SEQ ID NO. 17) | GenBank: BAA22164.1 |
| 1 | madnfslhda lsgsgnpnpq gwpgawgnqp agaggypgas ypgaypgqap pgaypgqapp |
| 61 | gaypgapgay pgapapgvyp gppsgpgayp ssgqpsatga ypatgpygap agplivpynl |
| 121 | plpggvvprm litilgtvkp nanrialdfq rgndvafhfn prfnennrrv ivcntkldnn |
| 181 | wgreerqsvf pfesgkpfki qvlvepdhfk vavndahllq ynhrvkklne isklgisgdi |
| 241 | dltsasytmi |
| Galectin 3c (SEQ ID NO. 18) | GenBank: BAA22164.1 |
| 1 | plpggvvprm litilgtvkp nanrialdfq rgndvafhfn prfnennrrv ivcntkldnn |
| 61 | wgreerqsvf pfesgkpfki qvlvepdhfk vavndahllq ynhrvkklne isklgisgdi |
| 121 | dltsasytmi |
| Galectin 4 (SEQ ID NO. 19) | NCBI Reference Sequence: NP_006140.1 |
| 1 | mayvpapgyq ptynptlpyy qpipgglnvg msvyiqgvas ehmkrffvnf vvgqdpgsdv |
| 61 | afhfnprfdg wdkvvfntlq ggkwgseerk rsmpfkkgaa felvfivlae hykvvvngnp |
| 121 | fyeyghrlpl qmvthlqvdg dlqlqsinfi ggqplrpqgp pmmppypgpg hchqqlnslp |
| 181 | tmegpptfnp pvpyfgrlqg gltarrtiii kgyvpptgks fainfkvgss gdialhinpr |
| 241 | mgngtvvrns llngswgsee kkithnpfgp gqffdlsirc gldrfkvyan gqhlfdfahr |
| 301 | lsafqrvdtl eiqgdvtlsy vqi |
| Galectin 8 (SEQ ID NO. 20) | GenBank: AAF19370.1 |
| 1 | mmlslnnlqn iiynpvipyv gtipdqldpg tlivicghvp sdadrfqvdl qngssvkpra |
| 61 | dvafhfnprf kragcivent linekwgree itydtpfkre ksfeivimvl kdkfqvavng |
| 121 | khtllyghri gpekidtlgi ygkvnihsig fsfssdlqst qassleltei srenvpksgt |
| 181 | pqlslpfaar lntpmgpgrt vvvkgevnan aksfnvdlla gkskdialhl nprinikafv |
| 241 | rnsflqeswg eeernitsfp fspgmyfemi iycdvrefkv avngvhsley khrfkelssi |
| 301 | dtleingdih llevrsw |
| Keratin (SEQ ID NO. 21) | NCBI Reference Sequence: NP_006112.3 |
| 1 | msrqfssrsg yrsgggfssg sagiinyqrr ttssstrrsg ggggrfsscg ggggsfgagg |
| 61 | gfgsrslvnl ggsksisisv argggrgsgf gggyggggfg gggfggggfg gggiggggfg |
| 121 | gfgsggggfg gggfggggyg ggygpvcppg giqevtinqs llqplnveid peiqkvksre |
| 181 | reqikslnnq fasfidkvrf leqqnqvlqt kwellqqvdt strthnlepy fesfinnlrr |
| 241 | rvdqlksdqs rldselknmq dmvedyrnky edeinkrtna enefvtikkd vdgaymtkvd |
| 301 | lqakldnlqq eidfltalyq aelsqmqtqi setnvilsmd nnrsldldsi iaevkaqyed |
| 361 | iaqkskaeae slyqskyeel qitagrhgds vrnskieise lnrviqrlrs eidnvkkqis |
| 421 | nlqqsisdae qrgenalkda knklndleda lqqakedlar llrdyqelmn tklaldleia |
| 481 | tyrtllegee srmsgecapn vsysystsht tisgggsrgg ggggygsggs sygsgggsyg |
| 541 | sgggggggrg sygsggssyg sgggsygsgg gggghgsygs gsssggyrgg sggggggssg |
| 601 | grgsgggssg gsiggrgsss ggvkssggss svkfvsttys gvtr |
| Laminin (SEQ ID NO. 22) | GenBank: CAA78728.1 |
| 1 | mpalwlgccl cfslllpaar atsrrevcdc ngksrqcifd relhrqtgng frclncndnt |
| 61 | dgihcekckn gfyrhrerdr clpcncnskg slsarcdnsg rcsckpgvtg arcdrclpgf |
| 121 | hmltdagctq dqrlldskcd cdpagiagpc dagrcvckpa vtgercdrcr sgyynldggn |
| 181 | pegctqcfcy ghsascrssa eysvhkitst fhqdvdgwka vqrngspakl qwsqrhqdvf |
| 241 | ssaqrldpvy fvapakflgn qqvsygqsls fdyrvdrggr hpsandvile gaglritapl |
| 301 | mplgktlpcg ltktytfrln ehpsnnwspq lsyfeyrrll rnltalrira tygeystgyi |
| 361 | dnvtlisarp vsgapapwve qcicpvgykg qfcqdcasgy krdsarlgpf gtcipcncqg |
| 421 | ggacdpdtgd cysgdenpdi ecadcpigfy ndphdprsck pcpchngfsc svipeteevv |
| 481 | cnncppgvtg arcelcadgy fgdpfgehgp vrpcqpcqcn snvdpsasgn cdrltgrclk |
| 541 | cihntagiyc dqckagyfgd plapnpadkc racncnpmgs epvgcrsdgt cvckpgfggp |
| 601 | ncehgafscp acynqvkiqm dqfmqqlqrm ealiskaqgg dgvvpdtele grmqqaeqal |
| 661 | qdilrdaqis egasrslglq lakvrsqens yqsrlddlkm tvervralgs qyqnrvrdth |
| 721 | rfitqmqls1 aeseaslgnt nipasdhyvg pngfkslaqe atrlaeshve sasnmeqltr |
| 781 | etedyskqal slvrkalheg vgsgsgspdg avvqglvekl ektkslaqql treatqaeie |
| 841 | adrsyqhslr lldsysplqg vsdqsfqvee akrikqkads lsslvtrhmd efkrtqknlg |
| 901 | nwkeeaqqll qngksgreks dqllsranla ksraqealsm gnatfyeves ilknlrefdl |
| 961 | qvdnrkaeae eamkrlsyis qkvsdasdkt qqaeralgsa aadaqrakng agealeisse |
| 1021 | ieqeigslnl eanvtadgal amekglaslk semrevegel erkelefdtn mdavqmvite |
| 1081 | aqkvdtrakn agvtiqdtln tldgllhlmd qplsvdeegl vlleqklsra ktqinsqlrp |
| 1141 | mmseleerar qqrghlhlle tsidgiladv knlenirdnl ppgcyntqal eqq |
| Merosin (SEQ ID NO. 23) | NCBI Reference Sequence: NP_000417.2 |
| 1 | mpgaagvlll lllsgglggv qaqrpqqqrq sqahqqrglf pavlnlasna littnatcge |
| 61 | kgpemycklv ehvpgqpvrn pqcricnqns snpnqrhpit naidgkntww qspsikngie |
| 121 | yhyvtitldl qqvfqiayvi vkaansprpg nwilersldd veykpwqyha vtdtecltly |
| 181 | niyprtgpps yakddevict sfyskihple ngeihislin grpsaddpsp elleftsary |
| 241 | irlrfqrirt lnadlmmfah kdpreidpiv trryyysvkd isvggmcicy gharacpldp |
| 301 | atnksrcece hntcgdscdq ccpgfhqkpw ragtfltkte ceacnchgka eecyydenva |
| 361 | rrnlslnirg kyigggvcin ctqntaginc etctdgffrp kgvspnyprp cqpchcdpig |
| 421 | slnevcvkde kharrglapg schcktgfgg vscdrcargy tgypdckacn csglgskned |
| 481 | pcfgpcicke nveggdcsrc ksgffnlqed nwkgcdecfc sgvsnrcqss ywtygkiqdm |
| 541 | sgwyltdlpg rirvapqqdd ldspqqisis naearqalph syywsapapy lgnklpavgg |
| 601 | qltftisydl eeeeedterv lqlmiilegn dlsistaqde vylhpseeht nvlllkeesf |
| 661 | tihgthfpvr rkefmtvlan lkrvllqity sfgmdaifrl ssvnlesavs yptdgsiaaa |
| 721 | vevcqcppgy tgsscescwp rhrrvngtif ggicepcqcf ghaescddvt geclnckdht |
| 781 | ggpycdkclp gfygeptkgt sedcqpcacp lnipsnnfsp tchldrslgl icdgcpvgyt |
| 841 | gprcercaeg yfgqpsvpgg scqpcqcndn ldfsipgscd slsgsclick pgttgrycel |
| 901 | cadgyfgdav dakncqperc naggsfsevc hsqtgqcecr anvqgqrcdk ckagtfglqs |
| 961 | argcvpcncn sfgsksfdce esgqcwcqpg vtgkkcdrca hgyfnfqegg ctacecshlg |
| 1021 | nncdpktgrc icppntigek cskcapntwg hsittgckac ncstvgsldf qcnvntgqcn |
| 1081 | chpkfsgakc tecsrghwny prcnlcdcfl pgtdattcds etkkcscsdq tgqctckvnv |
| 1141 | egihcdrcrp gkfgldaknp lgcsscycfg tttqcseakg lirtwvtlka eqtilplvde |
| 1201 | alqhtttkgi vfqhpeivah mdlmredlhl epfywklpeq fegkklmayg gklkyaiyfe |
| 1261 | areetgfsty npqviirggt pthariivrh maapligqlt rheiemteke wkyygddprv |
| 1321 | hrtvtredfl dilydihyil ikatygnfmr qsriseisme vaeqgrgttm tppadliekc |
| 1381 | dcplgysgls ceaclpgfyr lrsqpggrtp gptlgtcvpc qcnghsslcd petsicqncq |
| 1441 | hhtagdfcer calgyygivk glpndcqqca cplisssnnf spscvaegld dyrctacprg |
| 1501 | yegqycerca pgytgspgnp ggscqececd pygslpvpcd pvtgfctcrp gatgrkcdgc |
| 1561 | khwharegwe cvfcgdectg lllgdlarle qmvmsinltg plpapykmly glenmtqelk |
| 1621 | hllspqrape rliqlaegnl ntlvtemnel ltratkvtad geqtgqdaer tntrakslge |
| 1681 | fikelardae avnekaikln etlgtrdeaf ernleglqke idqmikelrr knletqkeia |
| 1741 | edelvaaeal lkkvkklfge srgeneemek dlrekladyk nkvddawdll reatdkirea |
| 1801 | nrlfavnqkn mtalekkkea vesgkrqien tlkegndild eanrladein siidyvediq |
| 1861 | tklppmseel ndkiddlsqe ikdrklaekv sqaeshaaql ndssavldgi ldeaknisfn |
| 1921 | ataafkaysn ikdyideaek vakeakdlah eatklatgpr gllkedakgc lqksfrilne |
| 1981 | akklandvke nedhlnglkt rienadarng dllrtlndtl gklsaipndt aaklqavkdk |
| 2041 | arqandtakd vlaqitelhq nldglkknyn kladsvaktn avvkdpsknk iiadadatvk |
| 2101 | nleqeadrli dklkpikele dnlkknisei kelinqarkq ansikvsvss ggdcirtykp |
| 2161 | eikkgsynni vvnvktavad nllfylgsak fidflaiemr kgkvsflwdv gsgvgrveyp |
| 2221 | dltiddsywy rivasrtgrn gtisvraldg pkasivpsth hstsppgyti ldvdanamlf |
| 2281 | vggltgklkk adavrvitft gcmgetyfdn kpiglwnfre kegdckgctv spqvedsegt |
| 2341 | iqfdgegyal vsrpirwypn istvmfkfrt fsssallmyl atrdlrdfms veltdghikv |
| 2401 | sydlgsgmas vvsnqnhndg kwksftlsri qkqanisivd idtnqeenia tsssgnnfgl |
| 2461 | dlkaddkiyf gglptlrnls mkarpevnlk kysgclkdie isrtpynils spdyvgvtkg |
| 2521 | cslenvytvs fpkpgfvels pvpidvgtei nlsfstknes giillgsggt papprrkrrq |
| 2581 | tgqayyvill nrgrlevhls tgartmrkiv irpepnlfhd grehsvhver trgiftvqvd |
| 2641 | enrrymqnlt veqpievkkl fvggappefq psplrnippf egciwnlvin svpmdfarpv |
| 2701 | sfknadigrc ahqklreded gaapaeiviq pepvptpafp tptpvlthgp caaesepall |
| 2761 | igskqfglsr nshiaiafdd tkvknrltie levrteaesg llfymarinh adfatvqlrn |
| 2821 | glpyfsydlg sgdthtmipt kindgqwhki kimrskqegi lyvdgasnrt ispkkadild |
| 2881 | vvgmlyvggl pinyttrrig pvtysidgcv rnlhmaeapa dleqptssfh vgtcfanaqr |
| 2941 | gtyfdgtgfa kavggfkvgl dllvefefrt ttttgvllgi ssqkmdgmgi emideklmfh |
| 3001 | vdngagrfta vydagvpghl cdgqwhkvta nkikhrielt vdgnqveaqs pnpastsadt |
| 3061 | ndpvfvggfp ddlkqfgltt sipfrgcirs lkltkgtgkp levnfakale lrgvqpvscp |
| 3121 | an |
| Mucin (SEQ ID NO. 24) | GenBank: AAA60019.1 |
| 1 | mtpgtqspff llllltvltv vtgsghasst pggeketsat qrssvpsste knaysmtssv |
| 61 | lsshspgsgs sttqgqdvtl apatepasgs aatwgqdvts vpvtrpalgs ttppandvts |
| 121 | apdnkpapgs tappahgvts apdtrpapgs tappahgvts apdtrpapgs tappahgvts |
| 181 | apdtrpapgs tappahgvts apdtrpapgs tappahgvts apdtrpapgs tappahgvts |
| 241 | apdtrpapgs tappahgvts apdtrpapgs tappahgvts apdtrpapgs tappahgvts |
| 301 | apdtrpapgs tappahgvts apdtrpapgs tappahgvts apdtrpapgs tappahgvts |
| 361 | apdtrpapgs tappahgvts apdtrpapgs tappahgvts apdtrpapgs tappahgvts |
| 421 | apdtrpapgs tappahgvts apdtrpapgs tappahgvts apdtrpapgs tappahgvts |
| 481 | apdtrpapgs tappahgvts apdtrpapgs tappahgvts apdtrpapgs tappahgvts |
| 541 | apdtrpapgs tappahgvts apdtrpapgs tappahgvts apdtrpapgs tappahgvts |
| 601 | apdtrpapgs tappahgvts apdtrpapgs tappahgvts apdtrpapgs tappahgvts |
| 661 | apdtrpapgs tappahgvts apdtrpapgs tappahgvts apdtrpapgs tappahgvts |
| 721 | apdtrpapgs tappahgvts apdtrpapgs tappahgvts apdtrpapgs tappahgvts |
| 781 | apdtrpapgs tappahgvts apdtrpapgs tappahgvts apdtrpapgs tappahgvts |
| 841 | apdtrpapgs tappahgvts apdtrpapgs tappahgvts apdtrpapgs tappahgvts |
| 901 | apdtrpapgs tappahgvts apdtrpapgs tappahgvts apdnrpalgs tappvhnvts |
| 961 | asgsasgsas tlvhngtsar atttpaskst pfsipshhsd tpttlashst ktdassthhs |
| 1021 | svppltssnh stspqlstgv sffflsfhis nlqfnssled pstdyyqelq rdisemflqi |
| 1081 | ykqggflgls nikfrpgsvv vqltlafreg tinvhdvetq thqykteaas rynltisdvs |
| 1141 | vsdvpfpfsa qsgagvpgwg iallvlvcvl valaivylia lavcqcrrkn ygqldifpar |
| 1201 | dtyhpmseyp tyhthgryvp psstdrspye kvsagnggss lsytnpavaa asanl |
| Nidogen-1 (SEQ ID NO. 25) | GenBank: CAI22681.1 |
| 1 | mlasssrira awtralllpl llagpvgcls rqelfpfgpg qgdleledgd dfvspalels |
| 61 | galrfydrsd idavyvttng iiatseppak eshpglfppt fgavapflad ldttdglgkv |
| 121 | yyredlspsi tqraaecvhr gfpeisfqps savvvtwesv apyqgpsrdp dqkgkrntfq |
| 181 | avlassdsss yaiflypedg lqfhttfskk ennqvpavva fsqgsvgflw ksngaynifa |
| 241 | ndresvenla kssnsgqqgv wvfeigspat tngvvpadvi lgtedgaeyd dededydlat |
| 301 | trlgledvgt tpfsykalrr ggadtysvps vlsprraate rplgpptert rsfqlavetf |
| 361 | hqqhpqvidv deveetgvvf syntdsrqtc annrhqcsvh aecrdyatgf ccscvagytg |
| 421 | ngrqcvaegs pqrvngkvkg rifvgssqvp ivfentdlhs yvvmnhgrsy taistipetv |
| 481 | gysllplapv ggiigwmfav eqdgfkngfs itggeftrqa evtfvghpgn lvikqrfsgi |
| 541 | dehghltidt elegrvpqip fgssvhiepy telyhystsv itssstreyt vteperdgas |
| 601 | psriytyqwr qtitfqecvh ddsrpalpst qqlsvdsvfv lynqeekilr yalsnsigpv |
| 661 | regspdalqn pcyigthgcd tnaacrpgpr tqftcecsig frgdgrtcyd idecseqpsv |
| 721 | cgshticnnh pgtfrcecve gyqfsdegtc vavvdqrpin ycetglhncd ipqraqciyt |
| 781 | ggssytcscl pgfsgdgqac qdvdecqpsr chpdafcynt pgsftcqckp gyqgdgfrcv |
| 841 | pgevektrcq herehilgaa gatdpqrpip pglfvpecda hghyaptqch gstgycwcvd |
| 901 | rdgrevegtr trpgmtppcl stvappihqg pavptavipl ppgthllfaq tgkierlple |
| 961 | gntmrkteak aflhvpakvi iglafdcvdk mvywtditep sigraslhgg epttiirqdl |
| 1021 | gspegiavdh lgrnifwtds nldrievakl dgtqrrvlfe tdlvnprgiv tdsvrgnlyw |
| 1081 | tdwnrdnpki etsymdgtnr rilvqddlgl pngltfdafs sqlcwvdagt nraeclnpsq |
| 1141 | psrrkalegl qypfavtsyg knlyftdwkm nsvvaldlai sketdafqph kqtrlygitt |
| 1201 | alsqcpqghn ycsvnnggct hlclatpgsr tcrcpdntlg vdcieqk |
| Nidogen-2 (SEQ ID NO. 26) | GenBank: CAA11418.1 |
| 1 | megdrvagrp vlsslpvlll lqllmlraaa lhpdelfphg eswgdqllqe gddessavvk |
| 61 | lanplhfyea rfsnlyvgtn giistqdfpr etqyvdydfp tdfpaiapfl adidtshgrg |
| 121 | rvlyredtsp avlglaaryv ragfprsarf tpthaflatw eqvgayeevk rgalpsgeln |
| 181 | tfqavlasdg sdsyalflyp anglqflgtr pkesynvqlq lparvgfcrg eaddlksegp |
| 241 | yfsltsteqs vknlyqlsnl gipgvwafhi gstspldnvr paavgdlsaa hssvplgrsf |
| 301 | shatalesdy nednldyydv neeeaeylpg epeealnghs sidvsfqskv dtkpleesst |
| 361 | ldphtkegts lgevggpdlk gqvepwdere trspappevd rdslapswet pppypengsi |
| 421 | qpypdggpvp semdvppahp eeeivlrsyp asdhttplsr gtyevgledn igsntevfty |
| 481 | naanketceh nhrqcsrhaf ctdyatgfcc hcqskfygng khclpegaph rvngkvsghl |
| 541 | hvghtpvhft dvdlhayivg ndgraytais hipqpaaqal lpltpigglf gwlfalekpg |
| 601 | sengfslaga afthdmevtf ypgeetvrit qtaegldpen ylsiktniqg qvpyvpanft |
| 661 | ahispykely hysdstvtst ssrdysltfg ainqtwsyri hqnityqvcr haprhpsfpt |
| 721 | tqqlnvdrvf alyndeervl rfavtnqigp vkedsdptpv npcydgshmc dttarchpgt |
| 781 | gvdytcecas gyqgdgrncv denecatgfh rcgpnsvcin lpgsyrcecr sgyefaddrh |
| 841 | tcilitppan pcedgshtca pagqarcvhh ggstfscacl pgyagdghqc tdvdecsenr |
| 901 | chpaatcynt pgsfscrcqp gyygdgfqci pdstssltpc eqqqrhaqaq yaypgarfhi |
| 961 | pqcdeqgnfl plqchgstgf cwcvdpdghe vpgtqtppgs tpphcgpspe ptqrpptice |
| 1021 | rwrenllehy ggtprddqyv pqcddlghfi plqchgksdf cwcvdkdgre vqgtrsqpgt |
| 1081 | tpaciptvap pmvrptprpd vtppsvgtfl lytqgqqigy lplngtrlqk daaktllslh |
| 1141 | gsiivgidyd crermvywtd vagrtisrag lelgaepeti vnsglispeg laidhirrtm |
| 1201 | ywtdsvldki esalldgser kvlfytdlvn praiavdpir gnlywtdwnr eapkietssl |
| 1261 | dgenrrilin tdiglpnglt fdpfskllcw adagtkklec tlpdgtgrry iqnnlkypfs |
| 1321 | ivsyadhfyh tdwrrdgvvs vnkhsgqftd eylpeqrshl ygitavypyc ptgrk |
| Osteopontin (SEQ ID NO. 27) | GenBank: AAA59974.1 |
| 1 | mriavicfcl lgitcaipvk qadsgsseek qlynkypdav atwlnpdpsq kqnllapqtl |
| 61 | psksneshdh mddmddeddd dhvdsqdsid sndsddvddt ddshqsdesh hsdesdelvt |
| 121 | dfptdlpate vftpvvptvd tydgrgdsvv yglrskskkf rrpdiqypda tdeditshme |
| 181 | seelngayka ipvaqdlnap sdwdsrgkds yetsqlddqs aethshkqsr lykrkandes |
| 241 | nehsdvidsq elskvsrefh shefhshedm lvvdpkskee dkhlkfrish eldsassevn |
| Superfibronectin (SEQ ID NO. 28) | NCBI Reference Sequence: NP_002017.1 |
| 1 | mlrgpgpgll llavqclgta vpstgasksk rqaqqmvqpq spvaysqskp gcydngkhyq |
| 61 | inqqwertyl gnalvctcyg gsrgfncesk peaeetcfdk ytgntyrvgd tyerpkdsmi |
| 121 | wdctcigagr grisctianr cheggqsyki gdtwrrphet ggymlecvcl gngkgewtck |
| 181 | piaekcfdha agtsyvvget wekpyqgwmm vdctclgegs gritctsrnr cndqdtrtsy |
| 241 | rigdtwskkd nrgnllqcic tgngrgewkc erhtsvqtts sgsgpftdvr aavyqpqphp |
| 301 | qpppyghcvt dsgvvysvgm qwlktqgnkq mlctclgngv scqetavtqt yggnsngepc |
| 361 | vlpftyngrt fyscttegrq dghlwcstts nyeqdqkysf ctdhtvlvqt rggnsngalc |
| 421 | hfpflynnhn ytdctsegrr dnmkwcgttq nydadqkfgf cpmaaheeic ttnegvmyri |
| 481 | gdqwdkqhdm ghmmrctcvg ngrgewtcia ysqlrdqciv dditynvndt fhkrheeghm |
| 541 | lnctcfgqgr grwkcdpvdq cqdsetgtfy qigdswekyv hgvryqcycy grgigewhcq |
| 601 | plqtypsssg pvevfitetp sqpnshpiqw napqpshisk yilrwrpkns vgrwkeatip |
| 661 | ghlnsytikg lkpgvvyegq lisiqqyghq evtrfdfttt ststpvtsnt vtgettpfsp |
| 721 | lvatsesvte itassfvvsw vsasdtvsgf rveyelseeg depqyldlps tatsvnipdl |
| 781 | lpgrkyivnv yqisedgeqs lilstsqtta pdappdptvd qvddtsivvr wsrpqapitg |
| 841 | yrivyspsve gsstelnlpe tansvtlsdl qpgvqyniti yaveenqest pvviqqettg |
| 901 | tprsdtvpsp rdlqfvevtd vkvtimwtpp esavtgyrvd vipvnlpgeh gqrlpisrnt |
| 961 | faevtglspg vtyyfkvfav shgreskplt aqqttkldap tnlqfvnetd stvlvrwtpp |
| 1021 | raqitgyrlt vgltrrgqpr qynvgpsysk yplrnlqpas eytvslvaik gnqespkatg |
| 1081 | vfttlqpgss ippyntevte ttivitwtpa prigfklgvr psqggeapre vtsdsgsivv |
| 1141 | sgltpgveyv ytiqvlrdgq erdapivnkv vtplspptnl hleanpdtgv ltvswerstt |
| 1201 | pditgyritt tptngqqgns leevvhadqs sctfdnlspg leynvsvytv kddkesvpis |
| 1261 | dtiipavppp tdlrftnigp dtmrvtwapp psidltnflv ryspvkneed vaelsispsd |
| 1321 | navvltnllp gteyvvsvss vyeqhestpl rgrqktglds ptgidfsdit ansftvhwia |
| 1381 | pratitgyri rhhpehfsgr predrvphsr nsitltnitp gteyvvsiva lngreespll |
| 1441 | igqqstvsdv prdlevvaat ptslliswda pavtvryyri tygetggnsp vqeftvpgsk |
| 1501 | statisglkp gvdytitvya vtgrgdspas skpisinyrt eidkpsqmqv tdvqdnsisv |
| 1561 | kwlpssspvt gyrvtttpkn gpgptktkta gpdqtemtie glqptveyvv svyaqnpsge |
| 1621 | sqplvqtavt nidrpkglaf tdvdvdsiki awespqgqvs ryrvtysspe dgihelfpap |
| 1681 | dgeedtaelq glrpgseytv svvalhddme sqpligtqst aipaptdlkf tqvtptslsa |
| 1741 | qwtppnvqlt gyrvrvtpke ktgpmkeinl apdsssvvvs glmvatkyev svyalkdtlt |
| 1801 | srpaqgvvtt lenvspprra rvtdatetti tiswrtktet itgfqvdavp angqtpiqrt |
| 1861 | ikpdvrsyti tglqpgtdyk iylytlndna rsspvvidas taidapsnlr flattpnsll |
| 1921 | vswqpprari tgyiikyekp gspprevvpr prpgvteati tglepgteyt iyvialknnq |
| 1981 | ksepligrkk tdelpqlvtl phpnlhgpei ldvpstvqkt pfvthpgydt gngiqlpgts |
| 2041 | gqqpsvgqqm ifeehgfrrt tppttatpir hrprpyppnv gqealsqtti swapfqdtse |
| 2101 | yiischpvgt deeplqfrvp gtstsatltg ltrgatynii vealkdqqrh kvreevvtvg |
| 2161 | nsvneglnqp tddscfdpyt vshyavgdew ermsesgfkl lcqclgfgsg hfrcdssrwc |
| 2221 | hdngvnykig ekwdrqgeng qmmsctclgn gkgefkcdph eatcyddgkt yhvgeqwqke |
| 2281 | ylgaicsctc fggqrgwrcd ncrrpggeps pegttgqsyn qysqryhqrt ntnvncpiec |
| 2341 | fmpldvqadr edsre |
| SPARC/Osteonectin (SEQ ID NO. 29) | GenBank: AAA60993.1 |
| 1 | mrawiffllc lagralaapq ealpdetevv eetvaevtev svganpvqve vgefddgaee |
| 61 | teeevvaenp cqnhhckhgk vceldenntp mcvcqdptsc papigefekv csndnktfds |
| 121 | schffatkct legtkkghkl hldyigpcky ippcldselt efplrmrdwl knvlvtlyer |
| 181 | dednnlltek qklrvkkihe neklleagdh pvellardfe knynmyifpv hwqfgqldqh |
| 241 | pidgylshte laplraplip mehcttrlfe tcdldndkyi aldewagcfg ikqkdidkdl |
| 301 | vi |
| Tenascin-C (SEQ ID NO. 30) | GenBank: CAI15110.1 |
| 1 | mgamtqllag vflaflalat eggvlkkvir hkrqsgvnat lpeenqpvvf nhvyniklpv |
| 61 | gsqcsvdles asgekdlapp sepsesfqeh tvdgenqivf thriniprra cgcaaapdvk |
| 121 | ellsrleele nlvsslreqc tagagcclqp atgrldtrpf csgrgnfste gcgcvcepgw |
| 181 | kgpncsepec pgnchlrgrc idgqcicddg ftgedcsqla cpsdcndqgk cvngvcicfe |
| 241 | gyagadcsre icpvpcseeh gtcvdglcvc hdgfagddcn kplclnncyn rgrcvenecv |
| 301 | cdegftgedc selicpndcf drgrcingtc yceegftged cgkptcphac htqgrceegq |
| 361 | cvcdegfagv dcsekrcpad chnrgrcvdg rcecddgftg adcgelkcpn gcsghgrcvn |
| 421 | gqcvcdegyt gedcsqlrcp ndchsrgrcv egkcvceqgf kgydcsdmsc pndchqhgrc |
| 481 | vngmcvcddg ytgedcrdrq cprdcsnrgl cvdgqcvced gftgpdcael scpndchgqg |
| 541 | rcvngqcvch egfmgkdcke qrcpsdchgq grcvdgqcic hegftgldcg qhscpsdcnn |
| 601 | lgqcvsgrci cnegysgedc sevsppkdlv vtevteetvn lawdnemrvt eylvvytpth |
| 661 | egglemqfrv pgdqtstiiq elepgveyfi rvfailenkk sipvsarvat ylpapeglkf |
| 721 | ksiketsvev ewdpldiafe tweiifrnmn kedegeitks lrrpetsyrq tglapgqeye |
| 781 | islhivknnt rgpglkrvtt trldapsqie vkdvtdttal itwfkplaei dgieltygik |
| 841 | dvpgdrttid ltedenqysi gnlkpdteye vslisrrgdm ssnpaketft tgldaprnlr |
| 901 | rvsqtdnsit lewrngkaai dsyrikyapi sggdhaevdv pksqqattkt tltglrpgte |
| 961 | ygigvsavke dkesnpatin aateldtpkd lqvsetaets ltllwktpla kfdryrlnys |
| 1021 | lptgqwvgvq lprnttsyvl rglepgqeyn vlltaekgrh kskparvkas teqapelenl |
| 1081 | tvtevgwdgl rlnwtaadqa yehfiiqvqe ankveaarnl tvpgslravd ipglkaatpy |
| 1141 | tvsiygviqg yrtpvlsaea stgetpnlge vvvaevgwda lklnwtapeg ayeyffiqvq |
| 1201 | eadtveaaqn ltvpgglrst dlpglkaath ytitirgvtq dfsttplsve vlteevpdmg |
| 1261 | nltvtevswd alrlnwttpd gtydqftiqv qeadqveeah nltvpgslrs meipglragt |
| 1321 | pytvtlhgev rghstrplav evvtedlpql gdlavsevgw dglrlnwtaa dnayehfviq |
| 1381 | vqevnkveaa qnltlpgslr avdipgleaa tpyrvsiygv irgyrtpvls aeastakepe |
| 1441 | ignlnvsdit pesfnlswma tdgifetfti eiidsnrlle tveynisgae rtahisglpp |
| 1501 | stdfivylsg lapsirtkti satattealp llenitisdi npygftvswm asenafdsfl |
| 1561 | vtvvdsgkll dpqeftlsgt qrklelrgli tgigyevmvs gftqghqtkp lraeivteae |
| 1621 | pevdnllvsd atpdgfrlsw tadegvfdnf vlkirdtkkq sepleitlla pertrditgl |
| 1681 | reateyeiel ygiskgrrsq tvsaiattam gspkevifsd itensatvsw raptaqvesf |
| 1741 | rityvpitgg tpsmvtvdgt ktqtrlvkli pgveylvsii amkgfeesep vsgsfttald |
| 1801 | gpsglvtani tdsealarwq paiatvdsyv isytgekvpe itrtvsgntv eyaltdlepa |
| 1861 | teytlrifae kgpqksstit akfttdldsp rdltatevqs etalltwrpp rasvtgyllv |
| 1921 | yesvdgtvke vivgpdttsy sladlspsth ytakiqalng plrsnmiqti fttigllypf |
| 1981 | pkdcsqamln gdttsglyti ylngdkaeal evfcdmtsdg ggwivflrrk ngrenfyqnw |
| 2041 | kayaagfgdr reefwlgldn lnkitaqgqy elrvdlrdhg etafavydkf svgdaktryk |
| 2101 | lkvegysgta gdsmayhngr sfstfdkdtd saitncalsy kgafwyrnch rvnlmgrygd |
| 2161 | nnhsqgvnwf hwkghehsiq faemklrpsn frnlegrrkr a |
| Tenascin-R (SEQ ID NO. 31) | NCBI GenBank: CAA66709.1 |
| 1 | mgadgetvvl knmligvnli llgsmikpse cqlevtterv qrqsveeegg ianyntsske |
| 61 | qpvvfnhvyn invpldnlcs sgleasaeqe vsaedetlae ymgqtsdhes qvtfthrinf |
| 121 | pkkacpcass aqvlqellsr iemlerevsv lrdqcnancc qesaatgqld yiphcsghgn |
| 181 | fsfescgcic negwfgkncs epycplgcss rgvcvdgqci cdseysgddc selrcptdcs |
| 241 | srglcvdgec vceepytged crelrcpgdc sgkgrcangt clceegyvge dcgqrqclna |
| 301 | csgrgqceeg lcvceegyqg pdcsavappe dlrvagisdr sielewdgpm avteyvisyq |
| 361 | ptalgglqlq qrvpgdwsgv titelepglt ynisvyavis nilslpitak vathlstpqg |
| 421 | lqfktitett vevqwepfsf sfdgweisfi pknneggvia qvpsdvtsfn qtglkpgeey |
| 481 | ivnvvalkeq arspptsasv stvidgptqi lvrdvsdtva fvewipprak vdfillkygl |
| 541 | vggeggrttf rlqpplsqys vqalrpgsry evsvsavrgt nesdsattqf tteidapknl |
| 601 | rvgsrtatsl dlewdnseae vqeykvvyit lageqyhevl vprgigpttr atltdlvpgt |
| 661 | eygvgisavm nsqqsvpatm narteldspr dlmvtasset sisliwtkas gpidhyritf |
| 721 | tpssgiasev tvpkdrtsyt ltdlepgaey iisvtaergr qqslestvda ftgfrpishl |
| 781 | hfshvtsssv nitwsdpspp adrlilnysp rdeeeemmev sldatkrhav lmglqpatey |
| 841 | ivnlvavhgt vtsepivgsi ttgidppkdi tisnvtkdsv mvswsppvas fdyyrvsyrp |
| 901 | tqvgrldssv vpntvtefti trlnpateye islnsvrgre eserictlvh tamdnpvdli |
| 961 | atnitpteal lqwkapvgev enyvivlthf avagetilvd gvseefrlvd llpsthytat |
| 1021 | myatngplts gtistnfstl ldppanltas evtrqsalis wqppraeien yvltykstdg |
| 1081 | srkelivdae dtwirlegll entdytvllq aaqdttwssi tstafttggr vfphpqdcaq |
| 1141 | hlmngdtlsg vypiflngel sqklqvycdm ttdgggwivf qrrqngqtdf frkwadyrvg |
| 1201 | fgnvedefwl gldnihrits qgryelrvdm rdgqeaafas ydrfsvedsr nlyklrigsy |
| 1261 | ngtagdslsy hqgrpfsted rdndvavtnc amsykgawwy knchrtnlng kygesrhsqg |
| 1321 | inwyhwkghe fsipfvemkm rpynhrlmag rkrqslqf |
| Testican 1/SPOCK1 (SEQ ID NO. 32) | NCBI Reference Sequence: NP_004589.1 |
| 1 | mpaiavlaaa aaawcflqve srhldalagg agpnhgnfld ndqwlstvsq ydrdkywnrf |
| 61 | rdddyfrnwn pnkpfdqald pskdpclkvk csphkvcvtq dyqtalcvsr khllprqkkg |
| 121 | nvaqkhwvgp snlvkckpcp vaqsamvcgs dghsytskck lefhacstgk slatlcdgpc |
| 181 | pclpepeppk hkaersactd kelrnlasrl kdwfgalhed anrvikptss ntaqgrfdts |
| 241 | ilpickdslg wmfnkldmny dllldpsein aiyldkyepc ikplfnscds fkdgklsnne |
| 301 | wcycfqkpgg lpcqnemnri qklskgksll gafiprcnee gyykatqchg stgqcwcvdk |
| 361 | ygnelagsrk qgaysceeeq etsgdfgsgg svvllddley erelgpkdke gklrvhtrav |
| 421 | teddededdd kedevgyiw |
| Testican 2/SPOCK2 (SEQ ID NO. 33) | GenBank: AAH23558.1 |
| 1 | mrapgcgrlv lpllllaaaa laegdakglk egetpgnfme deqwlssisq ysgkikhwnr |
| 61 | frdeveddyi kswednqqgd ealdttkdpc qkvkcsrhkv ciaqgyqram cisrkklehr |
| 121 | ikqptvklhg nkdsickpch maqlasvcgs dghtyssvck leqqaclssk qlavrcegpc |
| 181 | pcpteqaats tadgkpetct gqdladlgdr lrdwfqllhe nskqngsass vagpasgldk |
| 241 | slgasckdsi gwmfskldts adlfldqtel aainldkyev cirpffnscd tykdgrvsta |
| 301 | ewcfcfwrek ppclaeleri qiqeaakkkp gifipscded gyyrkmqcdq ssgdcwcvdq |
| 361 | lgleltgtrt hgspdcddiv gfsgdfgsgv gwedeeeket eeageeaeee egeageaddg |
| 421 | gyiw |
| Thrombospondin-4 (SEQ ID NO. 34) | GenBank: CAA79635.1 |
| 1 | mlaprgaavl llhlvlqrwl aagaqatpqv fdllpsssqr lnpgallpvl tdpalndlyv |
| 61 | istfklqtks satifglyss tdnskyfeft vmgrlskail rylkndgkvh lvvfnnlqla |
| 121 | dgrrhrillr lsnlqrgags lelyldciqv dsvhnlpraf agpsqkpeti elrtfqrkpq |
| 181 | dfleelklvv rgslfqvasl qdcflqqsep laatgtgdfn rqflgqmtql nqllgevkdl |
| 241 | lrqqvketsf lrntiaecqa cgplkfqspt pstvvapapp apptrpprrc dsnpcfrgvq |
| 301 | ctdsrdgfqc gpcpegytgn gitcidvdec kyhpcypgvh cinlspgfrc dacpvgftgp |
| 361 | mvqgvgisfa ksnkqvctdi decrngacvp nsicvntlgs yrcgpckpgy tgdqirgckv |
| 421 | erncrnpeln pcsvnaqcie erqgdvtcvc gvgwagdgyi cgkdvdidsy pdeelpcsar |
| 481 | nckkdnckyv pnsgqedadr dgigdacded adgdgilneq dncvlihnvd qrnsdkdifg |
| 541 | dacdnclsvl nndqkdtdgd grgdacdddm dgdgiknild ncpkfpnrdq rdkdgdgvgd |
| 601 | acdscpdvsn pnqsdvdndl vgdscdtnqd sdgdghqdst dncptvinsa qldtdkdgig |
| 661 | decdddddnd gipdlvppgp dncrlvpnpa qedsnsdgvg dicesdfdqd qvidridvcp |
| 721 | enaevtltdf rayqtvgldp egdaqidpnw vvlnqgmeiv qtmnsdpgla vgytafngvd |
| 781 | fegtfhvntq tdddyagfif gyqdsssfyv vmwkqteqty wqatpfrava epgiqlkavk |
| 841 | sktgpgehlr nslwhtgdts dqvrllwkds rnvgwkdkvs yrwflqhrpq vgyirvrfye |
| 901 | gselvadsgv tidttmrggr lgvfcfsqen iiwsnlkyrc ndtipedfqe fqtqnfdrfd |
| 961 | n |
| Vitronectin (SEQ ID NO. 35) | GenBank: ADL14521.1 |
| 1 | maplrpllil allawvalad qesckgrcte gfnvdkkcqc delcsyyqsc ctdytaeckp |
| 61 | qvtrgdvftm pedeytvydd geeknnatvh eqvggpslts dlqaqskgnp eqtpvlkpee |
| 121 | eapapevgas kpegidsrpe tlhpgrpqpp aeeelcsgkp fdaftdlkng slfafrgqyc |
| 181 | yeldekavrp gypklirdvw giegpidaaf trincqgkty lfkgsqywrf edgvldpdyp |
| 241 | rnisdgfdgi pdnvdaalal pahsysgrer vyffkgkqyw eyqfqhqpsq eecegsslsa |
| 301 | vfehfammqr dswedifell fwgrtsagtr qpqflsrdwh gvpgqvdaam agriyisgma |
| 361 | prpslakkqr frhrnrkgyr sqrghsrgrn qnsrrpsrat wlslfssees nlgannyddy |
| 421 | rmdwlvpatc epiqsvfffs gdkyyrvnlr trrvdtvdpp yprsiaqywl gcpapghl |

Illustrations, Figures, specification, and other diagrams may contain abbreviations for the various ECm components tested. Meaning of the abbreviations are set forth below in Table 2:

TABLE 2

| ECM Abbreviation | ECM Name |
|---|---|
| Agr | Recombinant Rat Agrin |
| Bigly | Recombinant Human Biglycan |
| Bre | Recombinant Human Brevican |
| CI | Purified Human Collagen Type I |
| CII | Purified Human Collagen Type II |
| Dec | Recombinant Human Decorin |
| Fib | Fibrinogen |
| Gal-1 | Recombinant Human Galectin-1 |
| Gal-3 | Recombinant Human Galectin-3 |
| Gal-4 | Recombinant Human Galectin-4 |
| Gal-8 | Recombinant Human Galectin-8 |
| Ker | Keratin |
| Nid-2 | Recombinant Human Nidogen-2 |
| Osteo | Recombinant Human Osteopontin/OPN |
| Ten-R | Recombinant Human Tenascin R |
| Test-1 | Recombinant Human Testican 1/SPOCK1 |
| Test-2 | Recombinant Human Testican 2/SPOCK2 |
| Throm-4 | Recombinant Human Thrombospondin-4 |
| Vit | Human Vitronectin |

### EQUIVALENTS

Those skilled in the art will recognize, or be able to ascertain using no more than routine experimentation, many equivalents to the specific embodiments of the invention described herein. The scope of the present invention is not intended to be limited to the above Description, but rather is as set forth in the following claims:

What is claimed is:

1. An array of polypeptides, the array comprising: a solid support and a plurality of adhesion sets, wherein each adhesion set comprises two or more different polypeptides comprising a polypeptide sequence associated with the extracellular matrix or a functional fragment thereof, and wherein the adhesion sets are attached to the solid support at an addressable location of the array.

2. The array of claim 1, wherein the solid support is a slide optionally coated with a polymer.

3. The array of claim 1, wherein the polymer is polyacrylamide.

4. The array of claim 1, wherein at least one adhesion set comprises two different polypeptides attached to a solid support.

5. The array of claim 1, wherein the two or more of the different polypeptides comprise at least 10 contiguous amino acids chosen from: collagen I, collagen II, collagen III, collagen IV, collagen V, collagen VI, fibronectin, laminin, merosin, tenascin-R, chondroitin sulfate, agreccan, elastin, keratin, mucin, superfibronectin, F-spondin, nidogen-2, heparin sulfate, biglycan, decorin, galectin 1, galectin 3, galectin 3c, galectin 4, galectin 8, thrombospondin-4, osteopontin, osteonectin, testican 1, testican 2, fibrin, tenascin-C, nidogen-1, vitronectin, rat agrin, hyaluronan, and brevican.

6. The array of claim 4, wherein the at least one adhesion set comprises at least 90% sequence identity to two different polypeptides chosen from: osteopontin, thrombospondin-4, fibronectin, laminin, galectin 3, and galectin 8.

7. The array of claim 6, wherein the at least one adhesion set comprises at least 90% sequence identity to fibronectin and laminin.

8. The array of claim 6, wherein the at least one adhesion set comprises at least 90% sequence identity to fibronectin and galectin 3.

9. The array of claim 6, wherein the at least one adhesion set comprises at least 90% sequence identity to fibronectin and galectin 8.

10. The array of claim 6, wherein the at least one adhesion set comprises at least 90% sequence identity to thrombospondin-4 and galectin 8.

11. The array of claim 4, wherein the at least one adhesion set comprises at least at least 90% sequence identity to osteopontin.

12. The array of claim 1, wherein each adhesion set consists of a pair of different polypeptides associated with the extracellular matrix.

13. The array of claim 1, wherein the array is free of animal-derived ECM material, embryonic fibroblasts, or material deposited from Engelbreth-Holm-Swarm (EHS) mouse sarcoma cells.

14. The array of claim 1, wherein the array comprises at least about 700 adhesion sets.

15. The array of claim 1, further comprising one or a plurality of mammalian cells.

16. The array of claim 15, wherein the one or a plurality of mammalian cells contains at least one lung cell.

17. The array of claim 15, wherein the cell sample contains at least one cancer cell or one stem cell.

18. The array of claim 17, wherein the cancer cell is derived from the cancer of the adrenal gland, bladder, bone, bone marrow, brain, spine, breast, cervix, gall bladder, ganglia, gastrointestinal tract, stomach, colon, heart, kidney, liver, lung, muscle, ovary, pancreas, parathyroid, penis, prostate, salivary glands, skin, spleen, testis, thymus, thyroid, or uterus.

19. The array of claim 17, wherein the stem cell is an embryonic stem cell, an adipose-derived stem cell, a mesenchymal stem cell, an umbilical stem cell or a pluripotent stem cell.

20. The array of claim 1, wherein the two or more different polypeptides are attached to the solid support via passive electrostatic non-covalent binding.

21. A system comprising the array of claim 1 and a cell culture vessel.

22. The system of claim 21, further comprising at least one or a plurality of cells.

23. The system of claim 22, wherein the at least one or a plurality of cells are derived from cancer of the adrenal gland, bladder, bone, bone marrow, brain, spine, breast, cervix, gall bladder, ganglia, gastrointestinal tract, stomach, colon, heart, kidney, liver, lung, muscle, ovary, pancreas, parathyroid, penis, prostate, salivary glands, skin, spleen, testis, thymus, thyroid, or uterus.

24. The system of claim 22, further comprising cell media free of at least one or a combination of: serum, animal-derived ECM material, embryonic fibroblasts, or material deposited from Engelbreth-Holm-Swarm (EHS) mouse sarcoma cell.

25. The system of claim 22, wherein the at least one or a plurality of cells is a stem cell chosen from: an embryonic stem cell, an adipose-derived stem cell, a mesenchymal stem cell, an umbilical stem cell or a pluripotent stem cell.

26. A kit comprising the array of claim 1.

27. The kit of claim 26 further comprising at least one of the following: cell media, a volume of fluorescent stain or dye, a cell sample, and a set of instructions, optionally accessible remotely through an electronic medium.

28. A method of identifying an adhesion signature of a cell sample comprising: contacting a cell sample to the array of claim 1; and determining a quantity of cells bound to one or a plurality of adhesion sets.

29. The method of claim 28, wherein the cell sample contains at least one cell from a biopsy.

30. A method of inducing differentiation of a cell comprising contacting a cell sample to the array of claim 1.

31. The method of claim 30, wherein the cell is a stem cell chosen from: an embryonic stem cell, an adipose-derived stem cell, a mesenchymal stem cell, an umbilical stem cell or a pluripotent stem cell.

32. The method of claim 30, wherein the step of contacting comprises exposing the cell sample to the array of claim 1 for a sufficient period of time for differentiation of a cell to a hepatic or pancreatic lineage.

33. A method of culturing a cell comprising contacting a cell sample to the array of claim 1 in the presence of cell media.

34. The method of claim 33, wherein the cell sample is derived from a primary lineage of a cancer cells or stem cells.

35. The method of claim 33, wherein the cell sample comprises one or a plurality of stem cells chosen from: an embryonic stem cell, an adipose-derived stem cell, a mesenchymal stem cell, an umbilical stem cell or a pluripotent stem cell is a pluripotent stem cell or embryonic stem cell.

36. The method of claim 33, wherein the cell is passaged at least about 30 times.

37. The method of claim 33, wherein the cell media is free of at least one or a combination of: serum, animal-derived ECM material, embryonic fibroblasts, or material deposited from Engelbreth-Holm-Swarm (EHS) mouse sarcoma cell.

38. The method of claim 33, wherein the cell sample comprises one or a plurality of primary hepatocytes.

39. The method of claim 33, wherein the array of claim 1 comprises at least one adhesion set comprising at least 10 contiguous amino acids of Collagen 1 and Agreccan or at least 10 contiguous amino acids of Collagen IV and Nidogen-1.

40. A method of diagnosing a hyperproliferative disease comprising:
(a) contacting a cell sample to the array of claim 1;
(b) quantifying one or more adhesion values;
(c) determining one or more adhesion signatures of the cell sample based upon the adhesion values; and
(d) comparing the adhesion signature of the cell sample to an adhesion signature of a control cell sample.

41. The method of claim 40, wherein the hyperproliferative disease is metastatic lung cancer.

42. A method of prognosing a clinical outcome of a subject comprising:
(a) contacting a cell sample to the array of claim 1;
(b) quantifying one or more adhesion values;
(c) determining one or more adhesion signatures of the cell sample based upon the adhesion values; and
(d) correlating the adhesion signature to an adhesion signature of a cell sample associated with a clinical outcome.

43. A method of determining patient responsiveness to a therapy comprising:
(a) contacting a cell sample to the array of claim 1;
(b) quantifying one or more adhesion values;
(c) determining one or more adhesion signatures of the cell sample based upon the adhesion values; and, optionally,
(d) comparing the one or more adhesion signatures to one or more adhesion signature of a control cell sample.

44. A method of isolating a cell comprising: contacting a cell sample to the array of claim 1.

45. A method of adhering hepatocytes derived from a primary lineage of human liver cells comprising contacting the hepatocytes to the array of claim 1.

46. A method of sorting a mixture of cell types, wherein the method comprises: contacting a mixture of cell types to the array of claim 1.

47. The method of claim 46, wherein the method further comprises the step of determining one or more adhesion signatures of the cell sample based upon the adhesion values.

48. The method of claim 46, wherein the method further comprises the step of comparing the one or more adhesion signatures to one or more adhesion signature of a control cell type.
